SEQUENCE LISTING (191 sequences)

SEQ ID NO: 1 - DNA, 4527 bp, La Crosse virus
agtagtgtactaccaagtatagataacgtttgaatattaaagttttgaatcaaagccaaagatgatttgtatattggtgctaattacagttgcagctgcaagcccagtgtatcaaaggtgtttccaagatggggctatagtgaagcaaaacccatccaaagaagcagttacagaggtgtgcctgaaagatgatgttagcatgatcaaaacagaggccaggtatgtaagaaatgcaacaggagttttttcaaataatgtcgcaataaggaaatggctagtctctgattggcatgattgcaggcctaagaagatcgttgggggacacatcaatgtaatagaagttggtgatgacctgtcactccatactgaatcatatgtttgcagcgcagattgtaccataggtgtagacaaagagactgcacaggtcaggcttcagacagataccacaaatcattttgaaattgcaggcactactgtgaagtcaggatggttcaagagcacgacatatataactcttgatcaaacttgcgaacaccttaaagtttcctgcggcccaaaatctgtacagttccatgcctgcttcaatcagcatatgtcttgcgtcagatttttacacaggacaatattgcctggctctatagccaattccatatgtcagaatatcgaaatcataattttagttacacttactctattaatctttatattgttaagcattttaagtaagacttatatatgttatttattaatgcctatattcatccccatagcatatatatacggtataatttacaataagtcgtgcaaaaaatgcaaattatgtggcttagtgtatcatccattcacagagtgtggcacacattgtgtctgtggtgcccgctatgatacttcagatagaatgaaactgcatagagcttctggattgtgccctggttataaaagcctaagagctgccagagtcatgtgcaagtcgaaagggcctgcatcaatattgtctataattactgcggtactggtcttaacctttgtgacaccaatcaactccatggttttaggagagagtaaagaaacctttgaacttgaagatcttccagacgacatgttggaaatggcatcgagaataaattcttattatctcacctgtatcttgaattatgctgtaagctggggtcttgttatcattggattgttgatcgggctgctttttaagaaataccagcacagattcttaaatgtttacgcaatgtactgtgaagaatgtgacatgtatcatgacaagtctgggttgaaaagacatggtgatttcaccaacaaatgcagacagtgcacatgtggtcaatatgaagatgctgcaggtttgatggctcacaggaaaacctataactgcttagtgcagtacaaagcaaagtggatgatgaacttcctgataatttacatattcttaattttgatcaaagattctgctatagttgtacaagctgctggaactgacttcaccacctgcctagagactgagagtataaattggaactgcactgggccatttttgaacctcgggaattgccaaaagcaacaaaagaaagaaccttacaccaacattgcaactcagttaaagggactaaaggcaatttccgtactagatgtccctataataacagggataccagatgatattgcgggtgctttaagatatatagaagagaaggaagatttccatgtccagctaactatagaatatgcgatgttaagcaaatactgtgactattatacccaattctcagataactcaggatacagtcagacaacatggagagtgtacttaaggtctcatgattttgaagcctgtatactatatccaaatcagcacttttgcagatgtgtaaaaaatggtgagaagtgcagcagctccaattgggactttgccaatgaaatgaaagattattactctgggaaacaaacaaagtttgacaaggacttaaatctagccctaacagctttgcatcatgccttcagggggacctcatctgcatatatagcaacaatgctctcaaaaaagtccaatgatgacttgattgcatacacaaataagataaaaacaaaattcccaggtaatgcattgttgaaggctataatagattatatagcatatatgaaaagtttgccaggtatggcaaatttcaaatatgatgaattctgggatgaattactgtacaaacccaacccagcaaaggcctcaaaccttgctagaggaaaggagtcatcttacaacttcaaactagcaatttcatcaaagtctataaaaacctgcaagaatgttaaggatgttgcctgcttatcgccaaggtcaggtgctatatatgcttcaataattgcgtgtggtgaacccaatgggccaagtgtgtataggaaaccatcaggtggtgtattccaatctagcactgatcggtctatatactgcttgctggatagccattgtctagaagaatttgaggccatcggccaggaggagctggatgcggtaaagaaatccaaatgttgggaaattgaatatcctgacgtaaagctcatccaagaaggcgatgggactaaaagctgtagaatgaaagattctgggaactgcaatgttgcaactaacagatggccagtgatacaatgtgagaatgacaaattttactactcagagcttcaaaaagattatgacaaagctcaagatattggtcactattgcttaagccctggatgtactactgtccggtaccctattaatccaaagcacatctctaactgtaattggcaagtaagcagatctagcatagcgaagatagatgtgcacaatattgaggatattgagcaatataagaaagctataactcagaaacttcaaacgagcctatctctattcaagtatgcaaaaacaaaaaacttgccgcacatcaaaccaatttataaatatataactatagaaggaacagaaactgcagaaggtatagagagtgcatacattgaatcagaagtacctgcattggctgggacatctatcggattcaaaatcaattctaaagagggcaagcacttgctagatgttatagcatatgtaaaaagtgcctcatactcttcagtgtatacaaaattgtactcaactggcccaacatcagggataaatactaaacatgatgaattgtgtactggcccatgcccagcaaatatcaatcatcaggttgggtggctgacatttgcaagagagaggacaagctcatggggatgcgaagagtttggttgcctggctgtaagtgatgggtgtgtatttggatcatgccaagatataataaaagaagaactatctgtctataggaaggagaccgaggaagtgactgatgtagaactgtgtttgacattttcagacaaaacatactgtacaaacttaaaccctgttacccctattataacagatctatttgaggtacagttcaaaactgtagagacctacagcttgcctagaattgttgctgtgcaaaaccatgagattaaaattgggcaaataaatgatttaggagtttactctaagggttgtgggaatgttcaaaaggtcaatggaactatttatggcaatggagttcccagatttgactacttatgccatttagctagcaggaaggaagtcattgttagaaaatgcttcgacaatgattaccaagcatgcaaatttcttcaaagccctgctagttacagacttgaagaagacagtggcactgtgaccataattgactacaaaaagattttaggaacaatcaagatgaaggcaattttaggagatgtcaaatataaaacatttgctgatagtgtcgatataaccgcagaagggtcatgcaccggctgtattaactgcttcgaaaatatccattgcgaattaacgttgcacaccacaattgaagccagctgcccaattaaaagctcgtgcacagtatttcatgacaggattcttgtgactccaaatgaacacaaatatgcattgaaaatggtgtgcacagaaaagccagggaacacactcacaattaaagtctgcaatactaaagttgaagcatctatggcccttgtagacgcaaagcctatcatagaactagcaccagttgatcagacagcatatataagagaaaaagatgaaaggtgtaaaacttggatgtgtagggtaagagatgaaggactgcaggtcatcttggagccatttaaaaatttatttggatcttatattgggatattttacacatttattatatctatagtagtattattggttattatctatgtactactacctatatgctttaagttaagggatacccttagaaagcatgaagatgcatataagagagagatgaaaattagataggggatctatgcagaacaaaattgagtcctgtattatatacttctatttgtagtatagctgttgttaagtggggggtggggaactaacaacagcgtaaatttattttgcaaacattattttatacttggtagcacactact

SEQ ID NO: 2 - protein, 299 aa, La Crosse virus
MICILVLITVAAASPVYQRCFQDGAIVKQNPSKEAVTEVCLKDDVSMIKTEARYVRNATGVFSNNVAIRKWLVSDWHDCRPKKIVGGHINVIEVGDDLSLHTESYVCSADCTIGVDKETAQVRLQTDTTNHFEIAGTTVKSGWFKSTTYITLDQTCEHLKVSCGPKSVQFHACFNQHMSCVRFLHRTILPGSIANSICQNIEIIILVTLTLLIFILLSILSKTYICYLLMPIFIPIAYIYGIIYNKSCKKCKLCGLVYHPFTECGTHCVCGARYDTSDRMKLHRASGLCPGYKSLRAAR

SEQ ID NO: 3 - DNA, 984 bp, La Crosse virus
agtagtgtaccccacttgaatactttgaaaataaattgttgttgactgttttttacctaaggggaaattatcaagagtgtgatgtcggatttggtgttttatgatgtcgcatcaacaggtgcaaatggatttgatcctgatgcagggtatatggacttctgtgttaaaaatgcagaattactcaaccttgctgcagttaggatcttcttcctcaatgccgcaaaggccaaggctgctctctcgcgtaagccagagaggaaggctaaccctaaatttggagagtggcaggtggaggttatcaataatcattttcctggaaacaggaacaacccaattggtaacaacgatcttaccatccacagattatctgggtatttagccagatgggtccttgatcagtataacgagaatgatgatgagtctcagcacgagttgatcagaacaactattatcaacccaattgctgagtctaatggtgtaggatgggacagtgggccagagatctatctatcattctttccaggaacagaaatgtttttggaaactttcaaattctacccgctgaccattggaattcacagagtcaagcaaggcatgatggaccctcaatacctgaagaaggccttaaggcaacgctatggcactctcacagcagataagtggatgtcacagaaggttgcagcaattgctaagagcctgaaggatgtagagcagcttaaatggggaaaaggaggcctgagcgatactgctaaaacattcctgcagaaatttggcatcaggcttccataaatatggcatgaggcattcaaattaggttctaaattctaaatttatatatgtcaatttgattaattggttatccaaaagggttttcttaagggaacccacaaaaatagcagctaaatgggtgggtggtaggggacagcaaaaaactataaatcaggtcataaataaaataaaatgtattcagtggggcacactact

SEQ ID NO: 4 - protein, 235 aa, La Crosse virus
MSDLVFYDVASTGANGFDPDAGYMDFCVKNAELLNLAAVRIFFLNAAKAKAALSRKPERKANPKFGEWQVEVINNHFPGNRNNPIGNNDLTIHRLSGYLARWVLDQYNENDDESQHELIRTTIINPIAESNGVGWDSGPEIYLSFFPGTEMFLETFKFYPLTIGIHRVKQGMMDPQYLKKALRQRYGTLTADKWMSQKVAAIAKSLKDVEQLKWGKGGLSDTAKTFLQKFGIRLP

SEQ ID NO: 5 - DNA, 6980 bp, La Crosse virus
agtagtgtacccctatctacaaaacttacagaaaattcagtcatatcacaatatatgcataatggactatcaagagtatcaacaattcttggctaggattaatactgcaagggatgcatgtgtagccaaggatatcgatgttgacctattaatggccagacatgattattttggtagagagctgtgcaagtccttaaatatagaatataggaatgatgtaccatttgtagatataattttggatataaggcccgaagtagacccattaaccatagatgcaccacatattaccccagacaattatctatatataaataatgtgttatatatcatagattataaggtctctgtatcgaatgaaagcagtgttataacatatgacaaatattatgagttaactagggacatatccgatagattaagtattccaatagaaatagttatcgtccgtatagaccctgtaagtaaggatttgcatattaactctgatagatttaaagaactttaccctacaatagtggtggatataaacttcaatcaatttttcgacttaaaacaattactctatgaaaaattcggtgatgatgaagaattcctattgaaagttgcacatggtgacttcactcttacagcaccctggtgcaagactgggtgccctgaattttggaaacaccccatttataaagaatttaaaatgagtatgccagtacctgagcggaggctctttgaagaatctgtcaagttcaatgcttatgaatctgagagatggaatactaacttggttaaaatcagagaatatacaaagaaagactattcagagcatatttcaaaatctgcaaaaaatattttcctggctagtggattttataagcagccaaataagaatgagattagtgaggggtggacattaatggttgagagggttcaagatcagagagaaatctcaaaatctctccatgaccagaaacctagcatacattttatatggggagcccataacccaggaaatagtaataatgcaaccttcaaactcatattgctttcaaagtccttacaaagcataaaaggtatatcaacttacacagaagcgttcaaatctttaggaaaaatgatggatattggagataaggctattgagtatgaagaattctgcatgtccctaaaaagcaaagcaagatcatcatggaagcaaataatgaacaaaaaattagagcctaaacaaataaacaatgcccttgttttatgggaacagcagtttatggtaaataatgacctgatagacaaaagtgagaagttgaaattattcaaaaatttctgcggtataggcaaacacaagcaattcaagaataaaatgctagaggatctagaagtgtcaaagcccaaaatattagactttgatgacgcaaatatgtatctagctagcctaaccatgatggaacagagtaagaagatattgtccaaaagcaatgggttgaagccagataattttatactgaatgaatttggatccaaaatcaaagatgctaataaagaaacatatgacaatatgcacaaaatatttgagacaagatattggcaatgtatatccgacttctctactctgatgaaaaatatcttatctgtgtcccaatataacaggcacaacacatttaggatagctatgtgtgctaataacaatgtctttgctatagtatttccttcggctgacataaaaactaagaaagcaactgtagtttatagcattatagtgctgcataaagaggaagaaaacatattcaacccaggatgtttgcacggcacatttaagtgtatgaatgggtatatttccatatctagagctataaggctagataaagagaggtgccagagaattgtttcctcacctggactgtttttaacaacttgcctactattcaaacatgataatccaactctagtgatgagcgatattatgaatttttctatatacactagcctgtctatcacaaagagtgttctatctttaacagagccagcacgctacatgattatgaactcattagctatctccagcaatgttaaggactatatagcagagaaattttccccttacacaaagacactgttcagtgtctatatgactagactaattaaaaatgcttgctttgatgcttatgaccagagacagcgtgtccaacttagagatatatatttatctgattatgacataacccaaaaaggtattaaagacaatagagagctaacaagtatatggttccctggtagtgtaacattaaaggagtatttaacacaaatatacttaccattttattttaatgctaaaggactacatgagaagcaccatgtcatggtggatctagcaaagactatattagaaatagagtgcgaacagagggaaaacataaaggagatatggtctacaaattgtaccaaacagacagtgaaccttaaaattttgatccattccttgtgcaagaatttactagcagacacttcaagacacaaccacttgcggaacagaatagaaaataggaacaattttagaaggtctataacaactatttcaacatttacaagttcaaagtcttgcctcaaaataggggactttagaaaagagaaagagctgcagtcagttaaacagaagaaaatcttagaggtgcagagtcgcaaaatgagattagcaaacccaatgttcgtgacagatgaacaagtatgccttgaagttgggcactgcaattatgagatgctgaggaatgctatgccgaattatacagattatatatcaactaaagtatttgataggttatatgagttattagataaaggagttttgacagacaagcctgttatagagcaaataatggatatgatggtcgaccacaaaaagttctatttcacatttttcaataaaggccagaaaacgtcaaaggatagagagatattcgttggagaatatgaagctaaaatgtgtatgtacgcagttgagagaatagcaaaagaaagatgtaaattaaatcctgatgaaatgatatctgagccgggtgatggcaagttgaaggtgttggagcaaaaatcagaacaagaaattcgattcttggtcgagactacaaggcaaaagaatcgtgaaatcgatgaggcaattgaagcattagctgcagaaggatatgagagtaatctagaaaaaattgaaaagctttcacttggcaaagcaaagggcctaaagatggaaataaatgcagatatgtctaaatggagtgctcaggatgttttttataaatatttctggctcatagccttagaccctatcctctacccacaggaaaaagagagaatattatactttatgtgcaactacatggataaagaattgatactgccagatgaattattattcaatttgctggaccaaaaagttgcataccagaatgatataatagctactatgactaatcaattaaattcaaatacagttctgataaagagaaattggctccaagggaatttcaactacacctcaagttacgtccatagctgcgcaatgtctgtgtataaagaaatattaaaagaggccataacattactagacgggtctatattagtcaactcattagtccattcggatgataaccaaacatcgataacaatagttcaggataagatggaaaatgataaaattatagattttgcaatgaaagaatttgagagagcctgtttgacatttggatgccaagcaaatatgaaaaagacatatgtaacaaattgcataaaagagtttgtttcattatttaacttgtacggcgaacccttttcaatatatggcagattcctattaacatctgtgggtgattgtgcctatatagggccttatgaagatttagctagtcgaatatcatcagcccagacagccataaagcatggttgtccacccagtctagcatgggtgtccatagcaataagtcattggatgacctctctgacatacaacatgctaccagggcagtcaaatgacccaattgattatttccctgcagaaaataggaaggatatccctatagaattgaatggtgtattagatgctccattgtcaatgattagtacagttggattggaatctgggaatttatacttcttgataaagttgttgagcaaatataccccggtcatgcagaaaagagagtcagtagtcaaccaaatagctgaagttaagaactggaaggtcgaggatctaacagacaatgaaatatttagacttaaaatactcagatatttagttctagatgcagagatggaccctagtgatattatgggtgagacaagcgacatgagagggaggtctattttgacacctagaaaattcacaacagcaggcagtttaaggaaattatattctttcagtaagtaccaggatagactgtcttcccctggaggcatggttgaattgttcacttatttgcttgagaaacctgagttgttagtgactaaaggggaagatatgaaagattatatggaatctgtgatattccgatataattccaaaaggttcaaagaaagtttgtcaatacagaacccagcacaattatttatagaacagatattgttctcacataagcccataatagacttttctggtatcagggacaaatatataaacctacatgatagtagagctctagagaaggaacctgacatattaggaaaagtaacatttacagaggcttatagattattaatgagggacctgtctagcctagaactaaccaatgatgacattcaagtaatttattcttacataatacttaatgaccctatgatgataactattgcaaacacacatatattgtcaatatacgggagtcctcaacggaggatgggcatgtcctgttcaacgatgccagaatttagaaatttaaaattaatacatcattccccagccttagttttgagagcatatagtaaaaataatcctgacatccagggtgctgatcccacggaaatggctagagatttagttcatctgaaagagtttgttgagaacacaaatttagaagaaaaaatgaaagttaggattgctataaatgaagcagagaaaggacaacgggatatagtctttgaactaaaagagatgactagattttatcaggtttgctatgagtatgtcaaatctacagaacacaagataaaagtcttcattctcccgacaaaatcatacacaacaacagatttctgttcactcatgcaggggaatttaataaaagataaagagtggtacacagttcactacctaaaacagatattgtctggtggccataaagccataatgcagcataatgccactagtgagcaaaatattgcttttgagtgtttcaaattaattacccattttgcagactcattcatagattcattatctaggtcagcttttttgcagttgataatagatgaattcagttataaagatgtgaaggttagcaaactttatgacataataaagaatgggtataatcgaactgacttcataccattgctttttagaactggcgatttaagacaagctgacttagacaagtatgatgctatgaaaagtcatgagagggttacatggaatgattggcaaacatctcgtcacttggacatgggctcaattaatctaacaataaccggttacaatagatcaataacaataatcggagaagataacaaattgacatatgcagaattatgtctgactaggaaaactcctgagaatataactataagtggcagaaaattgctaggtgcaaggcatggacttaaatttgaaaatatgtccaaaatccaaacatacccaggcaattattatataacatatagaaagaaagatcgccaccagtttgtataccagatacattctcatgaatcaataacaaggaggaatgaagagcatatggctatcaggaccagaatatacaatgaaataactccagtatgtgtagttaacgttgcagaggtggatggggaccaacgtatattgataagatctttagactatctaaataatgatatattttctctttcaaggattaaagtcgggcttgacgaatttgcaacaataaaaaaagcacactttagtaaaatggtctcatttgaaggacccccaattaagacagggctcctcgaccttactgaattgatgaaatctcaagatttgcttaaccttaattatgataatataaggaatagcaacttgatatctttttcaaaattgatttgctgtgaggggtcagataatataaatgatgggttagagtttctgtccgatgaccctatgaactttacagagggtgaagcaatacattcaacaccgatctttaatatatattactcaaaaagaggagaaagacatatgacatacaggaatgcaattaaattactgatagaaagagaaactaagatttttgaagaagctttcacattcagtgagaatggcttcatatcgccagagaatcttggttgcttagaagcagtagtatcattaataaaattgttgaaaactaatgagtggtccacagttatagataaatgtattcatatatgtttaataaagaatggtatggatcacatgtaccattcatttgatgtccctaaatgttttatggggaatcctatcactagagacatgaattggatgatgtttagagaattcatcaatagtttaccagggacagatataccaccatggaatgtcatgacagagaacttcaaaaagaaatgtattgctctgataaactctaagttagaaacacagagagatttctcagaattcactaaactgatgaaaaaggaaggtgggaggagtaatatagaatttgattagtagttatgagtttacagagaacctacaattaggctataaatttgggagggttttggaaattggctaaaattcaaaaagagggggattaacagcaactgtataaatttgtagataggggcacactact

SEQ ID NO: 6 - protein, 2263 aa, La Crosse virus
MDYQEYQQFLARINTARDACVAKDIDVDLLMARHDYFGRELCKSLNIEYRNDVPFVDIILDIRPEVDPLTIDAPHITPDNYLYINNVLYIIDYKVSVSNESSVITYDKYYELTRDISDRLSIPIEIVIVRIDPVSKDLHINSDRFKELYPTIVVDINFNQFFDLKQLLYEKFGDDEEFLLKVAHGDFTLTAPWCKTGCPEFWKHPIYKEFKMSMPVPERRLFEESVKFNAYESERWNTNLVKIREYTKKDYSEHISKSAKNIFLASGFYKQPNKNEISEGWTLMVERVQDQREISKSLHDQKPSIHFIWGAHNPGNSNNATFKLILLSKSLQSIKGISTYTEAFKSLGKMMDIGDKAIEYEEFCMSLKSKARSSWKQIMNKKLEPKQINNALVLWEQQFMVNNDLIDKSEKLKLFKNFCGIGKHKQFKNKMLEDLEVSKPKILDFDDANMYLASLTMMEQSKKILSKSNGLKPDNFILNEFGSKIKDANKETYDNMHKIFETRYWQCISDFSTLMKNILSVSQYNRHNTFRIAMCANNNVFAIVFPSADIKTKKATVVYSIIVLHKEEENIFNPGCLHGTFKCMNGYISISRAIRLDKERCQRIVSSPGLFLTTCLLFKHDNPTLVMSDIMNFSIYTSLSITKSVLSLTEPARYMIMNSLAISSNVKDYIAEKFSPYTKTLFSVYMTRLIKNACFDAYDQRQRVQLRDIYLSDYDITQKGIKDNRELTSIWFPGSVTLKEYLTQIYLPFYFNAKGLHEKHHVMVDLAKTILEIECEQRENIKEIWSTNCTKQTVNLKILIHSLCKNLLADTSRHNHLRNRIENRNNFRRSITTISTFTSSKSCLKIGDFRKEKELQSVKQKKILEVQSRKMRLANPMFVTDEQVCLEVGHCNYEMLRNAMPNYTDYISTKVFDRLYELLDKGVLTDKPVIEQIMDMMVDHKKFYFTFFNKGQKTSKDREIFVGEYEAKMCMYAVERIAKERCKLNPDEMISEPGDGKLKVLEQKSEQEIRFLVETTRQKNREIDEAIEALAAEGYESNLEKIEKLSLGKAKGLKMEINADMSKWSAQDVFYKYFWLIALDPILYPQEKERILYFMCNYMDKELILPDELLFNLLDQKVAYQNDIIATMTNQLNSNTVLIKRNWLQGNFNYTSSYVHSCAMSVYKEILKEAITLLDGSILVNSLVHSDDNQTSITIVQDKMENDKIIDFAMKEFERACLTFGCQANMKKTYVTNCIKEFVSLFNLYGEPFSIYGRFLLTSVGDCAYIGPYEDLASRISSAQTAIKHGCPPSLAWVSIAISHWMTSLTYNMLPGQSNDPIDYFPAENRKDIPIELNGVLDAPLSMISTVGLESGNLYFLIKLLSKYTPVMQKRESVVNQIAEVKNWKVEDLTDNEIFRLKILRYLVLDAEMDPSDIMGETSDMRGRSILTPRKFTTAGSLRKLYSFSKYQDRLSSPGGMVELFTYLLEKPELLVTKGEDMKDYMESVIFRYNSKRFKESLSIQNPAQLFIEQILFSHKPIIDFSGIRDKYINLHDSRALEKEPDILGKVTFTEAYRLLMRDLSSLELTNDDIQVIYSYIILNDPMMITIANTHILSIYGSPQRRMGMSCSTMPEFRNLKLIHHSPALVLRAYSKNNPDIQGADPTEMARDLVHLKEFVENTNLEEKMKVRIAINEAEKGQRDIVFELKEMTRFYQVCYEYVKSTEHKIKVFILPTKSYTTTDFCSLMQGNLIKDKEWYTVHYLKQILSGGHKAIMQHNATSEQNIAFECFKLITHFADSFIDSLSRSAFLQLIIDEFSYKDVKVSKLYDIIKNGYNRTDFIPLLFRTGDLRQADLDKYDAMKSHERVTWNDWQTSRHLDMGSINLTITGYNRSITIIGEDNKLTYAELCLTRKTPENITISGRKLLGARHGLKFENMSKIQTYPGNYYITYRKKDRHQFVYQIHSHESITRRNEEHMAIRTRIYNEITPVCVVNVAEVDGDQRILIRSLDYLNNDIFSLSRIKVGLDEFATIKKAHFSKMVSFEGPPIKTGLLDLTELMKSQDLLNLNYDNIRNSNLISFSKLICCEGSDNINDGLEFLSDDPMNFTEGEAIHSTPIFNIYYSKRGERHMTYRNAIKLLIERETKIFEEAFTFSENGFISPENLGCLEAVVSLIKLLKTNEWSTVIDKCIHICLIKNGMDHMYHSFDVPKCFMGNPITRDMNWMMFREFINSLPGTDIPPWNVMTENFKKKCIALINSKLETQRDFSEFTKLMKKEGGRSNIEFD

SEQ ID NO: 7 - DNA, 25 bp, Artificial Sequence
  Antisense primer derived from M segment of LACV genome
cgatcaacaatccaatgataacaag

SEQ ID NO: 8 - DNA, 22 bp, Artificial Sequence
  Sense primer derived from M segment of LACV genome
tggaaatggcatcgagaataaa

SEQ ID NO: 9 - DNA, 39 bp, Artificial Sequence
  Probe derived from M segment of LACV genome
attatctcacctgtatcttgaattatgctgtaagctggg

SEQ ID NO: 10 - DNA, 23 bp, Artificial Sequence
  Sense primer derived from S segment of LACV genome
gtctcagcacgagttgatcagaa

SEQ ID NO: 11 - DNA, 22 bp, Artificial Sequence
  Antisense primer derived from S segment of LACV genome
aatggtcagcgggtagaatttg

SEQ ID NO: 12 - DNA, 25 bp, Artificial Sequence
  Probe derived from S segment of LACV genome
tggtgtaggatgggacagtgggcca

SEQ ID NO: 13 - DNA, 21 bp, Artificial Sequence
  Sense primer derived from L segment of LACV genome
aaagtcgggcttgacgaattt

SEQ ID NO: 14 - DNA, 23 bp, Artificial Sequence
  Antisense primer derived from L segment of LACV genome
cggacagaaactctaacccatca

SEQ ID NO: 15 - DNA, 25 bp, Artificial Sequence
  Probe derived from L segment of LACV genome
cccccaattaagacagggctcctcg

SEQ ID NO: 16 - DNA, 25 bp, Artificial Sequence
  Synthetic oligonucleotide specific for LACV sequence
catgaggcattcaaattaggttcta

SEQ ID NO: 17 - protein, 174 aa, La Crosse virus
VMCKSKGPASILSIITAVLVLTFVTPINSMVLGESKETFELEDLPDDMLEMASRINSYYLTCILNYAVSWGLVIIGLLIGLLFKKYQHRFLNVYAMYCEECDMYHDKSGLKRHGDFTNKCRQCTCGQYEDAAGLMAHRKTYNCLVQYKAKWMMNFLIIYIFLILIKDSAIVVQA

SEQ ID NO: 18 - protein, 968 aa, La Crosse virus
AGTDFTTCLETESINWNCTGPFLNLGNCQKQQKKEPYTNIATQLKGLKAISVLDVPIITGIPDDIAGALRYIEEKEDFHVQLTIEYAMLSKYCDYYTQFSDNSGYSQTTWRVYLRSHDFEACILYPNQHFCRCVKNGEKCSSSNWDFANEMKDYYSGKQTKFDKDLNLALTALHHAFRGTSSAYIATMLSKKSNDDLIAYTNKIKTKFPGNALLKAIIDYIAYMKSLPGMANFKYDEFWDELLYKPNPAKASNLARGKESSYNFKLAISSKSIKTCKNVKDVACLSPRSGAIYASIIACGEPNGPSVYRKPSGGVFQSSTDRSIYCLLDSHCLEEFEAIGQEELDAVKKSKCWEIEYPDVKLIQEGDGTKSCRMKDSGNCNVATNRWPVIQCENDKFYYSELQKDYDKAQDIGHYCLSPGCTTVRYPINPKHISNCNWQVSRSSIAKIDVHNIEDIEQYKKAITQKLQTSLSLFKYAKTKNLPHIKPIYKYITIEGTETAEGIESAYIESEVPALAGTSIGFKINSKEGKHLLDVIAYVKSASYSSVYTKLYSTGPTSGINTKHDELCTGPCPANINHQVGWLTFARERTSSWGCEEFGCLAVSDGCVFGSCQDIIKEELSVYRKETEEVTDVELCLTFSDKTYCTNLNPVTPIITDLFEVQFKTVETYSLPRIVAVQNHEIKIGQINDLGVYSKGCGNVQKVNGTIYGNGVPRFDYLCHLASRKEVIVRKCFDNDYQACKFLQSPASYRLEEDSGTVTIIDYKKILGTIKMKAILGDVKYKTFADSVDITAEGSCTGCINCFENIHCELTLHTTIEASCPIKSSCTVFHDRILVTPNEHKYALKMVCTEKPGNTLTIKVCNTKVEASMALVDAKPIIELAPVDQTAYIREKDERCKTWMCRVRDEGLQVILEPFKNLFGSYIGIFYTFIISIVVLLVIIYVLLPICFKLRDTLRKHEDAYKREMKIR

SEQ ID NO: 19 - protein, 92 aa, La Crosse virus
MMSHQQVQMDLILMQGIWTSVLKMQNYSTLLQLGSSSSMPQRPRLLSRVSQRGRLTLNLESGRWRLSIIIFLETGTTQLVTTILPSTDYLGI

SEQ ID NO: 20 - DNA, 25 bp, Artificial Sequence
  Forward primer derived from M segment of the LACV genome
ttgtacaagctgctggaactgactt

SEQ ID NO: 21 - DNA, 22 bp, Artificial Sequence
  Forward primer derived from M segment of the LACV genome
tgtggtgcccgctatgatactt

SEQ ID NO: 22 - DNA, 20 bp, Artificial Sequence
  Forward primer derived from M segment of the LACV genome
tgtggtgcccgctatgatac

SEQ ID NO: 23 - DNA, 21 bp, Artificial Sequence
  Forward primer derived from M segment of the LACV genome
ctgtggtgcccgctatgatac

SEQ ID NO: 24 - DNA, 20 bp, Artificial Sequence
  Forward primer derived from M segment of the LACV genome
ctgtggtgcccgctatgata

SEQ ID NO: 25 - DNA, 21 bp, Artificial Sequence
  Forward primer derived from M segment of the LACV genome
tctgtggtgcccgctatgata

SEQ ID NO: 26 - DNA, 20 bp, Artificial Sequence
  Forward primer derived from M segment of the LACV genome
tctgtggtgcccgctatgat

SEQ ID NO: 27 - DNA, 20 bp, Artificial Sequence
  Forward primer derived from M segment of the LACV genome
gtgtctgtggtgcccgctat

SEQ ID NO: 28 - DNA, 23 bp, Artificial Sequence
  Forward primer derived from M segment of the LACV genome
agacagtggcactgtgaccataa

SEQ ID NO: 29 - DNA, 24 bp, Artificial Sequence
  Forward primer derived from M segment of the LACV genome
agacagtggcactgtgaccataat

SEQ ID NO: 30 - DNA, 23 bp, Artificial Sequence
  Forward primer derived from M segment of the LACV genome
aagacagtggcactgtgaccata

SEQ ID NO: 31 - DNA, 24 bp, Artificial Sequence
  Forward primer derived from M segment of the LACV genome
aagacagtggcactgtgaccataa

SEQ ID NO: 32 - DNA, 25 bp, Artificial Sequence
  Forward primer derived from M segment of the LACV genome
aagacagtggcactgtgaccataat

SEQ ID NO: 33 - DNA, 24 bp, Artificial Sequence
  Forward primer derived from M segment of the LACV genome
gaagacagtggcactgtgaccata

SEQ ID NO: 34 - DNA, 25 bp, Artificial Sequence
  Forward primer derived from M segment of the LACV genome
agaagacagtggcactgtgaccata

SEQ ID NO: 35 - DNA, 25 bp, Artificial Sequence
  Probe derived from M segment of the LACV genome
ctgggccatttttgaacctcgggaa

SEQ ID NO: 36 - DNA, 24 bp, Artificial Sequence
  Probe derived from M segment of the LACV genome
ctgggccatttttgaacctcggga

SEQ ID NO: 37 - DNA, 24 bp, Artificial Sequence
  Probe derived from M segment of the LACV genome
cactgggccatttttgaacctcgg

SEQ ID NO: 38 - DNA, 23 bp, Artificial Sequence
  Probe derived from M segment of the LACV genome
ctgggccatttttgaacctcggg

SEQ ID NO: 39 - DNA, 25 bp, Artificial Sequence
  Probe derived from M segment of the LACV genome
tgaacctcgggaattgccaaaagca

SEQ ID NO: 40 - DNA, 25 bp, Artificial Sequence
  Probe derived from M segment of the LACV genome
tgcactgggccatttttgaacctcg

SEQ ID NO: 41 - DNA, 25 bp, Artificial Sequence
  Probe derived from M segment of the LACV genome
actgggccatttttgaacctcggga

SEQ ID NO: 42 - DNA, 24 bp, Artificial Sequence
  Probe derived from M segment of the LACV genome
actgggccatttttgaacctcggg

SEQ ID NO: 43 - DNA, 23 bp, Artificial Sequence
  Probe derived from M segment of the LACV genome
tgggccatttttgaacctcggga

SEQ ID NO: 44 - DNA, 25 bp, Artificial Sequence
  Probe derived from M segment of the LACV genome
tgggccatttttgaacctcgggaat

SEQ ID NO: 45 - DNA, 25 bp, Artificial Sequence
  Probe derived from M segment of the LACV genome
cactgggccatttttgaacctcggg

SEQ ID NO: 46 - DNA, 24 bp, Artificial Sequence
  Probe derived from M segment of the LACV genome
tgggccatttttgaacctcgggaa

SEQ ID NO: 47 - DNA, 23 bp, Artificial Sequence
  Probe derived from M segment of the LACV genome
tgtgcaagtcgaaagggcctgca

SEQ ID NO: 48 - DNA, 24 bp, Artificial Sequence
  Probe derived from M segment of the LACV genome
catgtgcaagtcgaaagggcctgc

SEQ ID NO: 49 - DNA, 24 bp, Artificial Sequence
  Probe derived from M segment of the LACV genome
tcatgtgcaagtcgaaagggcctg

SEQ ID NO: 50 - DNA, 24 bp, Artificial Sequence
  Probe derived from M segment of the LACV genome
atgtgcaagtcgaaagggcctgca

SEQ ID NO: 51 - DNA, 25 bp, Artificial Sequence
  Probe derived from M segment of the LACV genome
tcatgtgcaagtcgaaagggcctgc

SEQ ID NO: 52 - DNA, 24 bp, Artificial Sequence
  Probe derived from M segment of the LACV genome
taaccgcagaagggtcatgcaccg

SEQ ID NO: 53 - DNA, 21 bp, Artificial Sequence
  Probe derived from M segment of the LACV genome
ccgcagaagggtcatgcaccg

SEQ ID NO: 54 - DNA, 23 bp, Artificial Sequence
  Probe derived from M segment of the LACV genome
aaccgcagaagggtcatgcaccg

SEQ ID NO: 55 - DNA, 25 bp, Artificial Sequence
  Probe derived from M segment of the LACV genome
ataaccgcagaagggtcatgcaccg

SEQ ID NO: 56 - DNA, 22 bp, Artificial Sequence
  Probe derived from M segment of the LACV genome
accgcagaagggtcatgcaccg

SEQ ID NO: 57 - DNA, 23 bp, Artificial Sequence
  Probe derived from M segment of the LACV genome
cagaagggtcatgcaccggctgt

SEQ ID NO: 58 - DNA, 21 bp, Artificial Sequence
  Probe derived from M segment of the LACV genome
cgcagaagggtcatgcaccgg

SEQ ID NO: 59 - DNA, 25 bp, Artificial Sequence
  Reverse primer derived from M segment of the LACV genome
agtccctttaactgagttgcaatgt

SEQ ID NO: 60 - DNA, 25 bp, Artificial Sequence
  Reverse primer derived from M segment of the LACV genome
aaggttaagaccagtaccgcagtaa

SEQ ID NO: 61 - DNA, 22 bp, Artificial Sequence
  Reverse primer derived from M segment of the LACV genome
gtgtgcaacgttaattcgcaat

SEQ ID NO: 62 - DNA, 22 bp, Artificial Sequence
  Reverse primer derived from M segment of the LACV genome
tgtggtgtgcaacgttaattcg

SEQ ID NO: 63 - DNA, 22 bp, Artificial Sequence
  Reverse primer derived from M segment of the LACV genome
tcaattgtggtgtgcaacgtta

SEQ ID NO: 64 - DNA, 23 bp, Artificial Sequence
  Reverse primer derived from M segment of the LACV genome
tcaattgtggtgtgcaacgttaa

SEQ ID NO: 65 - DNA, 21 bp, Artificial Sequence
  Reverse primer derived from M segment of the LACV genome
tcaattgtggtgtgcaacgtt

SEQ ID NO: 66 - DNA, 24 bp, Artificial Sequence
  Reverse primer derived from M segment of the LACV genome
tcaattgtggtgtgcaacgttaat

SEQ ID NO: 67 - DNA, 23 bp, Artificial Sequence
  Forward primer derived from the S segment of the LACV genome
tctcagcacgagttgatcagaac

SEQ ID NO: 68 - DNA, 23 bp, Artificial Sequence
  Forward primer derived from the S segment of the LACV genome
ctcagcacgagttgatcagaaca

SEQ ID NO: 69 - DNA, 23 bp, Artificial Sequence
  Forward primer derived from the S segment of the LACV genome
tcagcacgagttgatcagaacaa

SEQ ID NO: 70 - DNA, 22 bp, Artificial Sequence
  Forward primer derived from the S segment of the LACV genome
tctacccgctgaccattggaat

SEQ ID NO: 71 - DNA, 24 bp, Artificial Sequence
  Forward primer derived from the S segment of the LACV genome
gagtgtgatgtcggatttggtgtt

SEQ ID NO: 72 - DNA, 24 bp, Artificial Sequence
  Forward primer derived from the S segment of the LACV genome
agtctcagcacgagttgatcagaa

SEQ ID NO: 73 - DNA, 24 bp, Artificial Sequence
  Forward primer derived from the S segment of the LACV genome
gtctcagcacgagttgatcagaac

SEQ ID NO: 74 - DNA, 24 bp, Artificial Sequence
  Forward primer derived from the S segment of the LACV genome
tctcagcacgagttgatcagaaca

SEQ ID NO: 75 - DNA, 24 bp, Artificial Sequence
  Forward primer derived from the S segment of the LACV genome
ctcagcacgagttgatcagaacaa

SEQ ID NO: 76 - DNA, 22 bp, Artificial Sequence
  Forward primer derived from the S segment of the LACV genome
tcagcacgagttgatcagaaca

SEQ ID NO: 77 - DNA, 21 bp, Artificial Sequence
  Forward primer derived from the S segment of the LACV genome
tctacccgctgaccattggaa

SEQ ID NO: 78 - DNA, 22 bp, Artificial Sequence
  Forward primer derived from the S segment of the LACV genome
tacccgctgaccattggaattc

SEQ ID NO: 79 - DNA, 24 bp, Artificial Sequence
  Forward primer derived from the S segment of the LACV genome
caagagtgtgatgtcggatttggt

SEQ ID NO: 80 - DNA, 23 bp, Artificial Sequence
  Forward primer derived from the S segment of the LACV genome
aagagtgtgatgtcggatttggt

SEQ ID NO: 81 - DNA, 23 bp, Artificial Sequence
  Forward primer derived from the S segment of the LACV genome
cctgatgcagggtatatggactt

SEQ ID NO: 82 - DNA, 24 bp, Artificial Sequence
  Forward primer derived from the S segment of the LACV genome
tgcagggtatatggacttctgtgt

SEQ ID NO: 83 - DNA, 24 bp, Artificial Sequence
  Forward primer derived from the S segment of the LACV genome
gatgagtctcagcacgagttgatc

SEQ ID NO: 84 - DNA, 25 bp, Artificial Sequence
  Forward primer derived from the S segment of the LACV genome
gagtctcagcacgagttgatcagaa

SEQ ID NO: 85 - DNA, 25 bp, Artificial Sequence
  Forward primer derived from the S segment of the LACV genome
agtctcagcacgagttgatcagaac

SEQ ID NO: 86 - DNA, 20 bp, Artificial Sequence
  Forward primer derived from the S segment of the LACV genome
tctacccgctgaccattgga

SEQ ID NO: 87 - DNA, 21 bp, Artificial Sequence
  Forward primer derived from the S segment of the LACV genome
ctacccgctgaccattggaat

SEQ ID NO: 88 - DNA, 21 bp, Artificial Sequence
  Forward primer derived from the S segment of the LACV genome
cgctgaccattggaattcaca

SEQ ID NO: 89 - DNA, 24 bp, Artificial Sequence
  Forward primer derived from the S segment of the LACV genome
cctgatgcagggtatatggacttc

SEQ ID NO: 90 - DNA, 25 bp, Artificial Sequence
  Forward primer derived from the S segment of the LACV genome
atgcagggtatatggacttctgtgt

SEQ ID NO: 91 - DNA, 25 bp, Artificial Sequence
  Probe derived from S segment of LACV genome
caagcaaggcatgatggaccctcaa

SEQ ID NO: 92 - DNA, 25 bp, Artificial Sequence
  Probe derived from S segment of LACV genome
tcaagcaaggcatgatggaccctca

SEQ ID NO: 93 - DNA, 25 bp, Artificial Sequence
  Probe derived from S segment of LACV genome
tgtcgcatcaacaggtgcaaatgga

SEQ ID NO: 94 - DNA, 21 bp, Artificial Sequence
  Probe derived from S segment of LACV genome
caatgccgcaaaggccaaggc

SEQ ID NO: 95 - DNA, 23 bp, Artificial Sequence
  Probe derived from S segment of LACV genome
atgccgcaaaggccaaggctgct

SEQ ID NO: 96 - DNA, 22 bp, Artificial Sequence
  Probe derived from S segment of LACV genome
ccgcaaaggccaaggctgctct

SEQ ID NO: 97 - DNA, 24 bp, Artificial Sequence
  Probe derived from S segment of LACV genome
ccgcaaaggccaaggctgctctct

SEQ ID NO: 98 - DNA, 21 bp, Artificial Sequence
  Probe derived from S segment of LACV genome
atgccgcaaaggccaaggctg

SEQ ID NO: 99 - DNA, 21 bp, Artificial Sequence
  Probe derived from S segment of LACV genome
tgccgcaaaggccaaggctgc

SEQ ID NO: 100 - DNA, 23 bp, Artificial Sequence
  Probe derived from S segment of LACV genome
caatgccgcaaaggccaaggctg

SEQ ID NO: 101 - DNA, 24 bp, Artificial Sequence
  Probe derived from S segment of LACV genome
aggccaaggctgctctctcgcgta

SEQ ID NO: 102 - DNA, 23 bp, Artificial Sequence
  Probe derived from S segment of LACV genome
cgcaaaggccaaggctgctctct

SEQ ID NO: 103 - DNA, 24 bp, Artificial Sequence
  Probe derived from S segment of LACV genome
ccaaggctgctctctcgcgtaagc

SEQ ID NO: 104 - DNA, 24 bp, Artificial Sequence
  Probe derived from S segment of LACV genome
caaaggccaaggctgctctctcgc

SEQ ID NO: 105 - DNA, 22 bp, Artificial Sequence
  Probe derived from S segment of LACV genome
aggccaaggctgctctctcgcg

SEQ ID NO: 106 - DNA, 25 bp, Artificial Sequence
  Probe derived from S segment of LACV genome
aaaggccaaggctgctctctcgcgt

SEQ ID NO: 107 - DNA, 23 bp, Artificial Sequence
  Probe derived from S segment of LACV genome
cttcctcaatgccgcaaaggcca

SEQ ID NO: 108 - DNA, 23 bp, Artificial Sequence
  Probe derived from S segment of LACV genome
tcttcctcaatgccgcaaaggcc

SEQ ID NO: 109 - DNA, 24 bp, Artificial Sequence
  Probe derived from S segment of LACV genome
aaggccaaggctgctctctcgcgt

SEQ ID NO: 110 - DNA, 24 bp, Artificial Sequence
  Probe derived from S segment of LACV genome
tcttcctcaatgccgcaaaggcca

SEQ ID NO: 111 - DNA, 25 bp, Artificial Sequence
  Probe derived from S segment of LACV genome
tcttcttcctcaatgccgcaaaggc

SEQ ID NO: 112 - DNA, 22 bp, Artificial Sequence
  Probe derived from S segment of LACV genome
tcaatgccgcaaaggccaaggc

SEQ ID NO: 113 - DNA, 25 bp, Artificial Sequence
  Probe derived from S segment of LACV genome
ttcttcctcaatgccgcaaaggcca

SEQ ID NO: 114 - DNA, 23 bp, Artificial Sequence
  Probe derived from S segment of LACV genome
cctcaatgccgcaaaggccaagg

SEQ ID NO: 115 - DNA, 25 bp, Artificial Sequence
  Probe derived from S segment of LACV genome
cttcctcaatgccgcaaaggccaag

SEQ ID NO: 116 - DNA, 24 bp, Artificial Sequence
  Probe derived from S segment of LACV genome
ttcttcctcaatgccgcaaaggcc

SEQ ID NO: 117 - DNA, 23 bp, Artificial Sequence
  Probe derived from S segment of LACV genome
ctcaatgccgcaaaggccaaggc

SEQ ID NO: 118 - DNA, 23 bp, Artificial Sequence
  Probe derived from S segment of LACV genome
ttcctcaatgccgcaaaggccaa

SEQ ID NO: 119 - DNA, 23 bp, Artificial Sequence
  Probe derived from S segment of LACV genome
tcctcaatgccgcaaaggccaag

SEQ ID NO: 120 - DNA, 21 bp, Artificial Sequence
  Probe derived from S segment of LACV genome
tcctcaatgccgcaaaggcca

SEQ ID NO: 121 - DNA, 23 bp, Artificial Sequence
  Probe derived from S segment of LACV genome
tcaatgccgcaaaggccaaggct

SEQ ID NO: 122 - DNA, 22 bp, Artificial Sequence
  Probe derived from S segment of LACV genome
caatgccgcaaaggccaaggct

SEQ ID NO: 123 - DNA, 25 bp, Artificial Sequence
  Probe derived from S segment of LACV genome
cttcttcctcaatgccgcaaaggcc

SEQ ID NO: 124 - DNA, 22 bp, Artificial Sequence
  Probe derived from S segment of LACV genome
ctcaatgccgcaaaggccaagg

SEQ ID NO: 125 - DNA, 22 bp, Artificial Sequence
  Probe derived from S segment of LACV genome
aatgccgcaaaggccaaggctg

SEQ ID NO: 126 - DNA, 22 bp, Artificial Sequence
  Probe derived from S segment of LACV genome
atgccgcaaaggccaaggctgc

SEQ ID NO: 127 - DNA, 20 bp, Artificial Sequence
  Probe derived from S segment of LACV genome
tgccgcaaaggccaaggctg

SEQ ID NO: 128 - DNA, 24 bp, Artificial Sequence
  Probe derived from S segment of LACV genome
ctcaatgccgcaaaggccaaggct

SEQ ID NO: 129 - DNA, 22 bp, Artificial Sequence
  Probe derived from S segment of LACV genome
cctcaatgccgcaaaggccaag

SEQ ID NO: 130 - DNA, 24 bp, Artificial Sequence
  Probe derived from S segment of LACV genome
cttcctcaatgccgcaaaggccaa

SEQ ID NO: 131 - DNA, 25 bp, Artificial Sequence
  Probe derived from S segment of LACV genome
tcttcctcaatgccgcaaaggccaa

SEQ ID NO: 132 - DNA, 22 bp, Artificial Sequence
  Probe derived from S segment of LACV genome
tcctcaatgccgcaaaggccaa

SEQ ID NO: 133 - DNA, 22 bp, Artificial Sequence
  Probe derived from S segment of LACV genome
ttcctcaatgccgcaaaggcca

SEQ ID NO: 134 - DNA, 24 bp, Artificial Sequence
  Probe derived from S segment of LACV genome
ttcctcaatgccgcaaaggccaag

SEQ ID NO: 135 - DNA, 23 bp, Artificial Sequence
  Probe derived from S segment of LACV genome
aggccaaggctgctctctcgcgt

SEQ ID NO: 136 - DNA, 25 bp, Artificial Sequence
  Probe derived from S segment of LACV genome
caaggctgctctctcgcgtaagcca

SEQ ID NO: 137 - DNA, 25 bp, Artificial Sequence
  Probe derived from S segment of LACV genome
ccaaggctgctctctcgcgtaagcc

SEQ ID NO: 138 - DNA, 25 bp, Artificial Sequence
  Probe derived from S segment of LACV genome
aggccaaggctgctctctcgcgtaa

SEQ ID NO: 139 - DNA, 21 bp, Artificial Sequence
  Probe derived from S segment of LACV genome
ccgcaaaggccaaggctgctc

SEQ ID NO: 140 - DNA, 25 bp, Artificial Sequence
  Probe derived from S segment of LACV genome
aaggctgctctctcgcgtaagccag

SEQ ID NO: 141 - DNA, 24 bp, Artificial Sequence
  Probe derived from S segment of LACV genome
aaggctgctctctcgcgtaagcca

SEQ ID NO: 142 - DNA, 24 bp, Artificial Sequence
  Probe derived from S segment of LACV genome
caaggctgctctctcgcgtaagcc

SEQ ID NO: 143 - DNA, 22 bp, Artificial Sequence
  Probe derived from S segment of LACV genome
cgcaaaggccaaggctgctctc

SEQ ID NO: 144 - DNA, 23 bp, Artificial Sequence
  Probe derived from S segment of LACV genome
ccgcaaaggccaaggctgctctc

SEQ ID NO: 145 - DNA, 25 bp, Artificial Sequence
  Probe derived from S segment of LACV genome
aaggccaaggctgctctctcgcgta

SEQ ID NO: 146 - DNA, 23 bp, Artificial Sequence
  Probe derived from S segment of LACV genome
aaggccaaggctgctctctcgcg

SEQ ID NO: 147 - DNA, 24 bp, Artificial Sequence
  Probe derived from S segment of LACV genome
cgcaaaggccaaggctgctctctc

SEQ ID NO: 148 - DNA, 24 bp, Artificial Sequence
  Probe derived from S segment of LACV genome
aaaggccaaggctgctctctcgcg

SEQ ID NO: 149 - DNA, 22 bp, Artificial Sequence
  Reverse primer derived from S segment of LACV genome
caatggtcagcgggtagaattt

SEQ ID NO: 150 - DNA, 22 bp, Artificial Sequence
  Reverse primer derived from S segment of LACV genome
ccaatggtcagcgggtagaatt

SEQ ID NO: 151 - DNA, 22 bp, Artificial Sequence
  Reverse primer derived from S segment of LACV genome
tccaatggtcagcgggtagaat

SEQ ID NO: 152 - DNA, 23 bp, Artificial Sequence
  Reverse primer derived from S segment of LACV genome
tccttcaggctcttagcaattgc

SEQ ID NO: 153 - DNA, 22 bp, Artificial Sequence
  Reverse primer derived from S segment of LACV genome
ctttgcggcattgaggaagaag

SEQ ID NO: 154 - DNA, 22 bp, Artificial Sequence
  Reverse primer derived from S segment of LACV genome
atggtcagcgggtagaatttga

SEQ ID NO: 155 - DNA, 21 bp, Artificial Sequence
  Reverse primer derived from S segment of LACV genome
ccaatggtcagcgggtagaat

SEQ ID NO: 156 - DNA, 21 bp, Artificial Sequence
  Reverse primer derived from S segment of LACV genome
tccaatggtcagcgggtagaa

SEQ ID NO: 157 - DNA, 20 bp, Artificial Sequence
  Reverse primer derived from S segment of LACV genome
tccaatggtcagcgggtaga

SEQ ID NO: 158 - DNA, 24 bp, Artificial Sequence
  Reverse primer derived from S segment of LACV genome
catccttcaggctcttagcaattg

SEQ ID NO: 159 - DNA, 21 bp, Artificial Sequence
  Reverse primer derived from S segment of LACV genome
tgcggcattgaggaagaagat

SEQ ID NO: 160 - DNA, 20 bp, Artificial Sequence
  Reverse primer derived from S segment of LACV genome
ttgcggcattgaggaagaag

SEQ ID NO: 161 - DNA, 21 bp, Artificial Sequence
  Reverse primer derived from S segment of LACV genome
ctttgcggcattgaggaagaa

SEQ ID NO: 162 - DNA, 24 bp, Artificial Sequence
  Reverse primer derived from S segment of LACV genome
gccactctccaaatttagggttag

SEQ ID NO: 163 - DNA, 23 bp, Artificial Sequence
  Reverse primer derived from S segment of LACV genome
cacctgccactctccaaatttag

SEQ ID NO: 164 - DNA, 23 bp, Artificial Sequence
  Reverse primer derived from S segment of LACV genome
tcagcgggtagaatttgaaagtt

SEQ ID NO: 165 - DNA, 22 bp, Artificial Sequence
  Reverse primer derived from S segment of LACV genome
tggtcagcgggtagaatttgaa

SEQ ID NO: 166 - DNA, 23 bp, Artificial Sequence
  Reverse primer derived from S segment of LACV genome
atggtcagcgggtagaatttgaa

SEQ ID NO: 167 - DNA, 23 bp, Artificial Sequence
  Reverse primer derived from S segment of LACV genome
aatggtcagcgggtagaatttga

SEQ ID NO: 168 - DNA, 23 bp, Artificial Sequence
  Reverse primer derived from S segment of LACV genome
caatggtcagcgggtagaatttg

SEQ ID NO: 169 - DNA, 20 bp, Artificial Sequence
  Reverse primer derived from S segment of LACV genome
ccaatggtcagcgggtagaa

SEQ ID NO: 170 - DNA, 24 bp, Artificial Sequence
  Reverse primer derived from S segment of LACV genome
atccttcaggctcttagcaattgc

SEQ ID NO: 171 - DNA, 24 bp, Artificial Sequence
  Reverse primer derived from S segment of LACV genome
tctacatccttcaggctcttagca

SEQ ID NO: 172 - DNA, 23 bp, Artificial Sequence
  Reverse primer derived from S segment of LACV genome
acctgccactctccaaatttagg

SEQ ID NO: 173 - DNA, 22 bp, Artificial Sequence
  Forward primer derived from L segment of LACV genome
taaagtcgggcttgacgaattt

SEQ ID NO: 174 - DNA, 22 bp, Artificial Sequence
  Forward primer derived from L segment of LACV genome
ttaaagtcgggcttgacgaatt

SEQ ID NO: 175 - DNA, 23 bp, Artificial Sequence
  Forward primer derived from L segment of LACV genome
ttaaagtcgggcttgacgaattt

SEQ ID NO: 176 - DNA, 23 bp, Artificial Sequence
  Forward primer derived from L segment of LACV genome
attaaagtcgggcttgacgaatt

SEQ ID NO: 177 - DNA, 24 bp, Artificial Sequence
  Forward primer derived from L segment of LACV genome
attaaagtcgggcttgacgaattt

SEQ ID NO: 178 - DNA, 22 bp, Artificial Sequence
  Forward primer derived from L segment of LACV genome
gattaaagtcgggcttgacgaa

SEQ ID NO: 179 - DNA, 23 bp, Artificial Sequence
  Forward primer derived from L segment of LACV genome
gattaaagtcgggcttgacgaat

SEQ ID NO: 180 - DNA, 24 bp, Artificial Sequence
  Forward primer derived from L segment of LACV genome
gattaaagtcgggcttgacgaatt

SEQ ID NO: 181 - DNA, 25 bp, Artificial Sequence
  Forward primer derived from L segment of LACV genome
gattaaagtcgggcttgacgaattt

SEQ ID NO: 182 - DNA, 22 bp, Artificial Sequence
  Forward primer derived from L segment of LACV genome
caaggattaaagtcgggcttga

SEQ ID NO: 183 - DNA, 23 bp, Artificial Sequence
  Forward primer derived from L segment of LACV genome
caaggattaaagtcgggcttgac

SEQ ID NO: 184 - DNA, 23 bp, Artificial Sequence
  Forward primer derived from L segment of LACV genome
tcaaggattaaagtcgggcttga

SEQ ID NO: 185 - DNA, 24 bp, Artificial Sequence
  Forward primer derived from L segment of LACV genome
tcaaggattaaagtcgggcttgac

SEQ ID NO: 186 - DNA, 24 bp, Artificial Sequence
  Forward primer derived from L segment of LACV genome
ttcaaggattaaagtcgggcttga

SEQ ID NO: 187 - DNA, 24 bp, Artificial Sequence
  Reverse primer derived from L segment of LACV genome
cggacagaaactctaacccatcat

SEQ ID NO: 188 - DNA, 25 bp, Artificial Sequence
  Reverse primer derived from L segment of LACV genome
cggacagaaactctaacccatcatt

SEQ ID NO: 189 - DNA, 24 bp, Artificial Sequence
  Reverse primer derived from L segment of LACV genome
tcggacagaaactctaacccatca

SEQ ID NO: 190 - DNA, 25 bp, Artificial Sequence
  Reverse primer derived from L segment of LACV genome
tcggacagaaactctaacccatcat

SEQ ID NO: 191 - DNA, 25 bp, Artificial Sequence
  Reverse primer derived from L segment of LACV genome
atcggacagaaactctaacccatca

(19) United States
(12) Patent Application Publication
(10) Pub. No.: US 2011/0150911 A1
(43) Pub. Date: Jun. 23, 2011

(54) Methods and reagents for treating, preventing and diagnosing bunyavirus infection

(76) Inventors: Qui-Lim Choo, El Cerrito, CA (US); Michael Houghton, Danville, CA (US); Elizabeth Scott, Sonoma, CA (US); Amy Weiner, Fairfield, CA (US)

(21) Appl. No.: 10/580,050

(22) PCT Filed: Nov. 19, 2004
(86) PCT No.: PCT/US04/39333

§ 371 Date: Aug. 10, 2007

Related U.S. Application Data
(60) Provisional application No. 60/523,572, filed on Nov. 19, 2003.
(60) Provisional application No. 60/541,617, filed on Feb. 3, 2004.

(51) Int. Cl. A61K 39/12; C07K 14/175; C07K 1/14; C07K 1/16; C07K 1/18; C07H 21/04; C12Q 1/70; A61P 37/04; A61P 31/14

(52) U.S. Cl. 424/186.1; 424/204.1; 424/192.1; 530/402; 530/350; 530/412; 530/413; 530/416; 530/417; 536/24.3; 536/24.33; 435/5

(57) ABSTRACT

Immunogenic compositions for use in treating, preventing and diagnosing infection caused by the California (CAL) serotype of the genus Bunyavirus, such as La Crosse virus (LACV), are disclosed. Also described are reagents for use in diagnostic assays.

### CROSS REFERENCE TO RELATED APPLICATIONS

The present application claims the benefit under 35 U.S.C. §119(e) of U.S. Provisional Application No. 60/523,572, filed Nov. 19, 2003, and U.S. Provisional Application No. 60/541,617, filed Feb. 2, 2004, which applications are incorporated herein by reference in their entireties.

### TECHNICAL FIELD

The present invention pertains generally to viruses from the family Bunyaviridae. In particular, the invention relates to immunogenic reagents derived from viruses of the California (CAL) serogroup of the genus Bunyavirus, such as La Crosse virus (LACV), snowshoe hare virus and Tahyna virus, including immunogenic polypeptides and nucleic acids for use in compositions for diagnosis, prevention and treatment of Bunyavirus infection. The invention also relates to vaccine compositions using inactivated or attenuated CAL viruses, such as inactivated or attenuated LACV.

### BACKGROUND

The family of viruses known as the Bunyaviridae includes the California (CAL) serogroup of viruses belonging to the genus Bunyavirus. The CAL viruses are mosquito-borne and infect various wild and domestic mammals, including humans and rodents. Representative members of the CAL serogroup include La Crosse virus (LACV), snowshoe hare virus and Tahyna virus. Each of the CAL viruses has a narrow range of mosquito and mammalian hosts and, until recently, a limited geographic distribution.

For example, the snowshoe hare virus is found in Canada, Alaska and the northern United States and primarily infects snowshoe hares. Tahyna virus, found in central Europe, causes periodic outbreaks of an influenza-like illness in humans, domestic animals and rabbits. LACV generally causes infection in humans and woodland rodents such as chipmunks and squirrels. Human LACV infections are often subclinical but clinical manifestations can range in severity from mild fever to aseptic meningitis or classical acute encephalitis. Infections occur most frequently in children and young adults during the summer months when mosquitoes are active. The virus is considered one of the most important mosquito-borne pathogens in North America. The Midwestern states of Minnesota, Wisconsin, Iowa, Illinois, Indiana and Ohio report over 90% of all cases in the United States. However, the range of LACV infections is expanding to other regions in the United States, including California, North Carolina and Tennessee and is expected to continue to expand. Epidemics of LACV encephalitis and meningitis raise concerns that transmission of the virus may occur through voluntary blood donations.

The CAL viruses are enveloped, minus-sense RNA viruses. The RNA of the viral genome is tripartite, consisting of three fragments generally designated as S, M and L for small, medium and large genome fragments, respectively. The M segment, approximately 4.5 kb, encodes two envelope glycoproteins (G1 and G2) and a nonstructural protein (NSm) in a single open reading frame. G1 contains the principal viral neutralizing epitopes. The S segment encodes a nucleocapsid protein, termed N, and a further nonstructural protein termed NSs, in overlapping reading frames. The L segment of the genome, approximately 6.5 kb in size, encodes an RNA-dependent RNA polymerase. For a further discussion of the Bunyavirus genome see, e.g., Fields Virology, Third Edition (B. N. Fields et al., eds) Lippincott-Raven Publishers, Philadelphia, Pa., chapters 47 and 48.

Vaccinia virus recombinants expressing both LACV G1 and G2 have been reported to generate a protective response directed primarily against G1, whereas vaccinia recombinants expressing only full-length G1 have been shown to be only partially effective at inducing a neutralizing response and at protecting mice from a potentially lethal challenge with LACV. Pekosz et al., J. Virol. (1995) 69:3475-3481. A truncated soluble form of LACV G1 prepared in a baculovirus system has also been reported to be protective in animal models via humoral immunity (i.e., neutralizing antibodies). Pekosz et al., J. Virol. (1995) 69:3475-3481. Plasmid DNA encoding LACV G1 and G2 has been reported to produce neutralizing antibodies in a mouse model of the disease, and to protect against challenge with LACV. However, immunization with DNA encoding LACV protein N yielded only a partial protective effect. Schuh et al., Hum. Gene Ther. (1999) 10:1649-1658; Pavlovic et al., Intervirology (2000) 43:312-321.

The diagnosis of LACV infection in humans has been established by the presence of LACV IgM and/or IgG antibodies in serum or cerebrospinal fluid (CSF) using indirect immunofluorescence. However, detection of antibodies is generally at from one to three weeks after the onset of infection. Moreover, nonspecific antigen-antibody reactions can occur and result in false-positive determinations. Hence, additional methods for successfully diagnosing LACV as well as other CAL serotype infection are greatly needed.

Nevertheless, to date, no effective prevention, treatment or diagnosis of CAL virus infection exists. Currently, public education and mosquito abatement programs are used to curb transmission of the virus. However, rapid intervention is critical in order to reduce the risk to humans. Thus, there remains an urgent need for effective vaccines, as well as for reagents for use as diagnostics for CAL infection, such as LACV infection.

### SUMMARY OF THE INVENTION

The present invention is based on the discovery of novel reagents and methods for treating and diagnosing CAL infection, such as LACV infection. The methods use attenuated or inactivated viruses, subunit compositions, and CAL virus proteins and polynucleotides to treat and detect infection. For example, LACV proteins, polynucleotides encoding the proteins, and combinations thereof, as well as antibodies produced therefrom, can be used in immunogenic compositions for preventing, treating and diagnosing LACV, as well as other CAL viral infections. Recombinant techniques can be used to produce the products described herein to provide protein preparations devoid of other molecules normally present, such as other viral contaminants and harmful proteins.

Accordingly, in one embodiment, the invention is directed to a subunit vaccine composition comprising one or more isolated CAL virus immunogens and a pharmaceutically acceptable vehicle. In certain embodiments, the one or more isolated immunogens are derived from La Crosse virus (LACV). The one or more immunogens are selected from the group consisting of (a) G1, (b) G2, (c) N, (d) NSm, (e) NSs, (f); immunogenic fragments of (b), (c), (d) or (e); and immunogenic analogs of (a), (b), (c), (d), (e) or (f). In certain embodiments, the immunogen comprises the sequence of amino acids depicted at about positions 474-1441 of FIGS. 1A-1E, such as at position 474, 475, 476, 477, 478, 479, 480, 481, 482, 483, 484, 485 to about amino acid 1441, such as to amino acid position 1430, 1431, 1432, 1433, 1434, 1435, 1436, 1437, 1438, 1439, 1440, 1441, or a sequence of amino acids with at least 75% sequence identity thereto, such as with at least 85% or 90% sequence identity thereto.

In additional embodiments, the immunogen comprises the sequence of amino acids depicted at about positions 1-1441 of FIGS. 1A-1E, such as at position 1, 2, 3, 4, 5, 6, 7, 8, 9, 10, 11, 12 to about amino acid 1441, such as to amino acid position 1430, 1431, 1432, 1433, 1434, 1435, 1436, 1437, 1438, 1439, 1440, 1441, or a sequence of amino acids with at least 75% sequence identity thereto, such as with at least 85% or 90% sequence identity thereto. In still further embodiments, the subunit vaccine comprises an immunogenic fusion polypeptide that comprises a LACV envelope polypeptide fused to at least one other CAL virus polypeptide.

In still further embodiments, the G1 polypeptide present in the subunit vaccine composition is one that has been produced recombinantly by expression of a polynucleotide encoding the sequence of amino acids found at positions 1-1441 or 474-1441 of FIGS. 1A-1E. In certain cases, expression of such constructs results in the production of a G1 and G2 polypeptide, with or without the intervening NSm sequence that naturally occurs within the full-length M segment (i.e., expression of the sequence encoding 1-1441) or a G1 polypeptide or a fragment of a G1 polypeptide (i.e., expression of the sequence encoding 474-1441). The coordinates of the G1 and/or G2 polypeptides produced by recombinant expression are not necessarily the coordinates of the polypeptide encoded by the polynucleotide sequence as proteolytic clipping and the like may occur. Accordingly, for the G1 polypeptide that is produced by recombinant expression, the N-terminus may be at about 474, such as at position 474, 475, 476, 477, 478, 479, 480, 481, 482, 483, 484, 485 . . . 490 . . . 500 . . . 510 . . . 525 . . . 550 . . . 575 . . . 600 . . . 650 . . . 700 . . . 750, or any N-terminus between e.g., 474-750, or beyond 750, even if the polynucleotide encodes a polypeptide with the N-terminus at 474. Additionally, the C-terminus will be at about amino acid 1441, such as amino acid 1250 . . . 1300 . . . 1350 . . . 1375 . . . 1400 . . . 1410 . . . 1420 . . . 1430, 1431, 1432, 1433, 1434, 1435, 1436, 1437, 1438, 1439, 1440, 1441, or any C-terminus between e.g., 1250 and 1441, even if the polynucleotide encodes a polypeptide with a C-terminus at 1441. Also intended to be encompassed are sequences of amino acids with at least 75% sequence identity to the sequences above, such as with at least 85% or 90% sequence identity thereto.

In additional embodiments, the invention is directed to an immunogenic composition comprising a CAL virus truncated G1 polypeptide. In certain embodiments, the CAL virus G1 polypeptide is derived from LACV. In particular embodiments, the truncated G1 polypeptide is truncated at a position between about amino acid position 1391 and the C-terminus of the native G1 envelope polypeptide, numbered relative to the G1 polypeptide depicted in FIGS. 1A-1E. In certain embodiments, the truncated G1 polypeptide comprises the sequence of amino acids depicted at about amino acid positions 474-1391 of FIGS. 1A-1E such as position 474, 475, 476, 477, 478, 479, 480, 481, 482, 483, 484, or 485 to about amino acid 1391, such as to amino acid 1389, 1390, 1391, or a sequence of amino acids with at least 75% sequence identity thereto, such as with at least 85% or 90% sequence identity thereto.

In still further embodiments, the truncated G1 polypeptide present in the immunogenic composition is one that has been produced recombinantly by expression of a polynucleotide encoding the sequence of amino acids found at positions 474-1391 of FIGS. 1A-1E. In certain cases, recombinant expression of such a construct results in the production of a truncated G1 polypeptide with coordinates that are different than the coordinates of the truncated G1 polypeptide encoded by the polynucleotide sequence due to proteolytic clipping that might occur during recombinant production. Accordingly, the N-terminus for the truncated G1 polypeptide may be at e.g., position 474, 475, 476, 477, 478, 479, 480, 481, 482, 483, 484, 485, . . . 490 . . . 500 . . . 510 . . . 525 . . . 550 . . . 575 . . . 600 . . . 650 . . . 700 . . . 750, or any N-terminus between e.g., 474-750, or beyond 750, even if the polynucleotide used to produce the molecule encodes amino acids 474-1391, and the C-terminus may be at, e.g., amino acid 1200 . . . 1250 . . . 1300 . . . 1325 . . . 1350 . . . 1360 . . . 1370 . . . 1375 . . . 1389, 1390, 1391. Also intended to be encompassed are those sequences with at least 75% sequence identity thereto, such as with at least 85% or 90% sequence identity thereto.

In still further embodiments, the invention is directed to an immunogenic composition comprising at least one isolated CAL virus immunogen, wherein the immunogen is produced intracellularly. In certain embodiments, the CAL virus immunogen is a LACV immunogen. In additional embodiments, the immunogen is one or more immunogens selected from the group consisting of (a) G1, (b) G2, (c) N, (d) NSm, (e) NSs, (f); immunogenic fragments of (a), (b), (c), (d) or (e); and immunogenic analogs of (a), (b), (c), (d), (e) or (f). In certain embodiments, the composition comprises a full-length G1 and/or a truncated G1 polypeptide. In yet further embodiments, the truncated G1 polypeptide comprises a deletion of all or part of a transmembrane binding domain. In additional embodiments, the truncated G1 polypeptide further comprises a deletion of all or part of the cytoplasmic tail. In yet further embodiments, the truncated G1 polypeptide comprises all or part of the cytoplasmic tail.

In certain embodiments, the intracellularly produced, truncated G1 polypeptide is truncated at a position between about amino acid position 1387 or about 1391 and the C-terminus of the native G1 envelope polypeptide, numbered relative to the G1 polypeptide depicted in FIGS. 1A-1E. In additional embodiments, the truncated G1 polypeptide comprises the sequence of amino acids depicted at about amino acid positions 474 to 1387 or about amino acid positions 474-1391 of FIGS. 1A-1E. For example, the N-terminus of the G1 polypeptide may be at position 460, 461, 462, 463, 464, 465, 466, 467, 468, 469, 470, 471, 472, 473, 474, 475, 476, 477, 478, 479, 480, 481, 482, 483, 484, or 485 and the C-terminus may be at about position be at, e.g., amino acid 1200 . . . 1250 . . . 1300 . . . 1325 . . . 1350 . . . 1360 . . . 1370 . . . 1375 . . . 1389, 1390, 1391, or a sequence of amino acids with at least 75% sequence identity to these sequences such as with at least 85% or 90% sequence identity thereto.

In further embodiments, the intracellularly produced, truncated G1 polypeptide comprises a deletion of amino acids 1388-1419 or amino acids 1392-1419, numbered relative to the G1 polypeptide depicted in FIGS. 1A-1E. In yet additional embodiments, the immunogenic composition comprises the protein product of a CAL virus M region. In certain embodiments, the immunogenic composition comprises the sequence of amino acids depicted at about positions 1-1441 or about positions 474-1441 of FIGS. 1A-1E.

In still further embodiments, the intracellular immunogen present in the composition is one that has been produced recombinantly by expression of a polynucleotide encoding the sequence of amino acids found at positions 1-1441, 474-1441, 474-1387 or 474-1391 of FIGS. 1A-1E. For example, in certain cases, expression of a construct encoding the entire M segment, i.e., expression of a construct encoding amino acids 1-1441 of FIGS. 1A-1E, results in the production of a G1 and G2 polypeptide, with or without the intervening NSm sequence that naturally occurs within the full-length M segment. Thus, for example, the expressed protein can be processed intracellularly to result in a G1/G2 complex lacking the NSm sequence. Moreover, the sequence for the G1 polypeptide or truncated G1 polypeptide may also be proteolytically cleaved during recombinant production to result in a sequence significantly shorter than the coding sequence originally present in the construct. Thus, the coordinates of the G1 and/or G2 polypeptides produced by recombinant expression are not necessarily the coordinates of the polypeptide encoded by the polynucleotide sequence. Accordingly, for the G1 polypeptide or C-terminally truncated G1 polypeptide, the N-terminus may be at position 474, 475, 476, 477, 478, 479, 480, 481, 482, 483, 484, 485, . . . 490 . . . 500 . . . 510 . . . 525 . . . 550 . . . 575 . . . 600 . . . 650 . . . 700 . . . 750, or any N-terminus between e.g., 474-750, or beyond 750, even if the polynucleotide encodes a polypeptide with the N-terminus at 474. Additionally, the C-terminus for the full-length molecule may be at amino acid 1250 . . . 1300 . . . 1350 . . . 1375 . . . 1400 . . . 1410 . . . 1420 . . . 1430, 1431, 1432, 1433, 1434, 1435, 1436, 1437, 1438, 1439, 1440, 1441, or any C-terminus between e.g., 1250 and 1441, even if the polynucleotide encodes a polypeptide with a C-terminus at 1441. The C-terminus of the C-terminally truncated molecule may be at position 1200 . . . 1250 . . . 1300 . . . 1325 . . . 1350 . . . 1360 . . . 1370 . . . 1375 . . . 1387 . . . 1388 . . . 1389, 1390, 1391.1387, 1389, 1390, 1391, 1392, 1393, 1394. Similarly, the polypeptide produced intracellularly from a polynucleotide encoding the entire M region will not necessarily begin with amino acid 1 as depicted in FIG. 1 and will not necessarily end at amino acid 1441, but may end at amino acid 1250 . . . 1300 . . . 1350 . . . 1375 . . . 1400 . . . 1410 . . . 1420 . . . 1430, 1431, 1432, 1433, 1434, 1435, 1436, 1437, 1438, 1439, 1440, 1441, or any C-terminus between e.g., 1250 and 1441, even if the polynucleotide encodes a polypeptide with a C-terminus at 1441.

In additional embodiments, the invention is directed to an immunogenic composition comprising an inactivated CAL virus, or an attenuated CAL virus, and a pharmaceutically acceptable vehicle. In certain embodiments, the CAL virus is LACV.

In further embodiments, the invention is directed to a method of treating or preventing CAL virus infection in a mammalian subject, such as LACV infection, comprising administering to the subject a therapeutically effective amount of any one of the compositions described above.

In additional embodiments, the invention is directed to a method of producing an immunogenic composition comprising the steps of

(a) providing an inactivated or attenuated CAL virus; and

(b) combining the inactivated or attenuated CAL virus with a pharmaceutically acceptable vehicle.

In further embodiments, the invention is directed to a method of producing a subunit vaccine composition comprising the steps of

(a) providing one or more CAL virus immunogens, wherein the one or more immunogens are selected from the group consisting of (a) G1, (b) G2, (c) N, (d) NSm, (e) NSs, (f); immunogenic fragments of (b), (c), (d) or (e); and immunogenic analogs of (a), (b), (c), (d), (e) or (f); and

(b) combining the CAL virus immunogen(s) with a pharmaceutically acceptable vehicle.

In additional embodiments, the invention is directed to a method of producing an immunogenic composition comprising the steps of

(a) providing a CAL virus immunogen, wherein said immunogen is produced intracellularly

(b) combining the CAL virus immunogen with a pharmaceutically acceptable vehicle.

In still further embodiments, the invention is directed to a method of producing an immunogenic composition comprising the steps of

(a) providing a CAL virus truncated G1 polypeptide, wherein the truncated G1 polypeptide is truncated at a position between amino acid position 1391 and the C-terminus of the native G1 envelope polypeptide, numbered relative to the G1 polypeptide depicted in FIGS. 1A-1E; and

(b) combining the CAL virus truncated G1 polypeptide with a pharmaceutically acceptable vehicle.

In additional embodiments, the invention is directed to a method for isolating an immunogenic CAL virus envelope polypeptide comprising:

(a) providing a population of mammalian host cells that express the envelope polypeptide intracellularly;

(b) recovering s membrane component of the cells;

(c) treating the membrane component with a non-ionic detergent, thereby to solubilize the membrane component and release the envelope polypeptide; and

(d) isolating the released envelope polypeptide.

In certain embodiments, the isolating step comprises at least one column purification step wherein the column is selected from the group consisting of a lectin affinity column, a hydroxyapatite column and an ion exchange column. In further embodiments, the isolating step comprises: (i) binding the released envelope polypeptide to the ion exchange column, such as a lectin affinity column; and (ii) eluting the bound envelope polypeptide from the ion exchange column. In certain embodiments, the ion exchange column is a cation exchange column. In any of these embodiments, the lectin affinity column can be a concanavalin A lectin column. Additionally, the mammalian cells can be CHO or HEK293 cells. In further embodiments, the CAL virus envelope polypeptide is a G1 and/or a G2 polypeptide, and optionally includes all or a portion of the NSm polypeptide.

In additional embodiments, the invention is directed to an immunogenic composition comprising an envelope polypeptide obtained by the method of intracellular production detailed above.

In yet further embodiments, the invention is directed to a CAL virus truncated G1 polypeptide, for example, a LACV truncated G1 polypeptide. In certain embodiments, the truncated G1 polypeptide is truncated at a position between amino acid position 1391 and the C-terminus of the native G1 envelope polypeptide, numbered relative to the G1 polypeptide depicted in FIGS. 1A-1E. In additional embodiments, the polypeptide comprises the sequence of amino acids depicted at amino acid positions 474-1391 of FIGS. 1A-1E.

In further embodiments, the invention is directed to an isolated oligonucleotide not more than 60 nucleotides in length comprising:

(a) a nucleotide sequence of at least 10 contiguous nucleotides from a probe or primer sequence depicted in any of FIG. 5, 6 or 7;

(b) a nucleotide sequence having 90% sequence identity to a nucleotide sequence of (a); or

(c) complements of (a) and (b).

In additional embodiments, the invention is directed to an isolated oligonucleotide selected from the group consisting of: (a) the oligonucleotide of SEQ ID NO:7, (b) the oligonucleotide of SEQ ID NO:8, (c) the oligonucleotide of SEQ ID NO:9, (d) the oligonucleotide of SEQ ID NO:10, (e) the oligonucleotide of SEQ ID NO:11, (f) the oligonucleotide of SEQ ID NO:12, (g) the oligonucleotide of SEQ ID NO:13, (h) the oligonucleotide of SEQ ID NO:14, (i) the oligonucleotide of SEQ ID NO:15, (j) SEQ ID NO:16, complements of (a), (b), (c), (d), (e), (f), (g), (h), (i) or (j), and reverse complements of (a), (b), (c), (d), (e), (f), (g), (h), (i) or (j):

In certain embodiments, the nucleotide sequence above is a probe sequence and further comprises a detectable label at the 5′-end and/or the 3′-end, such as a fluorescent label selected from the group consisting of 6-carboxyfluorescein (6-FAM), tetramethyl rhodamine (TAMRA), and 2′,4′,5′,7′,-tetrachloro-4-7-dichlorofluorescein (TET).

In additional embodiments, the invention is directed to a method for detecting CAL virus infection in a biological sample. The method comprises:

(a) isolating nucleic acid from a biological sample suspected of containing CAL virus RNA, wherein if CAL virus is present, said nucleic acid comprises a target sequence;

(b) reacting the CAL virus nucleic acid with a detectably labeled probe sufficiently complementary to and capable of hybridizing with the target sequence, wherein said reacting is done under conditions that provide for the formation of a probe/target sequence complex; and

(c) detecting the presence or absence of label as an indication of the presence or absence of the target sequence.

In additional embodiments, the invention is directed to a method for detecting La Crosse virus (LACV) infection in a biological sample. The method comprises:

(a) isolating nucleic acid from a biological sample suspected of containing LACV RNA, wherein if LACV is present, said nucleic acid comprises a target sequence;

(b) reacting the LACV nucleic acid with a detectably labeled probe sufficiently complementary to and capable of selectively hybridizing with the target sequence, wherein said reacting is done under conditions that provide for the formation of a probe/target sequence complex; and

(c) detecting the presence or absence of label as an indication of the presence or absence of the target sequence.

In certain embodiments, the probe is selected from the group consisting of (a) the oligonucleotide of SEQ ID NO:8, (b) the oligonucleotide of SEQ ID NO:9, (c) the oligonucleotide of SEQ ID NO:12, (d) the oligonucleotide of SEQ ID NO:16, complements of (a), (b), (c) or (d), and reverse complements of (a), (b), (c) or (d).

In additional embodiments, the invention is directed to a method for detecting CAL virus infection in a biological sample. The method comprises:

isolating nucleic acids from a biological sample suspected of containing CAL virus;

amplifying the nucleic acids using at least two primers wherein (a) each of the primers is not more than about 50 nucleotides in length and each of the primers is sufficiently complementary to a portion of the sense and antisense strands, respectively, of CAL virus isolated nucleic acid, if present, to hybridize therewith; and

detecting the presence of the amplified nucleic acids as an indication of the presence or absence of CAL virus in the sample.

In certain embodiments, the amplifying comprises RT-PCR, transcription-mediated amplification (TMA) or a fluorogenic 5′ nuclease assay, or a combination thereof. In additional embodiments, the amplifying uses a fluorogenic 5′ nuclease assay using the sense primer and the antisense primer and detecting is done using at least one detectably labeled probe sufficiently complementary to and capable of hybridizing with the CAL virus nucleic acid if present.

In yet further embodiments, the invention is directed to a method for detecting La Crosse virus (LACV) infection in a biological sample. The method comprises:

isolating nucleic acids from a biological sample suspected of containing LACV wherein if LACV is present, said nucleic acid comprises a target sequence;

amplifying the nucleic acids using at least two primers wherein (a) each of the primers is not more than about 50 nucleotides in length and each of the primers is sufficiently complementary to a portion of the sense and antisense strands, respectively, of LACV isolated nucleic acid, if present, to hybridize therewith, and further wherein at least one of the primers is capable of selectively hybridizing to the target sequence; and

detecting the presence of the amplified nucleic acids as an indication of the presence or absence of LACV in the sample.

In certain embodiments, the amplifying comprises RT-PCR, transcription-mediated amplification (TMA) or a fluorogenic 5′ nuclease assay, or a combination thereof. In additional embodiments, the amplifying uses a fluorogenic 5′ nuclease assay using the sense primer and the antisense primer and detecting is done using at least one detectably labeled probe sufficiently complementary to and capable of hybridizing with the LACV nucleic acid if present. In still further embodiments, one of the primers is selected from the group consisting of (a) the oligonucleotide of SEQ ID NO:8, (b) the oligonucleotide of SEQ ID NO:9, (c) the oligonucleotide of SEQ ID NO:12, (d) the oligonucleotide of SEQ ID NO:16, complements of (a), (b), (c) or (d), and reverse complements of (a), (b), (c) or (d).

In additional embodiments, the invention is directed to a method for detecting La Crosse virus (LACV) infection in a biological sample. The method comprises:

isolating nucleic acids from a biological sample suspected of containing LACV wherein if LACV is present, said nucleic acid comprises a target sequence;

amplifying the nucleic acids using at least two primers wherein (a) each of the primers is not more than about 50 nucleotides in length and each of the primers is sufficiently complementary to a portion of the sense and antisense strands, respectively, of LACV isolated nucleic acid, if present, to hybridize therewith; and

detecting thepresence of the amplified nucleic acids using at least one detectably labeled probe sufficiently complementary to and capable of hybridizing with the LACV nucleic acid if present, as an indication of the presence or absence of LACV in the sample, wherein at least one of the primers and/or the probe is capable of selectively hybridizing to the target sequence.

In certain embodiments, one of the primers is selected from the group consisting of (a) the oligonucleotide of SEQ ID NO:8, (b) the oligonucleotide of SEQ ID NO:9, (c) the oligonucleotide of SEQ ID NO:12, (d) the oligonucleotide of SEQ ID NO:16, complements of (a), (b), (c) or (d), and reverse complements of (a), (b), (c) or (d).

In yet additional embodiments, the invention is directed to a kit for detecting a CAL virus infection in a biological sample. The kit comprises:

primer oligonucleotides wherein the primer oligonucleotides are not more than about 60 nucleotides in length, wherein each of the primers is sufficiently complementary to a portion of the sense and antisense strands, respectively, to CAL virus nucleic acid to hybridize therewith; and

written instructions for identifying the presence of a CAL virus. In certain embodiments, the kit further comprises a polymerase and buffers. The kit can also comprise at least one detectably labeled probe oligonucleotide of not more than about 60 nucleotides in length and sufficiently complementary to and capable of hybridizing with CAL virus nucleic acid.

In additional embodiments, the invention is directed to a kit for detecting a La Crosse virus (LACV) infection in a biological sample. The kit comprises:

primer oligonucleotides wherein the primer oligonucleotides are not more than about 60 nucleotides in length, wherein each of the primers is sufficiently complementary to a portion of the sense and antisense strands, respectively, to LACV nucleic acid to hybridize therewith and further wherein at least one of the primers is capable of selectively hybridizing to LACV nucleic acid; and

written instructions for identifying the presence of a LACV. In certain embodiments, the kit further comprises a polymerase and buffers. In additional embodiments, one of the primers is selected from the group consisting of (a) the oligonucleotide of SEQ ID NO:8, (b) the oligonucleotide of SEQ ID NO:9, (c) the oligonucleotide of SEQ ID NO:12, (d) the oligonucleotide of SEQ ID NO:16, complements of (a), (b), (c) or (d), and reverse complements of (a), (b), (c) or (d). In yet further embodiments, the kit further comprises at least one detectably labeled probe oligonucleotide of not more than about 60 nucleotides in length and sufficiently complementary to and capable of hybridizing with LACV nucleic acid.

In another embodiment, the invention is directed to a kit for detecting a La Crosse virus (LACV) infection in a biological sample. The kit comprises:

primer oligonucleotides wherein the primer oligonucleotides are not more than about 60 nucleotides in length, wherein each of the primers is sufficiently complementary to a portion of the sense and antisense strands, respectively, to LACV nucleic acid to hybridize therewith;

at least one detectably labeled probe oligonucleotide of not more than about 60 nucleotides in length and sufficiently complementary to and capable of hybridizing with LACV nucleic acid, wherein at least one of the primers and/or the probe is capable of selectively hybridizing to the target sequence; and

written instructions for identifying the presence of LACV.

In certain embodiments, the kit further comprises a polymerase and buffers. In additional embodiments, one of the primers and/or probes is selected from the group consisting of (a) the oligonucleotide of SEQ ID NO:8, (b) the oligonucleotide of SEQ ID NO:9, (c) the oligonucleotide of SEQ ID NO: 12, (d) the oligonucleotide of SEQ ID NO:16, complements of (a), (b), (c) or (d), and reverse complements of (a), (b), (c) or (d).

These and other embodiments of the subject invention will readily occur to those of skill in the art in view of the disclosure herein.

### BRIEF DESCRIPTION OF THE FIGURES

FIGS. 1A-1E (SEQ ID NOS:1 and 2) show a representative nucleotide sequence and corresponding amino acid sequence for the La Crosse virus M segment, encoding the G1, G2 and NSm proteins. The sequence is from strain Human/78 (NCBI accession no. NC 004109). The boundaries between the proteins are shown by double slashes. The amino acid sequence for G2 spans amino acid position 1 to amino acid position 299 (nucleotide positions 62-958); the amino acid sequence for NSm runs from position 300 to about position 473 (nucleotide positions 959-1480) and includes the native leader for the G1 sequence. The amino acid sequence for G1 includes amino acids 474-1441 (nucleotide positions 1481-4383).

FIGS. 2A-2B (SEQ ID NOS: 3 and 4) show a representative nucleotide sequence for the La Crosse virus S segment and shows the corresponding amino acid sequences for the nucleocapsid (N) protein and the non-structural protein (NSs) which occur in overlapping reading frames. The sequence is from strain Human/78 (NCBI accession no. NC 004110).

FIGS. 3A-3H (SEQ ID NOS:5 and 6) show a representative nucleotide sequence and corresponding amino acid sequence for the La Crosse virus L segment, encoding the RNA-dependent RNA polymerase. The sequence is from strain Human/78 (NCBI accession no. NC 004108). The coding sequence for the polymerase is found at nucleotide positions 62-6849.

FIGS. 4A-4F show representative strategies using primers and probes for detection of LACV in nucleotide-based assays. FIG. 4A is a diagrammatic representation of the LACV viral genomic structure. FIGS. 4B-4F show representative nucleic acid-based assay formats.

FIGS. 5A-5O show representative forward (sense) and reverse (antisense) primers, as well as probes, derived from the M segment of the LACV genome, for use in diagnostic assays described herein. Forward primers are shown in FIGS. 5A-5E; reverse primers for use with the forward primers are shown on the corresponding lines in FIGS. 5K-5O; probes for use with the primer pairs shown in FIGS. 5A-5E and 5K-5O are shown on the corresponding lines in FIGS. 5F-5J.

FIGS. 6A-6O show representative forward (sense) and reverse (antisense) primers, as well as probes, derived from the S segment of the LACV genome, for use in diagnostic assays described herein. Forward primers are shown in FIGS. 6A-6E; reverse primers for use with the forward primers are shown on the corresponding lines in FIGS. 6K-6O; probes for use with the primer pairs shown in FIGS. 6A-6E and 6K-6O are shown on the corresponding lines in FIGS. 6F-6J.

FIGS. 7A-7F show representative forward (sense) and reverse (antisense) primers, as well as probes, derived from the L segment of the LACV genome, for use in diagnostic assays described herein. Forward primers are shown in FIGS. 7A-7B; reverse primers for use with the forward primers are shown on the corresponding lines in FIGS. 7E-7F; probes for use with the primer pairs shown in FIGS. 7A-7B and 7E-7F are shown on the corresponding lines in FIGS. 7C-7D.

FIG. 8 is a flow-chart for the purification of envelope proteins from intracellularly produced LACM; intracellularly produced truncated LACV G1 (LACV-G1-1391his-internal); and secreted truncated LACV G1 (LACV-G1-1391his).

FIGS. 9A and 9B are representations of Western blots of lysates of pCMVIII COS7 cells expressing LACM (M) or pCMVIII vector without inserts (C) probed with either mouse sera immunized with LACM purified protein (9A) or control pre-bleed sera (9B). Control lanes are on the left of each panel and LACM (M) lanes are on the right side of each panel. Chemicon mouse mAb against G1 (G1mAb) was used as a control to identify the LACV G1 protein (approximately 125 Kd).

FIGS. 10A and 10B are representations of Western blots of lysates of pCMVIII COS7 cells expressing LACM or pCMVIII vector without inserts (C) probed with either mouse sera immunized with internal LAC-G11391his purified protein (10A) or control pre-bleed sera (10B). Control lanes are on the left of each panel and LACM (M) lanes are on the right side of each panel. Chemicon mouse mAb against G1 (G1mAb) was used as a control to identify the LACV G1 protein (approximately 125 Kd).

FIGS. 11A and 11B are representations of Western blots of lysates of pCMVIII COS7 cells expressing LACM or pCMVIII vector without inserts (C) probed with either mouse sera immunized with secreted LAC-G11391his purified protein (11A) or control pre-bleed sera (11B). Control lanes are on the left of each panel and LACM (M) lanes are on the right side of each panel. Chemicon mouse mAb against G1 (G1mAb) was used as a control to identify the LACV G1 protein (approximately 125 Kd).

FIGS. 12A and 12B are representations of Western blots of lysates of pCMVIII COST cells expressing LACM probed with human (FIG. 12A) and mouse (FIG. 12B) antisera.

### DETAILED DESCRIPTION OF THE INVENTION

The practice of the present invention will employ, unless otherwise indicated, conventional methods of virology, chemistry, biochemistry, recombinant DNA techniques and immunology, within the skill of the art. Such techniques are explained fully in the literature. See, e.g., Fundamental Virology, 3rd Edition, vol. I & II (B. N. Fields and D. M. Knipe, eds.); Handbook of Experimental Immunology, Vols. I-IV (D. M. Weir and C. C. Blackwell eds., Blackwell Scientific Publications); T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entireties.

The following amino acid abbreviations are used throughout the text:
Alanine: Ala (A) Arginine: Arg (R)
Asparagine: Asn (N) Aspartic acid: Asp (D)
Cysteine: Cys (C) Glutamine: Gln (O)
Glutamic acid: Glu (E) Glycine: Gly (G)
Histidine: His (H) Isoleucine: Ile (I)
Leucine: Leu (L) Lysine: Lys (K)
Methionine: Met (M) Phenylalanine: Phe (F)
Proline: Pro (P) Serine: Ser (S)
Threonine: Thr (T) Tryptophan: Trp (W)
Tyrosine: Tyr (Y) Valine: Val (V)

### 1. DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a G1 polypeptide” includes a mixture of two or more such polypeptides, and the like.

The terms “polypeptide” and “protein” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation and the like. Furthermore, for purposes of the present invention, a “polypeptide” refers to a protein which includes modifications, such as deletions, additions and substitutions (generally conservative in nature), to the native sequence, so long as the protein maintains the desired activity. These modifications may be deliberate, as through site-directed mutagenesis, or may be accidental, such as through mutations of hosts which produce the proteins or errors due to PCR amplification.

A CAL polypeptide is a polypeptide, as defined above, derived from a virus of the CAL serotype of the genus Bunyavirus, including, without limitation, any of the various isolates of the California encephalitis group of viruses such as LACV, snowshoe hare virus, Tahyna virus, San Angelo virus, Lumbo virus and Inkoo virus; any of the various isolates of the Melao viruses such as Jamestown Canyon virus, South River virus, Keystone virus and Serra do Navio virus; as well as any of the isolates of the Trivittatus and Guaroa group of viruses. The polypeptide need not be physically derived from the particular isolate in question, but may be synthetically or recombinantly produced.

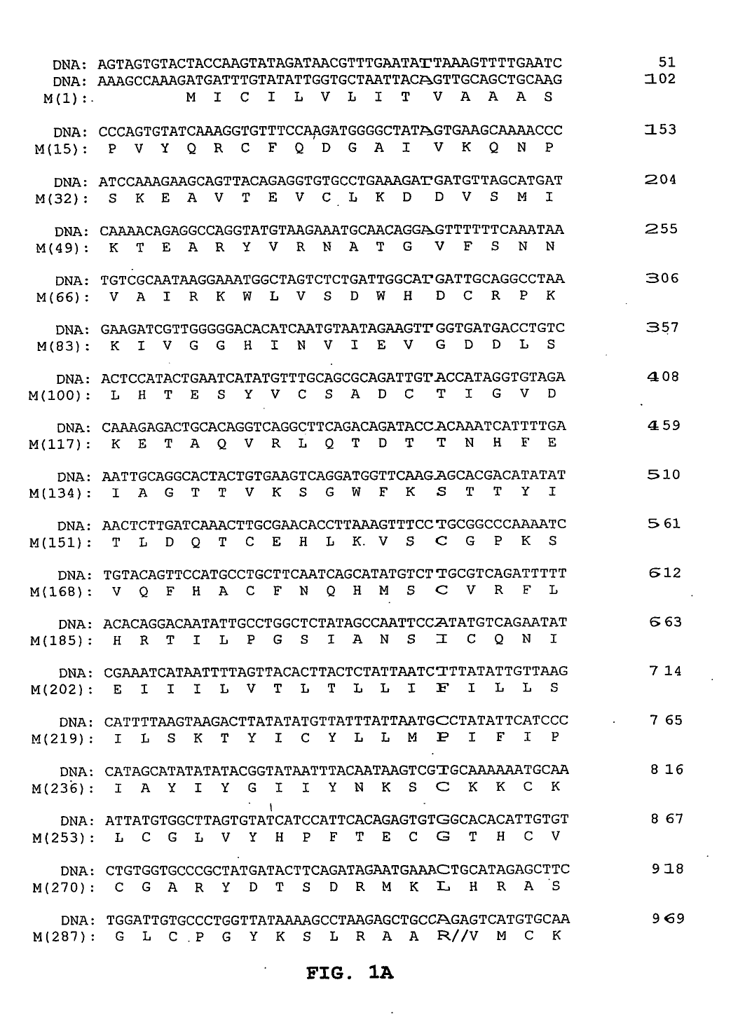

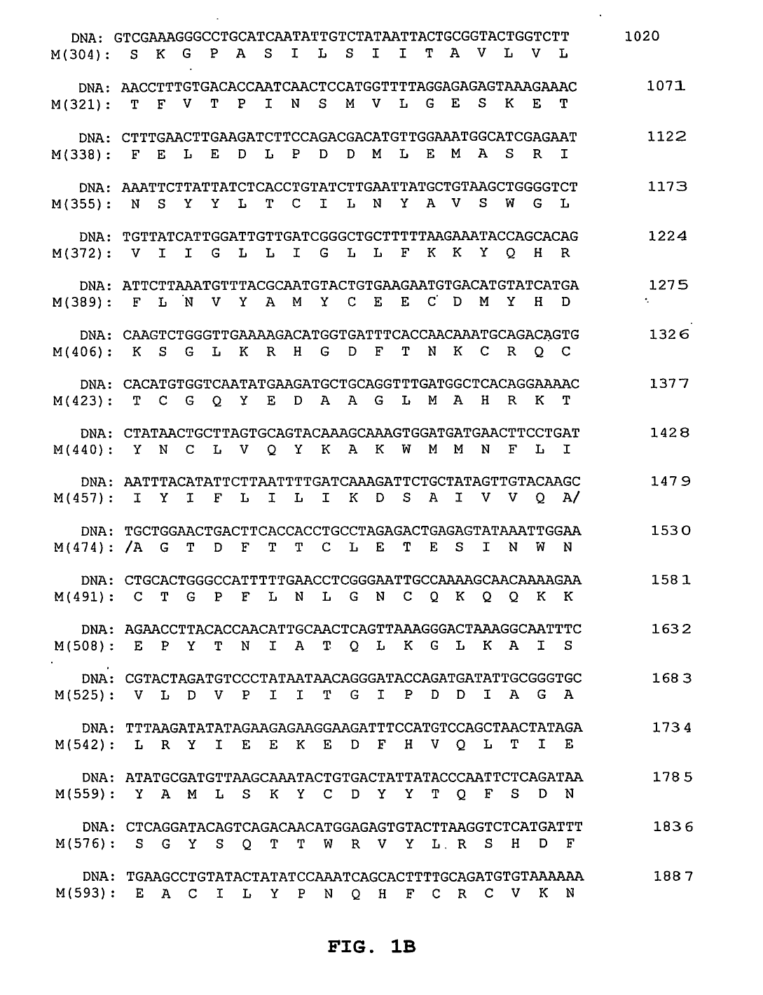

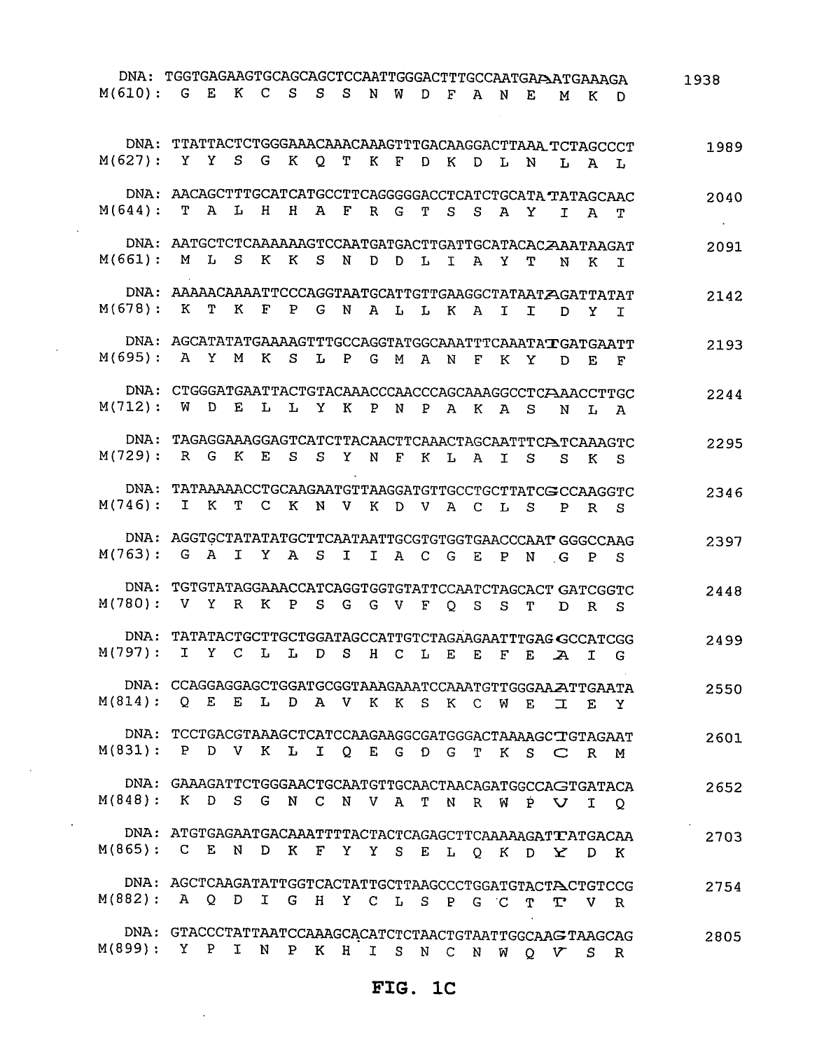

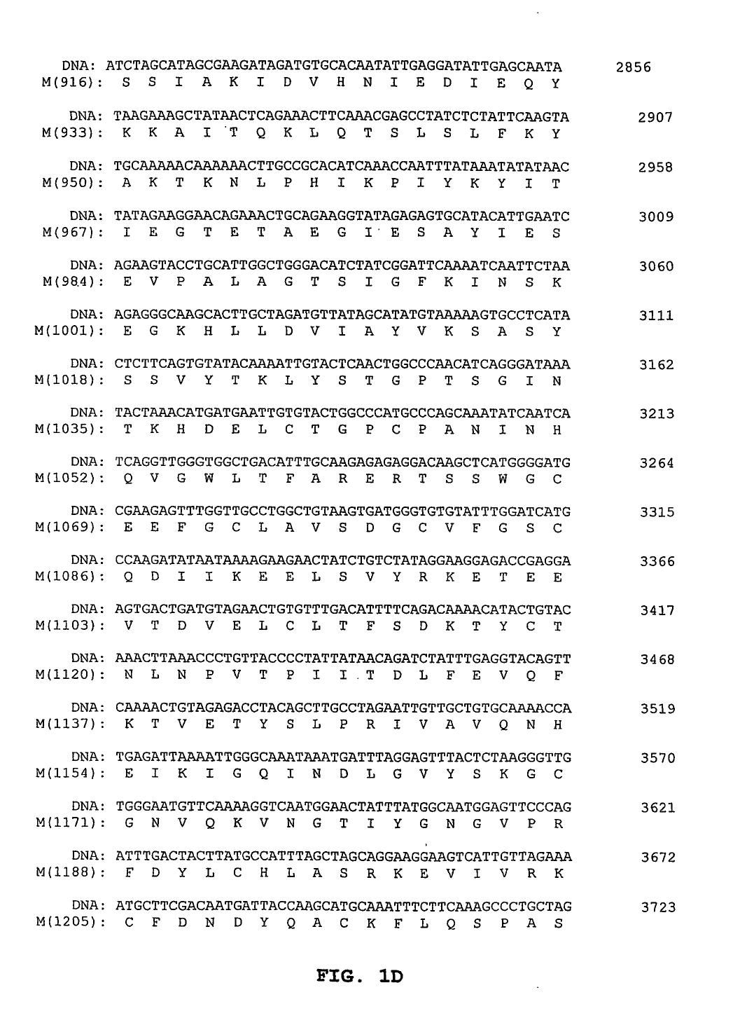

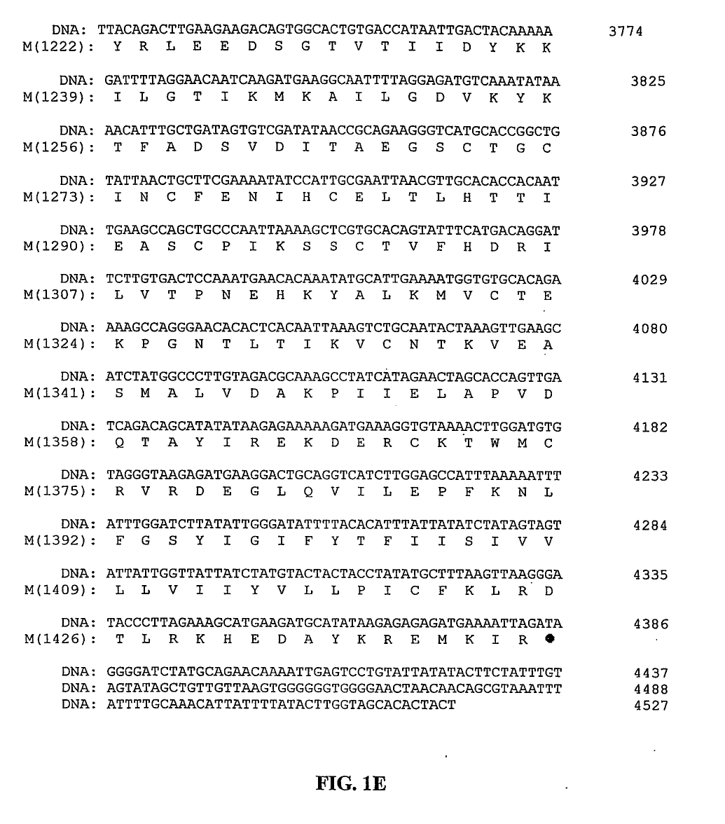

Sequences for polypeptides and the nucleic acid sequences encoding therefor for a number of CAL isolates are known. Representative sequences are presented in FIGS. 1-3 herein for LACV polypeptides. Similarly, representative snowshoe hare virus sequences are found in NCBI Accession numbers J02390 and K02539 (S and M regions, respectively). Representative Tahyna virus sequences are found in NCBI Accession numbers Z68497 and U47142 (each including sequences for the S region); and AF229129 and AF123485 (each including sequences for the M region). See, also Campbell et al., Virus Res. (1999) 61:137-144, for a comparison of M RNA among 15 CAL serogroup viruses.

The terms “analog” and “mutein” refer to biologically active derivatives of the reference molecule, that retain desired activity, such as immunoreactivity in assays described herein, and/or the capability of eliciting an immune response as defined below, such as the ability to elicit neutralizing antibodies. In general, the term “analog” refers to compounds having a native polypeptide sequence and structure with one or more amino acid additions, substitutions (generally conservative in nature) and/or deletions, relative to the native molecule, so long as the modifications do not destroy immunogenic activity and which are “substantially homologous” to the reference molecule as defined below. A number of conserved and variable regions are known between the various isolates and, in general, the amino acid sequences of epitopes derived from these regions will have a high degree of sequence homology, e.g., amino acid sequence homology of more than 50%, generally more than 60%-70%, when the two sequences are aligned. The term “mutein” refers to peptides having one or more peptide mimics (“peptoids”), such as those described in International Publication No. WO 91/04282. Preferably, the analog or mutein has at least the same immunoreactivity as the native molecule. Methods for making polypeptide analogs and muteins are known in the art and are described further below.

Particularly preferred analogs include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25, 50 or 75 conservative or non-conservative amino acid substitutions, or any integer between 5-75, so long as the desired function of the molecule remains intact. One of skill in the art can readily determine regions of the molecule of interest that can tolerate change by reference to Hopp/Woods and Kyte-Doolittle plots, well known in the art.

By “fragment” is intended a polypeptide consisting of only a part of the intact full-length polypeptide sequence and structure. The fragment can include a C-terminal deletion an N-terminal deletion, and/or an internal deletion of the native polypeptide.

By a “G polypeptide” is meant a polypeptide, as defined above, encoded by the M region of the CAL virus in question. As explained above, the M region encodes the G1 and G2 polypeptides, as well as the NSm polypeptide. The nucleotide and corresponding amino acid sequences for various CAL virus M regions are known. For example, the nucleotide sequence and corresponding amino acid sequence for a LACV M region is shown in FIG. 1 herein. Additionally, the M segment of a snowshoe hare virus is reported in NCBI Accession number K02539. The M regions from representative Tahyna viruses are reported in NCBI Accession numbers AF229129 and AF123485. See, also Campbell et al., Virus Res. (1999) 61:137-144, for a comparison of M RNA among 15 CAL serogroup viruses.

As explained above, G1 and/or G2 polypeptides for use with the present invention include the full-length or substantially full-length proteins, as well as fragments, fusions of G1 and G2 polypeptides, or mutants of the proteins, which include one or more epitopes such that immunological activity is retained. For example, a full-length LACV G2 polypeptide will normally include an amino acid sequence corresponding to the sequence depicted at amino acid position 1 to amino acid position 299 of FIG. 1 (nucleotide positions 62-958) and can optionally extend into the NSm region. A full-length G1 polypeptide will generally include at least an amino acid sequence corresponding to the sequence depicted at position 474 to amino acid position 1441 of FIG. 1 (nucleotide positions 1481-4383), and can optionally include the native signal sequence and all or part of the NSm sequence found upstream of the G1 sequence. Moreover, the polypeptide can include deletions of all or part of the transmembrane binding domain, with or without the cytoplasmic tail remaining intact. Representative G1 polypeptides for use with the present invention are detailed below.

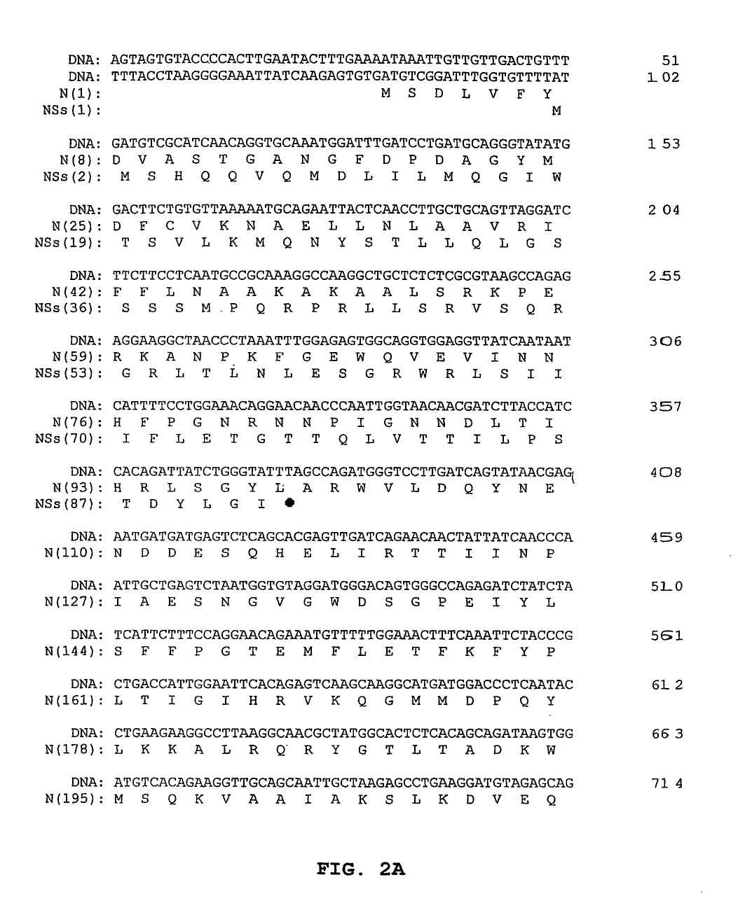

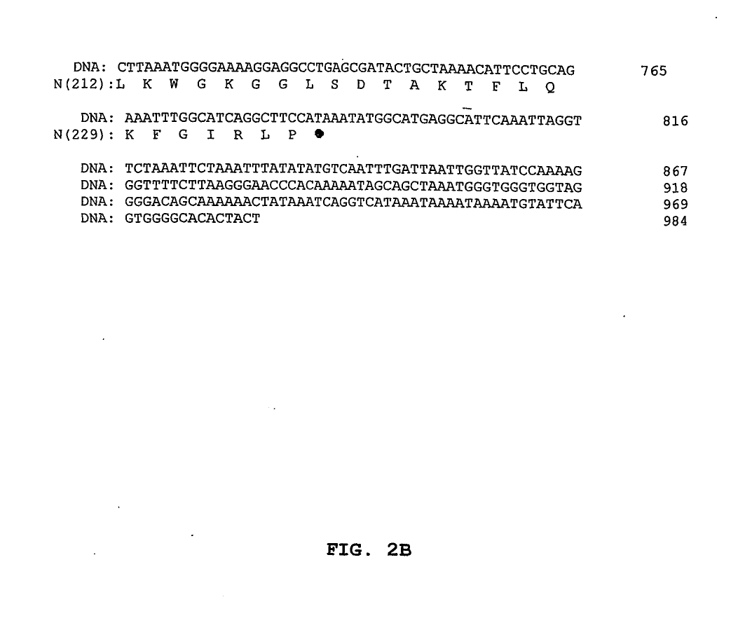

By “N polypeptide” and “NSs polypeptide” is meant the nucleocapsid and nonstructural polypeptides, respectively, derived from the S segment of a CAL genome. By “NSm polypeptide” is meant the nonstructural protein encoded by the M region of a CAL genome. As explained above, the nucleotide and corresponding amino acid sequences for various M regions are known. Similarly, the nucleotide and corresponding amino acid sequences for various CAL N and NSs polypeptides are known. For example, the nucleotide sequence and corresponding amino acid sequences for LACV N and NSs polypeptides are shown in FIG. 2 herein. Additionally, the S segment from a snowshoe hare virus is described in NCBI Accession no. J02390 and the S segment from representative Tahyna viruses are found in NCBI Accession numbers Z68497 and U47142. As explained above, N and NS polypeptides for use in the present invention include the full-length or substantially full-length proteins, as well as fragments, fusions or mutants of the proteins, which include one or more epitopes such that immunological activity is retained.

An “antigen” refers to a molecule, such as a polypeptide as defined above, containing one or more epitopes (either linear, conformational or both) that will stimulate a host's immune system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 145 amino acids. The term “antigen” denotes both subunit antigens, (i.e., antigens which are separate and discrete from a whole organism with which the antigen is associated in nature), as well as, killed, attenuated or inactivated viruses. Antibodies such as anti-idiotype antibodies, or fragments thereof, and synthetic peptide mimotopes, which can mimic an antigen or antigenic determinant, are also captured under the definition of antigen as used herein. Similarly, an oligonucleotide or polynucleotide that expresses an antigen or antigenic determinant in vivo, such as in nucleic acid immunization applications, is also included in the definition of antigen herein.

For purposes of the present invention, immunogens can be derived from any of several known CAL viruses, as described above, for example LACV. By “immunogenic fragment” is meant a fragment of a CAL polypeptide that includes one or more epitopes and thus elicits one or more of the immunological responses described herein. An “immunogenic fragment” of a particular CAL protein will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, that define an epitope, or any integer between 5 amino acids and the full-length sequence, provided that the fragment in question retains the ability to elicit an immunological response as defined herein.

The term “epitope” as used herein refers to a sequence of at least about 3 to 5, preferably about 5 to 10 or 15, and not more than about 500 amino acids (or any integer therebetween), which define a sequence that by itself or as part of a larger sequence, elicits an immunological response in the subject to which it is administered. Often, an epitope will bind to an antibody generated in response to such sequence. There is no critical upper limit to the length of the epitope, which may comprise nearly the full-length of the protein sequence, or even a fusion protein comprising two or more epitopes from the CAL virus molecule in question. An epitope for use in the subject invention is not limited to a polypeptide having the exact sequence of the portion of the parent protein from which it is derived. Indeed, viral gnomes are in a state of constant flux and contain several variable domains which exhibit relatively high degrees of variability between isolates. Thus the term “epitope” encompasses sequences identical to the native sequence, as well as modifications to the native sequence, such as deletions, additions and substitutions (generally conservative in nature).

Regions of a given polypeptide that include an epitope can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1985) Proc. Natl. Acad. Sci. USA 82:178-182; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., Proc. Natl. Acad. Sci. USA (1981) 78:3824-3828 for determining antigenicity profiles, and the Kyte-Doolittle technique, Kyte et al., J. Mol. Biol. (1982) 157:105-132 for hydropathy plots.

An “immunological response” to an antigen or composition is the development in a subject of a humoral and/or a cellular immune response to an antigen present in the composition of interest. For purposes of the present invention, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MEC molecules on their surface. A “cellular immune response” also refers to the production of cytokines, chemokines and other such molecules produced by activated T-cells and/or other white blood cells, including those derived from CD4+ and CD8+ T-cells.

A composition or vaccine that elicits a cellular immune response may serve to sensitize a vertebrate subject by the presentation of antigen in association with MEC molecules at the cell surface. The cell-mediated immune response is directed at, or near, cells presenting antigen at their surface. In addition, antigen-specific T-lymphocytes can be generated to allow for the future protection of an immunized host.

The ability of a particular immunogen to stimulate a cell-mediated immunological response may be determined by a number of assays, such as by lymphoproliferation (lymphocyte activation) assays, CTL cytotoxic cell assays, or by assaying for T-lymphocytes specific for the antigen in a sensitized subject. Such assays are well known in the art. See, e.g., Erickson et al., J. Immunol. (1993) 151:4189-4199; Doe et al., Eur. J. Immunol. (1994) 24:2369-2376. Recent methods of measuring cell-mediated immune response include measurement of intracellular cytokines or cytokine secretion by T-cell populations, or by measurement of epitope specific T-cells (e.g., by the tetramer technique) (reviewed by McMichael, A. J., and O'Callaghan, C. A., J. Exp. Med. (1998) 187:1367-1371; Mcheyzer-Williams et al, Immunol. Rev. (1996) 150:5-21; Lalvani et al., J. Exp. Med. (1997) 186:859-865.

Thus, an immunological response as used herein may be one that stimulates the production of antibodies (e.g., neutralizing antibodies that block CAL viruses from entering cells and/or replicating by binding to the pathogens, typically protecting cells from infection and destruction). The antigen of interest may also elicit production of CTLs. Hence, an immunological response may include one or more of the following effects: the production of antibodies by B-cells; and/or the activation of suppressor T-cells and/or δγ T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art. (See, e.g., Montefiori et al., J. Clin Microbiol. (1988) 26:231-235; Dreyer et al., AIDS Res Hum Retroviruses (1999) 15:1563-1571). Moreover, the immunogenicity of the various polypeptides and polynucleotides described herein can be tested in appropriate animal models. Acceptable animal models for studying CAL viruses are known in the art and include various mouse models such as mice lacking a functional interferon type 1 receptor (IFNAR-1) as described in, e.g., Schuh et al., Hum. Gene Ther. (1999) 10:1649-1658; and Pavlovic et al., Intervirology (2000) 43:312-321.

An “immunogenic composition” is a composition that comprises an antigenic molecule where administration of the composition to a subject results in the development in the subject of a humoral and/or a cellular immune response to the antigenic molecule of interest. The immunogenic composition can be introduced directly into a recipient subject, such as by injection, inhalation, oral, intranasal and mucosal (e.g., intra-rectally or intra-vaginally) administration. An “immunogenic composition” also denotes a composition for use in diagnostic assays, described further below.

By “subunit vaccine” is meant a vaccine composition that includes one or more selected antigens but not all antigens, derived from or homologous to, an antigen from a CAL virus, such as LACV. Such a composition is substantially free of intact virus or viral particles. Thus, a “subunit vaccine” can be prepared from at least partially purified (preferably substantially purified) immunogenic polypeptides from the pathogen, or analogs thereof. The method of obtaining an antigen included in the subunit vaccine can thus include standard purification techniques, recombinant production, or synthetic production.

“Substantially purified” generally refers to isolation of a substance (compound, polynucleotide, protein, polypeptide, polypeptide composition) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro-molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

By “equivalent antigenic determinant” is meant an antigenic determinant from different isolates or strains of a CAL virus which antigenic determinants are not necessarily identical due to sequence variation, but which occur in equivalent positions in the CAL virus sequence in question. In general the amino acid sequences of equivalent antigenic determinants will have a high degree of sequence homology, e.g., amino acid sequence homology of more than 30%, usually more than 40%, such as more than 60%, and even more than 80-90% homology, when the two sequences are aligned.

“Homology” refers to the percent identity between two polynucleotide or two polypeptide moieties. Two nucleic acid, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing normucleotidic backbones, for example, polyamide (e.g., peptide nucleic acids (PNAs)) and polymorpholino (commercially available from the Anti-Virals, Inc., Corvallis, Oregon, as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalidyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. In particular, DNA is deoxyribonucleic acid.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

A CAL virus polypeptide is produced “intracellularly” when it is found within the cell, either associated with components of the cell, such as in association with the endoplasmic reticulum (ER) or the Golgi Apparatus, or when it is present in the soluble cellular fraction. A CAL virus polypeptide is still considered to be produced “intracellularly” even if it is secreted into growth medium so long as sufficient amounts of the polypeptides remain present within the cell such that they can be purified from cell lysates using techniques described herein. Methods of intracellular production are described below, and include production in mammalian cells, production as vaccinia recombinants and the like. It has been found that when La Crosse glycoproteins are expressed in the native G2-NSm-G1 configuration, both G1 and G2 target the Golgi apparatus, but when expressed independently, G2 targets to the Golgi apparatus and G1 is retained in the endoplasmic reticulum, indicating that a G1-G2 association is required for Golgi targeting of G1. Disruption of the NSm region, e.g., with a foreign sequence, does not interfere with transport of the complex.

A “coding sequence” or a sequence which “encodes” a selected polypeptide, is a nucleic acid molecule which is transcribed and translated into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

“Operably linked” refers to an arrangement of elements wherein the components so described are configured so as to perform their desired function. Thus, a given promoter operably linked to a coding sequence is capable of effecting the expression of the coding sequence when the proper transcription factors, etc., are present. The promoter need not be contiguous with the coding sequence, so long as it functions to direct the expression thereof. Thus, for example, intervening untranslated yet transcribed sequences can be present between the promoter sequence and the coding sequence, as can transcribed introns, and the promoter sequence can still be considered “operably linked” to the coding sequence.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

A “control element” refers to a polynucleotide sequence which aids in the expression of a coding sequence to which it is linked. The term includes promoters, transcription termination sequences, upstream regulatory domains, polyadenylation signals, untranslated regions, including 5′-UTRs and 3′-UTRs and when appropriate, leader sequences and enhancers, which collectively provide for the transcription and translation of a coding sequence in a host cell.

A “promoter” as used herein is a regulatory region capable of binding RNA polymerase in a host cell and initiating transcription of a downstream (3′ direction) coding sequence operably linked thereto. For purposes of the present invention, a promoter sequence includes the minimum number of bases or elements necessary to initiate transcription of a gene of interest at levels detectable above background. Within the promoter sequence is a transcription initiation site, as well as protein binding domains (consensus sequences) responsible for the binding of RNA polymerase. Eucaryotic promoters will often, but not always, contain “TATA” boxes and “CAT” boxes.

A control sequence “directs the transcription” of a coding sequence in a cell when RNA polymerase will bind the promoter sequence and transcribe the coding sequence into mRNA, which is then translated into the polypeptide encoded by the coding sequence.

“Expression cassette” or “expression construct” refers to an assembly which is capable of directing the expression of the sequence(s) or gene(s) of interest. The expression cassette includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single-stranded DNA (e.g., a M13 origin of replication), at least one multiple cloning site, and a “mammalian” origin of replication (e.g., a SV40 or adenovirus origin of replication).

“Transformation” as used herein, refers to the insertion of an exogenous polynucleotide into a host cell, irrespective of the method used for insertion: for example, transformation by direct uptake, transfection, infection, and the like. For particular methods of transfection, see further below. The exogenous polynucleotide may be maintained as a nonintegrated vector, for example, an episome, or alternatively, may be integrated into the host genome.

By “nucleic acid immunization” is meant the introduction of a nucleic acid molecule encoding one or more selected immunogens into a host cell, for the in vivo expression of the immunogen. The nucleic acid molecule can be introduced directly into a recipient subject, such as by injection, inhalation, oral, intranasal and mucosal administration, or the like, or can be introduced ex vivo, into cells which have been removed from the host. In the latter case, the transformed cells are reintroduced into the subject where an immune response can be mounted against the immunogen encoded by the nucleic acid molecule.

An “antibody” intends a molecule that, through chemical or physical means, specifically binds to a polypeptide of interest. Thus, an anti-LACV G1 antibody is a molecule that specifically binds to an epitope of a LACV G1 protein. The term “antibody” as used herein includes antibodies obtained from both polyclonal and monoclonal preparations, as well as, the following: hybrid (chimeric) antibody molecules (see, for example, Winter et al., Nature (1991) 30:293-299; and U.S. Pat. No. 4,816,567); F(ab′)2 and F(ab) fragments; Fv molecules (non-covalent heterodimers, see, for example, Inbar et al., Proc Natl Acad Sci USA (1972) 69:2659-2662; and Ehrlich et al., Biochem (1980) 19:4091-4096); single-chain Fv molecules (sFv) (see, for example, Huston et al., Proc Natl Acad Sci USA (1988) 85:5879-5883); dimeric and trimeric antibody fragment constructs; minibodies (see, e.g., Pack et al., Biochem (1992) 31:1579-1584; Cumber et al., J Immunology (1992) 149B:120-126); humanized antibody molecules (see, for example, Riechmann et al., Nature (1988) 332:323-327; Verhoeyan et al., Science (1988) 239:1534-1536; and U.K. Patent Publication No. GB 2,276,169, published 21 Sep. 1994); and, any functional fragments obtained from such molecules, wherein such fragments retain immunological binding properties of the parent antibody molecule.

As used herein, a “solid support” refers to a solid surface such as a magnetic bead, latex bead, microtiter plate well, glass plate, nylon, agarose, acrylamide, and the like. “Immunologically reactive” means that the antigen in question will react specifically with anti-CAL virus antibodies present in a biological sample from a CAL virus-infected individual.

“Immune complex” intends the combination formed when an antibody binds to an epitope on an antigen.

A “DNA-dependent DNA polymerase” is an enzyme that synthesizes a complementary DNA copy from a DNA template. Examples are DNA polymerase I from E. coli and bacteriophage T7 DNA polymerase. All known DNA-dependent DNA polymerases require a complementary primer to initiate synthesis. Under suitable conditions, a DNA-dependent DNA polymerase may synthesize a complementary DNA copy from an RNA template.

A “DNA-dependent RNA polymerase” or a “transcriptase” is an enzyme that synthesizes multiple RNA copies from a double-stranded or partially-double stranded DNA molecule having a (usually double-stranded) promoter sequence. The RNA molecules (“transcripts”) are synthesized in the 5′ to 3′ direction beginning at a specific position just downstream of the promoter. Examples of transcriptases are the DNA-dependent RNA polymerase from E. coli and bacteriophages T7, T3, and SP6.

An “RNA-dependent DNA polymerase” or “reverse transcriptase” is an enzyme that synthesizes a complementary DNA copy from an RNA template. All known reverse transcriptases also have the ability to make a complementary DNA copy from a DNA template; thus, they are both RNA- and DNA-dependent DNA polymerases. A primer is required to initiate synthesis with both RNA and DNA templates.

“RNAse H” is an enzyme that degrades the RNA portion of an RNA:DNA duplex. These enzymes may be endonucleases or exonucleases. Most reverse transcriptase enzymes normally contain an RNAse H activity in addition to their polymerase activity. However, other sources of the RNAse H are available without an associated polymerase activity. The degradation may result in separation of RNA from a RNA:DNA complex. Alternatively, the RNAse H may simply cut the RNA at various locations such that portions of the RNA melt off or permit enzymes to unwind portions of the RNA.

As used herein, the term “target nucleic acid region” or “target nucleic acid” denotes a nucleic acid molecule with a “target sequence” to be amplified. The target nucleic acid may be either single-stranded or double-stranded and may include other sequences besides the target sequence, which may not be amplified. The term “target sequence” refers to the particular nucleotide sequence of the target nucleic acid which is to be amplified. The target sequence may include a probe-hybridizing region contained within the target molecule with which a probe will form a stable hybrid under desired conditions. The “target sequence” may also include the complexing sequences to which the oligonucleotide primers complex and extended using the target sequence as a template. Where the target nucleic acid is originally single-stranded, the term “target sequence” also refers to the sequence complementary to the “target sequence” as present in the target nucleic acid. If the “target nucleic acid” is originally double-stranded, the term “target sequence” refers to both the plus (+) and minus (−) strands.

The term “primer” or “oligonucleotide primer” as used herein, refers to an oligonucleotide which acts to initiate synthesis of a complementary nucleic acid strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is preferably single-stranded for maximum efficiency in amplification, but may alternatively be double-stranded. If double-stranded, the primer can first be treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis.

As used herein, the term “probe” or “oligonucleotide probe” refers to a structure comprised of a polynucleotide, as defined above, that contains a nucleic acid sequence complementary to a nucleic acid sequence present in the target nucleic acid analyte. The polynucleotide regions of probes may be composed of DNA, and/or RNA, and/or synthetic nucleotide analogs. Probes may be labeled in order to detect the target sequence. Such a label may be present at the 5′ end, at the 3′ end, at both the 5′ and 3′ ends, and/or internally. For example, when an “oligonucleotide probe” is to be used in a 5′ nuclease assay, such as the TaqMan™ technique, the probe will contain at least one fluorescer and at least one quencher which is digested by the 5′ endonuclease activity of a polymerase used in the reaction in order to detect any amplified target oligonucleotide sequences. In this context, the oligonucleotide probe will have a sufficient number of phosphodiester linkages adjacent to its 5′ end so that the 5′ to 3′ nuclease activity employed can efficiently degrade the bound probe to separate the fluorescers and quenchers. When an oligonucleotide probe is used in the TMA technique, it will be suitably labeled, as described below.

As used herein, the term “capture oligonucleotide” refers to an oligonucleotide that contains a nucleic acid sequence complementary to a nucleic acid sequence present in the target nucleic acid analyte such that the capture oligonucleotide can “capture” the target nucleic acid. One or more capture oligonucleotides can be used in order to capture the target analyte. The polynucleotide regions of a capture oligonucleotide may be composed of DNA, and/or RNA, and/or synthetic nucleotide analogs. By “capture” is meant that the analyte can be separated from other components of the sample by virtue of the binding of the capture molecule to the analyte. Typically, the capture molecule is associated with a solid support, either directly or indirectly.

It will be appreciated that the hybridizing sequences need not have perfect complementarity to provide stable hybrids. In many situations, stable hybrids will form where fewer than about 10% of the bases are mismatches, ignoring loops of four or more nucleotides. Accordingly, as used herein the term “complementary” refers to an oligonucleotide that forms a stable duplex with its “complement” under assay conditions, generally where there is about 90% or greater homology.

The terms “hybridize” and “hybridization” refer to the formation of complexes between nucleotide sequences which are sufficiently complementary to form complexes via Watson-Crick base pairing. Where a primer “hybridizes” with target (template), such complexes (or hybrids) are sufficiently stable to serve the priming function required by, e.g., the DNA polymerase to initiate DNA synthesis.

As used herein, the term “binding pair” refers to first and second molecules that specifically bind to each other, such as complementary polynucleotide pairs capable of forming nucleic acid duplexes. “Specific binding” of the first member of the binding pair to the second member of the binding pair in a sample is evidenced by the binding of the first member to the second member, or vice versa, with greater affinity and specificity than to other components in the sample. The binding between the members of the binding pair is typically noncovalent. Unless the context clearly indicates otherwise, the terms “affinity molecule” and “target analyte” are used herein to refer to first and second members of a binding pair, respectively.

The terms “specific-binding molecule” and “affinity molecule” are used interchangeably herein and refer to a molecule that will selectively bind, through chemical or physical means to a detectable substance present in a sample. By “selectively bind” is meant that the molecule binds preferentially to the target of interest or binds with greater affinity to the target than to other molecules. For example, a DNA molecule will bind to a substantially complementary sequence and not to unrelated sequences. An oligonucleotide that “specifically binds” to a LACV sequence denotes an oligonucleotide, e.g., a primer, probe or a capture oligonucleotide, that binds to a LACV sequence but does not bind to a sequence from a non-LACV CAL virus.

The “melting temperature” or “Tm” of double-stranded DNA is defined as the temperature at which half of the helical structure of DNA is lost due to heating or other dissociation of the hydrogen bonding between base pairs, for example, by acid or alkali treatment, or the like. The Tₘ of a DNA molecule depends on its length and on its base composition. DNA molecules rich in GC base pairs have a higher Tₘ than those having an abundance of AT base pairs. Separated complementary strands of DNA spontaneously reassociate or anneal to form duplex DNA when the temperature is lowered below the Tₘ. The highest rate of nucleic acid hybridization occurs approximately 25 degrees C. below the Tₘ. The Tₘ may be estimated using the following relationship: Tₘ=69.3+0.41 (GC) % (Mannur et al. (1962) J. Mol. Biol. 5:109-118).

As used herein, a “biological sample” refers to a sample of tissue or fluid isolated from a subject, including but not limited to, for example, blood, plasma, serum, fecal matter, urine, bone marrow, bile, spinal fluid, lymph fluid, samples of the skin, external secretions of the skin, respiratory, intestinal, and genitourinary tracts, tears, saliva, milk, blood cells, organs, biopsies and also samples of in vitro cell culture constituents including but not limited to conditioned media resulting from the growth of cells and tissues in culture medium, e.g., recombinant cells, and cell components.

As used herein, the terms “label” and “detectable label” refer to a molecule capable of detection, including, but not limited to, radioactive isotopes, fluorescers, chemiluminescers, chromophores, enzymes, enzyme substrates, enzyme cofactors, enzyme inhibitors, semiconductor nanoparticles, dyes, metal ions, metal sols, ligands (e.g., biotin, strepavidin or haptens) and the like. The term “fluorescer” refers to a substance or a portion thereof which is capable of exhibiting fluorescence in the detectable range. Particular examples of labels which may be used under the invention include, but are not limited to, horse radish peroxidase (IMP), fluorescein, FITC, rhodamine, dansyl, umbelliferone, dimethyl acridinium ester (DMAE), Texas red, luminol, NADPH and α-β-galactosidase.

The terms “effective amount” or “pharmaceutically effective amount” of an immunogenic composition, as provided herein, refer to a nontoxic but sufficient amount of the composition to provide the desired response, such as an immunological response, and optionally, a corresponding therapeutic effect. The exact amount required will vary from subject to subject, depending on the species, age, and general condition of the subject, the severity of the condition being treated, and the particular macromolecule of interest, mode of administration, and the like. An appropriate “effective” amount in any individual case may be determined by one of ordinary skill in the art using routine experimentation.

The term “treatment” as used herein refers to either (1) the prevention of infection or reinfection (prophylaxis), or (2) the reduction or elimination of symptoms of the disease of interest (therapy).

By “mammalian subject” is meant any mammal susceptible to the particular CAL virus infection in question. Such mammals include, without limitation, humans and other primates, including non-human primates such as chimpanzees and other apes and monkey species; rodents such as chipmunks, squirrels and laboratory animals including mice, rats and guinea pigs; rabbits, hares (such as the snowshoe hare); and domestic animals such as dogs and cats. The term does not denote a particular age. Thus, both adult and newborn subjects are intended to be covered. The invention described herein is intended for use in any of the above mammalian species, since the immune systems of all of these mammals operate similarly.

### 2. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery of reagents and methods for preventing, treating and diagnosing infection caused by the CAL serogroup of viruses, such as LACV infection. The methods use attenuated or inactivated viruses, or subunit compositions, to treat or prevent infection. Moreover, polypeptides and polynucleotides derived from CAL viruses can be used in diagnostic assays to identify infected subjects.

The methods are also useful for detecting CAL virus in blood samples, including without limitation, in whole blood, serum and plasma. Thus, the methods can be used to diagnose CAL virus infection in a subject, as well as to detect CAL virus contamination in donated blood samples. Aliquots from individual donated samples or pooled samples can be screened for the presence of CAL virus and those samples or pooled samples contaminated with CAL virus can be eliminated before they are combined. In this way, a blood supply substantially free of CAL virus contamination can be provided.

In order to further an understanding of the invention, a more detailed discussion is provided below regarding CAL viruses, various CAL polypeptide and polynucleotide immunogens for use in the subject compositions and methods, as well as production of the proteins, antibodies thereto and methods of using the proteins and antibodies.

CAL Virus Polypeptides and Polynucleotides

As explained above, the CAL serogroup family of viruses belongs to the Bunyavirus genus and are enveloped, minus-sense RNA viruses. The RNA of the viral genome is tripartite, consisting of three fragments generally designated as S, M and L for small, medium and large genome fragments, respectively. The M segment encodes two envelope glycoproteins, termed G1 and G2, and a nonstructural protein (NSm), in a single open reading frame. The S segment encodes a nucleocapsid protein, termed N and a further nonstructural protein termed NSs, in overlapping reading frames. The L segment of the genome encodes an RNA-dependent RNA polymerase.

Several distinct CAL viruses are found in association with specific mammalian hosts worldwide. Polypeptides and polynucleotides derived from any of the various isolates of the CAL serogroup will find use herein, including without limitation, any of the California encephalitis group of viruses such as LACV, snowshoe hare virus, Tahyna virus, San Angelo virus, Lumbo virus and Inkoo virus; any of the various isolates of the Melao viruses such as Jamestown Canyon virus, South River virus, Keystone virus and Serra do Navio virus; as well as any of the isolates of the Trivittatus and Guaroa group of viruses.

Sequences for viral polypeptides and the nucleic acid sequences encoding these polypeptides for a number of CAL virus isolates are known. Representative sequences are presented in FIGS. 1-3 herein for LACV polypeptides. Similarly, representative snowshoe hare virus sequences are found in NCBI Accession numbers J02390 and K02539 (S and M regions, respectively). Representative Tahyna virus sequences are found in NCBI Accession numbers Z68497 and U47142 (each including sequences for the S region); and AF229129 and AF123485 (each including sequences for the M region). See, also Campbell et al., Virus Res. (1999) 61:137-144, for a comparison of M RNA among 15 CAL serogroup viruses.

Thus, immunogens for use in subunit vaccines and diagnostics include those derived from one or more of the above regions from any CAL virus strain or isolate. Either the full-length proteins, fragments thereof containing epitopes of the full-length proteins, as well as fusions of the various regions or fragments thereof, will find use in the subject compositions and methods. Thus, for example, immunogens for use in such compositions can be derived from the G1 and/or G2 envelope regions of any of these CAL isolates. Immunogenic fragments of the envelope proteins, which comprise epitopes may be used in the subject compositions and methods. For example, fragments of the G1 and/or G2 polypeptide can comprise from about 5 contiguous amino acids to nearly the full-length of the molecule, such as 6, 10, 25, 50, 75, 100, 200, 250, 300, 350, 400, 450 or more contiguous amino acids of a G1 and/or G2 polypeptide, or any integer between the stated numbers. Additionally, the entire M region, including G1, G2 and NSm, as well as complexes of the G1 and G2 polypeptides, with or without NSm, or epitopes from the G1 polypeptide fused to epitopes of the G2 polypeptide with or without NSm, can be used in the subject compositions and methods.

Moreover, the G1 and/or G2 polypeptides for use herein may lack all or a portion of the transmembrane binding domain and/or the cytoplasmic tail found in the C-terminus of the envelope. Thus, the present invention contemplates the use of envelope polypeptides which retain the transmembrane binding domain and cytoplasmic tail, as well as polypeptides which lack all or a portion of the transmembrane binding domain and/or the cytoplasmic tail. The location of such domains can be readily determined using computer programs and algorithms well known in the art, such as the Kyte-Doolittle technique, Kyte et al., J. Mol. Biol. (1982) 157:105-132. A representative transmembrane binding domain from the La Crosse virus G1 envelope polypeptide occurs at approximately positions 1391-1419 of FIG. 1 and a representative cytoplasmic tail occurs at approximately positions 1420-1441 of FIG. 1. Such deleted or truncated constructs can include, but need not include, either homologous or heterologous signal sequences as described further below.

With respect to the La Crosse virus G1 envelope, particular transmembrane deletions for use herein include deletions of all or any portion of the transmembrane binding domain, as well as adjacent portions of the G1 protein. Thus, deletions can include for example, deletions of amino acids corresponding to positions 1388-1419, as well as deletions beginning at, for example, the amino acid corresponding to amino acid 1388, 1389, 1390, 1391, 1392, 1393, 1394, 1395 . . . 1400 . . . 1405 . . . 1410 of FIG. 1 and extending up to the amino acid corresponding to amino acid 1415, 1416, 1417, 1418, 1419, of FIG. 1, or any subset of these deletions, such as a deletion of amino acids 1389-1419, 1390-1419, 1391-1419, 1393-1419, and the like. Additionally, the deletions can extend into the cytoplasmic tail, such that all or a portion of the tail is removed. Such a G1 construct, lacking all or part of the transmembrane binding region and some or all of the cytoplasmic tail is represented by a G1 polypeptide including the sequence of amino acids corresponding to positions 474-1387 of FIG. 1, as well as a construct including amino acids corresponding to positions 474-1390 or 474-1391 of FIG. 1. It is to be understood that corresponding regions from other CAL viruses and other La Crosse isolates, in addition to the isolate from which the envelope sequences in FIG. 1 derives, are intended to be covered and one of skill in the art can readily determine the transmembrane and cytoplasmic regions based on a comparison with FIG. 1 herein.

As explained above, immunogens including the entire M region, i.e., G2-NSm-G1, can be used in the compositions and methods of the invention. A representative M region from the La Crosse virus is shown at amino acid positions 1-1441 of FIG. 1. It is to be understood that corresponding regions from other CAL viruses and other La Crosse isolates, in addition to the isolate from which the M region in FIG. 1 derives, are intended to be covered by the present invention. Additionally, the G1 polypeptide included in the M region construct can be truncated as explained above, to remove all or a portion of the transmembrane binding region and some or all of the cytoplasmic tail. Truncations and deletions can be any one of those described above.

Additionally, the N and NSs polypeptides, epitopes thereof, as well as analogs and fusions of these polypeptides will find use herein. Fusion molecules including more than one epitope derived from more than one region of the CAL genome will also find use with the present invention. If a fusion is produced, the polypeptides need not be organized in the same order as found in the native virus. Thus, for example, a G2 polypeptide can be fused to the N-terminus of a G1 polypeptide, etc.

Polynucleotides and polypeptides for use with the present invention can be obtained using standard techniques. For example, polynucleotides encoding the various immunogenic polypeptides can be isolated from a genomic library derived from nucleic acid sequences present in, for example, the plasma, serum, or tissue homogenate of a CAL virus-infected individual. Additionally, nucleic acid can be obtained directly from the CAL virus in question. Several members of the CAL family of viruses are available from the ATCC as follows: LACV (ATCC Accession No. VR-744); snowshoe hare virus (ATCC Accession No. VR-711); Tahyna virus (ATCC Accession No. VR-745); San Angelo virus (ATCC Accession No. VR-723); Lumbo virus (ATCC Accession No. VR-401); Inkoo virus (ATCC Accession No. VR-729); Melao virus (ATCC Accession No. VR-761); Jamestown Canyon virus (ATCC Accession No. VR-712); Keystone virus (ATCC Accession No. VR-722); Trivittatus (ATCC Accession No. VR-402); and Guaroa virus (ATCC Accession No. VR-394).

Alternatively, CAL virus can be isolated from infected mosquitoes, such as from Aedes albopictus, as described in e.g., Gerhardt et al., Emerging Infectious Diseases (2001) 7:807-811. Once obtained, the virus can be propagated using known techniques, such as described in Pekosz et al., J. Virol. (1995) 69:3475-3481. Generally, CAL viruses are grown in Vero or BHK cell-lines. An amplification method such as PCR can be used to amplify polynucleotides from either CAL virus genomic RNA or cDNA encoding therefor. Alternatively, polynucleotides can be synthesized in the laboratory, for example, using an automatic synthesizer.

Polynucleotides can comprise coding sequences for the various polypeptides which occur naturally or can include artificial sequences which do not occur in nature. These polynucleotides can be ligated to form a coding sequence for a fusion protein, if desired, using standard molecular biology techniques.

Once coding sequences have been prepared or isolated, such sequences can be cloned into any suitable vector or replicon. Numerous cloning vectors are known to those of skill in the art, and the selection of an appropriate cloning vector is a matter of choice. Suitable vectors include, but are not limited to, plasmids, phages, transposons, cosmids, chromosomes or viruses which are capable of replication when associated with the proper control elements. The coding sequence is then placed under the control of suitable control elements, depending on the system to be used for expression. Thus, the coding sequence can be placed under the control of a promoter, ribosome binding site (for bacterial expression) and, optionally, an operator, so that the DNA sequence of interest is transcribed into RNA by a suitable transformant. The coding sequence may or may not contain a signal peptide or leader sequence which can later be removed by the host in post-translational processing. See, e.g., U.S. Pat. Nos. 4,431,739; 4,425,437; 4,338,397.

If present, the signal sequence can be the native leader found in association with the CAL virus polypeptide of interest. For example, if the CAL virus polypeptide being expressed is the CAL virus G1 polypeptide, all or a portion of the native G1 leader sequence can be included. If a portion of the native G1 leader is present, the construct can include a polynucleotide sequence coding, for example, at least the G1 sequence of amino acids beginning at amino acid position 434 of FIG. 1, such as the sequence of amino acids beginning at amino acid position 400 . . . 375 . . . 350 . . . , 329, 328, 327, 326, 325, 324 . . . 310 . . . 305 . . . 300, or any integer between 434 and 300.

Alternatively, a heterologous signal sequence can be present which can increase the efficiency of secretion. A number of representative leader sequences are known in the art and include, without limitation, the yeast α-factor leader, the TPA signal peptide, the Ig signal peptide, and the like. Sequences for these and other leader sequences are well known in the art.

In addition to control sequences, it may be desirable to add regulatory sequences which allow for regulation of the expression of the sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector. For example, enhancer elements may be used herein to increase expression levels of the constructs. Examples include the SV40 early gene enhancer (Dijkema et al. (1985) EMBO J. 4:761), the enhancer/promoter derived from the long terminal repeat (LTR) of the Rous Sarcoma Virus (Gorman et al. (1982) Proc. Natl. Acad. Sci. USA 79:6777) and elements derived from human CMV (Boshart et al. (1985) Cell 41:521), such as elements included in the CMV intron A sequence (U.S. Pat. No. 5,688,688). The expression cassette may further include an origin of replication for autonomous replication in a suitable host cell, one or more selectable markers, one or more restriction sites, a potential for high copy number and a strong promoter.

An expression vector is constructed so that the particular coding sequence is located in the vector with the appropriate regulatory sequences, the positioning and orientation of the coding sequence with respect to the control sequences being such that the coding sequence is transcribed under the “control” of the control sequences (i.e., RNA polymerase which binds to the DNA molecule at the control sequences transcribes the coding sequence). Modification of the sequences encoding the molecule of interest may be desirable to achieve this end. For example, in some cases it may be necessary to modify the sequence so that it can be attached to the control sequences in the appropriate orientation; i.e., to maintain the reading frame. The control sequences and other regulatory sequences may be ligated to the coding sequence prior to insertion into a vector. Alternatively, the coding sequence can be cloned directly into an expression vector which already contains the control sequences and an appropriate restriction site.

As explained above, it may also be desirable to produce mutants or analogs of the polypeptide of interest. Mutants or analogs of CAL virus polynucleotides and polypeptides for use in the subject compositions may be prepared by the deletion of a portion of the sequence encoding the molecule of interest, by insertion of a sequence, and/or by substitution of one or more nucleotides within the sequence. Techniques for modifying nucleotide sequences, such as site-directed mutagenesis, and the like, are well known to those skilled in the art. See, e.g., Sambrook et al., supra; Kunkel, T. A. (1985) Proc. Natl. Acad. Sci. USA (1985) 82:448; Geisselsoder et al. (1987) BioTechniques 5:786; Zoller and Smith (1983) Methods Enzymol. 100:468; Dalbie-McFarland et al. (1982) Proc. Natl. Acad. Sci. USA 79:6409.

In order to facilitate recombinant expression, the molecule of interest can be expressed as a fusion protein, such as a fusion with, e.g., a 50 kDa E. coli maltose binding protein, a fusion with a yeast superoxide dismutase (SOD) or fragment thereof, or as a ubiquitin fusion protein.

The molecules can be expressed in a wide variety of systems, including insect, mammalian, bacterial, viral and yeast expression systems, all well known in the art. For example, insect cell expression systems, such as baculovirus systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit). Similarly, bacterial and mammalian cell expression systems are well known in the art and described in, e.g., Sambrook et al., supra. Yeast expression systems are also known in the art and described in, e.g., Yeast Genetic Engineering (Barr et al., eds., 1989) Butterworths, London.

A number of appropriate host cells for use with the above systems are also known. For example, mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human embryonic kidney cells, human hepatocellular carcinoma cells (e.g., Hep G2), Madin-Darby bovine kidney (“MDBK”) cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alis, Aedes aegypti, Autographa californica, Bombyx mori, Drosophila melanogaster, Spodoptera frugiperda, and Trichoplusia

Nucleic acid molecules comprising nucleotide sequences of interest can be stably integrated into a host cell genome or maintained on a stable episomal element in a suitable host cell using various gene delivery techniques well known in the art. See, e.g., U.S. Pat. No. 5,399,346.

Depending on the expression system and host selected, the molecules are produced by growing host cells transformed by an expression vector described above under conditions whereby the protein is expressed. The expressed protein is then isolated from the host cells and purified. If the expression system secretes the protein into growth media, the product can be purified directly from the media. If it is not secreted, it can be isolated from cell lysates. The selection of the appropriate growth conditions and recovery methods are within the skill of the art.

For representative methods for obtaining CAL virus sequences recombinantly, see, e.g., Bupp et al., Virology (1996) 220:485 490; Pekosz et al., J. Virol. (1995) 69:3475-3481. Once produced, the various polypeptides and polynucleotides can be formulated into subunit vaccine compositions for use as prophylactics or therapeutics, or used in diagnostic assays, as described below.

One particularly preferred method of producing the CAL virus proteins recombinantly involves intracellular production. Secreted proteins do not always retain the native conformation and may include modified glycosylation patterns. Thus, purification of intracellularly produced CAL virus proteins from cells rather than from culture medium can be used in order to preserve the native conformation and to produce proteins that display improved biological properties. The molecules so produced may perform better in assays and may be more immunoreactive and therefore provide improved diagnostic reagents, as compared to their secreted counterparts. While not wishing to be bound by any particular theory, the intracellularly expressed forms of CAL virus proteins may more closely resemble the native viral proteins due to the carbohydrate motifs present on the molecules, while the secreted glycoproteins may contain modified carbohydrate moieties or glycosylation patterns. Furthermore, the intracellularly produced forms may be conformationally different than the secreted forms.

Intracellular forms of the CAL virus proteins can be produced using the recombinant methods described above. Particularly desirable is the intracellular production of LACV full-length G1 or truncated G1, as well as the LACV G2-NSm-G1 fusion encoded by the LACV M region. Particular truncations to G1 are detailed above and include the deletion of all or part of the transmembrane binding domain and/or the cytoplasmic tail. Production in mammalian hosts, such as but not limited to production in CHO and HEK293 cells, is particularly desirable.

In order to produce the protein intracellularly, transformed cells are cultured for an amount of time such that the majority of protein is expressed intracellularly and not secreted. The cells are then disrupted using chemical, physical or mechanical means, which lyse the cells yet keep the CAL virus polypeptides substantially intact and the proteins are recovered from the intracellular extract. Intracellular proteins can also be obtained by removing components from the cell wall or membrane, e.g., by the use of detergents or organic solvents, such that leakage of the CAL virus polypeptides occurs. Such methods are known to those of skill in the art and are described in, e.g., Protein Purification Applications: A Practical Approach, (E. L. V. Harris and S. Angal, Eds., 1990).

For example, methods of disrupting cells for use with the present invention include but are not limited to: sonication or ultrasonication; agitation; liquid or solid extrusion; heat treatment, freeze-thaw; desiccation; explosive decompression; osmotic shock; treatment with lytic enzymes including proteases such as trypsin, neuraminidase and lysozyme; alkali treatment; and the use of detergents and solvents such as bile salts, sodium dodecylsulphate, Triton, NP40 and CHAPS. The particular technique used to disrupt the cells is largely a matter of choice and will depend on the cell type in which the polypeptide is expressed, culture conditions and any pretreatment used. Preferably, for the production of the recombinant CAL virus polypeptides of interest, the cells are treated with a hypotonic solution (i.e. a solution having an ionic strength less than physiological saline, e.g., 10 mM Tris-HCl) to lyse the outer membrane.

Following disruption of the cells, insoluble cellular components are separated from the soluble cell contents, generally by centrifugation, and the intracellularly produced polypeptides can be recovered with the insoluble portion, which contains substantially all of the membrane component of the cells. The insoluble portion is then treated with a non-ionic detergent, such as surfactant consisting of the octyl- or nonylphenoxy polyoxyethanols (for example the commercially available Triton series, particularly Triton X-100), polyoxyethylene sorbitan esters (Tween series) and polyoxyethylene ethers or esters, in order to solubilize the membrane component and release the immunogenic CAL virus polypeptide, such as a CAL virus full-length or truncated G1, or the entire CAL virus M region, i.e., a G2-NSm-G1 fusion polypeptide. The released polypeptide is then further purified, using standard purification techniques such as but not limited to, one or more column chromatography purification steps, such as but not limited to ion-exchange chromatography, size-exclusion chromatography, electrophoresis, HPLC, immunoabsorbent techniques, affinity chromatography, immunoprecipitation, and the like.

For example, one method for obtaining the intracellular CAL virus polypeptides of the present invention involves affinity purification, such as by immunoaffinity chromatography using antibodies specific for the desired CAL virus antigen, or by lectin affinity chromatography. Particularly preferred lectin resins are those that recognize mannose moieties such as but not limited to resins derived from Galanthus nivalis agglutinin (GNA), Lens culinaris agglutinin (LCA or lentil lectin), Pisum sativum agglutinin (PSA or pea lectin), Narcissus pseudonarcissus agglutinin (NPA), Allium ursinum agglutinin (AUA) and concanavalin A (ConA) resins. The choice of a suitable affinity resin is within the skill in the art. After affinity purification, the polypeptides can be further purified using conventional techniques well known in the art, such as by using an ion exchange column, such as a cation or anion exchange column, (e.g., SP-Sepharose). Additional columns can also be used in the process, e.g., a hydroxyapatite column, for example, under high salt buffer conditions. Alternatively, purification can be done using an ion exchange column, such as a cationic or anionic exchange column, only. Preferably, a non-ionic detergent is maintained in the buffers during the purification process. These techniques provide for a highly purified antigen that can subsequently be used in vaccine compositions as well as highly sensitive diagnostic reagents.

Particular methods for isolating intracellularly expressed CAL virus polypeptides are presented in the examples using ConA as the lectin column. Another method of isolating intracellularly expressed CAL virus polypeptides, such as CAL virus envelope polypeptides prepared in HEK293 or CHO cells, is as follows.

(1) Cell detergent extraction. Frozen transfected 293 or CHO cells are thawed and lysed by suspension in a 10 mM Tris-HCl, pH 8.0 buffer followed by douncing in a Kontes glass dounce in an ice bucket. After centrifugation, the membrane pellet is resuspended in 100 mm Tris-HCl, pH 8.0 buffer containing 4% Triton X-100 detergent and again dounced in an ice bucket. After centrifugation, the supernatant is diluted with an equal volume of 2 M NaCl and centrifuged again. The resulting supernatant, referred to as a Triton X-100 extract, is frozen at −80 C.

(2) GNA lectin chromatography. The Triton X-100 extract is thawed and filtered with 5 μm and 1 μm filters then applied to a Galanthus nivalis lectin agarose (GNA) column previously equilibrated with 25 m phosphate buffer, pH 6.8, containing 1 M NaCl and 2.0% Triton X-100 detergent. The column is washed with 25 mM phosphate buffer, pH 6.8, containing 1 M NaCl and 0.1% Triton X-100 detergent. The CAL virus polypeptide is eluted with 1 M methyl-d-alpha-manoside in 25 mM phosphate buffer, pH 6.8, containing 1 M NaCl and 0.1% Triton X-100 detergent.

(3) HAP chromatography. GNA eluate material is concentrated and then diluted to reduce the NaCl content to 200 mM. It is then applied to a hydroxyapatite (HAP) equilibrated with 25 mM phosphate buffer, pH 6.8, containing 200 mM NaCl and 0.1% Triton X-100 detergent. The flow-through material is collected and dialyzed against 25 mM phosphate buffer, pH 6.0, containing 0.1% Triton X-100 detergent overnight at 4° C.

(4) SP chromatography. The dialyzed CAL virus polypeptide is applied to a SP sepharose high performance column previously equilibrated in 25 mM phosphate buffer, pH 6.0, containing 0.1% Triton X-100 detergent. The polypeptide is eluted with 25 mM phosphate buffer, pH 6.0, containing 0.5 M NaCl and 0.1% Triton X-100 detergent.

Alternatively, rather than recombinantly produced, the CAL virus polypeptides can be provided as crude cell lysates of CAL virus-infected cells using methods well known in the art Generally, such methods entail extracting proteins from infected cells using such techniques such as sonication or ultrasonication; agitation; liquid or solid extrusion; heat treatment; freeze-thaw techniques; explosive decompression; osmotic shock; proteolytic digestion such as treatment with lytic enzymes including proteases such as pepsin, trypsin, neuraminidase and lysozyme; alkali treatment; pressure disintegration; the use of detergents and solvents such as bile salts, sodium dodecylsulphate, TRITON, NP40 and CHAPS; fractionation, and the like. The particular technique used to disrupt the cells is largely a matter of choice and will depend on the type of cell, culture conditions and any pre-treatment used. Following disruption of the cells, cellular debris can be removed, generally by centrifugation and/or dialysis.

The immunogens present in such lysates can be further purified if desired, using standard purification techniques such as but not limited to, column chromatography, ion-exchange chromatography, size-exclusion chromatography, electrophoresis, HPLC, immunoadsorbent techniques, affinity chromatography, immunoprecipitation, and the like.

The immunogens may also be synthesized chemically, using conventional peptide synthesis techniques. See, e.g., See, e.g., J. M. Stewart and J. D. Young, Solid Phase Peptide Synthesis, 2nd Ed., Pierce Chemical Co., Rockford, Ill. (1984) and G. Barany and R. B. Merrifield, The Peptides: Analysis, Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Verlag, Berlin (1984) and E. Gross and J. Meienhofer, Eds., The Peptides: Analysis, Synthesis, Biology, supra, Vol. 1, for classical solution synthesis.

Inactivated (or Killed) CAL Virus Vaccines

The invention includes compositions comprising inactivated (or killed) CAL virus, such as inactivated LACV, and methods for the production thereof. Inactivated viral compositions can be used as prophylactic or therapeutic vaccines. Preferably the inactivated vaccine compositions comprise an amount of inactivated virus equivalent to a virus titer of from about 10³ to 10¹² plaque forming units (PFU) or 10³ to 10¹² tissue culture infectious dose 50 (TCID₅₀) per milliliter, preferably 10⁴ to 10¹⁰ PFU or TCID₅₀, even more preferably from about 10⁵ to 10⁹ PFU or TCID₅₀ per milliliter, or any dose within these stated ranges. The vaccine compositions comprise a sufficient amount of the virus antigen to produce an immunological response in a mammal, as defined above. Such compositions are described more fully below.

Virus can be obtained directly from the ATCC as described above. Other sources of virus include plasma, serum, or tissue homogenates from CAL virus-infected individuals. Alternatively, CAL virus can be isolated from infected mosquitos, such as from Aedes albopictus, as described in e.g., Gerhardt et al., Emerging Infectious Diseases (2001) 7:807-811. Once obtained, the virus can be propagated using known techniques, such as described in Pekosz et al., J. Virol. (1995) 69:3475-3481. CAL viruses are generally cultured in either an adherent or suspension mammalian cell culture. Other cell cultures can be derived from avian (e.g., hen cells such as hen embryo cells (CEF cells)), amphibian, reptile, insect, or fish sources. Mammalian sources of cells include, but are not limited to, human or non-human primate (e.g., MRC-5 (ATCC CCL-171), WI-38 (ATCC CCL-75), human embryonic kidney cells (293 cells, typically transformed by sheared adenovirus type 5 DNA), VERO cells from monkey kidneys), horse, cow (e.g., MDBK cells), sheep, dog (e.g., MDCK cells from dog kidneys, ATCC CCL34 MDCK (NBL2) or MDCK 33016, deposit number DSM ACC 2219 as described in WO 97/37001), cat, and rodent (e.g., hamster cells such as BHK21-F, HKCC cells, or Chinese hamster ovary cells (CHO cells)), and may be obtained from a wide variety of developmental stages, including for example, adult, neonatal, fetal, and embryo.

In certain embodiments the cells are immortalized (e.g., PERC.6 cells are described, for example, in WO 01/38362 and WO 02/40665, incorporated by reference herein in their entireties, as well as deposited under ECACC deposit number 96022940), or any other cell type immortalized using the techniques described herein.

In preferred embodiments, mammalian cells are utilized, and may be selected from and/or derived from one or more of the following non-limiting cell types: fibroblast cells (e.g., dermal, lung), endothelial cells (e.g., aortic, coronary, pulmonary, vascular, dermal microvascular, umbilical), hepatocytes, keratinocytes, immune cells (e.g., T cell, B cell, macrophage, NK, dendritic), mammary cells (e.g., epithelial), smooth muscle cells (e.g., vascular, aortic, coronary, arterial, uterine, bronchial, cervical, retinal pericytes), melanocytes, neural cells (e.g., astrocytes), prostate cells (e.g., epithelial, smooth muscle), renal cells (e.g., epithelial, mesangial, proximal tubule), skeletal cells (e.g., chondrocyte, osteoclast, osteoblast), muscle cells (e.g., myoblast, skeletal, smooth, bronchial), liver cells, retinoblasts, and stromal cells. WO 97/37000 and WO 97/37001, incorporated by reference herein in their entireties, describe production of animal cells and cell lines capable of growth in suspension and in serum-free media and are useful in the production and replication of viruses.

Preferably, the CAL viruses of the invention are grown on VERO cells or BHK cells.

Culture conditions for the above cell types are well-described in a variety of publications. Alternatively, culture medium, supplements, and conditions may be purchased commercially, such as for example, as described in the catalog and additional literature of Cambrex Bioproducts (East Rutherford, N.J.).

In certain embodiments, the host cells used in the methods described herein are cultured in serum free and/or protein free media. A medium is referred to as a serum-free medium in the context of the present invention when there are no additives from serum of human or animal origin. Protein-free is understood to mean cultures in which multiplication of the cells occurs with exclusion of proteins, growth factors, other protein additives and non-serum proteins. The cells growing in such cultures naturally contain proteins themselves.

Known serum-free media include Iscove's medium, Ultra-CHO medium (BioWhittaker) or EX-CELL (JRH Bioscience). Ordinary serum-containing media include Eagle's Basal Medium (BME) or Minimum Essential Medium (MEM) (Eagle, Science, 130, 432 (1959)) or Dulbecco's Modified Eagle Medium (DMEM or EDM), which are ordinarily used with up to 10% fetal calf serum or similar additives. Optionally, Minimum Essential Medium (MEM) (Eagle, Science, 130, 432 (1959)) or Dulbecco's Modified Eagle Medium (DMEM or EDM) may be used without any serum containing supplement. Protein-free media like PF-CHO (JHR Bioscience), chemically-defined media like ProCHO 4CDM (BioWhittaker) or SMIF 7 (Gibco/BRL Life Technologies) and mitogenic peptides like PRIMACTONE, PEPTICASE or HyPep™ (all from Quest International) or lactalbumin hydrolyzate (Gibco and other manufacturers) are also adequately known in the prior art. The media additives based on plant hydrolyzates have the special advantage that contamination with viruses, mycoplasma or unknown infectious agents can be ruled out.

The cell culture conditions to be used for the desired application (temperature, cell density, pH value, etc.) are variable over a very wide range depending on the cell line employed and can readily be adapted to the requirements of the CAL virus in question.

Methods for propagating CAL virus in cultured cells (e.g., mammalian cells) includes the steps of inoculating the cultured cells with the particular CAL virus, cultivating the infected cells for a desired time period for virus propagation, such as for example as determined by virus titer or virus antigen expression (e.g., between 24 and 168 hours after inoculation) and collecting the propagated virus. The cultured cells are inoculated with the desired virus (measured by PFU or TCID₅₀) to cell ratio of 1:500 to 1:1, preferably 1:100 to 1:5, more preferably 1:50 to 1:10. The CAL virus is added to a suspension of the cells or is applied to a monolayer of the cells, and the virus is absorbed on the cells for at least 60 minutes but usually less than 300 minutes, preferably between 90 and 240 minutes at 25° C. to 40° C., preferably 28° C. to 37° C. The infected cell culture (e.g., monolayers) may be removed either by freeze-thawing or by enzymatic action to increase the viral content of the harvested culture supernatants. The harvested fluids are then either inactivated or stored frozen.

Methods of inactivating or killing viruses are known in the art. Such methods destroy the ability of the viruses to infect mammalian cells. Inactivation can be achieved using either chemical or physical means. Chemical means for inactivating a CAL virus include treatment of the virus with an effective amount of one or more of the following agents: detergents, formaldehyde, formalin, β-propiolactone, or UV light. Other methods of viral inactivation are known in the art, such as for example binary ethylamine, acetyl ethyleneimine, or gamma irradiation.

For example, β-propiolactone may be used at concentrations such as 0.01 to 0.5%, preferably at 0.5% to 0.2%, and still more preferably at 0.025 to 0.1%. The inactivating agent is added to virus-containing culture supernatants (virus material) prior to or after harvesting. The culture supernatants can be used directly or cells disrupted to release cell-associated virus prior to harvesting. Further, the inactivating agent may be added after culture supernatants have been stored frozen and thawed, or after one or more steps of purification to remove cell contaminants. β-propiolactone is added to the virus material, with the adverse shift in pH to acidity being controlled with sodium hydroxide (e.g., 1 N NaOH) or sodium bicarbonate solution. The combined inactivating agent-virus materials are incubated at temperatures from 4° C. to 37° C., for incubation times of preferably 24 to 72 hours.

Alternatively, binary ethyleneimine can be used to inactivate virus. One representative method of inactivating CAL virus is as follows. Binary ethyleneimine is made by mixing equal volumes of a 0.2 molar bromoethylamine hydrobromide solution with a 0.4 molar sodium hydroxide solution. The mixture is incubated at about 37° C. for 60 minutes. The resulting cyclized inactivant, binary ethyleneimine, is added to the virus materials at 0.5 to 4 percent, and preferably at 1 to 3 percent, volume to volume. The inactivating virus materials are held from about 4° C. to 37° C. for 24 to 72 hours with periodic agitation. At the end of this incubation 20 ml of a sterile 1 molar sodium thiosulfate solution was added to insure neutralization of the BEI. Diluted and undiluted samples of the inactivated virus materials are added to susceptible cell (tissue) culture (e.g., VERO) to detect any non-inactivated virus. The cultured cells are passaged multiple times and examined for the presence of CAL virus based on any of a variety of methods, such as, for example, cytopathic effect (CPE) and antigen detection (e.g., via fluoroscent antibody conjugates specific for CAL virus. Such tests allow determination of complete virus inactivation.

Methods of purification of inactivated virus are known in the art and may include one or more of gradient centrifugation, ultracentrifugation, continuous-flow ultracentrifugation and chromatography, such as ion exchange chromatography, size exclusion chromatography, and liquid affinity chromatography. See, J P Gregersen “Herstellung von Virussimpfstoffen aus Zellkulturen” Chapter 4.2 in Pharmazeutische Biotecnologie (eds. O. Kayser and R H Mueller) Wissenschaftliche Verlagsgesellschaft, Stuttgart, 2000. See also, O'Neil et al., Biotechnology (1993) 11:173-177; Prior et al., Pharmaceutical Technology (1995) 30-52; and Majhdi et al., J. Clinical Microbiol. (1995) 35:2937-2942.

Other examples of purification methods suitable for use in the invention include polyethylene glycol or ammonium sulfate precipitation (see, Trepanier et al., J. Virological Meth. (1981) 3:201-211; Hagen et al., Biotechnology Progress (1996) 12:406-412; and Carlsson et al., J. Virological Meth. (1994) 4727-36) as well as ultrafiltration and microfiltration (see, Pay et al., Develop. Biol. Standardization (1985) 60:171-174; Tsurumi et al., Polymer Journal (1990) 22:1085-1100; and Makino et al., Archives Virol. (1994) 139:87-96).

Preferably, the virus is purified using chromatography, such as ion exchange chromatography. Chromatic purification allows for the production of large volumes of virus-containing suspension. The viral product of interest can interact with the chromatic medium by a simple adsorption/desorption mechanism, and large volumes of sample can be processed in a single load. Contaminants which do not have affinity for the adsorbent pass through the column. The virus material can then be eluted in concentrated form.

Preferred anion exchange resins for use in the invention include DEAF, EMD TMAE. Preferred cation exchange resins may comprise a sulfonic acid-modified surface. In one embodiment, the virus is purified using ion exchange chromatography comprising a strong anion exchange resin (i.e. EMD TMAE) for the first step and EMD-SO₃ (cation exchange resin) for the second step. A metal-binding affinity chromatography step can optionally be included for further purification. (See, e.g., WO 97/06243).

A preferred resin for use in the invention is FRACTOGEL EMD. This synthetic methacrylate based resin has long, linear polymer chains (so-called “tentacles”) covalently attached. This “tentacle chemistry” allows for a large amount of sterically accessible ligands for the binding of biomolecules without any steric hindrance. This resin also has improved pressure stability.

Column-based liquid affinity chromatography is another preferred purification method. One example of a resin for use in this purification method is MATREK CELLUFINE SULFATE (MCS). MCS consists of a rigid spherical (approximately 45-105 μm diameter) cellulose matrix of 3,000 Dalton exclusion limit (its pore structure excludes macromolecules), with a low concentration of sulfate ester functionality on the 6-position of cellulose. Sulfate ester, the functional ligand, is relatively highly dispersed, thus presenting insufficient cationic charge density to allow for most soluble proteins to adsorb onto the bead surface. Therefore, the bulk of the protein found in typical virus pools (cell culture supernatants, i.e. pyrogens and most contaminating proteins, as well as nucleic acids and endotoxins) are washed from the column and a degree of purification of the bound virus is achieved.

The rigid, high-strength beads of MCS tend to resist compression. The pressure/flow characteristics of MCS permit high linear flow rates and allow high-speed processing, even in large columns, making it an easily scalable unit operation. In addition a chromatographic purification step, MCS provides increased assurance of safety and product sterility, avoiding excessive product handling and safety concerns. As endotoxins do not bind to it, the MCS purification step allows a rapid and contaminant-free depyrogenation. Gentle binding and elution conditions provide high capacity and product yield. The MCS resin therefore represents a simple, rapid, effective, and cost-saving means for concentration, purification and depyrogenation. In addition, MCS resins can be reused repeatedly.

The inactivated virus may be further purified by gradient centrifugation, preferably density gradient centrifugation. The density gradient centrifugation step may be performed using, for example, a swinging bucket rotor, a fixed angle rotor, or a vertical tube rotor. Preferably, the gradient centrifugation step is performed using a swinging bucket rotor. This type of rotor has a sufficiently long path-length to provide high quality separations, particularly with multicomponent samples. In addition, swinging bucket rotors have greatly reduced wall effects, and the contents do not reorient during acceleration and deceleration. Because of their longer path-length, separations take longer compared to fixed angle or vertical tube rotors. The prepared sucrose solutions are controlled via refractometer on their sucrose concentration.

Sucrose gradients for swinging bucket centrifuge tubes may be formed prior to centrifugation by the use of a gradient former (continuous/linear). The volume of sample which can be applied to the gradient in a swinging bucket rotor tube is a function of the cross-sectional area of the gradient that is exposed to the sample. If the sample volume is too high, there is not sufficient radial distance in the centrifuge tube for effective separation of components in a multicomponent sample.

An approximate sample volume for the swinging bucket rotor SW 28 is 1-5 ml per tube (with a tube diameter of 2.54 cm). The sample is applied to the gradient by pipetting the volume on top of the gradient. The blunt end of the pipette is placed at a 45-60° angle to the tube wall, approximately 2-3 mm above the gradient. The sample is injected slowly and allowed to run down the wall of the tube onto the gradient. After centrifugation, gradient fractions are recovered by carefully inserting a gauge needle into the bottom of the tube and collecting 2 ml fractions by pumping the liquid from the tube into falcon tubes. Sucrose density gradients suitable for use with this purification step include 15-60%, 15-50%, and 15-40%. Preferably, the sucrose density gradient is 15-40%.

In one embodiment, inactivated virus is purified by a method comprising a first step of chromatography purification and a second step of gradient centrifugation. Preferably the first step comprises liquid affinity chromatography, such as MCS and the second step comprises density gradient centrifugation using a swinging bucket rotor.

Additional purification methods which may be used to purify inactivated LACV virus include the use of a nucleic acid degrading agent, preferably a nucleic acid degrading enzyme, such as a nuclease having DNase and RNase activity, or an endonuclease, such as from Serratia marcescens, commercially available as BENZONASE, membrane adsorbers with anionic functional groups (e.g. SARTOBIND) or additional chromatographic steps with anionic functional groups (e.g. DEAE or TMAE). An ultrafiltration/dialfiltration and final sterile filtration step can also be added to the purification method.

The treatment of the virus with the nucleic acid degrading enzyme and inactivating agent can be performed by a sequential treatment or in a combined or simultaneous manner. Preferably, the nucleic acid degrading agent is added to the virus preparation prior to the addition of the inactivating agent.

The purified viral preparation of the invention is substantially free of contaminating proteins derived from the cells or cell culture and preferably comprises less than about 50 pg cellular nucleic acid/μg virus antigen. Still more preferably, the purified viral preparation comprises less than about 20 pg, and even more preferably, less than about 10 pg. Methods of measuring host cell nucleic acid levels in a viral sample are known in the art. Standardized methods approved or recommended by regulatory authorities such as the WHO or the FDA are preferred.

Attenuated CAL Virus Vaccines

The invention also includes compositions comprising attenuated CAL viruses. As used herein, attenuation refers to the decreased virulence of the CAL virus in a mammalian subject. The compositions can be used as prophylactics or therapeutics. Methods of attenuating viruses are known in the art. Such methods include serial passage of the virus in cultured cells as described above (e.g., mammalian cell culture, preferably BHK or VERO cells), until the virus demonstrates attenuated function. The temperature at which the virus is grown can be any temperature at which tissue culture passage attenuation occurs. Attenuated function of the virus after one or more passages in cell culture can be measured by one skilled in the art. Evidence of attenuated function may be indicated by decreased levels of viral replication or by decreased virulence in an animal model. Acceptable animal models for studying CAL viruses are known in the art and include various mouse models such as mice lacking a functional interferon type 1 receptor (IFNAR-1) as described in, e.g., Schuh et al., Hum. Gene Ther. (1999) 10:1649-1658; and Pavlovic et al., Intervirology (2000) 43:312-321.

Other methods of producing an attenuated CAL virus include passage of the virus in cell culture at suboptimal or “cold” temperatures and/or introduction of attenuating mutations into the CAL viral genome by random mutagenesis (e.g., chemical mutagenesis) or site specific-directed mutagenesis. Preparation and generation of attenuated RSV vaccines (the methods of which will generally be applicable to CAL virus) are disclosed in, for example, EP 0 640 128, U.S. Pat. No. 6,284,254, U.S. Pat. No. 5,922,326, U.S. Pat. No. 5,882,651.

The attenuated derivatives of CAL virus are produced in several ways, such as for example, by introduction of temperature sensitive-mutations either with or without chemical mutagenesis (e.g., 5-fluorouracil), by passage in culture at “cold” temperatures. Such cold adaptation includes passage at temperatures between about 20° C. to about 32° C., and preferably between temperatures of about 22° C. to about 30° C., and most preferably between temperatures of about 24° C. and 28° C. The cold adaptation or attenuation may be performed by passage at increasingly reduced temperatures to introduce additional growth restriction mutations. The number of passages required to obtain safe, immunizing attenuated virus is dependent at least in part on the conditions employed. Periodic testing of the CAL virus culture for virulence and immunizing ability in animals (e.g., mouse, primate) can readily determine the parameters for a particular combination of tissue culture and temperature. The attenuated vaccine will typically be formulated in a dose of from about 10³ to 10¹² PFU or 10³ to 10¹² tissue culture infectious dose 50 (TCID₅₀) per milliliter, preferably 10⁴ to 10¹⁰ PFU or TCID₅₀, even more preferably from about 10⁵ to 10⁹ PFU or TCID₅₀ per milliliter, or any dose within these stated ranges.

CAL virus can also be attenuated by mutating one or more of the various viral regions to reduce expression of the viral structural or nonstructural proteins. The attenuated CAL virus may comprises one or more additions, deletions or insertion in one or more of the regions of the viral genome. For example, the hydrophobic domains of CAL proteins are targets for genetic mutation to develop attenuated CAL virus vaccines. The hydrophobic domains are also targets for small molecule inhibitors of CAL viruses. The hydrophobic domains may also be used to generate antibodies specific to those regions to treat or prevent CAL virus infection. Transmembrane and hydrophobic regions of the CAL virus proteins are readily identified using programs well known in the art, such as the Kyte-Doolittle technique, Kyte et al., J. Mol. Biol. (1982) 157:105-132.

The virus is attenuated by means of an addition, deletion or substitution of one or more polynucleotides found in the region encoding for one or more of the hydrophobic domains.

Once attenuated, the virus is purified using techniques known in the art, such as described above with reference to inactivated viruses.

Split CAL Virus Vaccines

The invention also includes a composition comprising a split CAL virus formulation and methods for the manufacture thereof. This composition can be used as a prophylactic or therapeutic CAL virus vaccine.

Methods of splitting enveloped viruses and splitting agents are known in the art. See, for example, WO 02/28422, WO 02/067983, WO 02/074336, and WO 01/21151, each of which is incorporated herein by reference in its entirety. The splitting of the virus is carried out by disrupting or fragmenting whole virus, infectious (wild-type or attenuated) or non-infectious (for example inactivated), with a disrupting concentration of a splitting agent. The disruption results in a full or partial solubilization of the virus proteins, altering the integrity of the virus.

Preferably, the splitting agent is a non-ionic or an ionic surfactant. Examples of splitting agents useful in the invention include: bile acids and derivatives thereof, non-ionic surfactants, alkylglycosides or alkylthioglycosides and derivatives thereof, acyl sugars, sulphobetaines, betains, polyoxyethylenealkylethers, N,N-dialkyl-Glucamides, Hecameg, alkylphenoxypolyethoxyethanols, quaternary ammonium compounds, sarcosyl, CTAB (cetyl trimethyl ammonium bromide) or Cetavlon.

Preferably, the ionic surfactant is a cationic detergent. Cationic detergents suitable for use in the invention include detergents comprising a compound of the following formula:

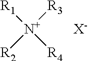

wherein

R₁, R₂ and R₃ are the same or different and each signifies alkyl or aryl, or

R₁ and R₂, together with the nitrogen atom to which these are attached form a 5- or 6-membered heterocyclic ring, and

R₃ signifies alkyl or aryl, or

R₁, R₂ and R₃ together with the nitrogen atom to which these are attached, signify a 5- or 6-membered heterocyclic ring, unsaturated at the nitrogen atom,

R₄ signifies alkyl or aryl, and

X signifies an anion.

Examples of such cationic detergents are cetyltrimethylammonium salts, such as ceytltrimethylammonium bromide (CTAB) and myristyltrimethylammonium salt.

Additional cationic detergents suitable for use in the invention include lipofectin, lipofectamine, and DOTMA.

Non-ionic surfactants suitable for use in the invention include one or more selected from the group consisting of the octyl- or nonylphenoxy polyoxyethanols (for example the commercially available Triton series), polyoxyethylene sorbitan esters (Tween series) and polyoxyethylene ethers or esters of the general formula (I):

HO(CH₂CH₂O)ₙ-A-R (I)

wherein n is 1-50, A is a bond or —C(O)—, R is C₁₋₅₀ alkyl or phenyl C₁₋₅₀ alkyl; and combinations of two or more of these.

The invention comprises a method of preparing a split CAL virus comprising contacting the CAL virus with a sufficient amount of splitting agent to disrupt the viral envelope. The loss of integrity after splitting renders the virus non-infectious. Once the disrupted viral envelope proteins are generally no longer associated with whole intact virions, other viral proteins are preferably fully or partially solubilized and are therefore not associated, or only in part associated, with whole intact virions after splitting.

The method of preparing a split CAL virus may further comprise removal of the splitting agents and some or most of the viral lipid material. The process may also include a number of different filtration and/or other separation steps such as ultracentrifugation, ultrafiltration, zonal centrifugation and chromatographic steps in a variety of combinations. The process may also optionally include an inactivation step (as described above) which may be carried out before or after the splitting. The splitting process may be carried out as a batch, continuous, or semi-continuous process.

Split CAL virus vaccines of the invention may include structural proteins, membrane fragments and membrane envelope proteins. Preferably, the split CAL virus preparations of the invention comprise at least half of the viral structural proteins.

One example of a method of preparing a split CAL virus formulation includes the following steps:

(i) propagation of the CAL virus in cell culture, such as VERO cells or BHK cells (see discussion above regarding culture of CAL virus);

(ii) harvesting CAL virus-containing material from the cell culture;

(iii) clarifying the harvested material to remove non-CAL virus material;

(iv) concentrating the harvested CAL virus;

(v) separating the whole CAL virus from non-virus material;

(vi) splitting the whole CAL virus using a suitable splitting agent in a density gradient centrifugation step; and

(vii) filtrating to remove undesired materials.

The above steps are preferably performed sequentially. The clarification step is preferably performed by centrifugation at a moderate speed. Alternatively, a filtration step may be used for example with a 0.2 μm membrane. The concentration step may preferably employ an adsorption method, for instance, using CaHPO₄. Alternatively, filtration may be used, for example ultrafiltration. A further separation step may also be used in the method of the invention. This further separation step is preferably a zonal centrifugation separation, and may optionally use a sucrose gradient. The sucrose gradient may further comprise a preservative to prevent microbial growth. The splitting step may also be performed in a sucrose gradient, wherein the sucrose gradient contains the splitting agent. The method may further comprise a sterile filtration step, optionally at the end of the process. Preferably, there is an inactivation step prior to the final filtration step.

Methods of preparing split CAL virus formulations may further include treatment of the viral formulation with a DNA digesting enzyme, as described above. Treatment of the CAL virus formulation with a DNA digesting enzyme may occur at any time in the purification and splitting process. Preferably, the CAL virus formulation is treated with a DNA digesting enzyme prior to use of a detergent. Still more preferably, the CAL virus formulation is treated with a DNA digesting enzyme prior to treatment with a cationic detergent, such as CTAB.

Once the split virus is made, the virus is purified using methods well known in the art, such as those methods described above with reference to inactivated viruses.

Virus-Like Particles Comprising CAL Virus Antigens

The CAL virus antigens of the invention may be formulated into Virus Like Particles (“VLPs”). As used herein, the term “virus-like particle” or “VLP” refers to a non-replicating, empty virus shell. VLPs are generally composed of one or more viral proteins, such as, but not limited to those proteins referred to as capsid, coat, shell, surface and/or envelope proteins, or particle-forming polypeptides derived from these proteins. VLPs can form spontaneously upon recombinant expression of the protein in an appropriate expression system, such as a eukaryotic or prokaryotic expression system. Upon expression, the structural proteins self-assemble to form particles. Alternatively, viral structural proteins may be isolated from whole virus and formulated with phospholipids. Such viral structural proteins are referred to herein as “particle-forming polypeptides”. The phrase “particle-forming polypeptide” includes a full-length or near full-length viral protein, as well as a fragment thereof, or a viral protein with internal deletion, which has the ability to form VLPs under conditions that favor VLP formation. Accordingly, the polypeptide may comprise the full-length sequence, fragments, truncated and partial sequences, as well as analogs and precursor forms of the reference molecule. The term therefore includes deletions, additions and substitutions to the sequence, so long as the polypeptide retains the ability to form a VLP. Thus, the term includes natural variations of the specified polypeptide since variations in coat proteins often occur between viral isolates. The term also includes deletions, addition and substitutions that do not naturally occur in the reference protein, so long as the protein retains the ability to form a VLP. Preferred substitutions are those which are conservative in nature, i.e., those substitutions that take place within a family of amino acids that are related in their side chains. Such substitutions are described above.

VLPs are not infectious because no viral genome is present, however, these non-replicating, virus capsids mimic the structure of native virions. Due to their structure, VLPs can display a large number of antigenic sites on their surface (similar to a native virus). VLPs offer an advantage to live or attenuated vaccines in that they are much safer to both produce and administer, since they are not infectious. VLPs have been shown to induce both neutralizing antibodies as well as T-cell responses and can be presented by both class I and II MHC pathways.

Methods for producing particular VLPs are known in the art and discussed more fully below. The presence of VLPs in a composition can be detected using conventional techniques known in the art, such as by electron microscopy, x-ray crystallography, and the like. See, e.g., Baker et al., Biophys. J. (1991) 60:1445-1456; Hagensee et al., J. Virol. (1994) 68:4503-4505. For example, cryoelectron microscopy can be performed on vitrified aqueous samples of the VLP preparation in question, and images recorded under appropriate exposure conditions.

The VLPs of the invention can be formed from any viral protein, particle-forming polypeptide derived from the viral protein, or combination of viral proteins or fragments thereof, that have the capability of forming particles under appropriate conditions. The requirements for the particle-forming viral proteins are that if the particle is formed in the cytoplasm of the host cell, the protein must be sufficiently stable in the host cell in which it is expressed such that formation of virus-like structures will result, and that the polypeptide will automatically assemble into a virus-like structure in the cell of the recombinant expression system used. If the protein is secreted into culture media, conditions can be adjusted such that VLPs will form. Furthermore, the particle-forming protein should not be cytotoxic in the expression host and should not be able to replicate in the host in which the VLP will be used.

Preferably, the VLPs comprise one or more CAL virus antigens selected from the group consisting of (a) G1, (b) G2, (c) N, (d) NSm, (e) NSs, (f); immunogenic fragments of (a), (b), (c), (d) or (e); and immunogenic analogs of (a), (b), (c), (d), (e) or (f). Preferably, the VLPs comprise at least G1, and may comprise the entire M region as described above. The VLPs of the invention comprise at least one particle-forming polypeptide. In one embodiment, the particle-forming polypeptide is selected from one or more LACV antigens. In another embodiment, the particle-forming polypeptide is selected from the structural protein of a non-LACV antigen, such as, for example, from another CAL virus or another unrelated virus.

Thus, chimeric VLPs comprising particle-forming polypeptides or portions thereof from a virus other than a CAL virus are also included in the invention. Such particle-forming polypeptides may comprise a full-length polypeptide from a non-CAL virus. Alternatively, a particle-forming fragment may be used.

In one embodiment, a fragment of a non-LACV particle-forming polypeptide and a fragment of a LACV viral antigen are fused together. For instance, such chimeric polypeptides may comprise the endodomain and transmembrane domain of a non-LACV particle-forming polypeptide and the ectodomain of a LACV viral antigen.

Methods and suitable conditions for forming particles from a wide variety of viral proteins are known in the art. VLPs have been produced, for example from proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, QB-phage (such as coat proteins), GA-phage, fr-phage, AP205 phage, and Ty (such as retrotransposon Ty protein p1). VLPs are discussed further in WO 03/024480, WO 03/024481, and Niikura et al., Virology (2002) 293:273-280; Lenz et al., J. Immunology (2001) 5246-5355; Pinto, et al., J. Infectious Diseases (2003) 188:327-338; and Gerber et al., J. Virology (2001) 75(10):4752-4760.

As explained above, VLPs can spontaneously form when the particle-forming polypeptide of interest is recombinantly expressed in an appropriate host cell. Thus, the VLPs for use in the present invention may be prepared using recombinant techniques, well known in the art and described in detail above. The particles are then isolated using methods that preserve the integrity thereof, such as by gradient centrifugation, e.g., cesium chloride (CsCl) and sucrose gradients, and the like (see, e.g., Kirnbauer et al., J. Virol. (1993) 67:6929-6936), ion exchange chromatography (including anion exchange chromatography such as DMAE and TMAE), hydroxyapatitie chromatography (see WO 00/09671), hydrophobic interaction chromatography, gel filtration chromatography and other filtration methods such as nanometric filtration and ultrafiltration.

VLP formulations of the invention may be further processed by methods known in the art to disassemble the VLPs into smaller, protein-containing moieties using a high concentration of reducing agent, followed by reassembly of the VLPs by either removal of the reducing agent or by addition of excess oxidant. The resulting reassembled VLPs may have improved homogeneity, stability and immunogenic properties. In addition, further therapeutic or prophylactic agents may be formulated into the VLPs upon reassembly. See McCarthy et al., J. Virology (1998) 72(1):32-41. See also WO 99/13056 and WO 01/42780. Reducing agents suitable for use in VLP disassembly include sulfhydryl reducing agents (such as glutathion, beta mercaptoethanol, dithiothreitol, dithioerythritol, cysteine, hydrogen sulfide and mixtures thereof) preferably contained in moderate to low ionic strength buffers. Sufficient exposure time of the VLPs to the reducing agent will be required to achieve a suitable amount of VLP disassembly.

VLPs may be formulated into immunogenic compositions as described below. The VLPs of the invention may formulated to enhance their stability. Additional components which may enhance the stability of a VLP formulation include salts, buffers, non-ionic surfactants and other stabilizers such as polymeric polyanion stabilizers. See WO 00/45841. The ionic strength of a solution comprising VLP particles may be maintained by the presence of salts. Almost any salt which can contribute to the control of the ionic strength may be used. Preferred salts which can be used to adjust ionic strength include physiologically acceptable salts such as NaCl, KCl, Na₂SO₄, (NH₄)₂SO₄, sodium phosphate and sodium citrate. Preferably, the salt component is present in concentrations of from about 0.10 M to 1 M. Very high concentrations are not preferred due to the practical limitations of parenteral injection of high salt concentrations. Instead, more moderate salt concentrations, such as more physiological concentrations of about 0.15 M to about 0.5 M with 0.15 M-0.32 M NaCl are preferred.

Buffers may also be used to enhance the stability of the VLP formulations of the invention. Preferably, the buffer optimizes the VLP stability while maintaining the pH range so that the formulation will not be irritating to the recipient. Buffers preferably maintain the pH of the vaccine formulation within a range of p/H 5.5-7.0, more preferably 6.0-6.5. Buffers suitable for vaccine formulations are known in the art and include, for example, histidine and imidazole. Preferably, the concentration of the buffer will range from about 2 mM to about 100 mM, more preferably 5 mM to about 20 mM. Phosphate containing buffers are generally not preferred when the VLP is adsorbed or otherwise formulated with an aluminum compound.

Non-ionic surfactants may be used to enhance the stability of the VLP formulations of the invention. Surfactants suitable for use in vaccine formulations are known in the art and include, for example, polyoxyethylene sorbital fatty acid esters (Polysorbates) such as Polysorbate 80 (e.g., TWEEN 80), Polysorbate 20 (e.g., TWEEN 20), polyoxyethylene alkyl ethers (e.g., Brij 35, Brij 58), as well as others, including Triton X-100, Triton X-114, NP-40, Span 85 and the Pluronic series of non-ionic surfactants (e.g., Pluronic 121). The surfactant is preferably present in a concentration of from about 0.0005% to about 0.5% (wt/vol).

Polymeric polyanion stabilizers may also be used to enhance the stability of the VLP formulations of the invention. Suitable polymeric polyanionic stabilizers for use in the invention comprise either a single long chain or multiple cross linked chains; either type possessing multiple negative charges along the chains when in solution. Examples of suitable polyanionic polymers include proteins, polyanions, peptides and polynucelic acids. Specific examples include carboxymethyl cellulose, heparin, polyamino acids (such as poly(Glu), poly(Asp), and Poly (Glu, Phe), oxidized glutathione, polynuceltodies, RNA, DNA and serum albumins. The concentration of the polymeric polyanion stabilizers is preferably from about 0.01% to about 0.5%, particularly about 0.05-0.1% (by weight).

Compositions Comprising CAL Viruses, Polypeptides and Polynucleotides

The invention provides compositions including the above-described CAL viruses (e.g., inactivated, attenuated and split), as well as CAL virus VLPs, CAL polypeptides (intracellularly produced or secreted) and/or polynucleotides. Compositions of the invention may comprise a pharmaceutically acceptable carrier. The carrier should not itself induce the production of antibodies harmful to the host. Pharmaceutically acceptable carriers are well known to those in the art. Such carriers include, but are not limited to, large, slowly metabolized, macromolecules, such as proteins, polysaccharides such as latex functionalized sepharose, agarose, cellulose, cellulose beads and the like, polylactic acids, polyglycolic acids, polymeric amino acids such as polyglutamic acid, polylysine, and the like, amino acid copolymers, and inactive virus particles.

Pharmaceutically acceptable salts can also be used in compositions of the invention, for example, mineral salts such as hydrochlorides, hydrobromides, phosphates, or sulfates, as well as salts of organic acids such as acetates, propionates, malonates, or benzoates. Especially useful protein substrates are serum albumins, keyhole limpet hemocyanin, immunoglobulin molecules, thyroglobulin, ovalbumin, tetanus toxoid, and other proteins well known to those of skill in the art. Compositions of the invention can also contain liquids or excipients, such as water, saline, glycerol, dextrose, ethanol, or the like, singly or in combination, as well as substances such as wetting agents, emulsifying agents, or pH buffering agents. Liposomes can also be used as a carrier for a composition of the invention and are described below.

If desired, co-stimulatory molecules which improve immunogen presentation to lymphocytes, such as B7-1 or B7-2, or cytokines such as GM-CSF, IL-2, and IL-12, can be included in a composition of the invention.

Optionally, adjuvants can also be included in a composition. Adjuvants which can be used include, but are not limited to: (1) aluminum salts (alum), such as aluminum hydroxide, aluminum phosphate, aluminum sulfate, etc.; (2) oil-in-water emulsion formulations (with or without other specific immunostimulating agents such as muramyl peptides (see below) or bacterial cell wall components), such as for example (a) MF59 (U.S. Pat. No. 6,299,884, incorporated herein by reference in its entirety; Chapter 10 in Vaccine design: the subunit and adjuvant approach, eds. Powell & Newman, Plenum Press 1995), containing 5% Squalene, 0.5% TWEEN 80™, and 0.5% SPAN 85™ (optionally containing various amounts of MTP-PE (see below), although not required) formulated into submicron particles using a microfluidizer such as Model 110Y microfluidizer (Microfluidics, Newton, Mass.), (b) SAF, containing 10% Squalane, 0.4% TWEEN 80™, 5% pluronic-blocked polymer L121, and thr-MDP either microfluidized into a submicron emulsion or vortexed to generate a larger particle size emulsion, and (c) RIBI™ adjuvant system (RAS), (Ribi Immunochem, Hamilton, Mont.) containing 2% Squalene, 0.2% TWEEN 80™, and one or more bacterial cell wall components from the group consisting of monophosphorylipid A (MPL), trehalose dimycolate (TDM), and cell wall skeleton (CWS), preferably MPL+CWS (DETOX™); (3) saponin adjuvants, such as QS21 or STIMULON™ (Cambridge Bioscience, Worcester, Mass.) may be used or particles generated therefrom such as ISCOMs (immunostimulating complexes), which ISCOMs may be devoid of additional detergent, see, e.g., International Publication No. WO 00/07621; (4) Complete Freund's Adjuvant (CFA) and Incomplete Freund's Adjuvant (IFA); (5) cytokines, such as interleukins (IL-1, IL-2, IL-4, IL-5, IL-6, IL-7, IL-12 (International Publication No. WO 99/44636), etc.), interferons (e.g., gamma interferon), macrophage colony stimulating factor (M-CSF), tumor necrosis factor (TNF), etc.; (6) detoxified mutants of a bacterial ADP-ribosylating toxin such as a cholera toxin (CT), a pertussis toxin (PT), or an E. coli heat-labile toxin (LT), particularly LT-K63 (where lysine is substituted for the wild-type amino acid at position 63) LT-R72 (where arginine is substituted for the wild-type amino acid at position 72), CT-S109 (where serine is substituted for the wild-type amino acid at position 109), and PT-K9/G129 (where lysine is substituted for the wild-type amino acid at position 9 and glycine substituted at position 129) (see, e.g., International Publication Nos. WO93/13202 and WO92/19265); (7) MPL or 3-O-deacylated MPL (3dMPL) (see, e.g., GB 2220221), EP-A-0689454, optionally in the substantial absence of alum when used with pneumococcal saccharides (see, e.g., International Publication No. WO 00/56358); (8) combinations of 3dMPL with, for example, QS21 and/or oil-in-water emulsions (see, e.g., EP-A-0835318, EP-A-0735898, EP-A-0761231; (9) oligonucleotides comprising CpG motifs (see, e.g., Roman et al. (1997) Nat. Med. 3:849-854; Weiner et al. (1997) Proc. Natl. Acad. Sci. USA 94:10833-10837; Davis et al. (1998) J. Immunol. 160:870-876; Chu et al. (1997) J. Exp. Med. 186:1623-1631; Lipford et al. (1997) Eur. J. Immunol. 27:2340-2344; Moldoveanu et al. (1988) Vaccine 16:1216-1224; Krieg et al. (1995) Nature 374:546-549; Klinman et al. (1996) Proc. Natl. Acad. Sci. USA 93:2879-2883; Ballas et al. (1996) J. Immunol. 157:1840-1845; Cowdery et al. (1996) J. Immunol. 156:4570-4575; Halpern et al. (1996) Cell Immunol. 167:72-78; Yamamoto et al. (1988) Jpn. J. Cancer Res. 79:866-873; Stacey et al. (1996) J. Immunol. 157:2116-2122; Messina et al. (1991) J. Immunol. 147:1759-1764; Yi et al. (1996) J. Immunol. 157:4918-4925; Yi et al. (1996) J. Immunol. 157:5394-5402; Yi et al. (1998) J. Immunol. 160:4755-4761; Yi et al. (1998) J. Immunol. 160:5898-5906; International Publication Nos. WO 96/02555, WO 98/16247, WO 98/18810, WO 98/40100, WO 98/55495, WO 98/37919 and WO 98/52581), such as those containing at least on CG dinucleotide, with cytosine optionally replaced with 5-methylcytosine; (10) a polyoxyethylene ether or a polyoxyethylene ester (see, e.g., International Publication No. WO 99/52549); (11) a polyoxyethylene sorbitan ester surfactant in combination with an octoxynol (see, e.g., International Publication No. WO 01/21207) or a polyoxyethylene alkyl ether or ester surfactant in combination with at least one additional non-ionic surfactant such as an octoxynol (see, e.g., International Publication No. WO 01/21152); (12) a saponin and an immunostimulatory oligonucleotide such as a CpG oligonucleotide (see, e.g., International Publication No. WO 00/62800); (13) an immunostimulant and a particle of metal salt (see, e.g., International Publication No. WO 00/23105); and (14) other substances that act as immunostimulating agents to enhance the effectiveness of the composition.

Muramyl peptides include, but are not limited to, N-acetyl-muramyl-L-threonyl-D-isoglutamine (thr-MDP), N-acteyl-normuramyl-L-alanyl-D-isogluatme (nor-MDP), -acetylmuramyl-L-alanyl-D-isogluatminyl-L-alanine-2-(1′-2′-dipalmitoyl-sn-glycero-3-hydroxyphosphoryloxy)-ethylamine (MTP-PE), etc.

Particularly preferred adjuvants for use in the compositions are submicron oil-in-water emulsions. Preferred submicron oil-in-water emulsions for use herein are squalene/water emulsions optionally containing varying amounts of MTP-PE, such as a submicron oil-in-water emulsions containing 4-5% w/v squalene, 0.25-1.0% w/v Tween 80 ™ (polyoxyelthylenesorbitan monooleate), and/or 0.25-1.0% Span 85™ (sorbitan trioleate), and optionally, N-acetylmuramyl-L-alanyl-D-isogluatminyl-L-alanine-2-(1′-2′-dipalmitoyl-sn-glycero-3-huydroxyphosphoryloxy)-ethylamine (MTP-PE), for example, the submicron oil-in-water emulsion known as “MF59” (International Publication No. WO 90/14837; U.S. Pat. Nos. 6,299,884 and 6,451,325, incorporated herein by reference in their entireties; and Ott et al., “MF59—Design and Evaluation of a Safe and Potent Adjuvant for Human Vaccines” in Vaccine Design: The Subunit and Adjuvant Approach (Powell, M. F. and Newman, M. J. eds.) Plenum Press, New York, 1995, pp. 277-296). MF59 contains 4-5% w/v Squalene (e.g., 4.3%), 0.25-0.5% w/v Tween 80™, and 0.5% w/v Span 85™ and optionally contains various amounts of MTP-PE, formulated into submicron particles using a microfluidizer such as Model 110Y microfluidizer (Microfluidics, Newton, Mass.). For example, MTP-PE may be present in an amount of about 0-500 μg/dose, more preferably 0-250 μg/dose and most preferably, 0-100 μg/dose. As used herein, the term “MF59-0” refers to the above submicron oil-in-water emulsion lacking MTP-PE, while the term MF59-MTP denotes a formulation that contains MTP-PE. For instance, “MF59-100” contains 100 μg MTP-PE per dose, and so on. MF69, another submicron oil-in-water emulsion for use herein, contains 4.3% w/v squalene, 0.25% w/v Tween 80™, and 0.75% w/v Span 85™ and optionally MTP-PE. Yet another submicron oil-in-water emulsion is MF75, also known as SAF, containing 10% squalene, 0.4% Tween 80™, 5% pluronic-blocked polymer L121, and thr-MDP, also microfluidized into a submicron emulsion. MF75-MTP denotes an MF75 formulation that includes MTP, such as from 100-400 μg MTP-PE per dose.

Submicron oil-in-water emulsions, methods of making the same and immunostimulating agents, such as muramyl peptides, for use in the compositions, are described in detail in International Publication No. WO 90/14837 and U.S. Pat. Nos. 6,299,884 and 6,451,325, incorporated herein by reference in their entireties.

Other preferred agents to include in the subject compositions are immunostimulatory molecules such as immunostimulatory nucleic acid sequences (ISS), including but not limited to, unmethylated CpG motifs, such as CpG oligonucleotides.

Oligonucleotides containing unmethylated CpG motifs have been shown to induce activation of B cells, NK cells and antigen-presenting cells (APCs), such as monocytes and macrophages. See, e.g., U.S. Pat. No. 6,207,646. Thus, adjuvants derived from the CpG family of molecules, CpG dinucleotides and synthetic oligonucleotides which comprise CpG motifs (see, e.g., Krieg et al. Nature (1995) 374:546 and Davis et al. J. Immunol. (1998) 160:870-876) such as any of the various immunostimulatory CpG oligonucleotides disclosed in U.S. Pat. No. 6,207,646, may be used in the subject methods and compositions. Such CpG oligonucleotides generally comprise at least 8 up to about 100 basepairs, preferably 8 to 40 basepairs, more preferably 15-35 basepairs, preferably 15-25 basepairs, and any number of basepairs between these values. For example, oligonucleotides comprising the consensus CpG motif, represented by the formula 5′-X₁CGX₂₋3′, where X₁ and X₂ are nucleotides and C is unmethylated, will find use as immunostimulatory CpG molecules. Generally, X₁ is A, G or T, and X₂ is C or T. Other useful CpG molecules include those captured by the formula 5′-X₁X₂CGX₃X₄, where X₁ and X₂ are a sequence such as GpT, GpG, GpA, ApA, ApT, ApG, CpT, CpA, CpG, TpA, TpT or TpG, and X₃ and X₄ are TpT, CpT, ApT, ApG, CpG, TpC, ApC, CpC, TpA, ApA, GpT, CpA, or TpG, wherein “p” signifies a phosphate bond. Preferably, the oligonucleotides do not include a GCG sequence at or near the 5′- and/or 3′ terminus. Additionally, the CpG is preferably flanked on its 5′-end with two purines (preferably a GpA dinucleotide) or with a purine and a pyrimidine (preferably, GpT), and flanked on its 3′-end with two pyrimidines, preferably a TpT or TpC dinucleotide. Thus, preferred molecules will comprise the sequence GACGTT, GACGTC, GTCGTT or GTCGCT, and these sequences will be flanked by several additional nucleotides. The nucleotides outside of this central core area appear to be extremely amendable to change.

Moreover, the CpG oligonucleotides for use herein may be double- or single-stranded. Double-stranded molecules are more stable in vivo while single-stranded molecules display enhanced immune activity. Additionally, the phosphate backbone may be modified, such as phosphorodithioate-modified, in order to enhance the immunostimulatory activity of the CpG molecule. As described in U.S. Pat. No. 6,207,646, CpG molecules with phosphorothioate backbones preferentially activate B-cells, while those having phosphodiester backbones preferentially activate monocytic (macrophages, dendritic cells and monocytes) and NK cells.

CpG molecules can readily be tested for their ability to stimulate an immune response using standard techniques, well known in the art. For example, the ability of the molecule to stimulate a humoral and/or cellular immune response is readily determined using the immunoassays described above. Moreover, the immunogenic compositions can be administered with and without the CpG molecule to determine whether an immune response is enhanced.

The CAL virus molecules may also be encapsulated, adsorbed to, or associated with, particulate carriers. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; and McGee et al., J. Microencap. (1996). One preferred method for adsorbing macromolecules onto prepared microparticles is described in International Publication No. WO 00/050006, incorporated herein by reference in its entirety.

Compositions for use in the invention will comprise a therapeutically effective amount of the desired CAL molecule or inactivated or attenuated CAL virus and any other of the above-mentioned components, as needed. By “therapeutically effective amount” is meant an amount of a protein or DNA encoding the same which will induce an immunological response, preferably a protective immunological response, in the individual to which it is administered, if the composition is to be used as a vaccine. Such a response will generally result in the development in the subject of an antibody-mediated and/or a secretory or cellular immune response to the composition. Usually, such a response includes but is not limited to one or more of the following effects; the production of antibodies from any of the immunological classes, such as immunoglobulins A, D, E, G or M; the proliferation of B and T lymphocytes; the provision of activation, growth and differentiation signals to immunological cells; expansion of helper T cell, suppressor T cell, and/or cytotoxic T cell and/or γδT cell populations.

Combinations of CAL Virus Preparations and Other Antigens

The invention further relates to vaccine formulations including one or more bacterial or viral antigens in combination with the CAL virus preparations. Antigens may be used alone or in any combination. The combinations may include multiple antigens from the same pathogen, multiple antigens from different pathogens or multiple antigens from the same and from different pathogens. Thus, bacterial, viral, and/or other antigens may be included in the same composition or may be administered to the same subject separately.

For example, the compositions can include one or more antigens from multiple CAL virus isolates, as well as from other encephalitis-causing viruses. Such viruses include, without limitation, West Nile Virus (WNV), Yellow Fever virus, Japanese Encephalitis virus, toscana virus, tick-borne encephalitis virus, rabies virus, Western Equine Encephalitis virus, Eastern Equine Encephalitis virus, Venezuelan Equine Encephalitis virus, St. Louis Encephalitis virus, Dengue virus, Russian Spring-Summer Encephalitis virus, Varicella Zoster virus, Herpes Simplex-2 virus, Epstein Barr virus, other human herpesviruses such as HHV6 and HHV7, among others. Preferred antigens to include with the present CAL virus preparations include those derived from WNV, St. Louis Encephalitis virus, Western Equine Encephalitis virus, Eastern Equine Encephalitis virus and Venezuelan Equine Encephalitis virus, with WNV and St. Louis Encephalitis virus preferred.

Non-limiting examples of bacterial pathogens which may be used in the invention include diphtheria (See, e.g., Chapter 3 of Vaccines, 1998, eds. Plotkin & Mortimer (ISBN 0-7216-1946-0), staphylococcus (e.g., Staphylococcus aureus as described in Kuroda et al. (2001) Lancet 357:1225-1240), cholera, tuberculosis, C. tetani, also known as tetanus (See, e.g., Chapter 4 of Vaccines, 1998, eds. Plotkin & Mortimer (ISBN 0-7216-1946-0), Group A and Group B streptococcus (including Streptococcus pneumoniae, Streptococcus agalactiae and Streptococcus pyogenes as described, for example, in Watson et al. (2000) Pediatr. Infect. Dis. J 19:331-332; Rubin et al. (2000) Pediatr Clin. North Am. 47:269-284; Jedrzejas et al. (2001) Microbiol Mol Biol Rev 65:187-207; Schuchat (1999) Lancet 353:51-56; GB patent applications 0026333.5; 0028727.6; 015640.7; Dale et al. (1999) Infect Dis Clin North Am 13:227-1243; Ferretti et al. (2001) PNAS USA 98:4658-4663), pertussis (See, e.g., Gusttafsson et al. (1996) N. Engl. J. Med. 334:349-355; Rappuoli et al. (1991) TIBTECH 9:232-238), meningitis, Moraxella catarrhalis (See, e.g., McMichael (2000) Vaccine 19 Suppl. 1:S101-107) and other pathogenic states, including, without limitation, Neisseria meningitidis (A, B, C, Y), Neisseria gonorrhoeae (See, e.g., WO 99/24578; WO 99/36544; and WO 99/57280), Helicobacter pylori (e.g., CagA, VacA, NAP, HopX, HopY and/or urease as described, for example, WO 93/18150; WO 99/53310; WO 98/04702) and Haemophilus influenza. Hemophilus influenza type B (HIB) (See, e.g., Costantino et al. (1999) Vaccine 17:1251-1263), Porphyromonas gingivalis (Ross et al. (2001) Vaccine 19:4135-4132) and combinations thereof.

Non-limiting examples of viral pathogens which may be used in the invention include meningitis, rhinovirus, influenza (Kawaoka et al., Virology (1990) 179:759-767; Webster et al., “Antigenic variation among type A influenza viruses,” p. 127-168. In: P. Palese and D. W. Kingsbury (ed.), Genetics of influenza viruses. Springer-Verlag, New York), respiratory syncytial virus (RSV), parainfluenza virus (PIV), rotavirus (e.g., VP1, VP2, VP3, VP4, VP6, VP7, NSP1, NSP2, NSP3, NSP4 or NSP5 and other rotavirus antigens, for example as described in WO 00/26380) and the like. Antigens derived from other viruses will also find use in the present invention, such as without limitation, proteins from members of the families Picornaviridae (e.g., polioviruses, etc. as described, for example, in Sutter et al. (2000) Pediatr Clin North Am 47:287-308; Zimmerman & Spann (1999) Am Fam Physician 59:113-118; 125-126); Caliciviridae; Togaviridae (e.g., rubella virus, etc.); Flaviviridae, including the genera flavivirus (e.g., yellow fever virus, Japanese encephalitis virus, serotypes of Dengue virus, tick borne encephalitis virus, West Nile virus, St. Louis encephalitis virus); pestivirus (e.g., classical porcine fever virus, bovine viral diarrhea virus, border disease virus); and hepacivirus (e.g., hepatitis A, B and C as described, for example, in U.S. Pat. Nos. 4,702,909; 5,011,915; 5,698,390; 6,027,729; and 6,297,048); Parvovirus (e.g., parvovirus B19); Coronaviridae; Reoviridae; Bimaviridae; Rhabodoviridae (e.g., rabies virus, etc. as described for example in Dressen et al. (1997) Vaccine 15 Suppl:s2-6; MMWR Morb Mortal Wkly Rep. 1998 January 16:47(1):12, 19); Filoviridae; Paramyxoviridae (e.g., mumps virus, measles virus, respiratory syncytial virus, etc. as described in Chapters 9 to 11 of Vaccines, 1998, eds. Plotkin & Mortimer (ISBN 0-7216-1946-0); Orthomyxoviridae (e.g., influenza virus types A, B and C, etc. as described in Chapter 19 of Vaccines, 1998, eds. Plotkin & Mortimer (ISBN 0-7216-1946-0)); Bunyaviridae; Arenaviridae; Retroviradae (e.g., HTLV-1; HTLV-11; HIV-1 (also known as HTLV-III, LAV, ARV, HTI, R₁ etc.)), including but not limited to antigens from the isolates HR/Illb, HIVSF2, HIVLAV, HIVI-AL, I-IIVMN, SF162); HIV-I CM235, HIV-I US4; HIV-2; simian immunodeficiency virus (SIV) among others. Additionally, antigens may also be derived from human papilloma virus (HPV) and the tick-borne encephalitis viruses. See, e.g. Virology, 3^{th} Edition (W. K. Joklik ed. 1988); Fundamental Virology, 2^{nd} Edition (B. N. Fields and D. M. Knipe, eds, 1991), for a description of these and other viruses.

Antigens may also be derived from the herpesvirus family, including proteins derived from herpes simplex virus (HSV) types 1 and 2, such as HSV-1 and HSV-2 glycoproteins gB, gD and gH; antigens derived from varicella zoster virus (VZV), Epstein-Barr virus (EBV) and cytomegalovirus (CMV) including CMV gB and gH (See, U.S. Pat. No. 4,689,225 and PCT Publication WO 89/07143); and antigens derived from other human herpesviruses such as HHV6 and HHV7. (See, e.g. Chee et al., Cytomegaloviruses (J. K. McDougall, ed., Springer-Verlag 1990) pp. 125-169, for a review of the protein coding content of cytomegalovirus; McGeoch et al., J. Gen. Virol. (1988) 69:1531-1574, for a discussion of the various HSV-1 encoded proteins; U.S. Pat. No. 5,171,568 for a discussion of HSV-1 and HSV-2 gB and gD proteins and the genes encoding therefore; Baer et al., Nature (1984) 310:207-211, for the identification of protein coding sequences in an EBV genome; and Davison and Scott, J. Gen. Virol. (1986) 67:1759-1816, for a review of VZV). Herpes simplex virus (HSV) rgD2 is a recombinant protein produced in genetically engineered Chinese hamster ovary cells. This protein has the normal anchor region truncated, resulting in a glycosylated protein secreted into tissue culture medium. The gD2 can be purified in the CHO medium to greater than 90% purity. Human immunodeficiency virus (HIV) env-2-3 is a recombinant form of the HIV enveloped protein produced in genetically engineered Saccharomyces cerevisae. This protein represents the entire protein region of HIV gp120 but is non-glycosylated and denatured as purified from the yeast. HIV gp120 is a fully glycosylated, secreted form of gp120 produced in CHO cells in a fashion similar to the gD2 above. Additional HSV antigens suitable for use in immunogenic compositions are described in PCT Publications WO 85/04587 and WO 88/02634, the disclosures of which are incorporated herein by reference in their entirety. Mixtures of gB and gD antigens, which are truncated surface antigens lacking the anchor regions, are particularly preferred.

Antigens from the hepatitis family of viruses, including hepatitis A virus (HAV) (See, e.g., Bell et al. (2000) Pediatr Infect Dis. J. 19:1187-1188; Iwarson (1995) APMIS 103:321-326), hepatitis B virus (HBV) (See, e.g., Gerlich et al. (1990) Vaccine 8 Suppl: S63-68 & 79-80), hepatitis C virus (HCV) (See, e.g., PCT/US88/04125, published European application number 318216), the delta hepatitis virus (HDV), hepatitis E virus (HMV) and hepatitis G virus (HGV), can also be conveniently used in the techniques described herein. By way of example, the viral genomic sequence of HCV is known, as are methods for obtaining the sequence. See, e.g., International Publication Nos. WO 89/04669; WO 90/11089; and WO 90/14436. Also included in the invention are molecular variants of such polypeptides, for example as described in PCT/US99/31245; PCT/US99/31273 and PCT/US99/31272. The HCV genome encodes several viral proteins, including E1 (also known as E) and E2 (also known as E2/NSI) and an N-terminal nucleocapsid protein (termed “core”) (see, Houghton et al., Hepatology (1991) 14:381-388, for a discussion of HCV proteins, including E1 and E2). Similarly, the sequence for the 8-antigen from HDV is known (see, e.g., U.S. Pat. No. 5,378,814) and this antigen can also be conveniently used in the present composition and methods. Additionally, antigens derived from HBV, such as the core antigen, the surface antigen, SAg, as well as the presurface sequences, pre-S1 and pre-S2 (formerly called pre-S), as well as combinations of the above, such as SAg/pre-S1, SAg/pre-S2, SAg/pre-S1/pre-S2, and pre-S1/pre-S2, will find use herein. See, e.g., “HBV Vaccines—from the laboratory to license: a case study” in Mackett, M. and Williamson, J. D., Human Vaccines and Vaccination, pp. 159-176, for a discussion of HBV structure; and U.S. Pat. Nos. 4,722,840, 5,098,704, 5,324,513, incorporated herein by reference in their entireties; Beames et al., J. Virol. (1995) 69:6833-6838, Birnbaum et al., J. Virol. (1990) 64:3319-3330; and Zhou et al., J. Virol. (1991) 65:5457-5464. Each of these proteins, as well as antigenic fragments thereof, will find use in the present composition and methods.

Influenza virus is another example of a virus for which the present invention will be particularly useful. Specifically, the envelope glycoproteins HA and NA of influenza A are of particular interest for generating an immune response. Numerous HA subtypes of influenza A have been identified (Kawaoka et al., Virology (1990) 179:759-767; Webster et al., “Antigenic variation among type A influenza viruses,” p. 127-168. In: P. Palese and D. W. Kingsbury (ed.), Genetics of influenza viruses. Springer-Verlag, New York). Thus, proteins derived from any of these isolates can also be used in the compositions and methods described herein.

Administration

The immunogenic compositions (both DNA and protein) can be prepared as injectables, either as liquid solutions or suspensions; solid forms suitable for solution in, or suspension in, liquid vehicles prior to injection may also be prepared. Thus, once formulated, the compositions are conventionally administered parenterally, e.g., by injection, either subcutaneously or intramuscularly. For example, the immunogen is preferably administered intramuscularly to a large mammal, such as a primate, for example, a baboon, chimpanzee, or human. Additional formulations suitable for other modes of administration include oral and pulmonary formulations, suppositories, and transdermal formulations, aerosol, intranasal, oral formulations, and sustained release formulations.

For suppositories, the vehicle composition will include traditional binders and carriers, such as, polyalkaline glycols, or triglycerides. Such suppositories may be formed from mixtures containing the active ingredient in the range of about 0.5% to about 10% (w/w), preferably about 1% to about 2%. Oral vehicles include such normally employed excipients as, for example, pharmaceutical grades of mannitol, lactose, starch, magnesium, stearate, sodium saccharin cellulose, magnesium carbonate, and the like. These oral vaccine compositions may be taken in the form of solutions, suspensions, tablets, pills, capsules, sustained release formulations, or powders, and contain from about 10% to about 95% of the active ingredient, preferably about 25% to about 70%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

Controlled or sustained release formulations are made by incorporating the active agent into carriers or vehicles such as liposomes, nonresorbable impermeable polymers such as ethylenevinyl acetate copolymers and Hytrel copolymers, swellable polymers such as hydrogels, or resorbable polymers such as collagen and certain polyacids or polyesters such as those used to make resorbable sutures. The immunogens can also be delivered using implanted mini-pumps, well known in the art.

The immunogens of the instant invention can also be administered via a carrier virus which expresses the same. Carrier viruses which will find use with the instant invention include but are not limited to the vaccinia and other pox viruses, adenovirus, and herpes virus. By way of example, vaccinia virus recombinants expressing the novel proteins can be constructed as follows. The DNA encoding the particular protein is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the instant protein into the viral genome. The resulting TK⁻ recombinant can be selected by culturing the cells in the presence of 5-bromodeoxyuridine and picking viral plaques resistant thereto.

The immunogens can be administered either to a mammal that is not infected with a CAL virus or can be administered to a CAL-infected mammal.

Dosage treatment may be a single dose schedule or a multiple dose schedule. Preferably, the effective amount is sufficient to bring about treatment or prevention of disease symptoms. The exact amount necessary will vary depending on the subject being treated; the age and general condition of the individual to be treated; the capacity of the individual's immune system to synthesize antibodies; the degree of protection desired; the severity of the condition being treated; the particular macromolecule selected and its mode of administration, among other factors. An appropriate effective amount can be readily determined by one of skill in the art. A “therapeutically effective amount” will fall in a relatively broad range that can be determined through routine trials using in vitro and in vivo models known in the ark

Thus, for example, if polypeptide immunogens are delivered, generally the amount administered will be about 0.1 μg to about 750 μg of immunogen per dose, or any amount between the stated ranges, such as 1 μg to about 500 μg, 5 μg to about 250 μg, 10 μg to about 100 μg, 10 μg to about 50 μg, such as 4, 5, 6, 7, 8, 10 . . . 20 . . . 25 . . . 30 . . . 35 . . . 40 . . . 50 . . . 60 . . . 70 . . . 80 . . . 90 . . . 100, etc., μg per dose.

In one embodiment, a lower concentration of viral antigen is used in the vaccine compositions of the invention. Such lower concentration vaccines may optionally comprise an adjuvant to boost the host immune response to the antigen. In such a “low dose” vaccine, the viral antigen is preferably present in a concentration of less than 15 μg antigen/dose, (i.e., less than 10, 7.5, 5 or 3 μg antigen/dose.

As explained above, expression constructs, such as constructs encoding individual CAL virus immunogens or fusions, may be used for nucleic acid immunization to stimulate an immunological response, such as a cellular immune response and/or humoral immune response, using standard gene delivery protocols. Methods for gene delivery are known in the art. See, e.g., U.S. Pat. Nos. 5,399,346, 5,580,859, 5,589,466, incorporated by reference herein in their entireties. Genes can be delivered either directly to the subject or, alternatively, delivered ex vivo, to cells derived from the subject and the cells reimplanted in the subject. For example, the constructs can be delivered as plasmid DNA, e.g., contained within a plasmid, such as pBR322, pUC, or ColE1.

Additionally, the expression constructs can be packaged in liposomes prior to delivery to the cells. Lipid encapsulation is generally accomplished using liposomes which are able to stably bind or entrap and retain nucleic acid. The ratio of condensed DNA to lipid preparation can vary but will generally be around 1:1 (mg DNA:micromoles lipid), or more of lipid. For a review of the use of liposomes as carriers for delivery of nucleic acids, see, Hug and Sleight, Biochim. Biophys. Acta. (1991) 1097:1-17; Straubinger et al., in Methods of Enzymology (1983), Vol. 101, pp. 512-527.

Liposomal preparations for use with the present invention include cationic (positively charged), anionic (negatively charged) and neutral preparations, with cationic liposomes particularly preferred. Cationic liposomes are readily available. For example, N[1-2,3-dioleyloxy)propyl]-N,N,N-triethyl-ammonium (DOTMA) liposomes are available under the trademark Lipofectin, from GIBCO BRL, Grand Island, N.Y. (See, also, Feigner et al., Proc. Natl. Acad. Sci. USA (1987) 84:7413-7416). Other commercially available lipids include transfectace (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA (1978) 75:4194-4198; PCT Publication No. WO 90/11092 for a description of the synthesis of DOTAP (1,2-bis(oleoyloxy)-3-(trimethylammonio)propane) liposomes. The various liposome-nucleic acid complexes are prepared using methods known in the art. See, e.g., Straubinger et al., in METHODS OF IMMUNOLOGY (1983), Vol. 101, pp. 512-527; Szoka et al., Proc. Natl. Acad. Sci. USA (1978) 75:4194-4198; Papahadjopoulos et al., Biochim. Biophys. Acta (1975) 394:483; Wilson et al., Cell (1979) 17:77); Deamer and Bangham, Biochim. Biophys. Acta (1976) 443:629; Ostro et al., Biochem. Biophys. Res. Commun. (1977) 76:836; Fraley et al., Proc. Natl. Acad. Sci. USA (1979) 76:3348); Enoch and Strittmatter, Proc. Natl. Acad. Sci. USA (1979) 76:145); Fraley et al., J. Biol. Chem. (1980) 255:10431; Szoka and Papahadjopoulos, Proc. Natl. Acad. Sci. USA (1978) 75:145; and Schaefer-Ridder et al., Science (1982) 215:166.

The DNA can also be delivered in cochleate lipid compositions similar to those described by Papahadjopoulos et al., Biochem. Biophys. Acta. (1975) 394:483-491. See, also, U.S. Pat. Nos. 4,663,161 and 4,871,488.

A number of viral based systems have been developed for gene transfer into mammalian cells. For example, retroviruses provide a convenient platform for gene delivery systems, such as murine sarcoma virus, mouse mammary tumor virus, Moloney murine leukemia virus, and Rous sarcoma virus. A selected gene can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman, BioTechniques (1989) 7:980-990; Miller, A. D., Human Gene Therapy (1990) 1:5-14; Scarpa et al., Virology (1991) 180:849-852; Burns et al., Proc. Natl. Acad. Sci. USA (1993) 90:8033-8037; and Boris-Lawrie and Temin, Cur. Opin. Genet. Develop. (1993) 3:102-109. Briefly, retroviral gene delivery vehicles of the present invention may be readily constructed from a wide variety of retroviruses, including for example, B, C, and D type retroviruses as well as spumaviruses and lentiviruses such as FIV, HIV, HIV-1, HIV-2 and SIV (see RNA Tumor Viruses, Second Edition, Cold Spring Harbor Laboratory, 1985). Such retroviruses may be readily obtained from depositories or collections such as the American Type Culture Collection (“ATCC”; 10801. University Blvd., Manassas, Va. 20110-2209), or isolated from known sources using commonly available techniques.

A number of adenovirus vectors have also been described, such as adenovirus Type 2 and Type 5 vectors. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BloTechniques (1988) 6:616-629; and Rich et al., Human Gene Therapy (1993) 4:461-476).

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the Alphavirus genus, such as but not limited to vectors derived from the Sindbis and Semliki Forest viruses, VEE, will also find use as viral vectors for delivering the gene of interest. For a description of Sindbis-virus derived vectors useful for the practice of the instant methods, see, Dubensky et al., J. Virol. (1996) 70:508-519; and International Publication Nos. WO 95/07995 and WO 96/17072.

Other vectors can be used, including but not limited to simian virus 40 and cytomegalovirus. Bacterial vectors, such as Salmonella ssp. Yersinia enterocolitica, Shigella spp., Vibrio cholerae, Mycobacterium strain BCG, and Listeria monocytogenes can be used. Minichromosomes such as MC and MC1, bacteriophages, cosmids (plasmids into which phage lambda cos sites have been inserted) and replicons (genetic elements that are capable of replication under their own control in a cell) can also be used.

The expression constructs may also be encapsulated, adsorbed to, or associated with, particulate carriers as described above. Such carriers present multiple copies of a selected molecule to the immune system and promote trapping and retention of molecules in local lymph nodes. The particles can be phagocytosed by macrophages and can enhance antigen presentation through cytokine release. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; and McGee et al., J. Microencap. (1996).

One preferred method for adsorbing macromolecules onto prepared microparticles is described in International Publication No. WO 00/050006, incorporated herein by reference in its entirety. Briefly, microparticles are rehydrated and dispersed to an essentially monomeric suspension of microparticles using dialyzable anionic or cationic detergents. Useful detergents include, but are not limited to, any of the various N-methylglucamides (known as MEGAs), such as heptanoyl-N-methylglucamide (MEGA-7), octanoyl-N-methylglucamide (MEGA-8), nonanoyl-N-methylglucamide (MEGA-9), and decanoyl-N-methyl-glucamide (MEGA-10); cholic acid; sodium cholate; deoxycholic acid; sodium deoxycholate; taurocholic acid; sodium taurocholate; taurodeoxycholic acid; sodium taurodeoxycholate; 3-[(3-cholamidopropyl)dimethylammonio]-1-propane-sulfonate (CHAPS); 3-[(3-cholamidopropyl) dimethylammonio]-2-hydroxy-1-propane-sulfonate (CHAPSO); -dodecyl-N,N-dimethyl-3-ammonio-1-propane-sulfonate (ZWITTERGENT 3-12); N,N-bis-(3-D-gluconeamidopropyl)-deoxycholamide (DEOXY-BIGCHAP); -octylglucoside; sucrose monolaurate; glycocholic acid/sodium glycocholate; laurosarcosine (sodium salt); glycodeoxycholic acid/sodium glycodeoxycholate; sodium dodceyl sulfate (SDS); 3-(trimethylsilyl)-1-propanesulfonic acid (DSS); cetrimide (CTAB, the principal component of which is hexadecyltrimethylammonium bromide); hexadecyltrimethylammonium bromide; dodecyltrimethylammonium bromide; hexadecyltrimethyl-ammonium bromide; tetradecyltrimethylammonium bromide; benzyl dimethyldodecylammonium bromide; benzyl dimethyl-hexadecylammonium chloride; and benzyl dimethyltetra-decylammonium bromide. The above detergents are commercially available from e.g., Sigma Chemical Co., St. Louis, Mo. Various cationic lipids known in the art can also be used as detergents. See Balasubramaniam et al., 1996, Gene Ther., 3:163-72 and Gao, X., and L. Huang. 1995, Gene Ther., 2:7110-722.

A wide variety of other methods can be used to deliver the expression constructs to cells. Such methods include DEAE dextran-mediated transfection, calcium phosphate precipitation, polylysine- or polyornithine-mediated transfection, or precipitation using other insoluble inorganic salts, such as strontium phosphate, aluminum silicates including bentonite and kaolin, chromic oxide, magnesium silicate, talc, and the like. Other useful methods of transfection include electroporation, sonoporation, protoplast fusion, liposomes, peptoid delivery, or microinjection. See, e.g., Sambrook et al., supra, for a discussion of techniques for transforming cells of interest; and Feigner, P. L., Advanced Drug Delivery Reviews (1990) 5:163-187, for a review of delivery systems useful for gene transfer. Methods of delivering DNA using electroporation are described in, e.g., U.S. Pat. Nos. 6,132,419; 6,451,002, 6,418,341, 6233,483, U.S. Patent Publication No. 2002/0146831; and International Publication No. WO/0045823, all of which are incorporated herein by reference in their entireties.

Moreover, the CAL polynucleotides can be adsorbed to, or entrapped within, an ISCOM. Classic ISCOMs are formed by combination of cholesterol, saponin, phospholipid, and immunogens, such as viral envelope proteins. Generally, the CAL molecules (usually with a hydrophobic region) are solubilized in detergent and added to the reaction mixture, whereby ISCOMs are formed with the CAL molecule incorporated therein. ISCOM matrix compositions are formed identically, but without viral proteins. Proteins with high positive charge may be electrostatically bound in the ISCOM particles, rather than through hydrophobic forces. For a more detailed general discussion of saponins and ISCOMs, and methods of formulating ISCOMs, see Barr et al. (1998) Adv. Drug Delivery Reviews 32:247-271 (1998); U.S. Pat. Nos. 4,981,684, 5,178,860, 5,679,354 and 6,027,732, incorporated herein by reference in their entireties; European Publ. Nos. EPA 109,942; 180,564 and 231,039; and Coulter et al. (1998) Vaccine 16:1243.

Additionally, biolistic delivery systems employing particulate carriers such as gold and tungsten, are useful for delivering the expression constructs of the present invention. The particles are coated with the construct to be delivered and accelerated to high velocity, generally under a reduced atmosphere, using a gun powder discharge from a “gene gun.” For a description of such techniques, and apparatuses useful therefore, see, e.g., U.S. Pat. Nos. 4,945,050; 5,036,006; 5,100,792; 5,179,022; 5,371,015; and 5,478,744.

The amount of CAL virus DNA delivered will generally be about 1 μg to 500 mg of DNA, such as 5 μg to 100 mg of DNA, e.g., 10 μg to 50 mg, or 100 μg to 5 mg, such as 20 . . . 30 . . . 40 . . . 50 . . . 60 . . . 100 . . . 200 μg and so on, to 500 μg DNA, and any integer between the stated ranges.

Administration of CAL viral, polypeptide or polynucleotide compositions can elicit a cellular immune response, and/or an anti-CAL antibody titer in the mammal that lasts for at least 1 week, 2 weeks, 1 month, 2 months, 3 months, 4 months, 6 months, 1 year, or longer. The compositions can also be administered to provide a memory response. If such a response is achieved, antibody titers may decline over time, however exposure to CAL virus or the particular immunogen results in the rapid induction of antibodies, e.g., within only a few days. Optionally, antibody titers can be maintained in a mammal by providing one or more booster injections of the CAL compositions, at e.g., 2 weeks, 1 month, 2 months, 3 months, 4 months, 5 months, 6 months, 1 year, or more after the primary injection.

Preferably, an antibody titer of at least 10, 100, 150, 175, 200, 300, 400, 500, 750, 1,000, 1,500, 2,000, 3,000, 5,000, 10,000, 20,000, 30,000, 40,000, 50,000 (geometric mean titer), or higher, is elicited, or any number between the stated titers, as determined using a standard immunoassay.

CAL Virus Antibodies

The CAL virus immunogens can be used to produce CAL-specific polyclonal and monoclonal antibodies. CAL-specific polyclonal and monoclonal antibodies specifically bind to CAL antigens. Polyclonal antibodies can be produced by administering the immunogen to a mammal, such as a mouse, a rabbit, a goat, or a horse. Serum from the immunized animal is collected and the antibodies are purified from the plasma by, for example, precipitation with ammonium sulfate, followed by chromatography, preferably affinity chromatography. Techniques for producing and processing polyclonal antisera are known in the art.

Monoclonal antibodies directed against CAL-specific epitopes present in the proteins can also be readily produced. Normal B cells from a mammal, such as a mouse, immunized with a CAL protein, can be fused with, for example, HAT-sensitive mouse myeloma cells to produce hybridomas. Hybridomas producing CAL-specific antibodies can be identified using RIA or ELISA and isolated by cloning in semi-solid agar or by limiting dilution. Clones producing CAL-specific antibodies are isolated by another round of screening.

It may be desirable to provide chimeric antibodies, especially if the antibodies are to be used in preventive or therapeutic pharmaceutical preparations, such as for providing passive protection against CAL infection, as well as in CAL diagnostic preparations. Chimeric antibodies composed of human and non-human amino acid sequences may be formed from the mouse monoclonal antibody molecules to reduce their immunogenicity in humans (Winter et al. (1991) Nature 30:293; Lobuglio et al. (1989) Proc. Nat. Acad. Sci. USA 86:4220; Shaw et al. (1987) J. Immunol. 138:4534; and Brown et al. (1987) Cancer Res. 47:3577; Riechmann et al. (1988) Nature 332:323; Verhoeyen et al. (1988) Science 239:1 534; and Jones et al. (1986) Nature 321:522; EP Publication No. 519,596, published 23 Dec. 1992; and U.K. Patent Publication No. GB 2,276,169, published 21 Sep. 1994).

Antibody molecule fragments, e.g., F(ab′)₂, Fv, and sFv molecules, that are capable of exhibiting immunological binding properties of the parent monoclonal antibody molecule can be produced using known techniques. Inbar et al. (1972) Proc. Nat. Acad. Sci. USA 69:2659; Hochman et al. (1976) Biochem 15:2706; Ehrlich et al. (1980) Biochem 19:4091; Huston et al. (1988) Proc. Nat. Acad. Sci. USA 85(16):5879; and U.S. Pat. Nos. 5,091,513 and 5,132,405, to Huston et al.; and 4,946,778, to Ladner et al.

In the alternative, a phage-display system can be used to expand monoclonal antibody molecule populations in vitro. Saiki, et al. (1986) Nature 324:163; Scharf et al. (1986) Science 233:1076; U.S. Pat. Nos. 4,683,195 and 4,683,202; Yang et al. (1995) J Mol Biol 254:392; Barbas, III et al. (1995) Methods: Comp. Meth Enzymol 8:94; Barbas, III et al. (1991) Proc Natl Acad Sci USA 88:7978.

Once generated, the phage display library can be used to improve the immunological binding affinity of the Fab molecules using known techniques. See, e.g., Figini et al. (1994) J. Mol. Biol. 239:68. The coding sequences for the heavy and light chain portions of the Fab molecules selected from the phage display library can be isolated or synthesized, and cloned into any suitable vector or replicon for expression. Any suitable expression system can be used, including those described above.

Antibodies which are directed against CAL virus epitopes, are particularly useful for detecting the presence of CAL virus or CAL virus antigens in a sample, such as a serum sample from a CAL virus-infected human. An immunoassay for a CAL virus antigen may utilize one antibody or several antibodies. An immunoassay for a CAL virus antigen may use, for example, a monoclonal antibody directed towards a CAL virus epitope, a combination of monoclonal antibodies directed towards epitopes of one CAL virus polypeptide, monoclonal antibodies directed towards epitopes of different CAL virus polypeptides, polyclonal antibodies directed towards the same CAL virus antigen, polyclonal antibodies directed towards different CAL virus antigens, or a combination of monoclonal and polyclonal antibodies. Immunoassay protocols may be based, for example, upon competition, direct reaction, or sandwich type assays using, for example, labeled antibody and are described further below. The labels may be, for example, fluorescent, chemiluminescent, or radioactive.

The CAL virus antibodies may further be used to isolate CAL particles or antigens by immunoaffinity columns. The antibodies can be affixed to a solid support by, for example, adsorption or by covalent linkage so that the antibodies retain their immunoselective activity. Optionally, spacer groups may be included so that the antigen binding site of the antibody remains accessible. The immobilized antibodies can then be used to bind CAL particles or antigens from a biological sample, such as blood or plasma. The bound CAL particles or antigens are recovered from the column matrix by, for example, a change in pH.

CAL Diagnostic Assays

As explained above, the CAL virus immunogens, antibodies and polynucleotides can be used in assays to identify CAL virus infection, such as LACV infection. Protein assays include Western blots; agglutination tests; enzyme-labeled and mediated immunoassays, such as ELISAs; biotin/avidin type assays; radioimmunoassays; immunoelectrophoresis; immunoprecipitation, and the like. The reactions generally include revealing labels such as fluorescent, chemiluminescent, radioactive, enzymatic labels or dye molecules, or other methods for detecting the formation of a complex between the mimetic and the antibody or antibodies reacted therewith.

The aforementioned assays generally involve separation of unbound antibody or antigen in a liquid phase from a solid phase support to which antigen-antibody complexes are bound. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

Typically, a solid support is first reacted with a solid phase component (e.g., one or more CAL virus antigens or antibodies) under suitable binding conditions such that the component is sufficiently immobilized to the support. Sometimes, immobilization to the support can be enhanced by first coupling to a protein with better binding properties. Suitable coupling proteins include, but are not limited to, macromolecules such as serum albumins including bovine serum albumin (BSA), keyhole limpet hemocyanin, immunoglobulin molecules, thyroglobulin, ovalbumin, and other proteins well known to those skilled in the art. Other molecules that can be used to bind the antigen or antibody to the support include polysaccharides, polylactic acids, polyglycolic acids, polymeric amino acids, amino acid copolymers, and the like. Such molecules and methods of coupling these molecules are well known to those of ordinary skill in the art. See, e.g., Brinkley, M. A. Bioconjugate Chem. (1992) 3:2-13; Hashida et al., J. Appl. Biochem. (1984) 6:56-63; and Anjaneyulu and Staros, International J. of Peptide and Protein Res. (1987) 30:117-124.

After reacting the solid support with the solid phase component, any non-immobilized solid-phase components are removed from the support by washing, and the support-bound component is then contacted with a biological sample suspected of containing the ligand component (i.e., CAL virus antigens or antibodies) under suitable binding conditions. After washing to remove any non-bound ligand, a secondary binder moiety is added under suitable binding conditions, wherein the secondary binder is capable of associating selectively with the bound ligand. The presence of the secondary binder can then be detected using techniques well known in the art.

More particularly, an ELISA method can be used, wherein the wells of a microtiter plate are coated with one or more CAL virus epitopes or antibodies according to the present invention. A biological sample containing or suspected of containing either anti-CAL virus immunoglobulin molecules or CAL virus antigens is then added to the coated wells. After a period of incubation sufficient to allow antigen-antibody binding, the plate(s) can be washed to remove unbound moieties and a detectably labeled secondary binding molecule added. The secondary binding molecule is allowed to react with any captured sample, the plate washed and the presence of the secondary binding molecule detected using methods well known in the art.

Thus, in one particular embodiment, the presence of bound CAL virus ligands from a biological sample can be readily detected using a secondary binder comprising an antibody directed against the antibody ligands. A number of anti-human immunoglobulin (Ig) molecules are known in the art which can be readily conjugated to a detectable enzyme label, such as horseradish peroxidase, alkaline phosphatase or urease, using methods known to those of skill in the art. An appropriate enzyme substrate is then used to generate a detectable signal. In other related embodiments, competitive-type ELISA techniques can be practiced using methods known to those skilled in the art.

Assays can also be conducted in solution, such that the CAL virus epitopes or antibodies and ligands specific for these molecules form complexes under precipitating conditions. In one particular embodiment, the molecules can be attached to a solid phase particle (e.g., an agarose bead or the like) using coupling techniques known in the art, such as by direct chemical or indirect coupling. The coated particle is then contacted under suitable binding conditions with a biological sample suspected of containing CAL virus antibodies or antigens. Cross-linking between bound antibodies causes the formation of complex aggregates which can be precipitated and separated from the sample using washing and/or centrifugation. The reaction mixture can be analyzed to determine the presence or absence of complexes using any of a number of standard methods, such as those immunodiagnostic methods described above.

In yet a further embodiment, an immunoaffinity matrix can be provided, wherein, for example, a polyclonal population of antibodies from a biological sample suspected of containing CAL virus antibodies is immobilized to a substrate. An initial affinity purification of the sample can be carried out using immobilized antigens. The resultant sample preparation will thus only contain anti-CAL virus moieties, avoiding potential nonspecific binding properties in the affinity support. A number of methods of immobilizing immunoglobulins (either intact or in specific fragments) at high yield and good retention of antigen binding activity are known in the art. Once the immunoglobulin molecules have been immobilized to provide an immunoaffinity matrix, labeled molecules are contacted with the bound antibodies under suitable binding conditions. After any non-specifically bound CAL virus epitope has been washed from the immunoaffinity support, the presence of bound antigen can be determined by assaying for label using methods known in the art.

The above-described assay reagents, including CAL virus polypeptides and/or antibodies thereto, the solid supports with bound reagents, as well as other detection reagents, can be provided in kits, with suitable instructions and other necessary reagents, in order to conduct the assays as described above. The kit may also include control formulations (positive and/or negative), labeled reagents when the assay format requires same and signal generating reagents (e.g., enzyme substrate) if the label does not generate a signal directly. Instructions (e.g., written, tape, VCR, CD-ROM, etc.) for carrying out the assay usually will be included in the kit. The kit can also contain, depending on the particular assay used, other packaged reagents and materials (i.e. wash buffers and the like). Standard assays, such as those described above, can be conducted using these kits.

Nucleic acid-based assays can be conducted using CAL virus oligonucleotides and polynucleotides described above. For example, probe-based assays, such as hybridization assays, can be conducted that utilize oligonucleotides from the CAL virus in question. These assays may also utilize nucleic acid amplification methods such as reverse transcriptase-polymerase chain reaction (RT-PCR), PCR and ligase chain reaction (LCR).

Thus, the various CAL virus polynucleotide sequences may be used to produce probes and primers which can be used in assays for the detection of nucleic acids in test samples. The probes and primers may be designed from conserved nucleotide regions of the polynucleotides of interest or from non-conserved nucleotide regions of the polynucleotide of interest. The design of such oligonucleotides is well within the skill of the routineer. Generally, nucleic acid probes are developed from non-conserved or unique regions when maximum specificity is desired, and nucleic acid probes are developed from conserved regions when assaying for nucleotide regions that are closely related to, for example, different CAL virus isolates.

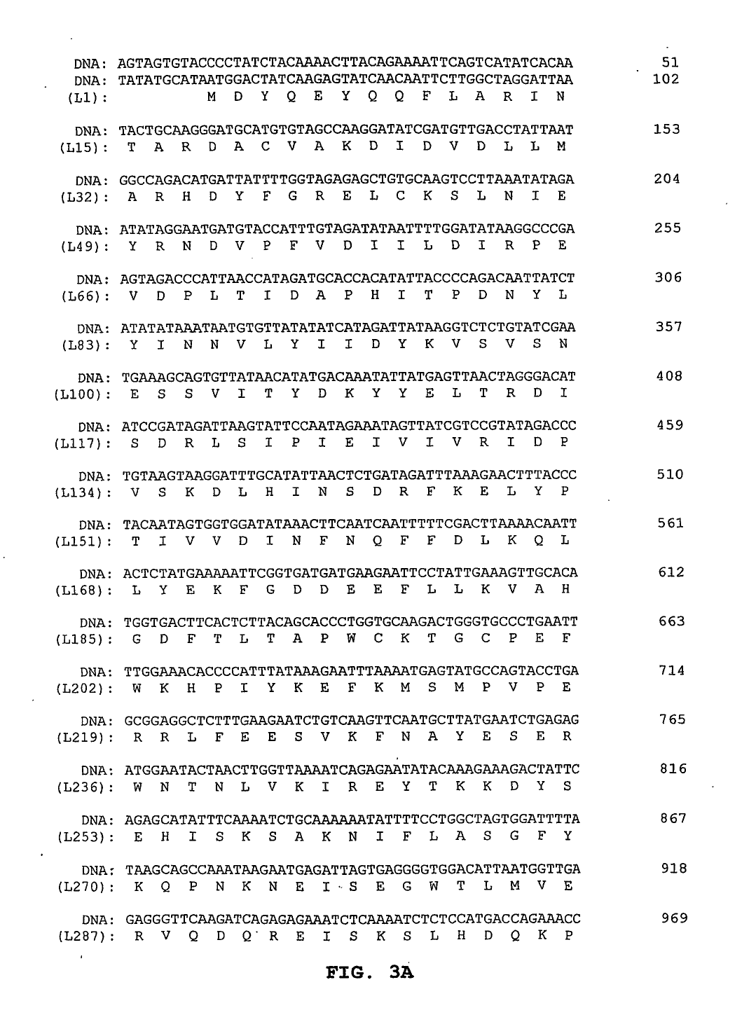

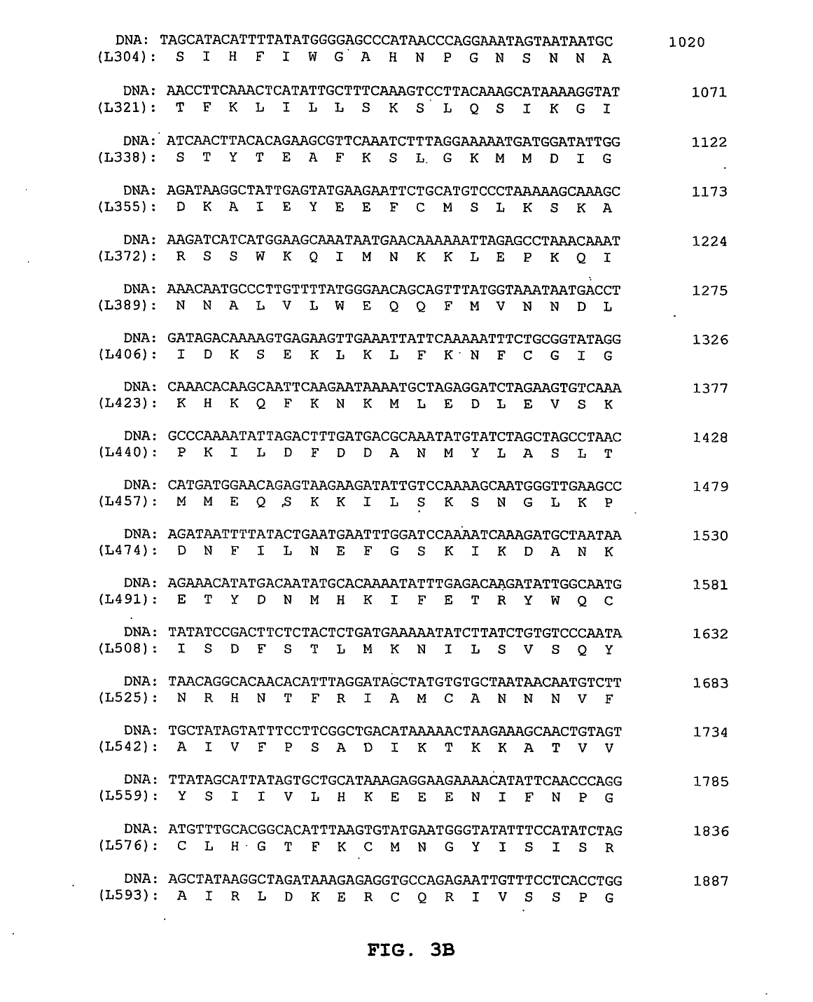

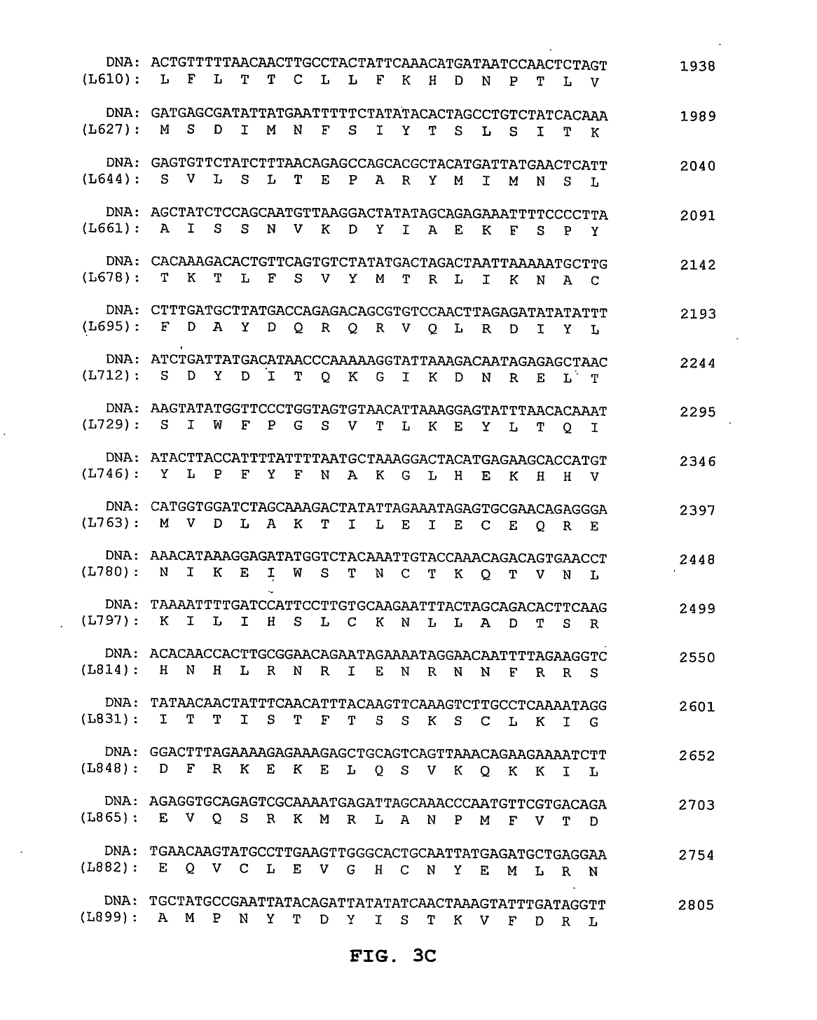

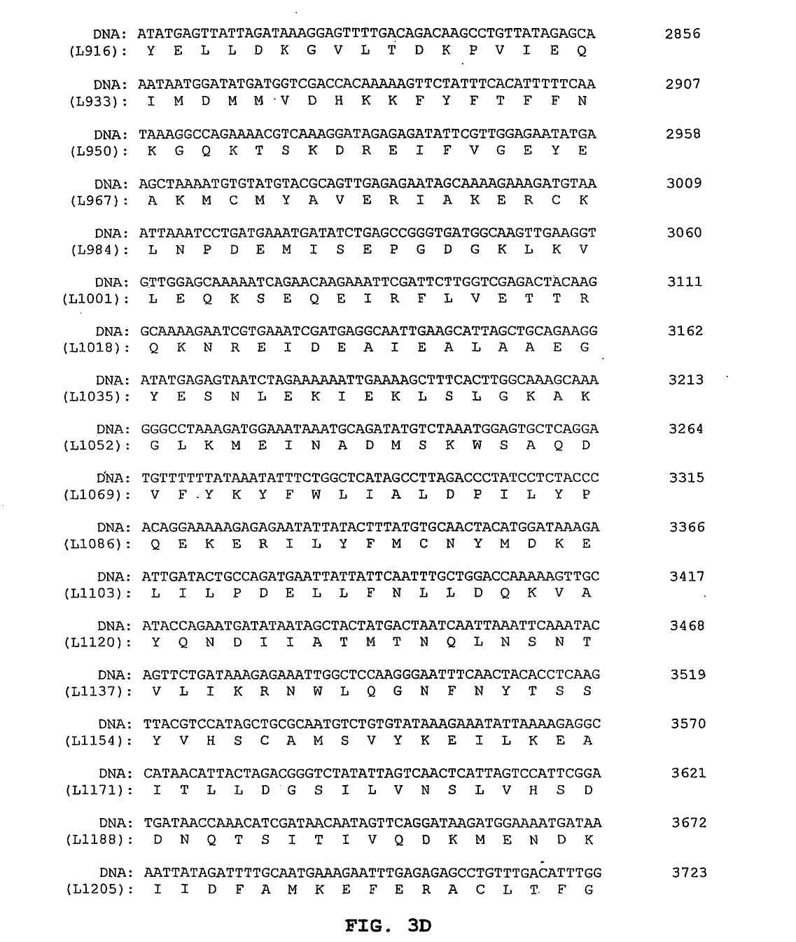

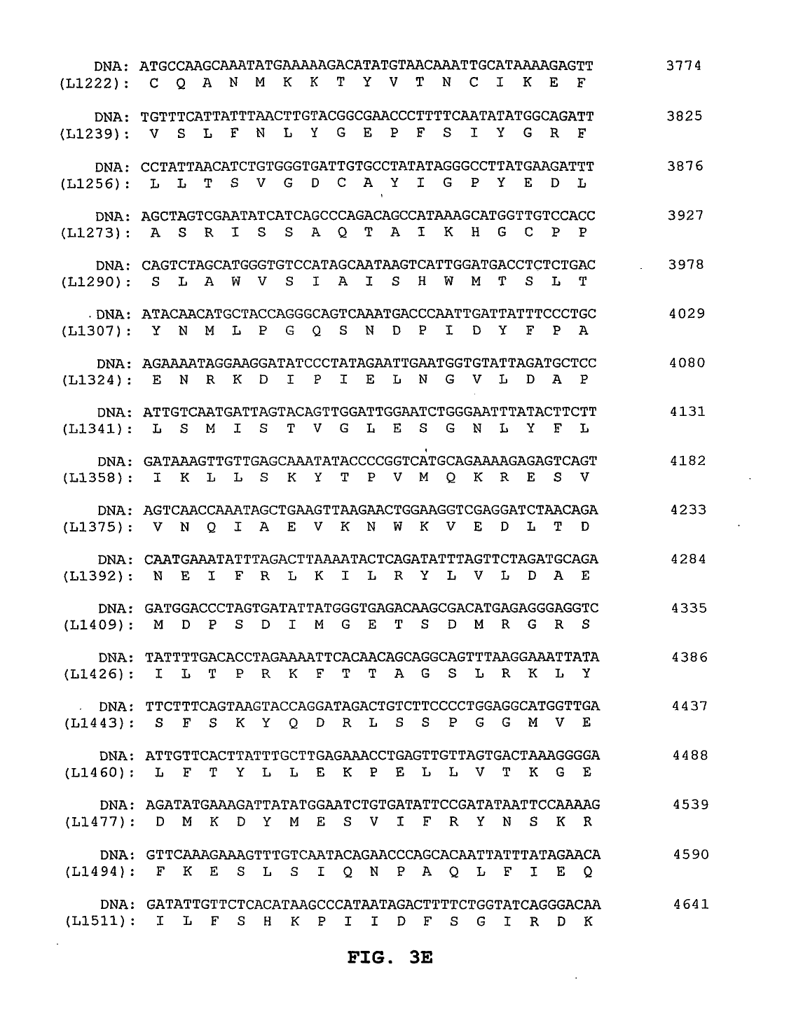

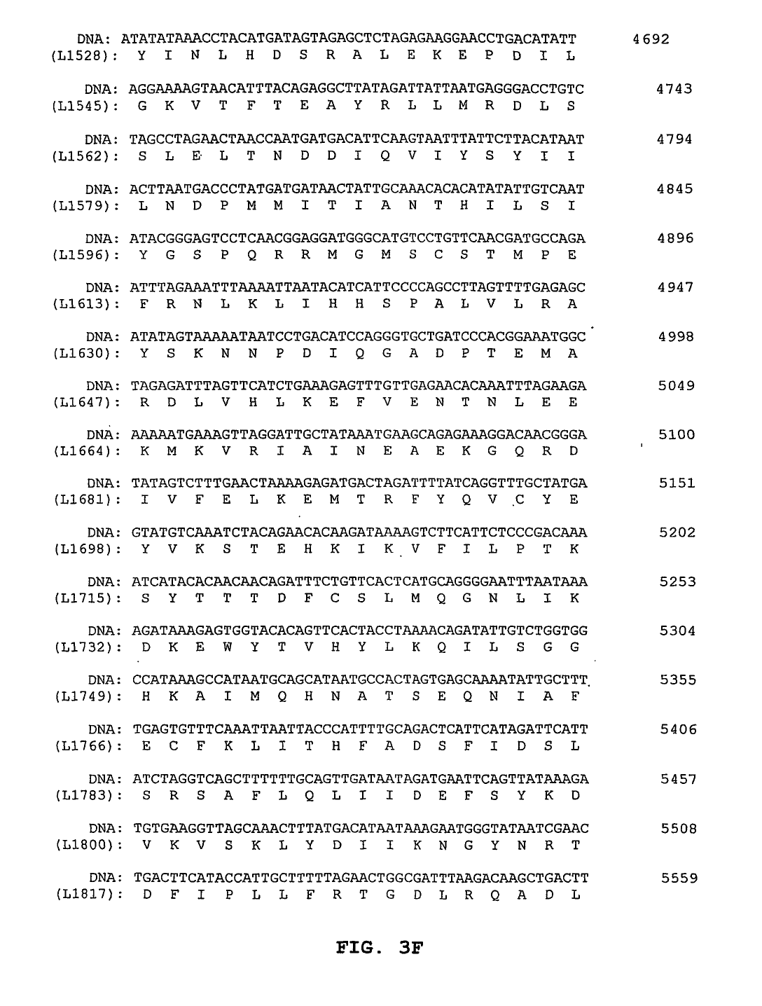

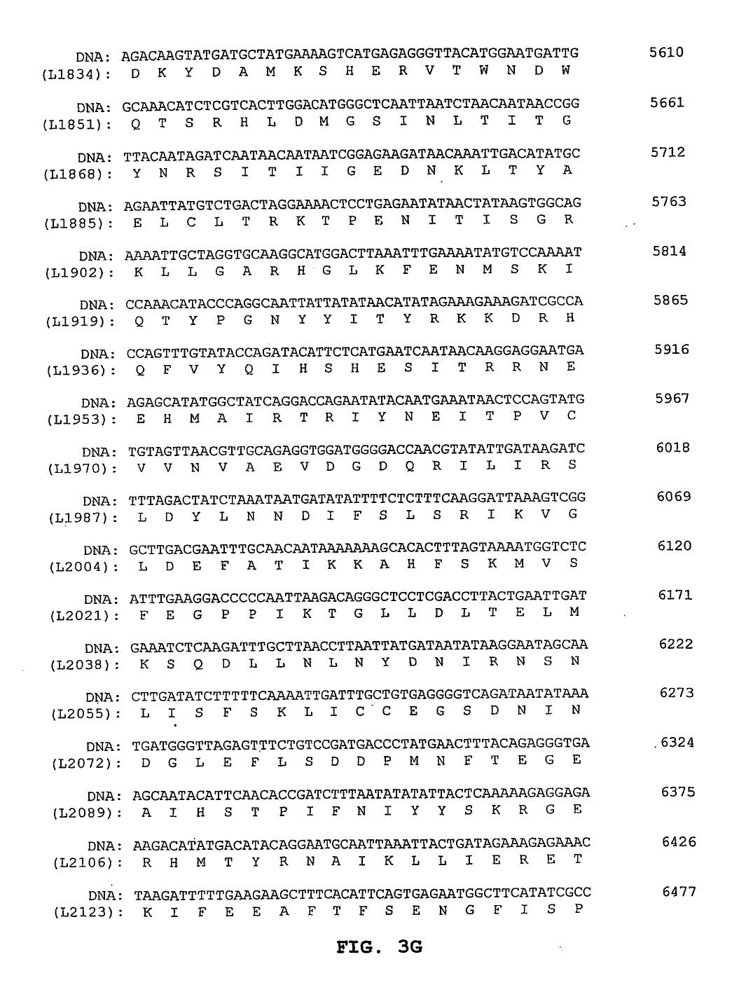

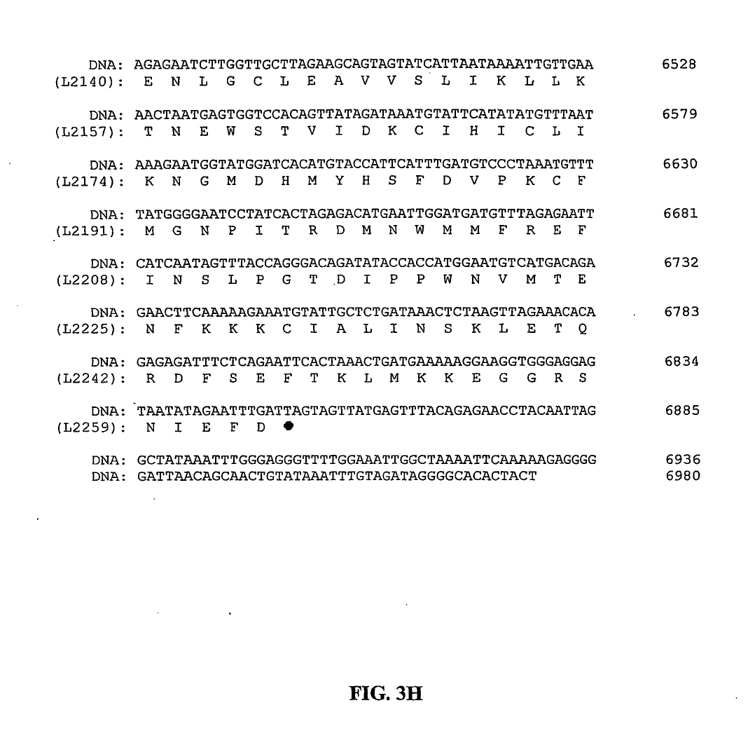

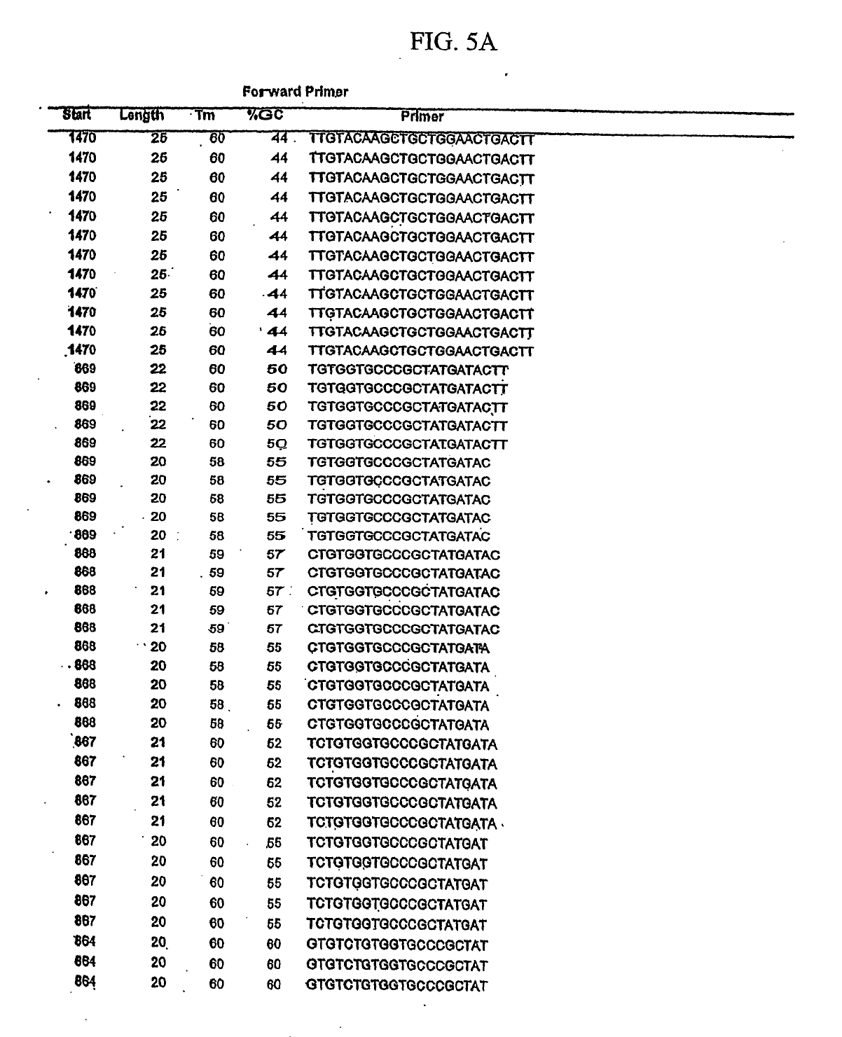

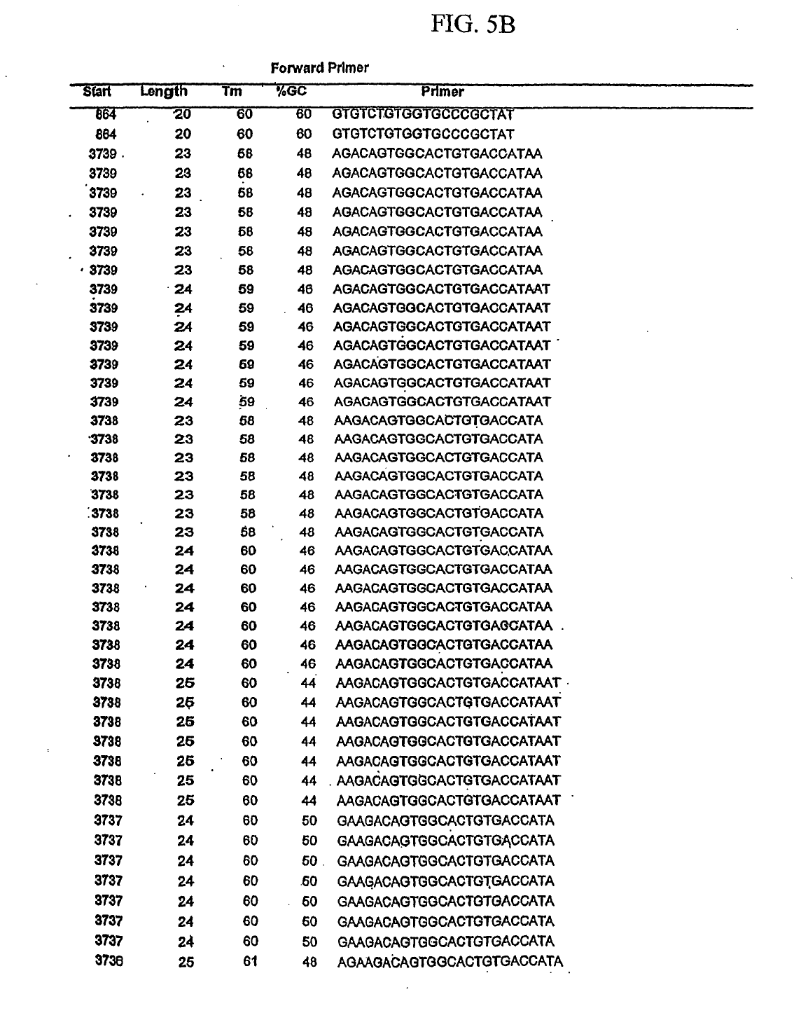

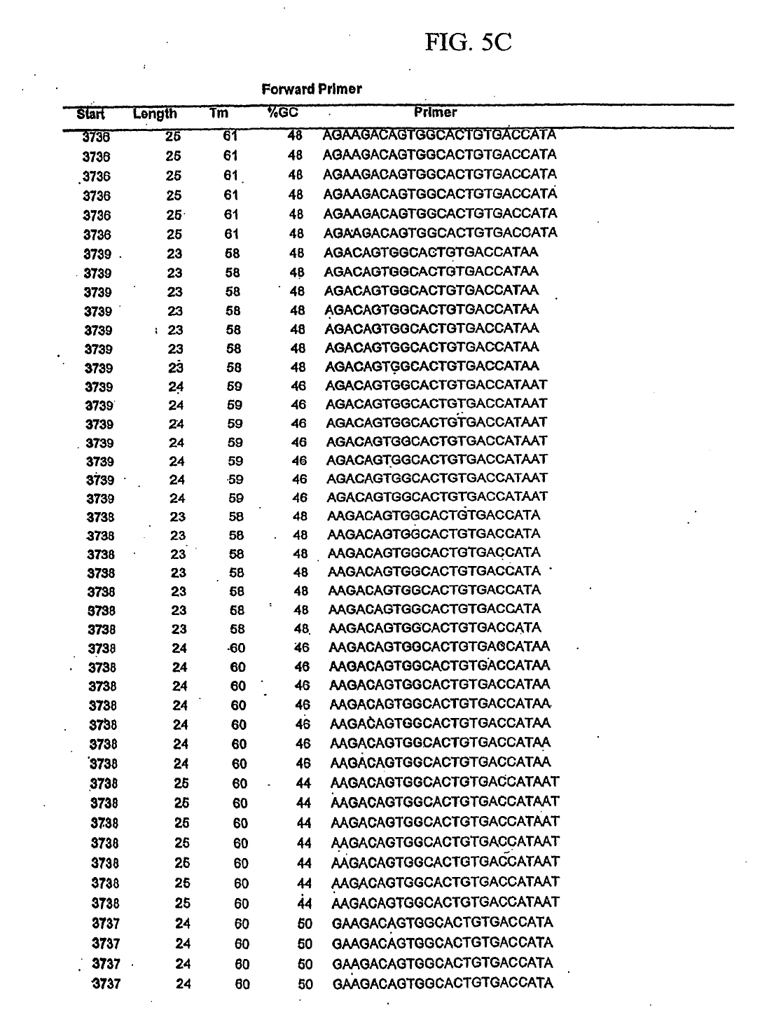

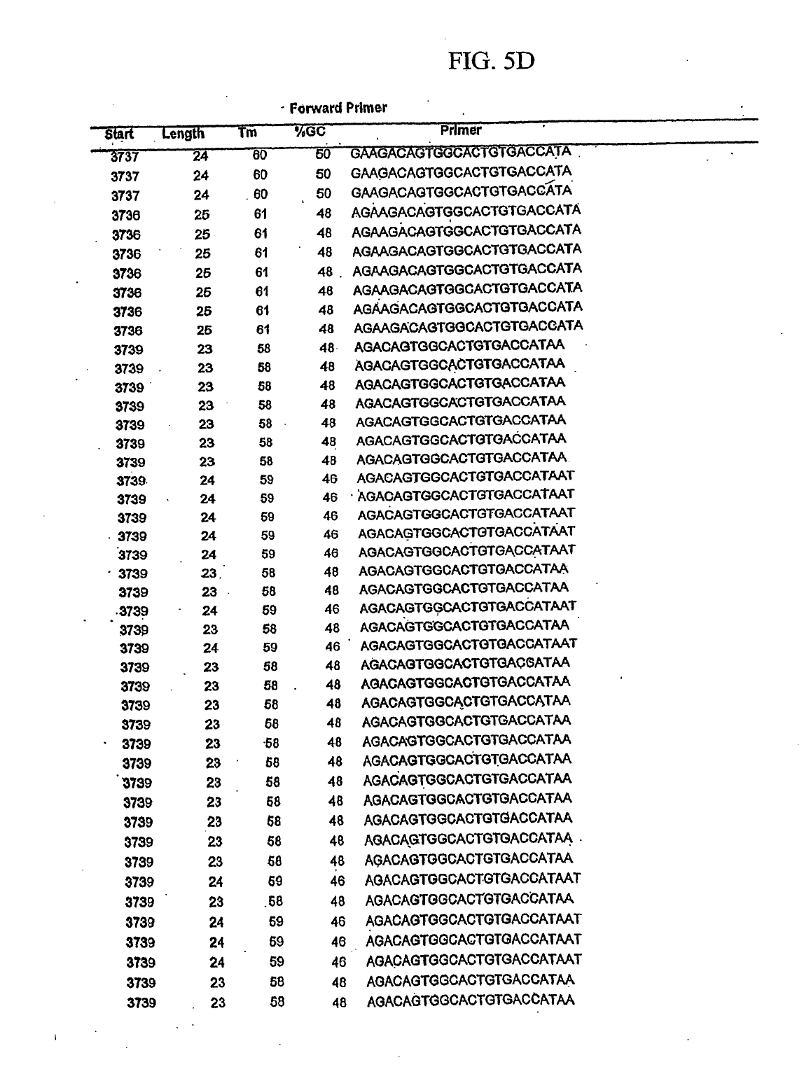

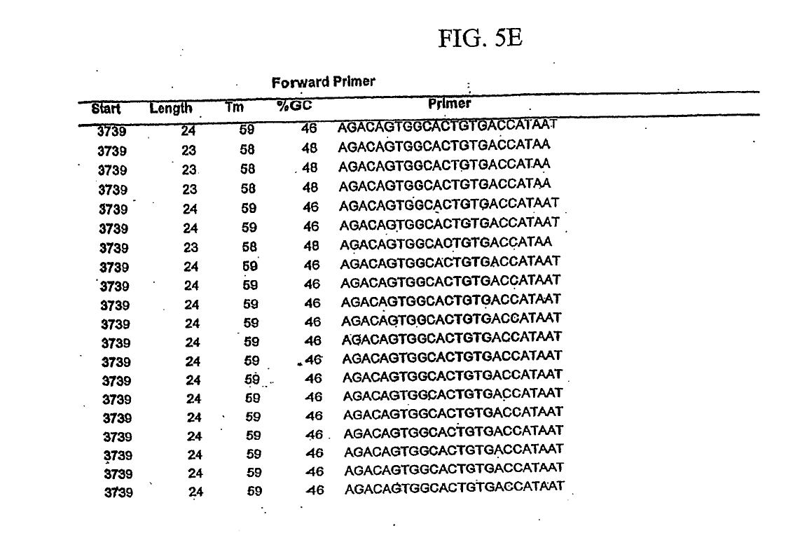

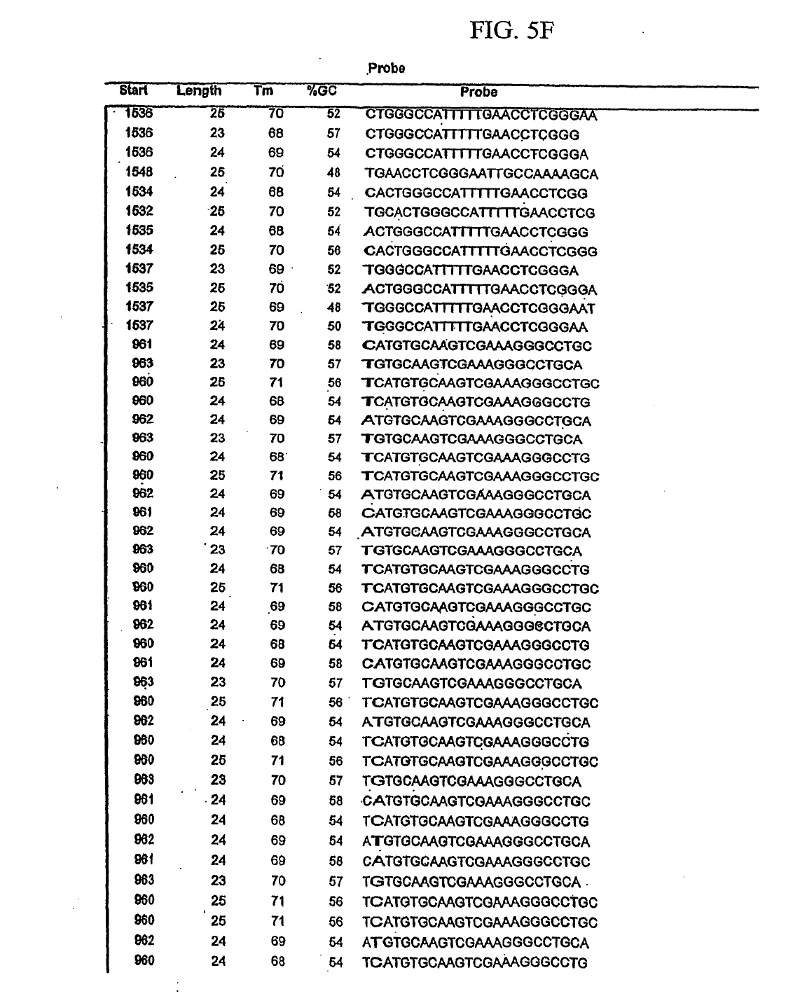

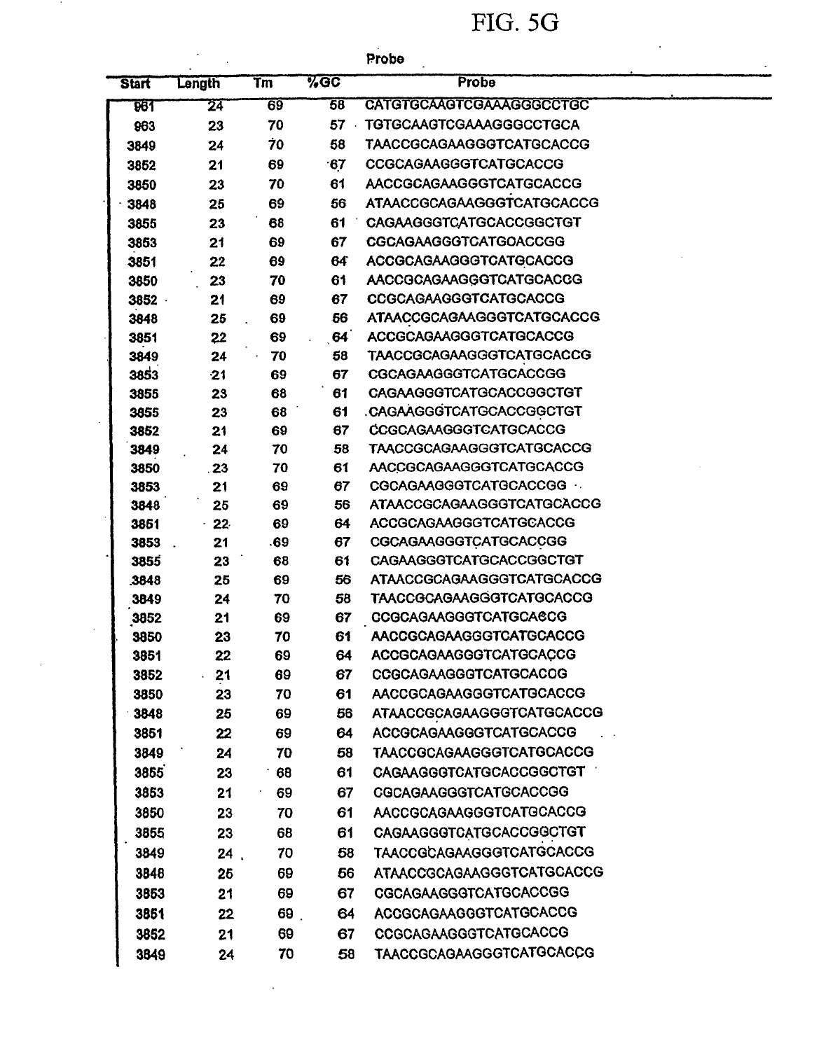

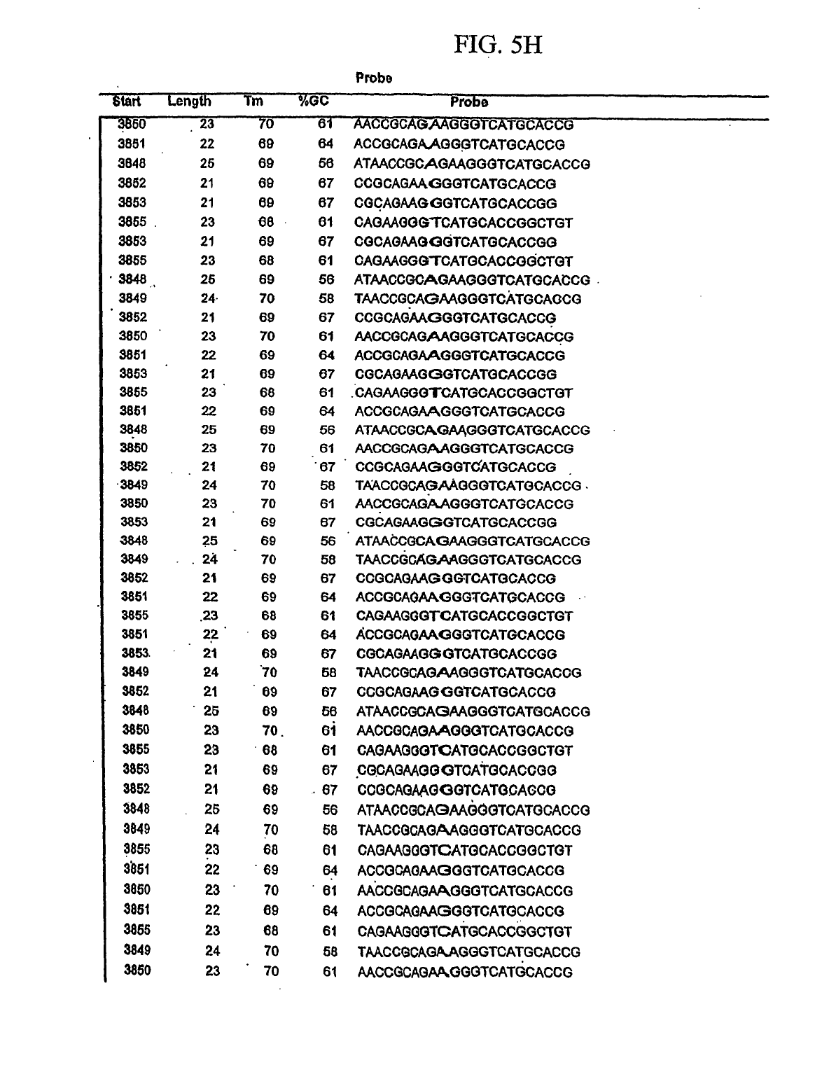

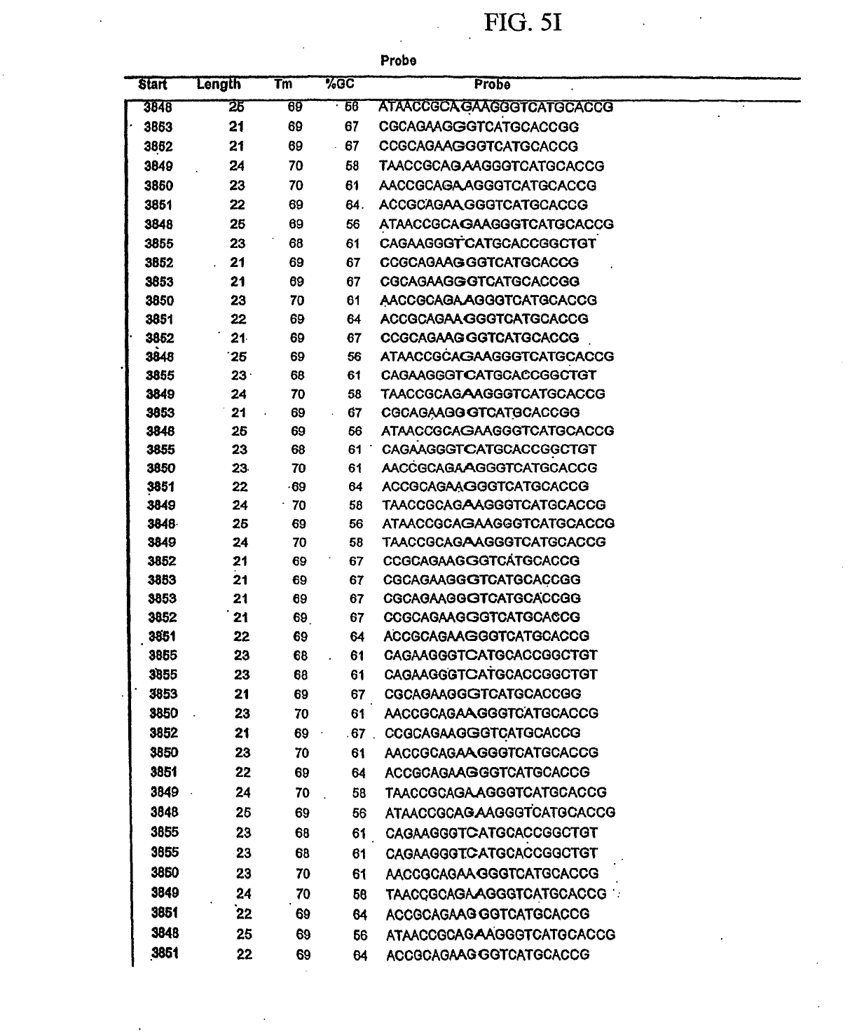

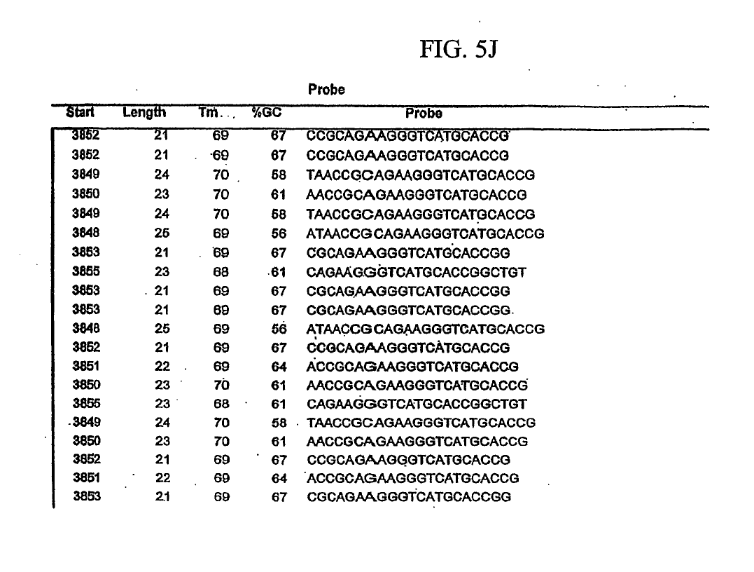

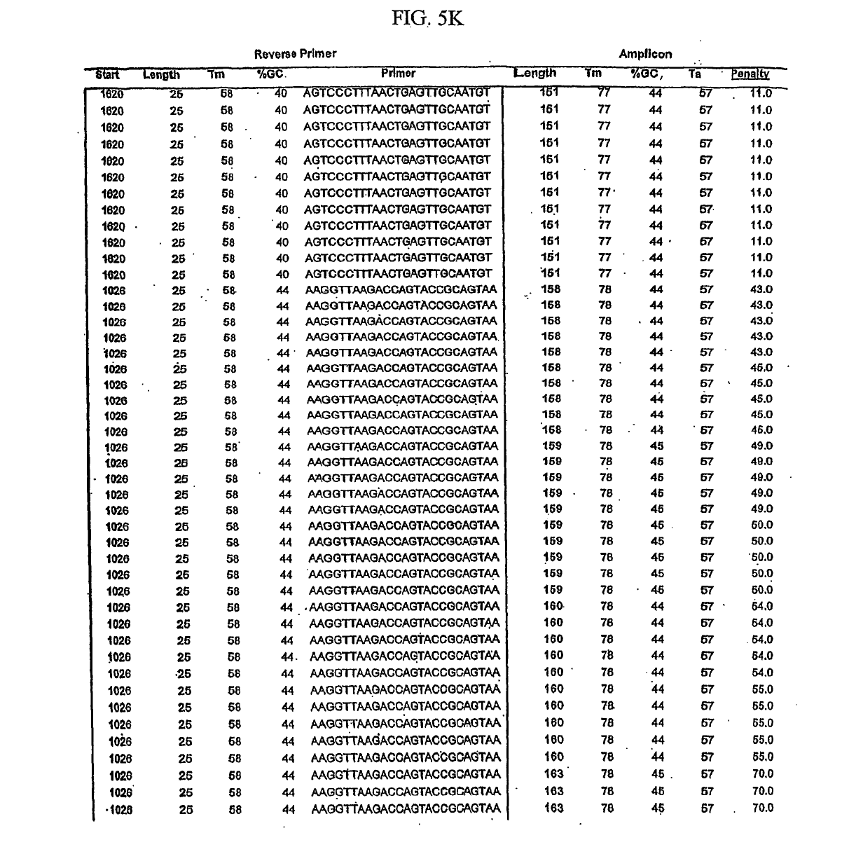

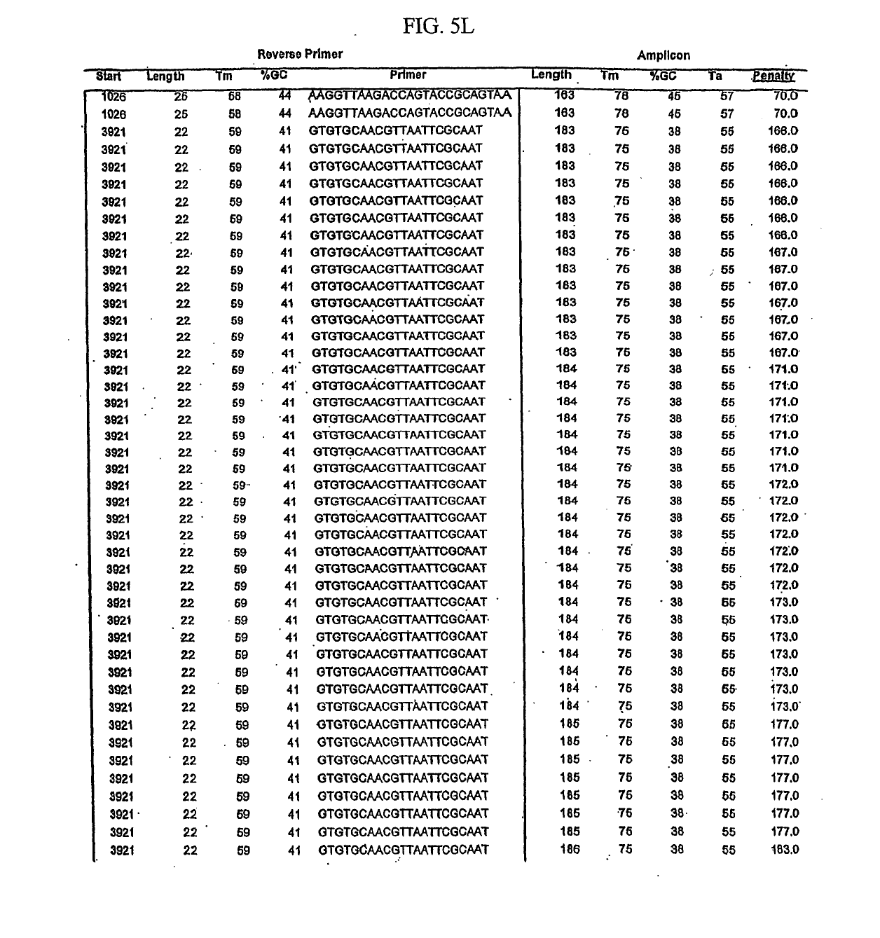

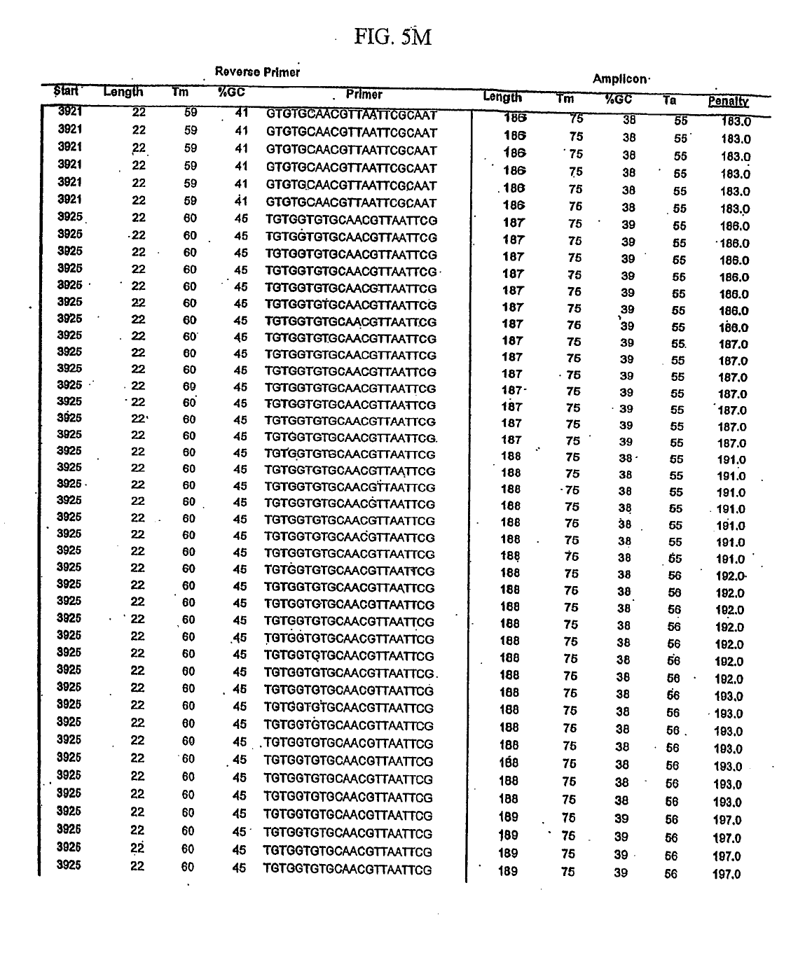

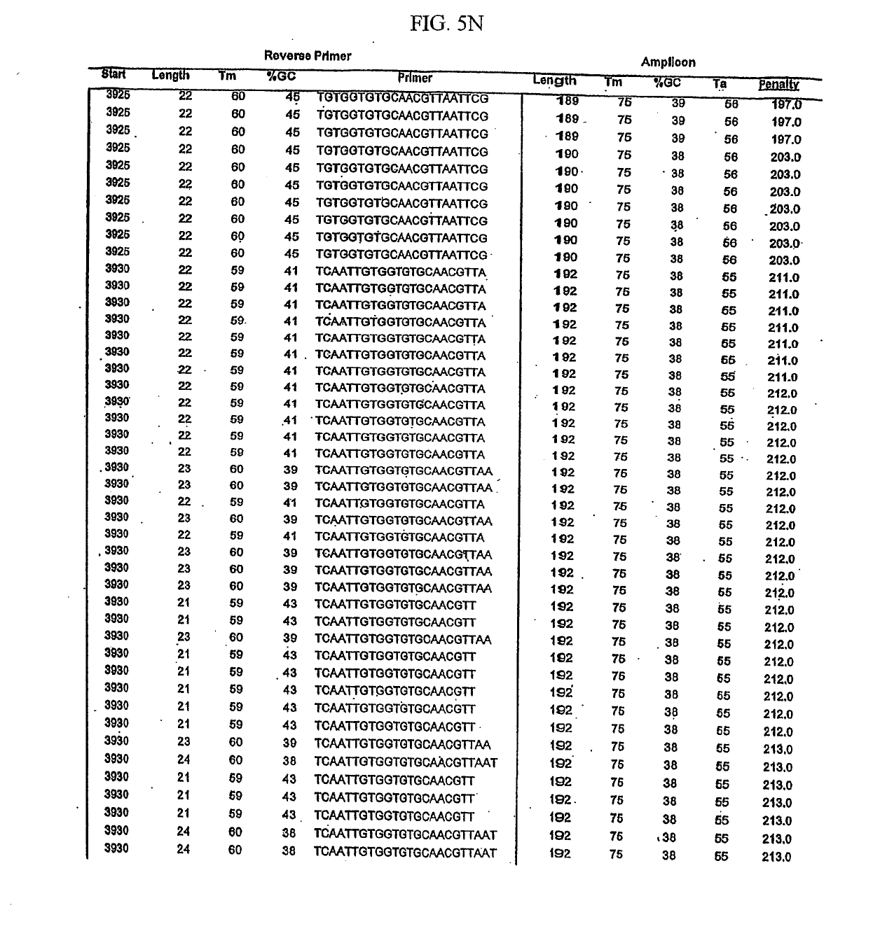

Representative LACV probes and primers derived from the M, S and L regions for use in the various assays are shown in FIGS. 5-7, respectively. The sequences and numbering are based on the sequences described in NCBI Accession nos. NC 004109 (FIG. 1), NC 004110 (FIG. 2) and NC 004108 (FIG. 3), respectively. In particular, FIGS. 5A-5O show representative primer/probe sets from the LACV M segment for use in the various nucleotide-based assays. Forward primers from the LACV M segment are shown in FIGS. 5A-5E; reverse primers for use with the forward primers are shown on the corresponding lines in FIGS. 5K-5O; and probes for use with the primer pairs shown in FIGS. 5A-5E and 5K-5O are shown on the corresponding lines in FIGS. 5F-5J. Thus, for example, the forward primer shown on line 1 of FIG. 5A (beginning at nucleotide position 1470) can be used with the reverse primer shown on line 1 of FIG. 5K (beginning at nucleotide position 1620), and the probe shown on line 1 of FIG. 5F (beginning at nucleotide position 1536), and so forth for the remaining primers and probes shown in FIGS. 5A-50.

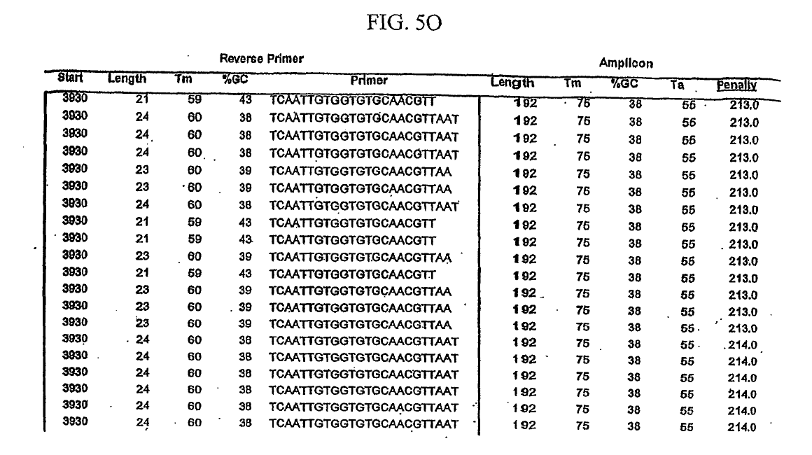

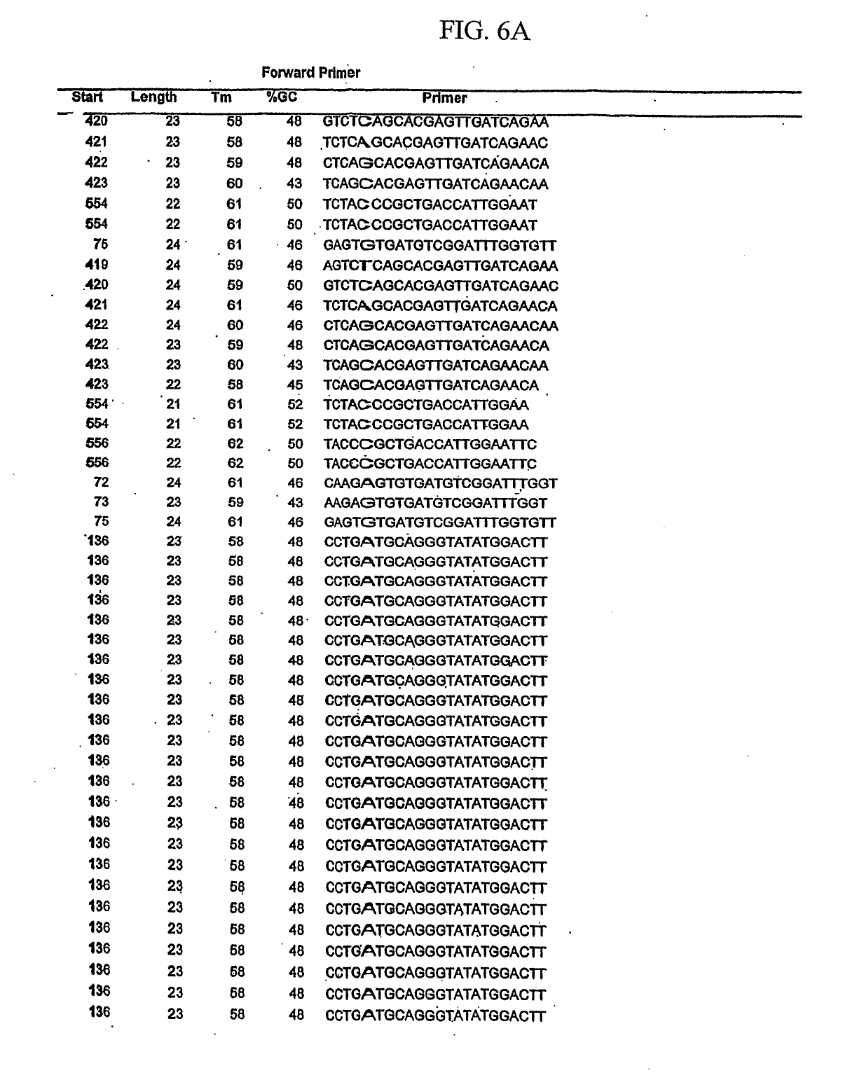

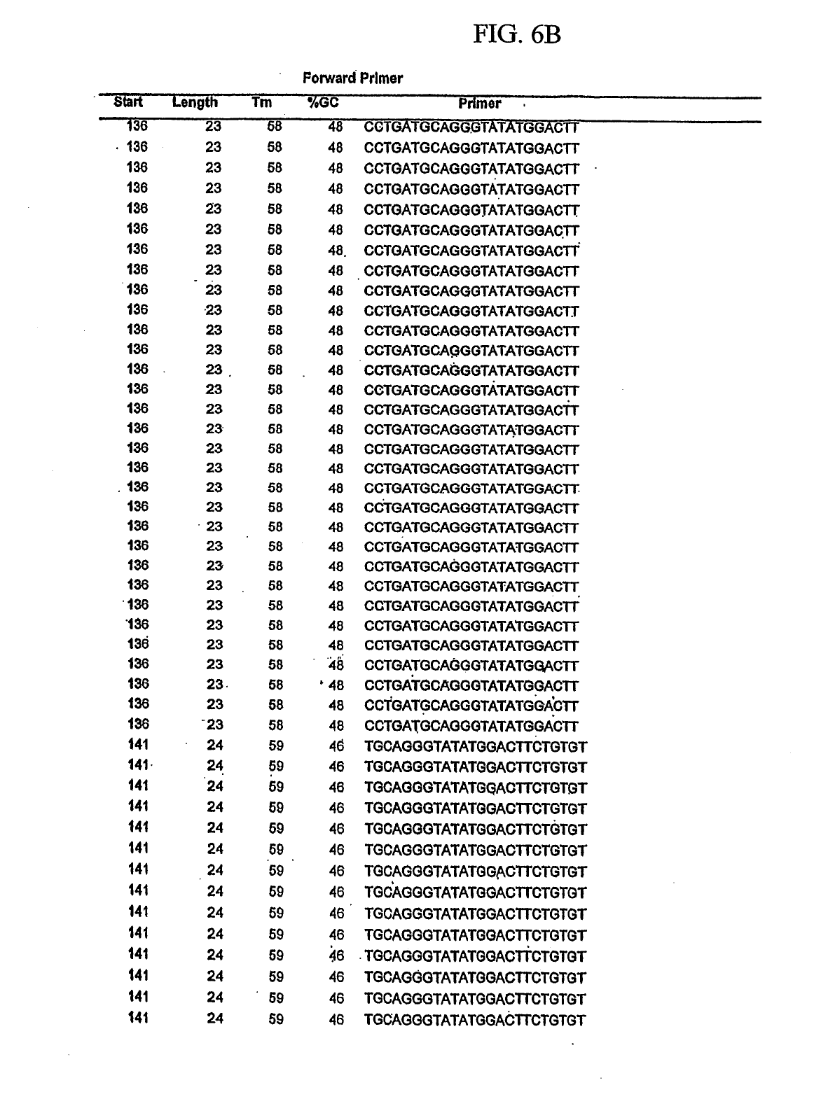

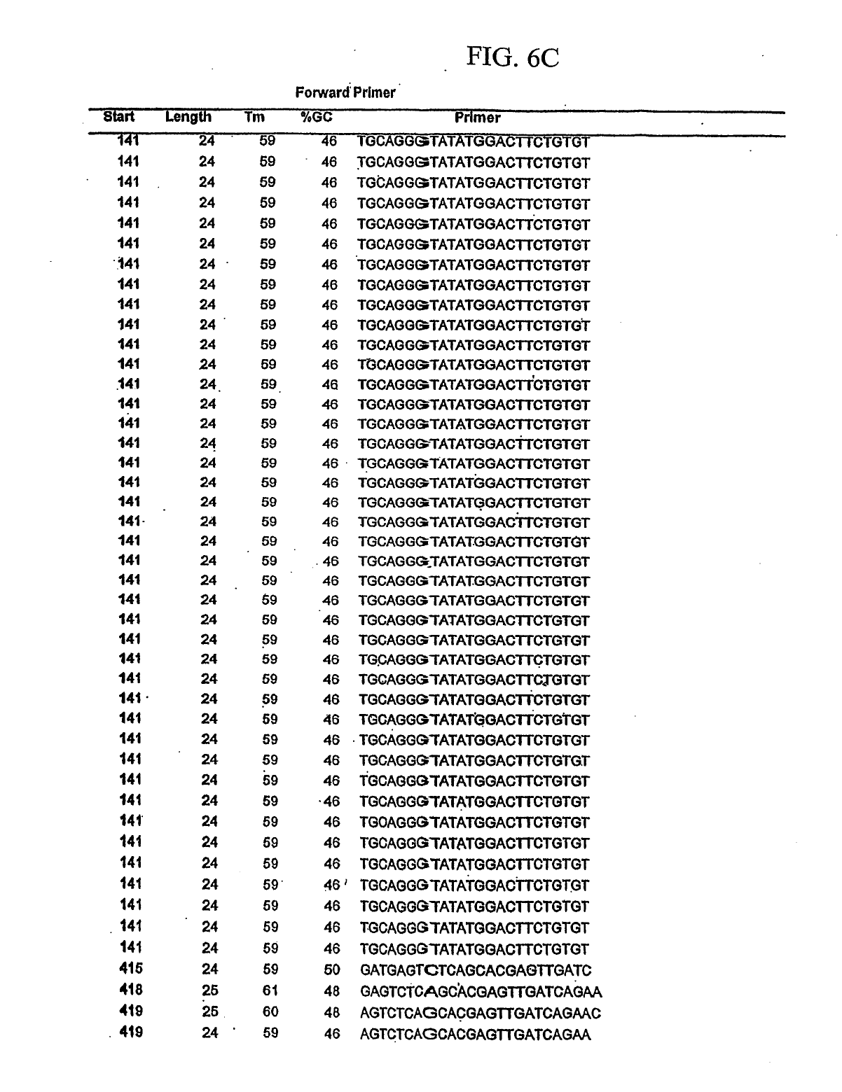

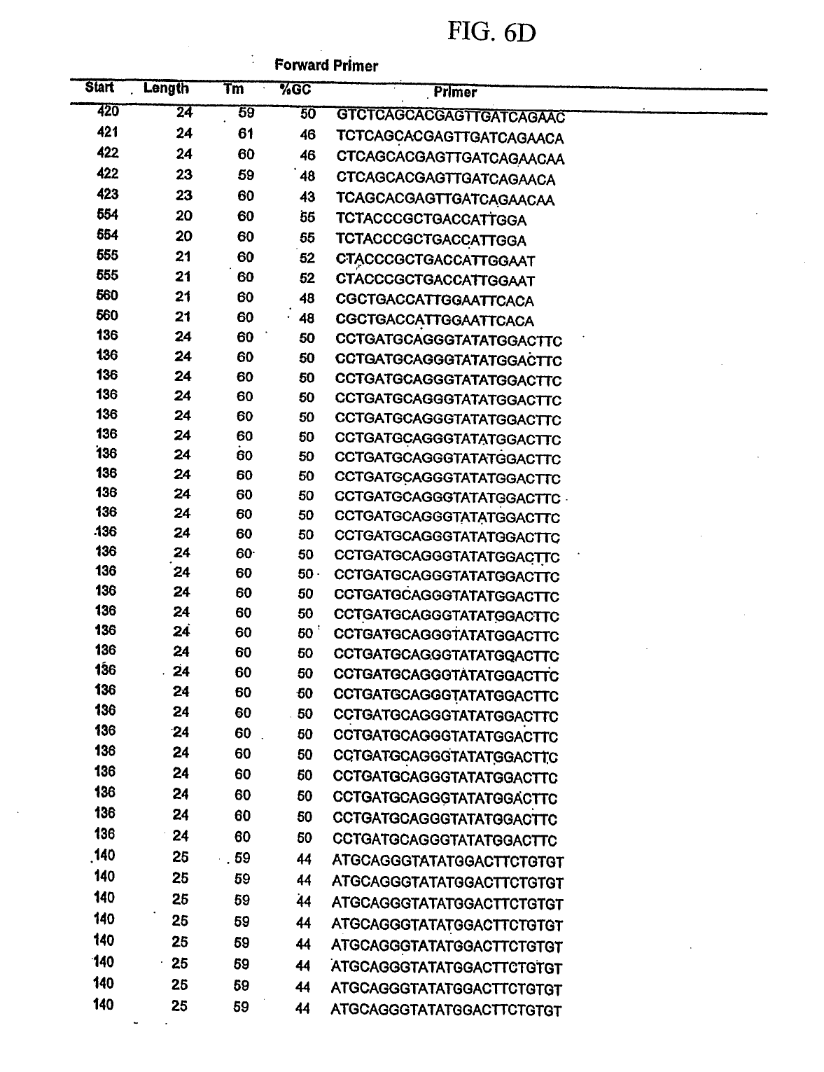

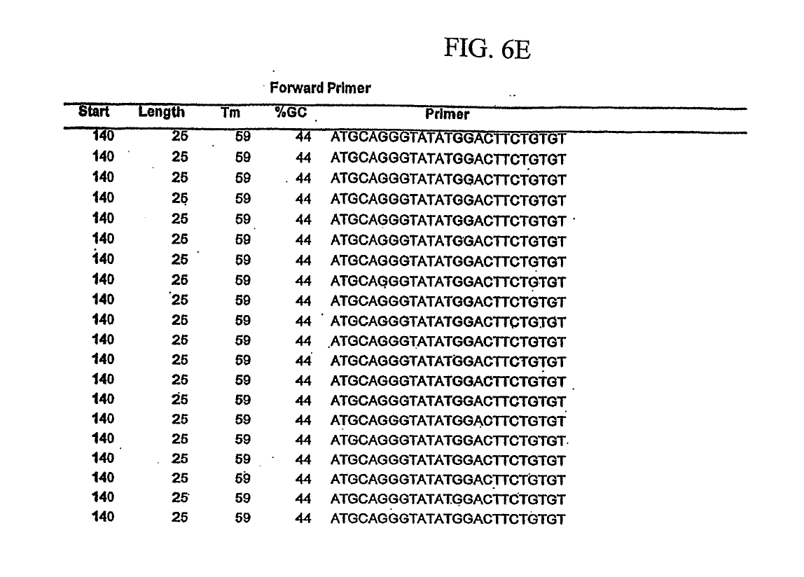

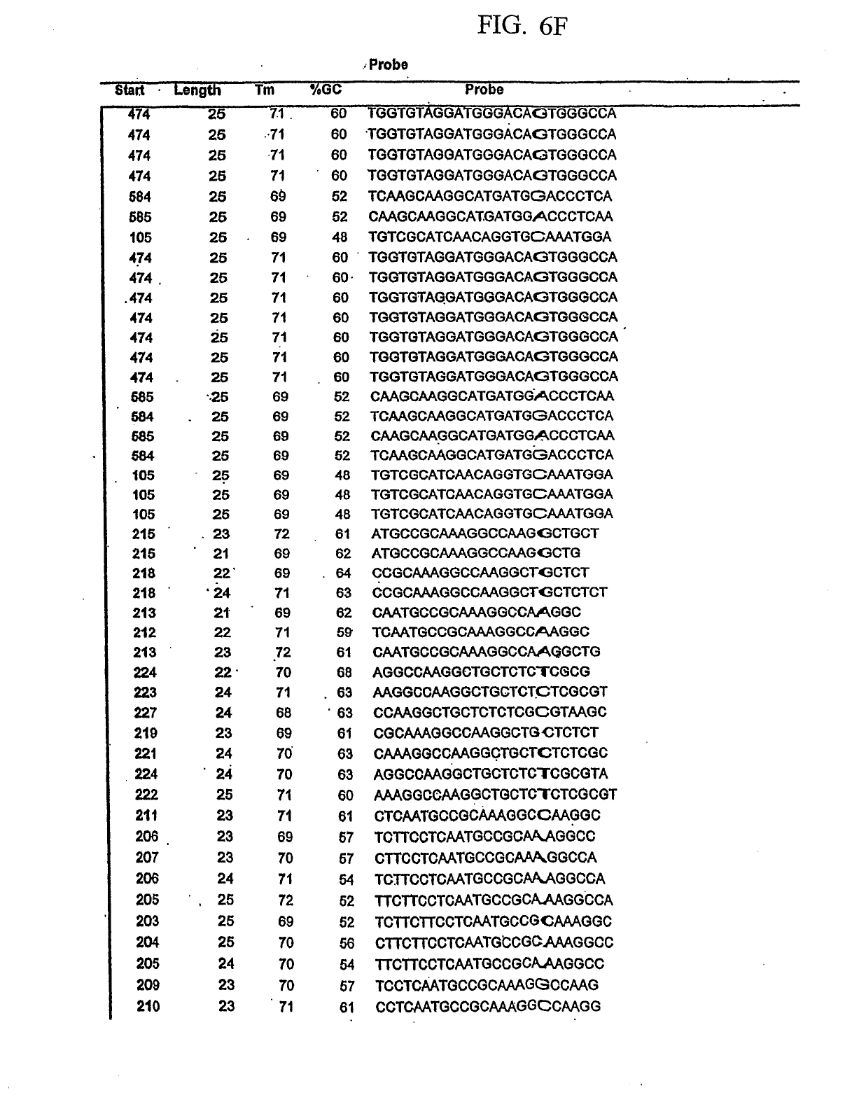

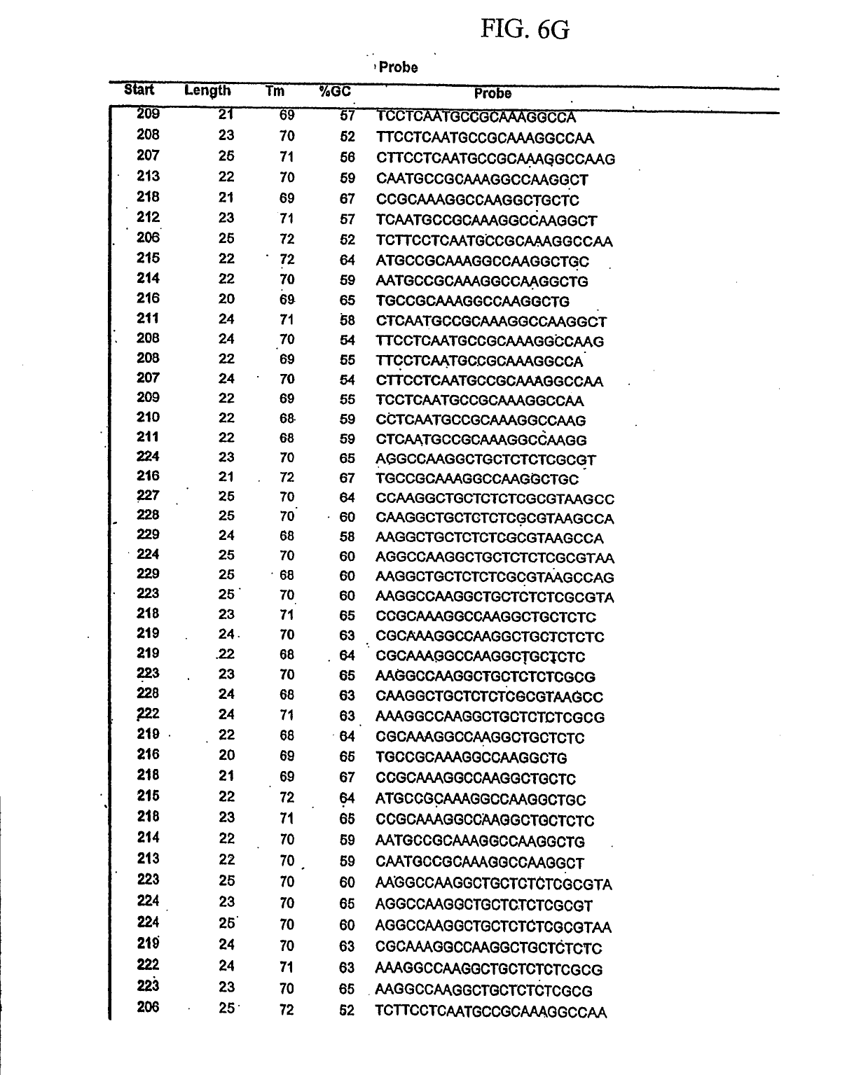

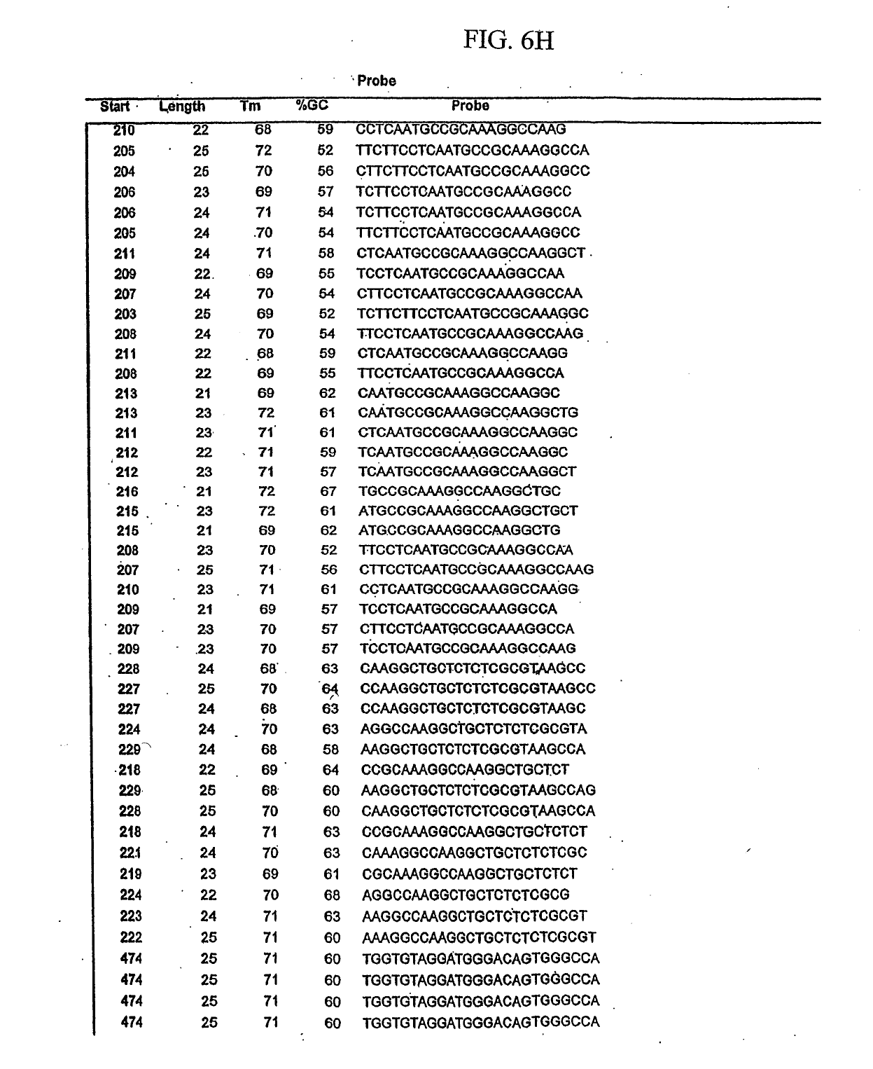

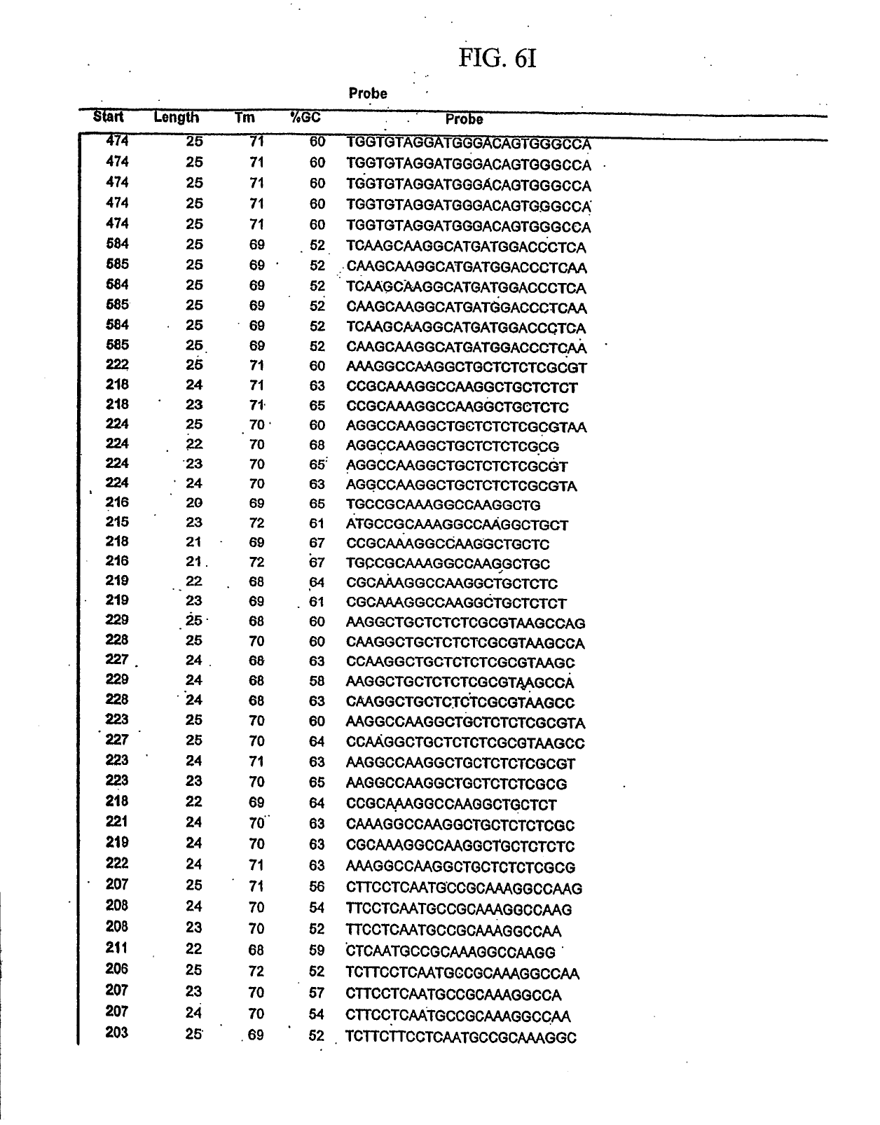

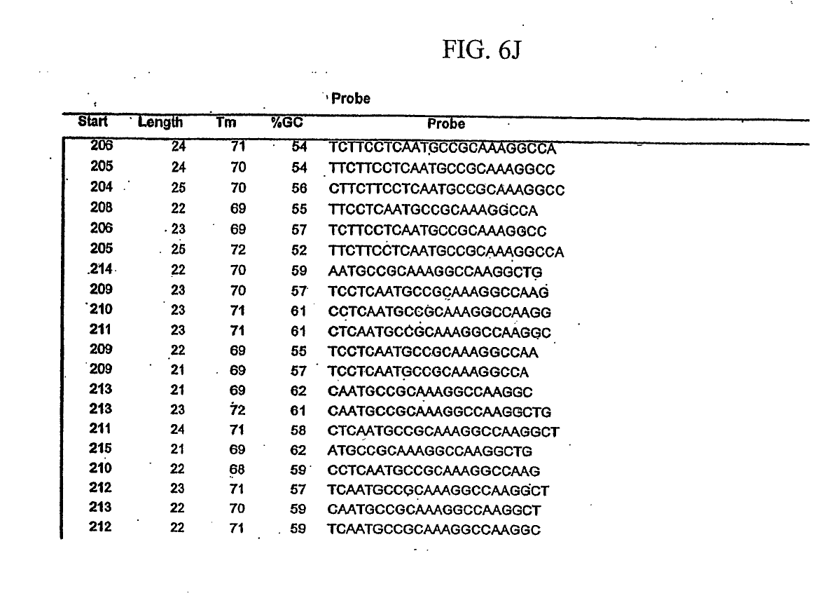

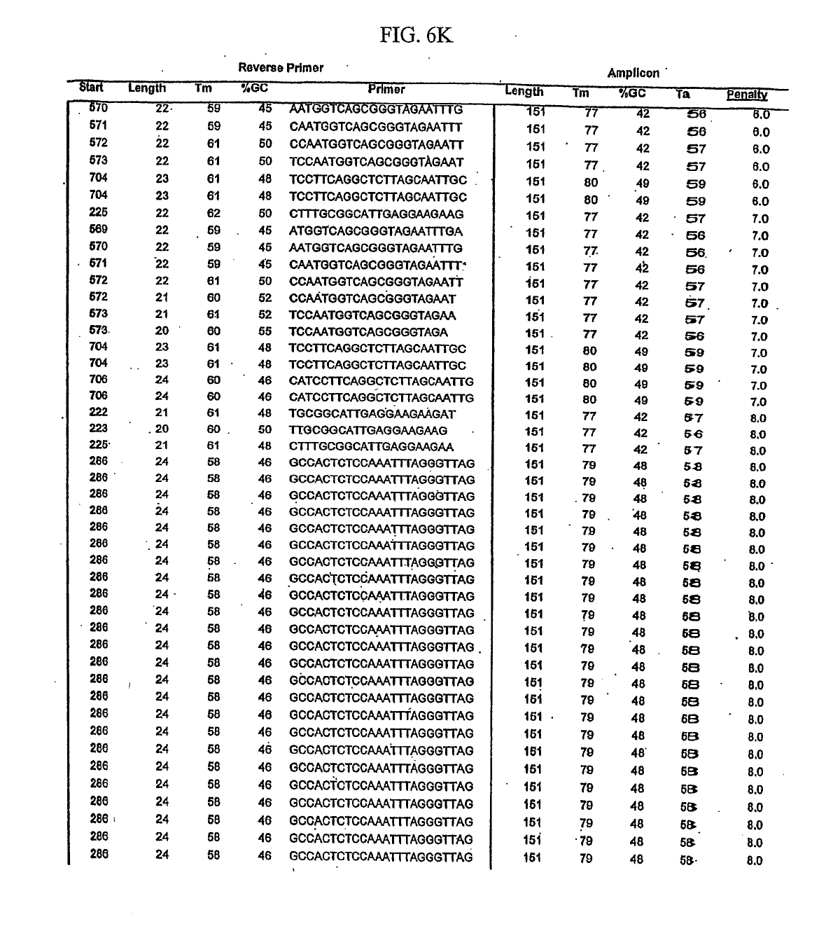

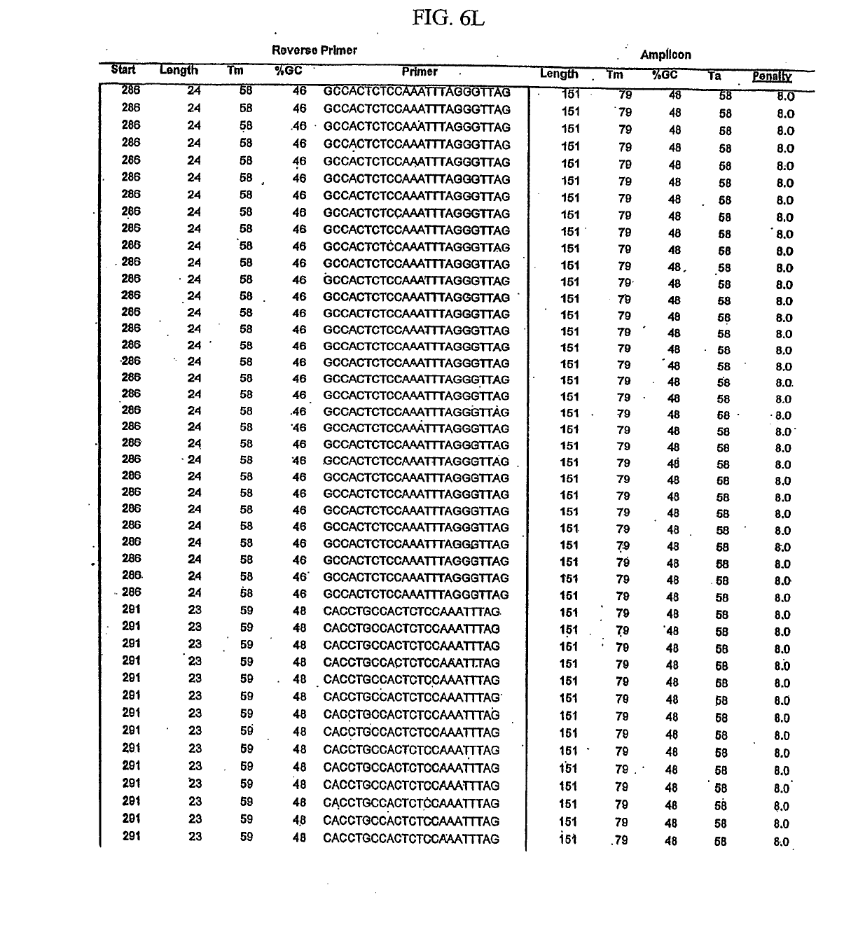

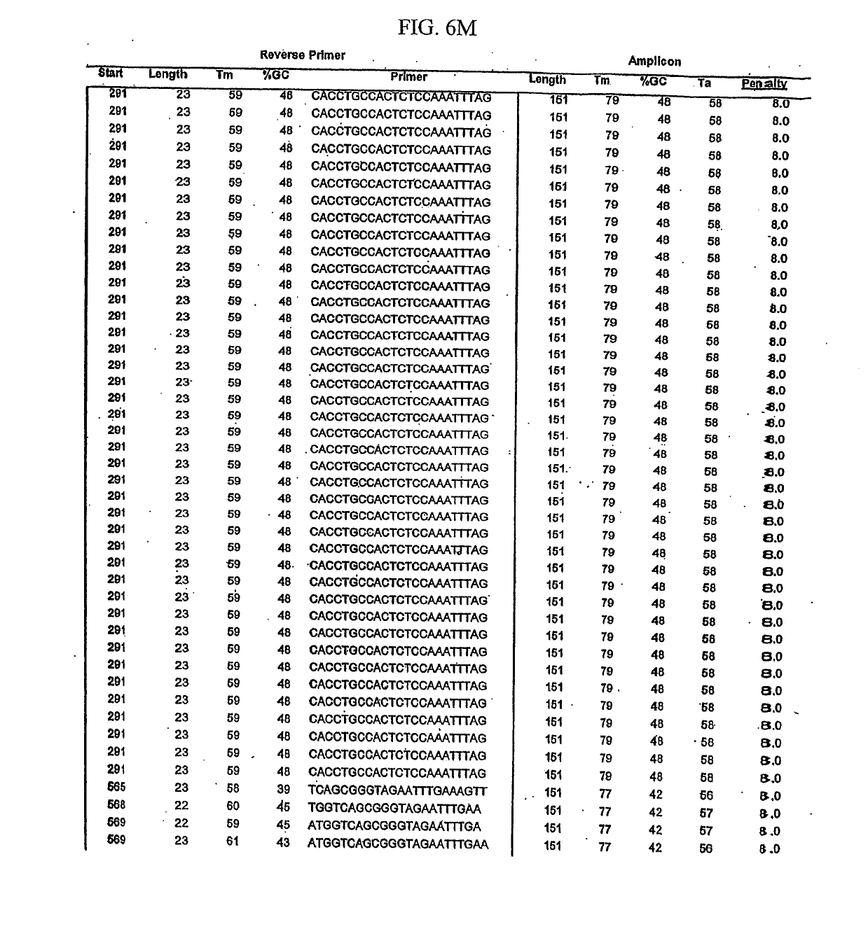

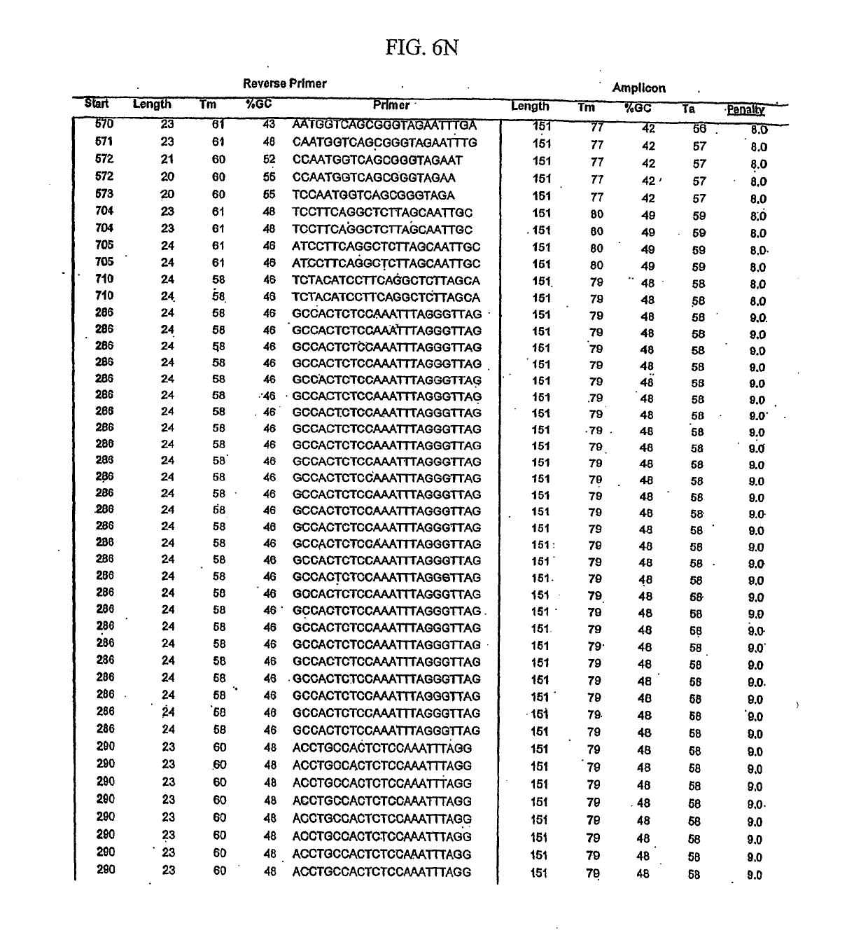

Similarly, FIGS. 6A-6O show representative primer/probe sets from the S segment of the LACV genome. Forward primers are shown in FIGS. 6A-6E; reverse primers for use with the forward primers are shown on the corresponding lines in FIGS. 6K-6O; and probes for use with the primer pairs shown in FIGS. 6A-6E and 6K-6O are shown on the corresponding lines in FIGS. 6F-6J. Thus, for example, the forward primer shown on line 1 of FIG. 6A (beginning at nucleotide position 420) can be used with the reverse primer shown on line 1 of FIG. 6K (beginning at nucleotide position 570), and the probe shown on line 1 of FIG. 6F (beginning at nucleotide position 474), and so forth for the remaining primers and probes shown in FIGS. 6A-6O.

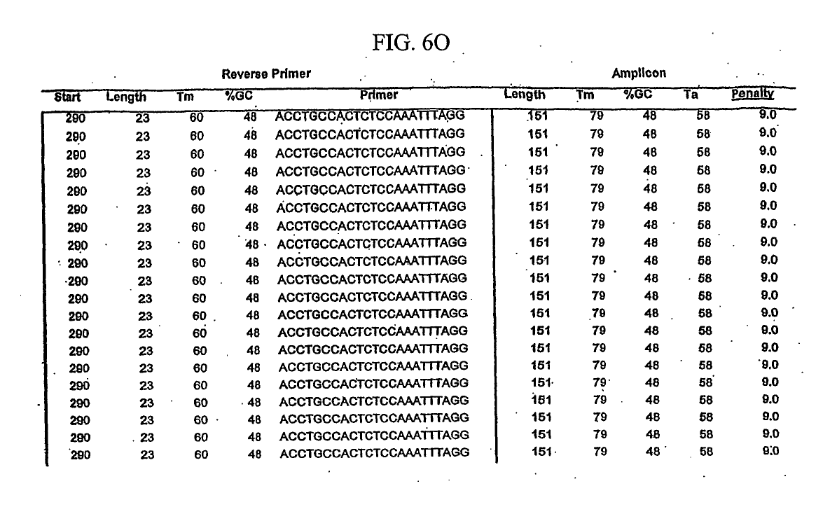

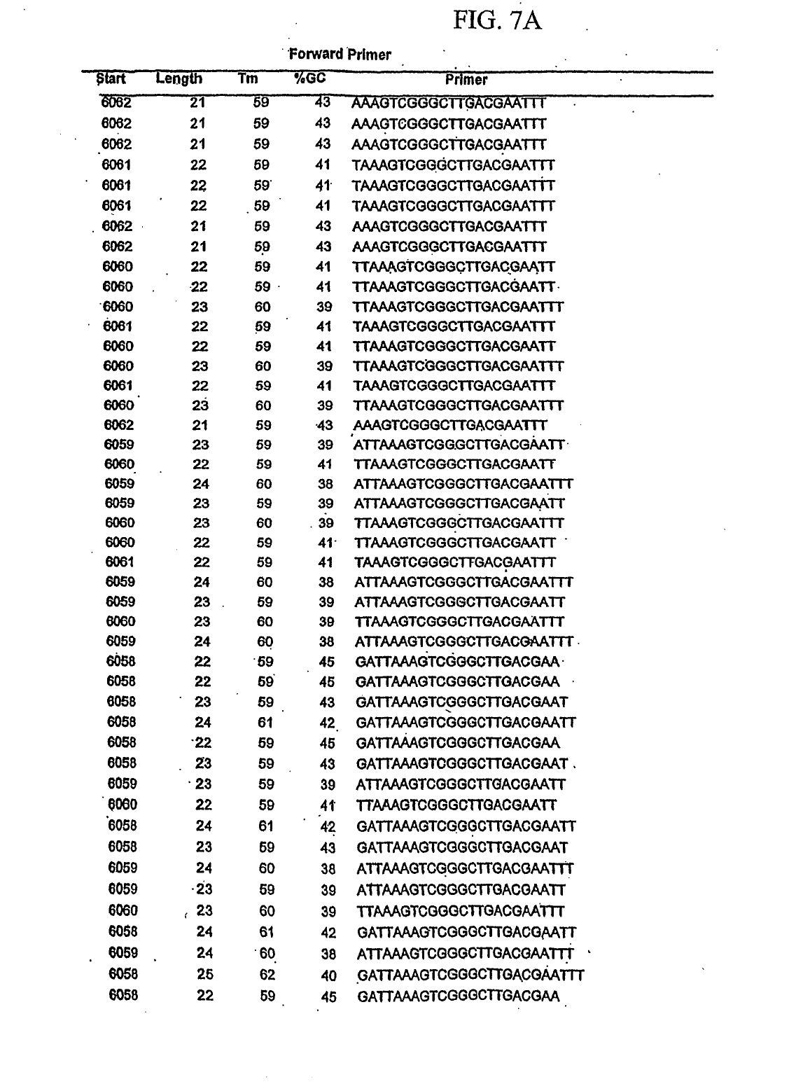

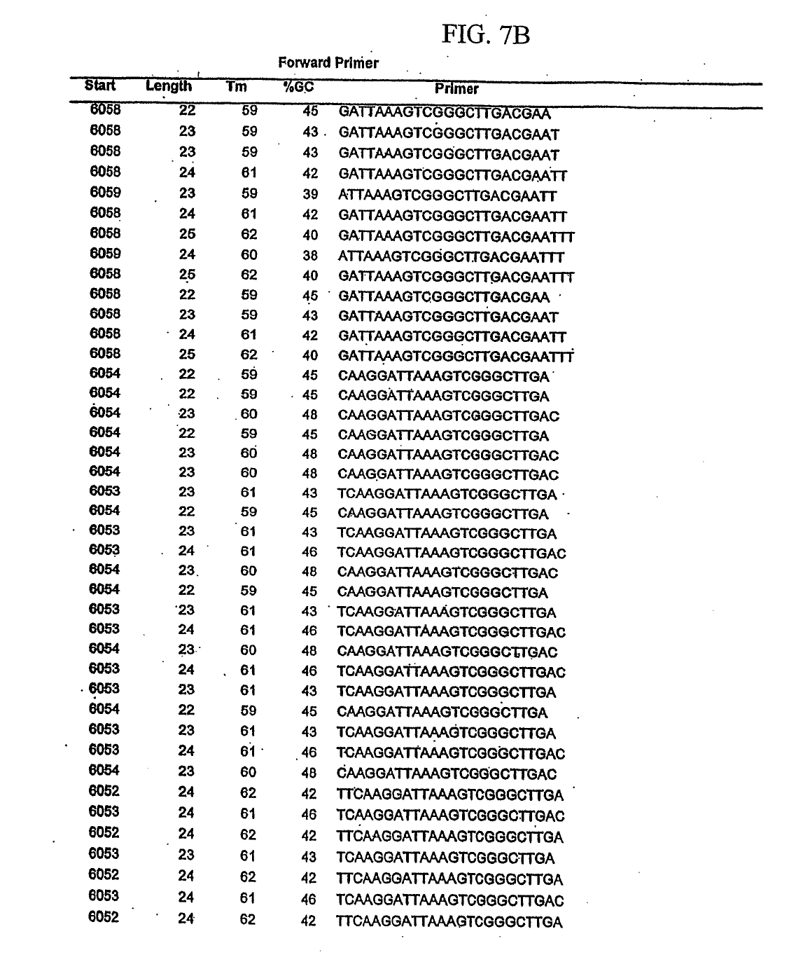

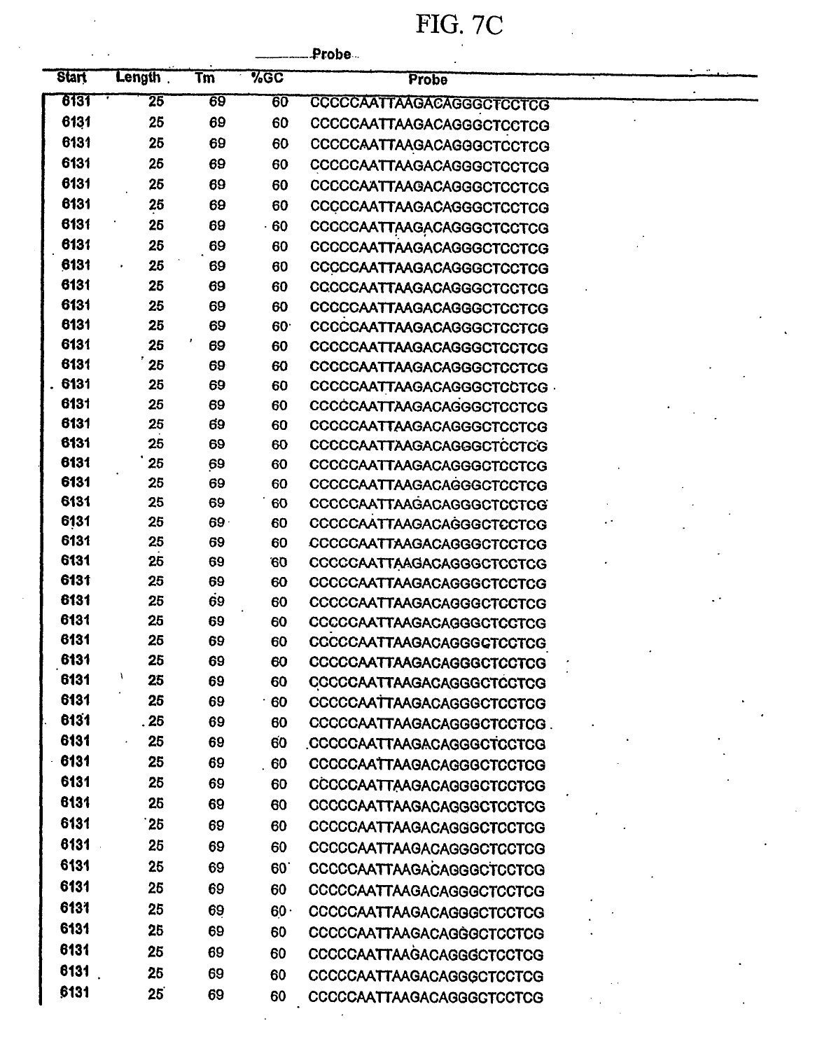

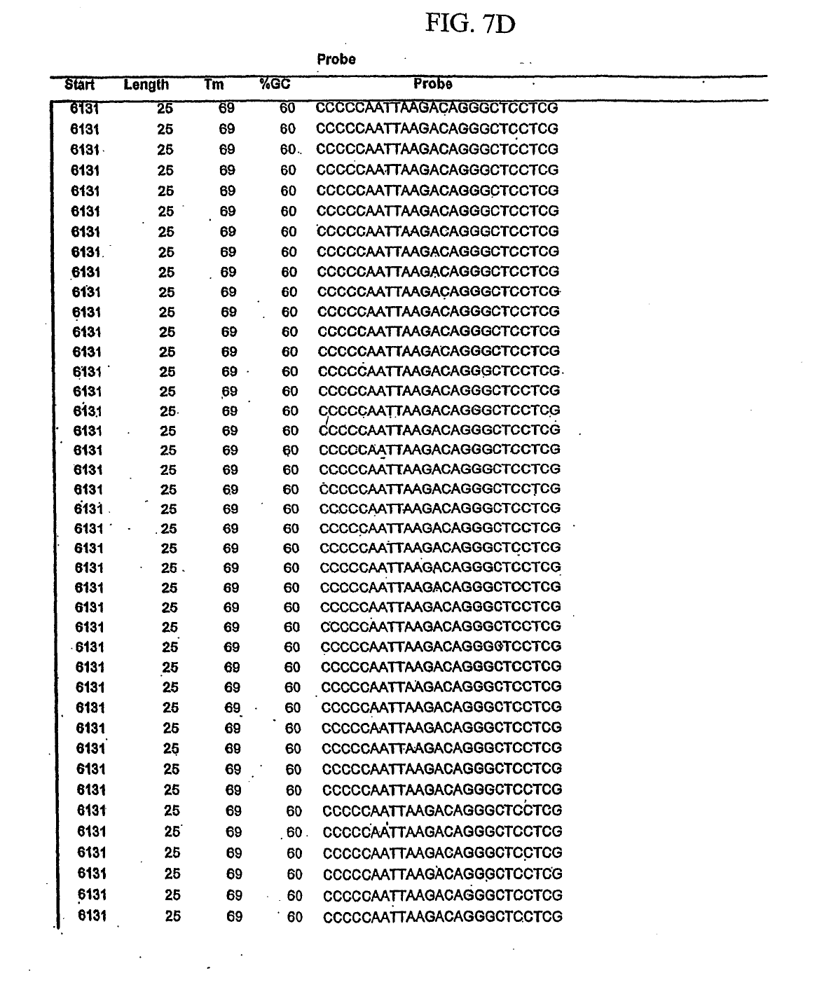

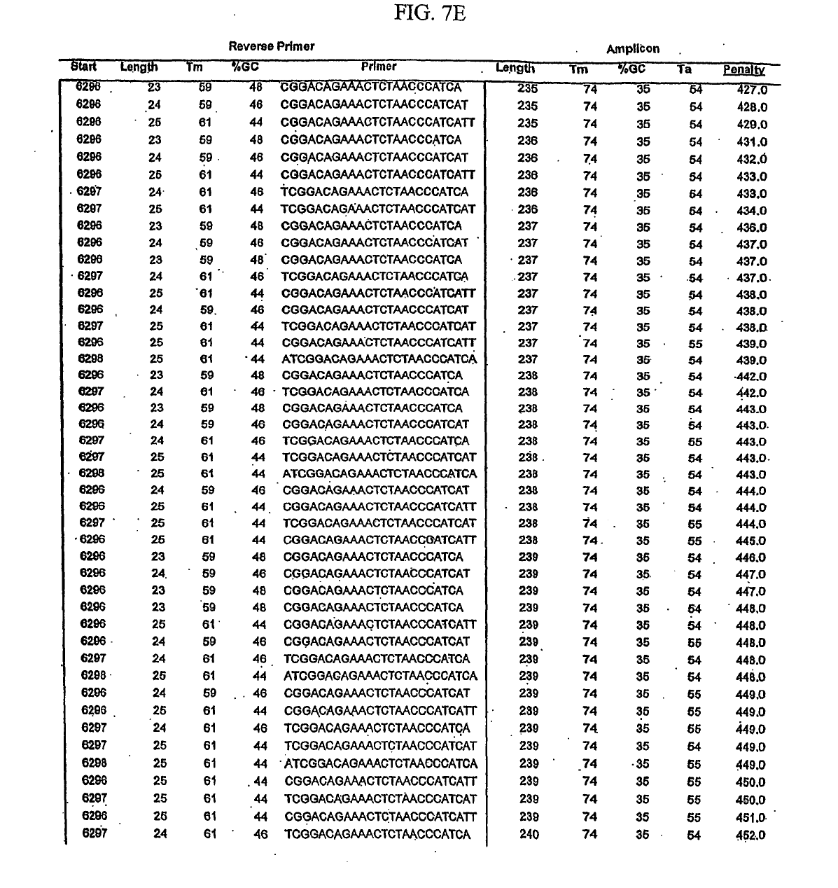

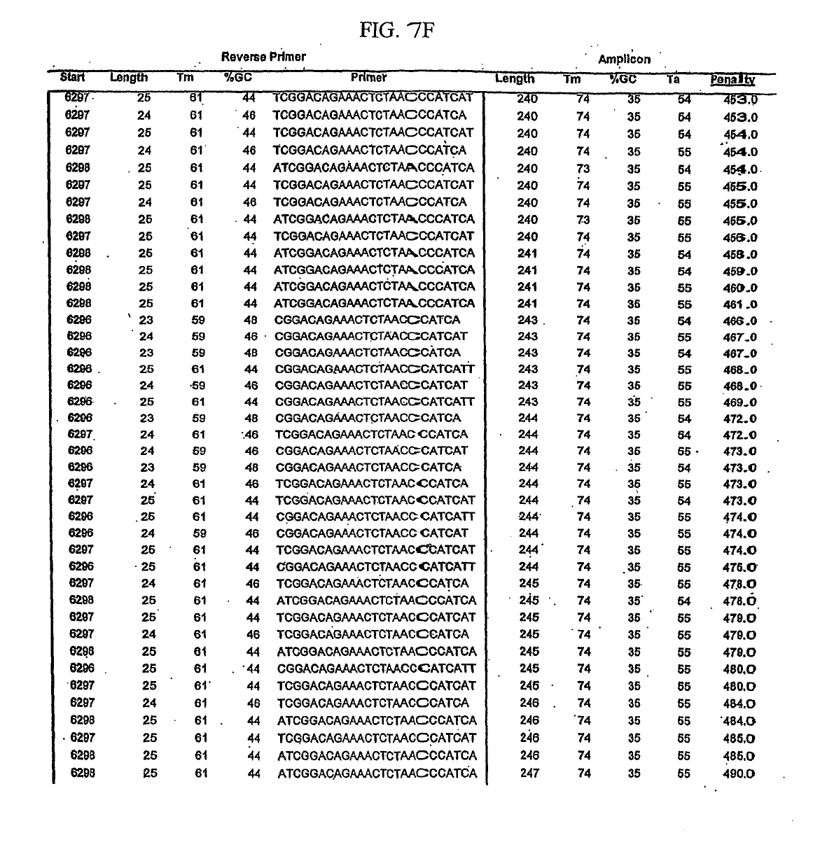

Additionally, FIGS. 7A-7F show representative primer/probe sets from the L segment of the LACV genome. Forward primers are shown in FIGS. 7A-7B; reverse primers for use with the forward primers are shown on the corresponding lines in FIGS. 7E-7F; and probes for use with the primer pairs shown in FIGS. 7A-7B and 7E-7F are shown on the corresponding lines in FIGS. 7C-7D. Thus, for example, the forward primer shown on line 1 of FIG. 7A (beginning at nucleotide position 6062) can be used with the reverse primer shown on line 1 of FIG. 7E (beginning at nucleotide position 6296), and the probe shown on line 1 of FIG. 7C (beginning at nucleotide position 6131), and so forth for the remaining primers and probes shown in FIGS. 7A-7F.

However, it is to be understood that the listed probes and primers are merely representative and other oligonucleotides from LACV, as well as oligonucleotides derived from other CAL viruses, will find use in the assays described herein. Moreover, oligonucleotides designated as primers herein, may be used as probes or capture oligonucleotides, and probes may be used as primers or capture oligonucleotides. One of skill in the art can readily determine appropriate primer and probe pairs, and optionally capture oligonucleotides, to use in order to detect LACV infection. Preferred primer and probe pairs from the LACV M sequence are the sense primer spanning positions 1470-1494, the antisense primer found at positions 1620-1596, and the probe found at positions 1534-1557, numbered relative to NCBI Accession No. NC 004109 (FIG. 1). A particularly preferred primer/probe set from the M segment is the use of an oligonucleotide spanning positions 1196-1172 as the antisense primer, i.e., the complement of nucleotides 1196-1172 shown in FIG. 1 having the sequence CGATCAACAATCCAATGATAACAAG (SEQ ID NO:7), a sense primer found at positions 1104-1125 of FIG. 1 having the sequence TGGAAATGGCATCGAGAATAAA (SEQ ID NO:8) and a probe with the nucleotide sequence spanning positions 1131-1169 having the sequence ATTATCTCACCTGTATCTTGAATTATGCTGTAAGCTGGG (SEQ ID NO:9) of FIG. 1. It has been found that the oligonucleotides found at positions 1104-1125 and 1131-1169, as designated above, are highly specific for the LACV sequence. These highly specific sequences can be used together, or individually, as primers, probes and/or capture oligonucleotides for specific detection of the LACV sequence as detailed further below.

Preferred primer and probe pairs from the LACV S sequence are the sense primer spanning positions 420-442 having the sequence GTCTCAGCACGAGTTGATCAGAA (SEQ ID NO:10), the antisense primer found at positions 570-549, i.e., the complement of nucleotides 570-549 shown in FIG. 2 having the sequence AATGGTCAGCGGGTAGAATTTG (SEQ ID NO:11), and the probe found at positions 474-498 having the sequence TGGTGTAGGATGGGACAGTGGGCCA (SEQ ID NO:12), numbered relative to NCBI Accession no. NC 004110 (FIG. 2). It has been found that the oligonucleotides found at positions 474-498 of FIG. 2 as designated above, and 796-820 of FIG. 2 having the sequence CATGAGGCATTCAAATTAGGTTCTA (SEQ ID NO:16), are highly specific for the LACV sequence. These highly specific sequences can be used together, or individually, as primers, probes and/or capture oligonucleotides for specific detection of the LACV sequence as detailed further below.

Preferred primer and probe pairs from the LACV L sequence are the sense primer spanning positions 6062-6082, having the sequence AAAGTCGGGCTTGACGAATT™ (SEQ ID NO:13) the antisense primer found at positions 6296-6274, i.e., the complement of nucleotides 6296-6274 shown in FIG. 3, having the sequence CGGACAGAAACTCTAACCCATCA (SEQ ID NO:14) and the probe found at positions 6131-6155, having the sequence CCCCCAATTAAGACAGGGCTCCTCG (SEQ ID NO:15), numbered relative to NCBI Accession no. NC 004108 (FIG. 3).

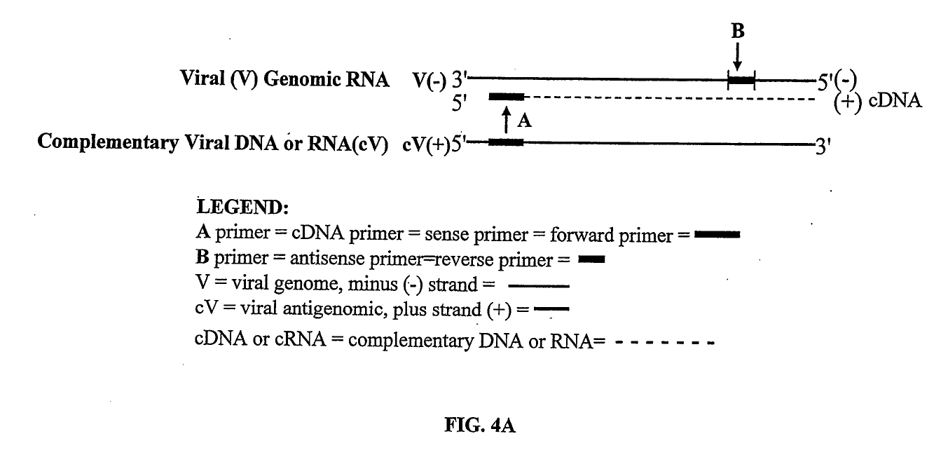

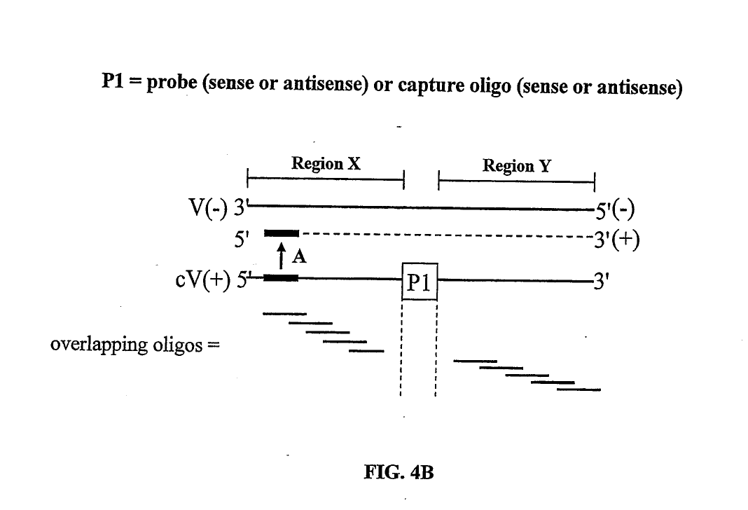

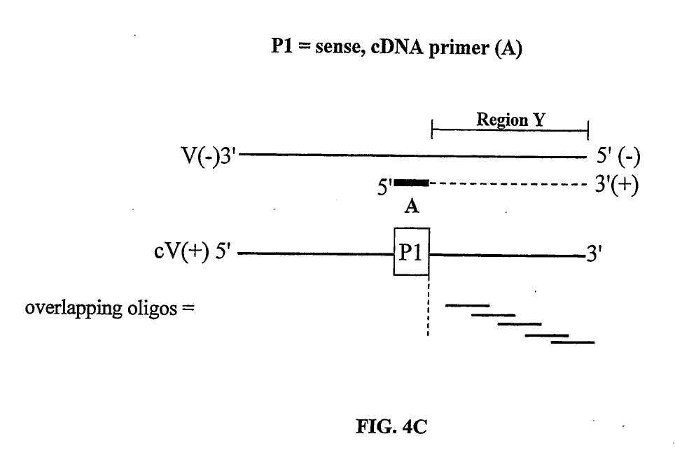

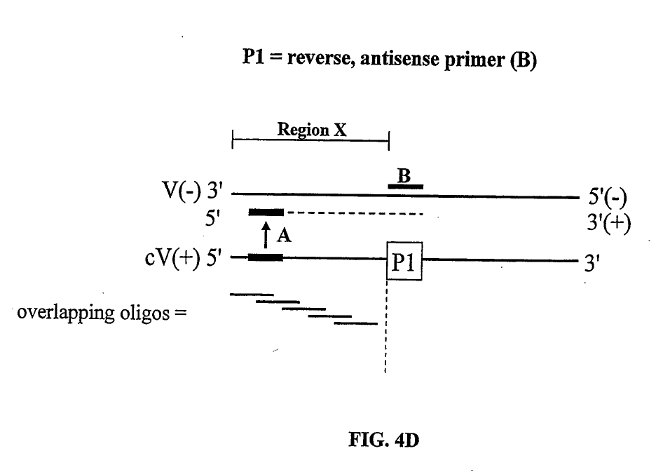

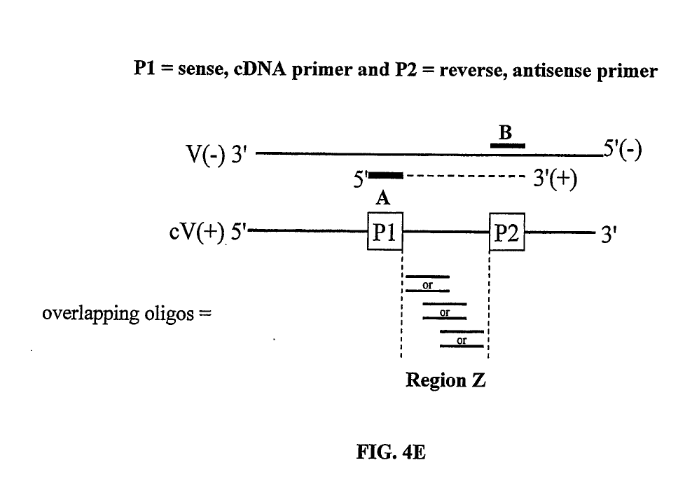

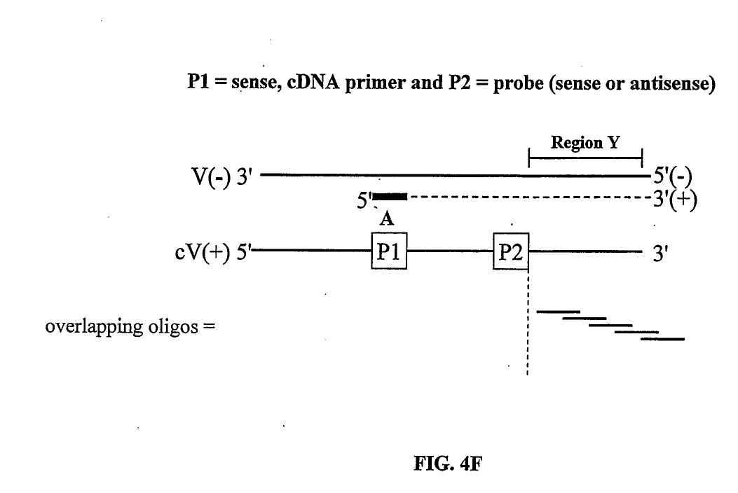

FIGS. 4A-4F show various strategies for using primers and probes to specifically detect LACV in nucleic acid-based assays. FIG. 4A depicts the viral genomic structure of LACV. As explained above, LACV-specific oligonucleotides can be used as probes for detection or capture of LACV nucleotides. Alternatively, the LACV-specific oligonucleotides can be used as primers for amplification, or can be used in combination with each other as primers or probes.

Various nucleic acid-based assays are described in detail below. For nucleic acid amplification testing (NAT) the antisense oligo can include a promoter sequence as described further below, such as the 17 promoter sequence at the 5′ end of the oligo. In this configuration, the sense primer would be the cDNA primer. For PCR, the antisense primer would be the reverse primer and the sense primer would be the cDNA primer.

For example, as shown in FIG. 4B, Probe (P1) serves as a probe. Any oligo in Region X could serve as a cDNA primer (A) and any oligo in Region Y could be an antisense primer (B). Sense primers would be in the antigenomic sequence (cV(+) strand). Antisense primers would be the viral genomic sequence (V(−) strand. In FIG. 4C, P1 serves as the sense primer, which is the cDNA primer. In this configuration, oligos in Region Y can be antisense primers or probes. Probes must lie between the two primers. In this configuration, the 3′ terminal antisense primer can only be a primer and not a probe. In FIG. 4D, P1 serves as a primer in the antisense orientation. Oligos in Region X can be cDNA (sense) primers or probes. Probes must lie between the two primers. The 5′ terminal sense primer can only be a primer and not a probe. In FIG. 4E, P1 serves as the sense primer (cDNA) primer and P2 is the antisense primer. Oligos between P1 and P2 can be probes. In FIG. 4F, P1 is a primer and P2 is a probe. As shown in FIG. 4F, if P1 is the cDNA (sense primer) and oligos downstream from P2 are used as antisense primers, then P2 can serve as a probe. Alternatively, if P2 serves as an antisense primer and oligos upstream of P1 serve as cDNA primers (sense), then P1 can serve as a probe

When utilizing a hybridization-based detection system, a nucleic acid probe is chosen that is complementary to a target nucleic acid sequence. By selection of appropriate conditions, the probe and the target sequence “selectively hybridize,” or bind, to each other to form a hybrid molecule. An oligonucleotide that “selectively hybridizes” to a LACV sequence under hybridization conditions described below, denotes an oligonucleotide, e.g., a primer, probe or a capture oligonucleotide, that binds to a LACV sequence but does not bind to a sequence from a non-LACV CAL virus. In one embodiment of the present invention, a nucleic acid molecule is capable of hybridizing selectively to a target sequence under moderately stringent hybridization conditions. In the context of the present invention, moderately stringent hybridization conditions allow detection of a target nucleic acid sequence of at least 14 nucleotides in length having at least approximately 70% sequence identity with the sequence of the selected nucleic acid probe. In another embodiment, such selective hybridization is performed under stringent hybridization conditions. Stringent hybridization conditions allow detection of target nucleic acid sequences of at least 14 nucleotides in length having a sequence identity of greater than 90% with the sequence of the selected nucleic acid probe. Hybridization conditions useful for probe/target hybridization where the probe and target have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Hybrid molecules can be formed, for example, on a solid support, in solution, and in tissue sections. The formation of hybrids can be monitored by inclusion of a reporter molecule, typically, in the probe. Such reporter molecules, or detectable elements include, but are not limited to, radioactive elements, fluorescent markers, and molecules to which an enzyme-conjugated ligand can bind.

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts and other hybridization solution components, the presence or absence of blocking agents in the hybridization solutions (e.g., formamide, dextran sulfate, and polyethylene glycol), hybridization reaction temperature and time parameters, as well as, varying wash conditions. The selection of a particular set of hybridization conditions is well known (see, for example, Sambrook, et al., Molecular Cloning: A Laboratory Manual, Second Edition, (1989) Cold Spring Harbor, N.Y.).

As explained above, the primers and probes may be used in polymerase chain reaction (PCR)-based techniques, such as RT-PCR, to detect CAL virus infection in biological samples. PCR is a technique for amplifying a desired target nucleic acid sequence contained in a nucleic acid molecule or mixture of molecules. In PCR, a pair of primers is employed in excess to hybridize to the complementary strands of the target nucleic acid. The primers are each extended by a polymerase using the target nucleic acid as a template. The extension products become target sequences themselves after dissociation from the original target strand. New primers are then hybridized and extended by a polymerase, and the cycle is repeated to geometrically increase the number of target sequence molecules. The PCR method for amplifying target nucleic acid sequences in a sample is well known in the art and has been described in, e.g., Innis et al. (eds.) PCR Protocols (Academic Press, NY 1990); Taylor (1991) Polymerase chain reaction: basic principles and automation, in PCR: A Practical Approach, McPherson et al. (eds.) IRL Press, Oxford; Saiki et al. (1986) Nature 324:163; as well as in U.S. Pat. Nos. 4,683,195, 4,683,202 and 4,889,818, all incorporated herein by reference in their entireties.

In particular, PCR uses relatively short oligonucleotide primers which flank the target nucleotide sequence to be amplified, oriented such that their 3′ ends face each other, each primer extending toward the other. The polynucleotide sample is extracted and denatured, preferably by heat, and hybridized with first and second primers that are present in molar excess. Polymerization is catalyzed in the presence of the four deoxyribonucleotide triphosphates (dNTPs dATP, dGTP, dCTP and dTTP) using a primer- and template-dependent polynucleotide polymerizing agent, such as any enzyme capable of producing primer extension products, for example, E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). This results in two “long products” which contain the respective primers at their 5′ ends covalently linked to the newly synthesized complements of the original strands. The reaction mixture is then returned to polymerizing conditions, e.g., by lowering the temperature, inactivating a denaturing agent, or adding more polymerase, and a second cycle is initiated. The second cycle provides the two original strands, the two long products from the first cycle, two new long products replicated from the original strands, and two “short products” replicated from the long products. The short products have the sequence of the target sequence with a primer at each end. On each additional cycle, an additional two long products are produced, and a number of short products equal to the number of long and short products remaining at the end of the previous cycle. Thus, the number of short products containing the target sequence grows exponentially with each cycle. Preferably, PCR is carried out with a commercially available thermal cycler, e.g., Perkin Elmer.

RNAs may be amplified by reverse transcribing the RNA into cDNA, and then performing PCR (RT-PCR), as described above. Alternatively, a single enzyme may be used for both steps as described in U.S. Pat. No. 5,322,770, incorporated herein by reference in its entirety. RNA may also be reverse transcribed into cDNA, followed by asymmetric gap ligase chain reaction (RT-AGLCR) as described by Marshall et al. (1994) PCR Meth. App. 4:80-84.

The Ligase Chain Reaction (LCR) is an alternate method for nucleic acid amplification. In LCR, probe pairs are used which include two primary (first and second) and two secondary (third and fourth) probes, all of which are employed in molar excess to target. The first probe hybridizes to a first segment of the target strand, and the second probe hybridizes to a second segment of the target strand, the first and second segments being contiguous so that the primary probes abut one another in 5′ phosphate-3′ hydroxyl relationship, and so that a ligase can covalently fuse or ligate the two probes into a fused product. In addition, a third (secondary) probe can hybridize to a portion of the first probe and a fourth (secondary) probe can hybridize to a portion of the second probe in a similar abutting fashion. If the target is initially double stranded, the secondary probes also will hybridize to the target complement in the first instance. Once the ligated strand of primary probes is separated from the target strand, it will hybridize with the third and fourth probes which can be ligated to form a complementary, secondary ligated product. It is important to realize that the ligated products are functionally equivalent to either the target or its complement. By repeated cycles of hybridization and ligation, amplification of the target sequence is achieved. This technique is described more completely in EPA 320,308 to K. Backman published Jun. 16, 1989 and EPA 439,182 to K. Backman et al., published Jul. 31, 1991, both of which are incorporated herein by reference.

Other known amplification methods which can be utilized herein include but are not limited to the so-called “NASBA” or “3SR” technique described by Guatelli et al., Proc. Natl. Acad. Sci. USA (1990) 87:1874-1878 and J. Compton, Nature (1991) 350:91-92 (1991); Q-beta amplification; strand displacement amplification (as described in Walker et al., Clin. Chem. (1996) 42:9-13 and EPA 684,315; target mediated amplification, as described in International Publication No. WO 93/22461, and the TaqMan™ assay.

The fluorogenic 5′ nuclease assay, known as the TaqMan™ assay (Perkin-Elmer), is a powerful and versatile PCR-based detection system for nucleic acid targets. Hence, primers and probes derived from conserved and/or non-conserved regions of the CAL virus genome in question can be used in TaqMan™ analyses to detect the presence of infection in a biological sample. Analysis is performed in conjunction with thermal cycling by monitoring the generation of fluorescence signals. The assay system dispenses with the need for gel electrophoretic analysis, and is capable of generating quantitative data allowing the determination of target copy numbers. For example, standard curves can be produced using serial dilutions of previously quantified CAL viral suspensions. A standard graph can be produced with copy numbers of each of the panel members against which sample unknowns can be compared.

The fluorogenic 5′ nuclease assay is conveniently performed using, for example, AmpliTaq Gold™ DNA polymerase, which has endogenous 5′ nuclease activity, to digest an internal oligonucleotide probe labeled with both a fluorescent reporter dye and a quencher (see, Holland et al., Proc. Natl. Acad. Sci. USA (1991) 88:7276-7280; and Lee et al., Nucl. Acids Res. (1993) 21:3761-3766). Assay results are detected by measuring changes in fluorescence that occur during the amplification cycle as the fluorescent probe is digested, uncoupling the dye and quencher labels and causing an increase in the fluorescent signal that is proportional to the amplification of target nucleic acid.

The amplification products can be detected in solution or using solid supports. In this method, the TaqMan™ probe is designed to hybridize to a target sequence within the desired PCR product. The 5′ end of the TaqMan™ probe contains a fluorescent reporter dye. The 3′ end of the probe is blocked to prevent probe extension and contains a dye that will quench the fluorescence of the 5′ fluorophore. During subsequent amplification, the 5′ fluorescent label is cleaved off if a polymerase with 5′ exonuclease activity is present in the reaction. Excision of the 5′ fluorophore results in an increase in fluorescence that can be detected.

For a detailed description of the TaqMan™ assay, reagents and conditions for use therein, see, e.g., Holland et al., Proc. Natl. Acad. Sci, U.S.A. (1991) 88:7276-7280; U.S. Pat. Nos. 5,538,848, 5,723,591, and 5,876,930, all incorporated herein by reference in their entireties.

Accordingly, the present invention relates to methods for amplifying a target CAL virus nucleotide sequence using a nucleic acid polymerase having 5′ to 3′ nuclease activity, one or more primers capable of hybridizing to the CAL virus target sequence, and an oligonucleotide probe capable of hybridizing to the CAL virus target sequence 3′ relative to the primer. During amplification, the polymerase digests the oligonucleotide probe when it is hybridized to the target sequence, thereby separating the reporter molecule from the quencher molecule. As the amplification is conducted, the fluorescence of the reporter molecule is monitored, with fluorescence corresponding to the occurrence of nucleic acid amplification. The reporter molecule is preferably a fluorescein dye and the quencher molecule is preferably a rhodamine dye.

While the length of the primers and probes can vary, the probe sequences are selected such that they have a higher melt temperature than the primer sequences. Preferably, the probe sequences have an estimated melt temperature that is about 10° C. higher than the melt temperature for the amplification primer sequences. Hence, the primer sequences are generally shorter than the probe sequences. Typically, the primer sequences are in the range of between 10-75 nucleotides long, more typically in the range of 20-45. The typical probe is in the range of between 10-50 nucleotides long, more typically 15-40 nucleotides in length. Representative primers and probes useful in TaqMan™ assays are described above.

The CAL virus sequences described herein may also be used as a basis for transcription-mediated amplification (TMA) assays. TMA provides a method of identifying target nucleic acid sequences present in very small amounts in a biological sample. Such sequences may be difficult or impossible to detect using direct assay methods. In particular, TMA is an isothermal, autocatalytic nucleic acid target amplification system that can provide more than a billion RNA copies of a target sequence. The assay can be done qualitatively, to accurately detect the presence or absence of the target sequence in a biological sample. The assay can also provide a quantitative measure of the amount of target sequence over a concentration range of several orders of magnitude. TMA provides a method for autocatalytically synthesizing multiple copies of a target nucleic acid sequence without repetitive manipulation of reaction conditions such as temperature, ionic strength and pH.

Generally, TMA includes the following steps: (a) isolating nucleic acid, including RNA, from the biological sample of interest suspected of being infected with CAL virus; and (b) combining into a reaction mixture (i) the isolated nucleic acid, (ii) first and second oligonucleotide primers, the first primer having a complexing sequence sufficiently complementary to the 3′ terminal portion of an RNA target sequence, if present (for example the (+) strand), to complex therewith, and the second primer having a complexing sequence sufficiently complementary to the 3′ terminal portion of the target sequence of its complement (for example, the (−) strand) to complex therewith, wherein the first oligonucleotide further comprises a sequence 5′ to the complexing sequence which includes a promoter, (iii) a reverse transcriptase or RNA and DNA dependent DNA polymerases, (iv) an enzyme activity which selectively degrades the RNA strand of an RNA-DNA complex (such as an RNAse H) and (v) an RNA polymerase which recognizes the promoter.

The components of the reaction mixture may be combined stepwise or at once. The reaction mixture is incubated under conditions whereby an oligonucleotide/target sequence is formed, including DNA priming and nucleic acid synthesizing conditions (including ribonucleotide triphosphates and deoxyribonucleotide triphosphates) for a period of time sufficient to provide multiple copies of the target sequence. The reaction advantageously takes place under conditions suitable for maintaining the stability of reaction components such as the component enzymes and without requiring modification or manipulation of reaction conditions during the course of the amplification reaction. Accordingly, the reaction may take place under conditions that are substantially isothermal and include substantially constant ionic strength and pH. The reaction conveniently does not require a denaturation step to separate the RNA-DNA complex produced by the first DNA extension reaction.

Suitable DNA polymerases include reverse transcriptases, such as avian myeloblastosis virus (AMV) reverse transcriptase (available from, e.g., Seilcagaku America, Inc.) and Moloney murine leukemia virus (MMLV) reverse transcriptase (available from, e.g., Bethesda Research Laboratories).

Promoters or promoter sequences suitable for incorporation in the primers are nucleic acid sequences (either naturally occurring, produced synthetically or a product of a restriction digest) that are specifically recognized by an RNA polymerase that recognizes and binds to that sequence and initiates the process of transcription whereby RNA transcripts are produced. The sequence may optionally include nucleotide bases extending beyond the actual recognition site for the RNA polymerase which may impart added stability or susceptibility to degradation processes or increased transcription efficiency. Examples of useful promoters include those which are recognized by certain bacteriophage polymerases such as those from bacteriophage T3, T7 or SP6, or a promoter from E. coli. These RNA polymerases are readily available from commercial sources, such as New England Biolabs and Epicentre.

Some of the reverse transcriptases suitable for use in the methods herein have an RNAse H activity, such as AMV reverse transcriptase. It may, however, be preferable to add exogenous RNAse H, such as E. coli RNAse H, even when AMV reverse transcriptase is used. RNAse H is readily available from, e.g., Bethesda Research Laboratories.

The RNA transcripts produced by these methods may serve as templates to produce additional copies of the target sequence through the above-described mechanisms. The system is autocatalytic and amplification occurs autocatalytically without the need for repeatedly modifying or changing reaction conditions such as temperature, pH, ionic strength or the like.

Detection may be done using a wide variety of methods, including direct sequencing, hybridization with sequence-specific oligomers, gel electrophoresis and mass spectrometry. these methods can use heterogeneous or homogeneous formats, isotopic or nonisotopic labels, as well as no labels at all.

One preferable method of detection is the use of target sequence-specific oligonucleotide probes described above. The probes may be used in hybridization protection assays (IRA). In this embodiment, the probes are conveniently labeled with acridinium ester (AE), a highly chemiluminescent molecule. See, e.g., Nelson et al. (1995) “Detection of Acridinium Esters by Chemiluminescence” in Nonisotopic Probing, Blotting and Sequencing, Kricka L. J. (ed) Academic Press, San Diego, Calif.; Nelson et al. (1994) “Application of the Hybridization Protection Assay (HPA) to PCR” in The Polymerase Chain Reaction, Mullis et al. (eds.) Birkhauser, Boston, Mass.; Weeks et al., Clin. Chem. (1983) 29:1474-1479; Berry et al., Clin. Chem. (1988) 34:2087-2090. One AE molecule is directly attached to the probe using a non-nucleotide-based linker arm chemistry that allows placement of the label at any location within the probe. See, e.g., U.S. Pat. Nos. 5,585,481 and 5,185,439. Chemiluminescence is triggered by reaction with alkaline hydrogen peroxide which yields an excited N-methyl acridone that subsequently collapses to ground state with the emission of a photon.

When the AE molecule is covalently attached to a nucleic acid probe, hydrolysis is rapid under mildly alkaline conditions. When the AE-labeled probe is exactly complementary to the target nucleic acid, the rate of AE hydrolysis is greatly reduced. Thus, hybridized and unhybridized AE-labeled probe can be detected directly in solution, without the need for physical separation.

HPA generally consists of the following steps: (a) the AE-labeled probe is hybridized with the target nucleic acid in solution for about 15 to about 30 minutes. A mild alkaline solution is then added and AE coupled to the unhybridized probe is hydrolyzed. This reaction takes approximately 5 to 10 minutes. The remaining hybrid-associated AE is detected as a measure of the amount of target present. This step takes approximately 2 to 5 seconds. Preferably, the differential hydrolysis step is conducted at the same temperature as the hybridization step, typically at 50 to 70° C. Alternatively, a second differential hydrolysis step may be conducted at room temperature. This allows elevated pHs to be used, for example in the range of 10-11, which yields larger differences in the rate of hydrolysis between hybridized and unhybridized AE-labeled probe. HPA is described in detail in, e.g., U.S. Pat. Nos. 6,004,745; 5,948,899; and 5,283,174, the disclosures of which are incorporated by reference herein in their entireties.

TMA is described in detail in, e.g., U.S. Pat. No. 5,399,491, the disclosure of which is incorporated herein by reference in its entirety. In one example of a typical assay, an isolated nucleic acid sample, suspected of containing a CAL virus target sequence, is mixed with a buffer concentrate containing the buffer, salts, magnesium, nucleotide triphosphates, primers, dithiothreitol, and spermidine. The reaction is optionally incubated at about 100° C. for approximately two minutes to denature any secondary structure. After cooling to room temperature, reverse transcriptase, RNA polymerase, and RNAse H are added and the mixture is incubated for two to four hours at 37° C. The reaction can then be assayed by denaturing the product, adding a probe solution, incubating 20 minutes at 60° C., adding a solution to selectively hydrolyze the =hybridized probe, incubating the reaction six minutes at 60° C., and measuring the remaining chemiluminescence in a luminometer.

In another aspect of the invention, two or more of the tests described above are performed to confirm the presence of the organism. For example, if the first test used transcription mediated amplification (TMA) to amplify the nucleic acids for detection, then an alternative nucleic acid testing (NAT) assay is performed, for example, by using PCR amplification, RT-PCR, and the like, as described herein. Thus, CAL virus can be specifically and selectively detected even when the sample contains other organisms, such as HIV and/or HCV, for example.

As is readily apparent, design of the assays described herein are subject to a great deal of variation, and many formats are known in the art. The above descriptions are merely provided as guidance and one of skill in the art can readily modify the described protocols, using techniques well known in the art.

The above-described assay reagents, including the primers, probes, solid support with bound probes, as well as other detection reagents, can be provided in kits, with suitable instructions and other necessary reagents, in order to conduct the assays as described above. The kit will normally contain in separate containers the combination of primers and probes (either already bound to a solid matrix or separate with reagents for binding them to the matrix), control formulations (positive and/or negative), labeled reagents when the assay format requires same and signal generating reagents (e.g., enzyme substrate) if the label does not generate a signal directly. Instructions (e.g., written, tape, VCR, CD-ROM, etc.) for carrying out the assay usually will be included in the kit. The kit can also contain, depending on the particular assay used, other packaged reagents and materials (i.e. wash buffers and the like). Standard assays, such as those described above, can be conducted using these kits.

### 3. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Materials and Methods

Enzymes were purchased from commercial sources, and used according to the manufacturers' directions. In the isolation of DNA fragments, except where noted, all DNA manipulations were done according to standard procedures. See, Sambrook et al., supra. Restriction enzymes, T₄ DNA ligase, E. coli, DNA polymerase II, Klenow fragment, and other biological reagents can be purchased from commercial suppliers and used according to the manufacturers' directions. Double stranded DNA fragments were separated on agarose gels. Sources for chemical reagents generally include Sigma Chemical Company, St. Louis, Mo.; Alrich, Milwaukee, Wis.; Roche Molecular Biochemicals, Indianapolis, Ind.

Transient Transfections

COST cells were fed with DMEM (+L-glutamine and 4.5 g/ml glucose, Cellgro, Herndon, Va., cat #10-017-CM) and 10% FCS were plated to ˜60% confluent (˜5×10⁶ cells in a T225 flask) on day one. For small scale transfections cells were grown in 100 mm plates. On day 2, cells were transfected with plasmid DNA by adding media containing LT1 transfection reagent (Mirus, Madison, Wis., TransIT-COS system, Cat # MIR 2300) and 1 μg/μl plasmid DNA. LT1 and DNA were prepared according to the manufacturers' instructions and incubated with cells for 20 minutes at room temperature. Briefly, media and LT1 were mixed and incubated for 5 to 20 minutes. DNA was then added to the media/LT1 mixture, pipetted up and down to mix, and incubated for 20 minutes. Media was removed from the cells and replaced with 30 ml of fresh DMEM/10% FCS. The DNA/LT 1 mixture was added to the cells for 48 hours at 37° C. Media was then removed and frozen at −80° C. (supernatant) and the cells were washed with PBS (without CA⁺⁺ and Mg⁺⁺. Cells were harvested by scraping the cells from the surface and pelleted by centrifugation at 4,000 rpm 10 minutes at 4° C. PBS was aspirated from the cell pellet and the pellet was frozen at −80° C.

Western Blots

Cells lysates were analyzed by electrophoresis on either 4-20% polyacrylamide gradient gels (except for the Endoglycosydase H (Endo H) digest) or on a 10% polyacrylamide gel containing 0.1% SDS (for the Endo H digest). Samples were prepared in the absence reducing agents. Proteins were transferred in 0.2 mm membrane for detection in Western blots by monoclonal antibody, human or mouse serum. Human or mouse anti-sera were preabsorded with COS7 cell lysates and normal human or goat serum to reduce non-specific background prior to probing Western blots. Briefly, control (untransfected) COS7 cells were lysed in 20 mM hepes pH 6.8, 1 mM EGTA, 1% Triton X100 and 1 protease inhibitor tablet (Roche Diagnostics, Cat. #1-873-580). The COS7 cell pellet was homogenized with 20 strokes in 2 ml Dounce homogenizer, centrifuged at 14,000 RPM for 5 min at 4° C. and stored at −80° C. Prior to probing each blot with antisera, each membrane was incubated for ≧30 minutes in low detergent blotto (see below). Preabsorbed mouse or human serum was diluted 1:100 with PBS/0.05% Tween 20/10% blotto and incubated with the membrane for 2 hours at room temperature with shaking. The membrane was rinsed four times for 5 minutes each in PBS/0.05% Tween 20. A 1:20,000 dilution of goat anti-mouse HRP conjugated (AMI4404, Biosource, Camarillo Calif.) in PBS/0.05% Tween 20/10% blotto, was added for 1 hour at room temperature with shaking, followed by four 5 minute rinses in PBS/0.05% Tween 20 in order to visualize the G1 mAb binding proteins. The ECL (Amersham) signal was detected by exposure to Kodak film.

Standard Reagents

Blotto (pH 8.0):

### 50 mM Tris; 2 mM Calcium Chloride Dihydrate; 80 mM Sodium Chloride; 5% Carnation Nonfat Dry Milk; 0.2% NP-40; 0.02% Sodium Azide; 0.02N Hydrochloric Acid

Tris-Glycine SDS Running Buffer pH 8.3 (Invitrogen, San Diego Calif., Cat # LC2675):

### 25 mM mM Tris Base; 192 mM Glycine; 0.1% SDS.

### Example 1

### Cloning LACV M Segment Polypeptides

All numbering in the examples is based on the LACM sequence presented in FIGS. 1A-1E (NCBI Accession No. 004109). The full-length open reading frame (ORF) of the M segment of the La Crosse virus (nucleotides 62 to 4383 of FIGS. 1A-1E) was synthetically made using overlapping oligonucleotides. The Kozak sequence was introduced immediately downstream from the Xho1 site and upstream from the initiator methionine to facilitate expression in mammalian cells. LACM was then subcloned into pCMVIII (described U.S. Pat. No. 6,602,705, incorporated herein by reference in its entirety) by insertion into the Xho1/Not1 sites to generate pCMVIII-LACM. Using LACM as a template, PCR was used to generate a truncated G1 (amino acids 474 to 1391) containing a C-terminal histidine tag (LACV-G1-1391his). The Kozak sequence followed by the kappa light chain leader sequence (Watson, M. Nuc. Acid Res. (1984)12:5145-5164) were introduced immediately upstream of amino acid 474 of the LACM coding sequence. Specifically, the Kozak and kappa light chain leader sequence were made from overlapping synthetic oligonucleotides and cloned into the Xho1/PinA1 sites of the LACM pCMVIII clone. The C-terminal amino acid of the truncated G1 construct, LAC-G1-1391his was amino acid 1391.

### Example 2

### Expression of Envelope Glycoproteins

Expression of envelope glycoprotein G1 in COS7 cells was demonstrated by Western blot using commercial mouse monoclonal antibodies (mAb) that bind to G1 (Chemicon, Temecula, Calif., MAB8760; Virostat, Portland, Me., Cat. #3591) or human convalescent sera from an individual infected with LACV. (See Materials and Methods for details). COS7 cells were transiently transfected with either pCMVIII-LACM or pCMVIII-G1-1391his. Lysates from cells, which did or did not contain plasmids expressing LACV envelope proteins, were electrophoresed on a 4-20% polyacrylamide, 0.1% SDS gel and blotted onto a 0.2 mm nitrocellulose membrane. A protein of approximately 125 Kd was identified in the pCMVIII-LACM lysates and a protein of slightly smaller size, as expected for the truncated protein, was identified in pCMVIII-G1-1391his lysates, respectively. Proteins of approximately 120 and 125 Kd were not observed in the control cell lysate. The size of the G1 was consistent with that reported in the literature (reviewed in Gonzalez-Scarano and Nathanson, Fields Virology, 1996, Chapter 48 “Bunyaviridae”). A protein of about 125 Kd was also identified on a Western blot containing purified LACM, see below, when probed with human sera #8 from a LACV-infected patient but not control normal serum. Taken together, these data indicate that proteins of approximately 125 and 120 Kd expressed in COS7 cells were the LACV G1 protein.

### Example 3

### Purification of LACV Envelope Antigens

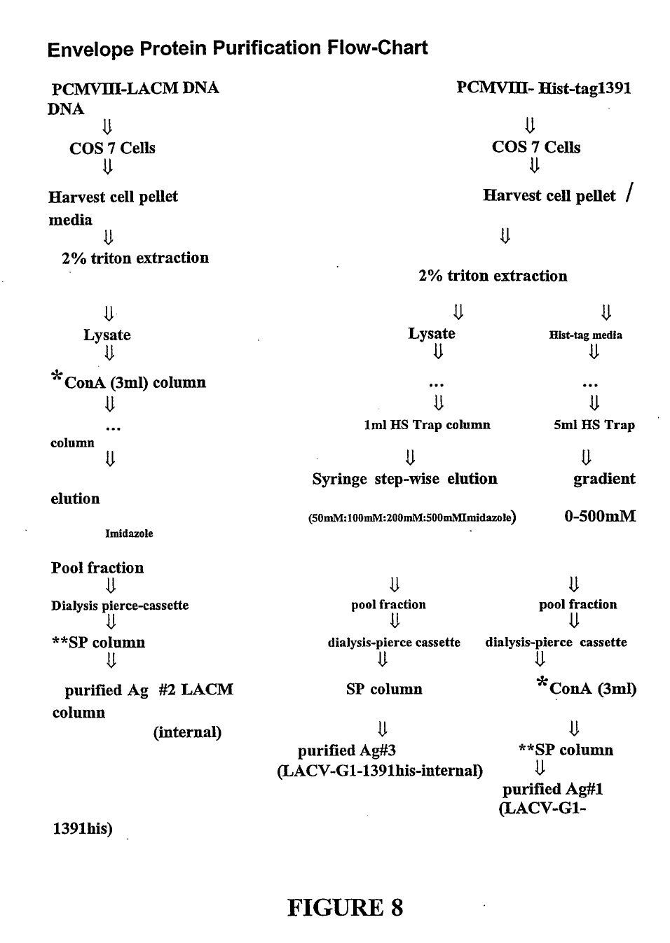

Large scale transient mammalian COS7 cell transfections were performed with the pCMVIII-LACM and pCMVIII-G1-1391his plasmid DNA as outlined in FIG. 8. Full length intracellular G1 and G2 (internal) was purified from cells expressing pCMVIII-LACM (amino acids 1-1441). Intracellular, truncated G1 was purified from cells expressing LACV-G1-1391his (G1-1391his internal, amino acids 474 to 1391). Secreted truncated G1 was purified from the media of COS7 cells transfected with pCMVIII-G1-1391his. LACM internal envelope glycoprotein(s) were extracted from cell pellets using Triton-X100, followed by ConA and SP column purification. G1-1391-his was purified both from cell pellets (internal form) and from cell culture media (secreted form). For secreted envelope, media was passed through a His trap column, followed by ConA and SP columns. For internal envelope G1-1391his, cell pellets were extracted by Triton X-100 followed by His trap column and SP column. The purification procedures for each of the glycoproteins are detailed below.

A. LACM

5 ml cell pellets from 40 transfected tissue culture 225 cm flasks were harvested, extracted in 20 ml (2% Triton X-100 in 50 mM Tris pH 8 with protease inhibitors, Roche complete, EDTA free Cat. #1-873-580) buffer, dounced-homogenized by 20 passes with loose pestle followed by another 20 passes with tight pestle, and centrifuged at 12,000 rpm for 20 minutes at 4° C. The Triton cell lysate supernatant (−22 ml) was then loaded at 4° C. onto a ConA column (AL-1003, Vector Laboratories, Burlingame, Calif.), 10 ml batchwise by gravity flow (0.5 ml/min) Immediately before loading, MgCl₂ and CaCl₂ were added to a final concentration of 1 mM each to the sample load. The column was washed with 6 ml of wash buffer (1M NaCl in 25 mM Tris pH 8, 0.1% Triton X-100, 1 mM CaCl, 1 mM MgCl with protease inhibitors) and eluted with 11 ml elution buffer (1M NaCl, 1M methyl mannopyranoside, 25 mM Tris pH 8, 0.1% Triton-X 100 with protease inhibitors) using a pump at flow rate of 1 ml/min. Eluted fractions (0.8 ml each) were collected. Fractions were sampled (8 μl) and analyzed by Western blot probed with 1:400 dilution of Chemicon MAB8760 against G1. Western-positive fractions were pooled and diluted at a ratio of 1:1 with SS-A buffer (20 mM sodium phosphate pH 6 0.1% TX-100), dialyzed in a slide-A-lyzer cassette (7K MWCO, Pierce Biotechnology, Rockford, Ill., Cat. #66710) cassette in SS-A buffer with two changes of buffers, 4 L each overnight at 4 C. The dialyzed material was then loaded onto a 4 ml SP-sepharose (fast flow) column (pre-equilibrated with SS-A) by gravity flow (0.5 ml/min). After washing with 15 ml SS-A, the SP column (#17-0729-01, Amersham) was eluded using a pump (1 ml/min) with 15 ml 0.5M NaCl in SS-A, followed by 15 ml 1 M NaCl in SS-A buffer. SP fractions (0.6 ml) were collected and analysed by Western blot as described above. Peak fractions #5, 6, 7 & 8 containing G1 from the 0.5 M NaCl elution were pooled and used to immunize mice as described below.

Since expressed internal G1, but not secreted G1, should be sensitive to endoglycosidase H which cleaves high mannose oligosaccharides from N-linked glycoproteins resident in the endoplasmic reticulum, purified internal LACM envelope protein(s) were digested with Endoglycosidase H (Endo H). A protein of ˜125 Kd was visualized by Western blot of a 10% polyacrylamide/0.1% SDS gel when incubated with a MAb against LAC G1 protein. As expected the ˜125 Kd protein was reduced in size after digestion with Endo H. In addition, the G1 also was reduced in size when treated with PNGaseF, which removes all N-linked glycosylation moieties from proteins. This data further demonstrated that the ˜125 Kd protein expressed in pCMVIII-LACM was the LAC G1 envelope glycoprotein.

B. LAC-G1-1391his (Secreted)

Approximately 1.2 L DMEM media were collected from 40 tissue culture T225 cm flasks of COST transfected cells. Protease inhibitors were added to the media, which was then filtered through a 0.45 μm filter. At 4° C., the media was loaded onto a 5 ml His Trap column (#17-5248-01 Amersham) pre-equilibrated with binding buffer (20 mM sodium phosphate pH 7.5/0.5 M Nacl) at a fast flow rate of 10 ml/min. The column was washed with 25 ml of binding buffer and then eluted in 48 ml imidazole gradient (zero to 0.5 M in binding buffer) at a flow rate of 1.5 ml/min. 1.5 ml fractions were collected then analyzed by Western blot. Western-positive fractions were pooled (˜10 ml) and dialyzed in a Pierce cassette overnight at 4° C. in 25 mM Tris pH 8/0.1% TX-100. The dialyzed material was then loaded onto a Con A (2.5 ml) column, pre-equilibrated with 25 mM Tris pH 8/0.1% Triton/1 mM MgCL₂/1 mM CaCL₂ by gravity flow. The column was washed with 7.5 ml of equilibration buffer and then eluted first with 1 M NaCl in equilibration buffer, followed with 1 M NaCl and 1 M methyl mannopyranoside in equilibration buffer without Ca++ and Mg++. Eluted fractions (0.8 ml each) were collected, sampled, and analyzed Western blot. Western-positive fractions were pooled (˜10 ml) from 1 M NaCl/MMP elution, diluted 1:1 with SS-A buffer and dialyzed against SS-A buffer in Pierce cassette overnight at 4° C. The dialyzed material was then loaded onto a 4 ml SP-sepharose column pre-equilibrated in SS-A by slow gravity flow. The SP column was washed with 10 ml SS-A and then eluted with 15 ml 0.5 M NaCl in SSA, followed by 1 M NaCl in SS-A. Eluted fractions (0.8 ml) were collected, sampled, and analyzed by Western blot. Peak material from 0.5 M NaCl elutions were pooled (#4, 5, & 6) and used to immunize mice as described below.

C. LAC-G1-1391his (Internal)

10 ml cell pellets from 80 COST tissue culture flasks (T225 cm) transfected with PCMVIII-G1-1391his were harvested, extracted in 40 ml 2% TritonX-100 buffer in (50 mM Tris pH7.5, with ROCHE protease inhibitors), dounce-homogenized, and centrifuged at 12,000 rpm for 20 min at 4° C. The Triton supernatant (45 ml) was filtered through a 0.45 μm filter and was loaded using a 5 ml syringe onto a 1 ml His Trap column (#17-5249-01, Amersham) pre-equilibrated with 50 mM Tris 7.5. The column was washed with 4 ml wash buffer (50 mM Tris pH 7.5, 0.5 M NaCl/0.1% TX100). 4 by 1 ml washes were collected. Using a step-wise elution method, the volume was eluted with 50 mM, 100 mM, 200 mM and 500 mM imidazole in wash buffer. Again at each elution step, 4 by 1 ml eluates were collected. The collected fractions were sampled and analyzed by Western blot. Peak fractions of 0.1 M and 0.2 M imidazole elutions were pooled and dialyzed in Pierce cassette overnight at 4° C. in SS-A buffer. The dialysate was centrifuged at 12,000 rpm for 20 min at 4° C. to eliminate precipitates. The supernatant was then loaded onto a SP-sepharose column, pre-equilibrated in SS-A. The SP column was washed with 10 ml SS-A and then eluted with 13 ml 0.5 M NaCl in SS-A, followed by 13 ml of 1 M NaCl in SS-A. Eluted SP fractions (˜0.9 ml each) were collected. Eluted fractions were analyzed by Western blot. Material from the peak fractions (#4, 5, & 6) from 0.5 M NaCl elutions were pooled and used to inoculate mice as described below.

### Example 4

### Immunogenicity of Purified LACV Antigens

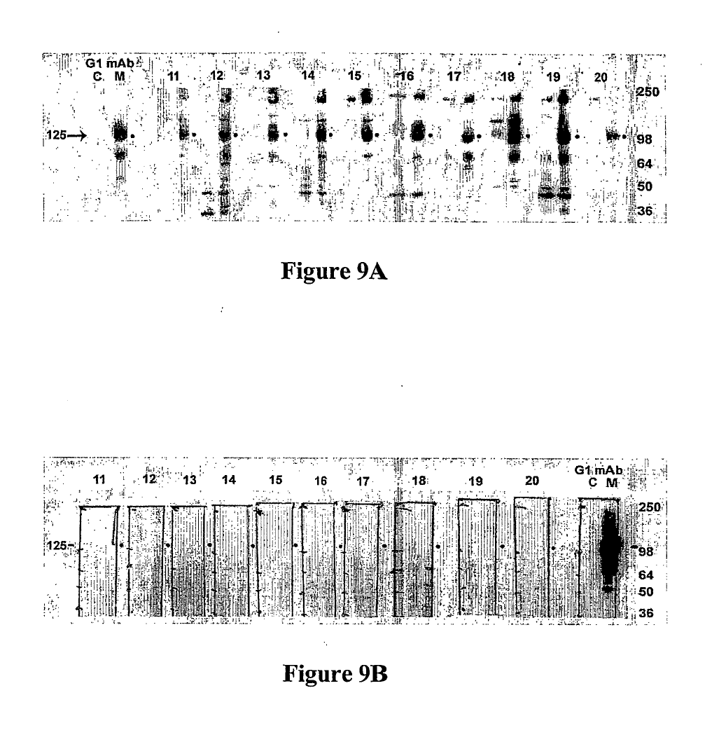

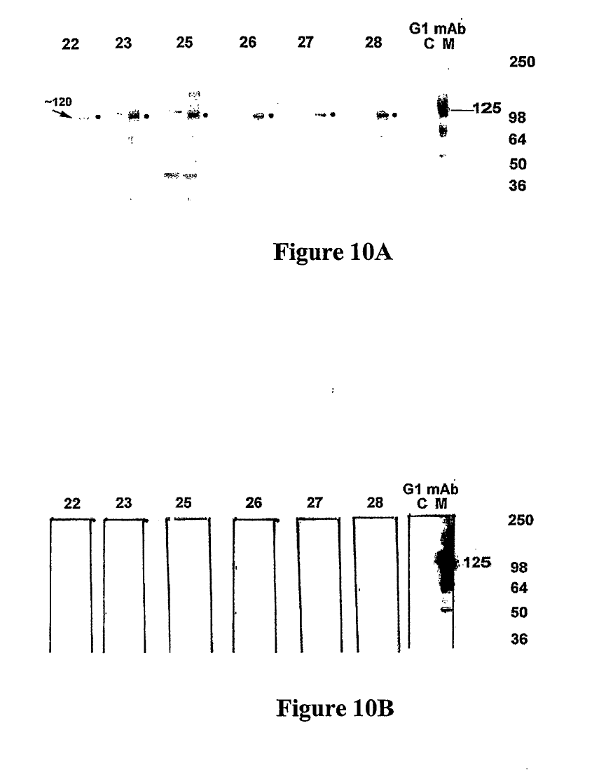

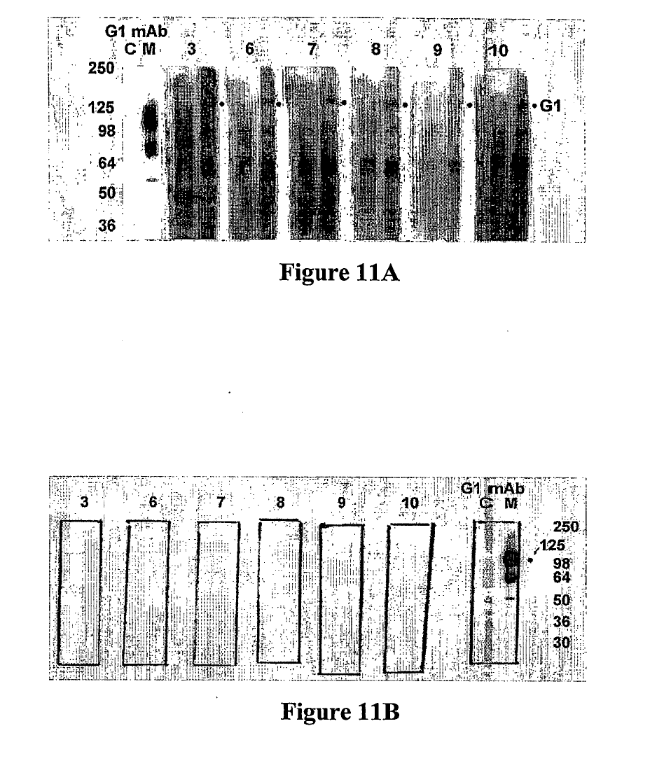

The immunogenicity of the three LACV antigens described above (internal LACM, internal G1-1391his and the secreted G1-1391his) was assessed in mice. Pooled fractions containing the individual antigen were mixed with an equal volume of either complete Freund's adjuvant for the first of two immunizations or an equal volume of incomplete Freund's adjuvant for the second immunization prior to IP inoculation into outbred albino CD-1 Swiss mice on weeks 0 and 2. FIGS. 9A, 10A and 11A show Western blots containing lysates from COST cells transfected with pCMVIII-LACM (right lane of each panel) or control vector without insert (left lane of each panel) probed with sera from individual mice immunized with either internal LAC-M (FIG. 9A), internal G1-1391his (FIG. 10A) or secreted G1-1391his (FIG. 11A) antigens. A protein of ˜125 Kd or ˜120 Kd, the same size as the G1 identified by a mAb to G1, was visualized by sera from mice immunized with either internal LACM or internal G1-1391his (FIGS. 9A and 10A), respectively, but not with the pre-immunization sera from the same mice (FIGS. 9B and 10B). A protein of higher molecular mass (>>125 Kd) reacted with sera from animals immunized with secreted G1-1391his (FIG. 11A), but not pre-immune sera from the same mice (FIG. 11B). This band may represent a more highly glycosylated form of G1 or a dimmer of G1. The data show that animals immunized with purified fractions of LACV antigens are immunogenic in mice.

Since the proteins were analyzed under non-reducing conditions, it is possible that G2 was expressed, but not detected at the expected size of 36 Kd in LACM lysates or in the purified protein preparation. This was confirmed as discussed below.

### Example 5

### Induction of Neutralizing Antibodies Using LACV Antigens

To assess whether the LACV envelope-specific antibodies in mouse sera contained virus neutralizing antibodies, a standard plaque reduction assay virus neutralizing titer assay was performed. Plaques will form in monolayers of cells after infection with LACV, which lyses infected cells. Therefore, antisera containing antibodies against LACV that will bind to the virus and block the virus from infecting cells (called neutralizing antibodies), will prevent the virus from lysing cells and a reduction in the number of plaques formed in the monolayer of cells will be observed. By counting the number of plaques on cell monolayers, the assay quantitatively measures the amount of virus neutralizing antibodies complexed with virus in any serum, tissue culture media, buffers or liquids.

Positive and negative controls were established for each assay. Positive controls consisted of cell monolayers infected with LACV of known titer (reference viral stock) in the presence of human serum containing or lacking virus neutralizing antibodies. Negative controls consisted of a serum control plate, to which no virus was added.

The specific method used was as follows. A six-well plate was prepared three days before the assay. Each well was plated with 3 ml of Vero cells at a cell density of 70,000 cells/ml. Serum samples were inactivated in a 53-59° C. water bath for 30 minutes. Two 96-well polypropylene plates were prepared by adding 72 μl of BA-1 diluent (1×M199/1% BSA) diluent to column 1. To the remainder of the columns (2-12), 90 p. 1 of BA-1 diluent was added. 48 μl of patient serum was added to column 1, one row per patient to both plates. The contents in first column were mixed and 30 μl transferred to the next column (four-fold dilutions). This was repeated until all dilutions were made from 1:2.5 to 1:5120.

Viruses were diluted separately in BA-1 with 8% fresh human serum to 30-90 PFU/0.1 ml. 90 μl of each virus was added to all the wells of their corresponding plate. Plates were incubated for two hours at 35-39° C. and 5% CO₂. Viral back titrations were prepared for the viruses by further diluting the test viral dilution to 10⁴ and 10⁻² viral dilutions. 100 μl of virus-serum mixtures was added to the drained six-well plates, one dilution per well. Control viral dilutions (back titrations) were inoculated in duplicate to separate plates and incubated for two hours in a 35-39° C. 5% CO₂ incubator.

Overlay media was made by mixing equal volumes of 2% agarose at 53-59° C. and 2% Ye-Lah medium at 40-44° C. and held in a 41-45° C. water bath. Prior to dispensing, 3 ml of a 7.5% solution of sodium bicarbonate was added per each 100 ml of overlay media. Wells were overlayed with 3 ml of the overlay media. After the agarose hardened, plates were placed upside down in a 35-39° C. 5% CO₂ incubator. A second 1% agarose overlay, containing 1.2 ml per 100 ml of a 0.33% neutral red solution, was done two days later for the La Crosse Virus. Plaques were counted and recorded at 24 and 48 hr after the addition of the second agarose overlay for the La Crosse Virus.

Plaques were counted and a virus neutralization titer was determined as follows. Plaques were counted in the viral back-titration and the average of duplicate wells at each dilution was calculated. The number of plaques in the test inoculum from those elements of the back-titration which yielded plaque counts of 30-100, were calculated. These were the most accurate counts. The cell control was inspected to confirm the integrity of the cell monolayer. Neutralization was defined as a 90% reduction of plaques. (If the back titration indicated that the test inoculum had 100 plaques, then the lowest serum dilution with 10 or fewer plaques, a reduction of 90% was the endpoint. In some cases it was necessary to cumulatively add plaques in order to determine endpoint.

The reciprocal of the dilution of serum that neutralizes the challenge inoculum represents the reportable titer. Stable high neutralizing antibody titers, a seroconversion or a >4 fold increases in antibody titer in a patient's appropriately timed acute and convalescent phase sera are accepted values.

As shown in Table 1, nine of ten mice immunized with LACM had virus neutralizing titers of between 1:2500 and 1:5120, while 0 of 5 prebleeds from the same set of mice had neutralizing titers. Seven of eight mice immunized with internal G1-1391his had neutralizing titers between 1:160 and 1:640, while 0 of 3 prebleeds for the same mice were positive for neutralizing antibodies (Table 1). The data clearly demonstrate that the internal antigens purified from either the full-length LACM ORF or the truncated G1-1391his-expressing cells generated a moderate to strong immune response by two weeks after the second immunization and that these antibodies contained virus neutralizing antibodies.

TABLE 1
Summary of Neutralizing Titer Data 2 Weeks Post 2nd Immunization

| | | | LAC-G1-1391his | | | | | |
|---|---|---|---|---|---|---|---|---|
| LACM | | | (internal) | | | LAC-G1-1391 (secreted) | | |
| mouse # | hyper | pre | mouse | hyper | pre | mouse | hyper | pre |
| 19 | 1:5120 | | 26 | 1:160 | 1:10 | 8 | <1:5 | |
| 18 | 1:5120 | 1:<5 | 22 | 1:160 | | 5 | <1:5 | |
| 11 | 1:2560 | | 27 | 1:160 | <1:5 | 1 | <1:5 | 1:<5 |
| 20 | >1:5120 | 1:<5 | 23 | 1:640 | <1:5 | 3 | <1:5 | 1:<5 |
| 12 | 1:2560 | | 28 | 1:640 | <1:5 | 2 | <1:5 | 1:<5 |
| 14 | 1:2560 | | 25 | 1:640 | | 4 | <1:5 | |
| 13 | >1:5120 | 1:<5 | 30 | 1:640 | | 6 | 1:5 | |
| 15 | >1:5120 | 1:<5 | 29 | <1:5 | | 7 | <1:5 | |
| 17 | >1:5120 | 1:<5 | | | | | | |
| 16 | 1:<5 | | | | | | | |

### Example 6

### Expression of LACV G2

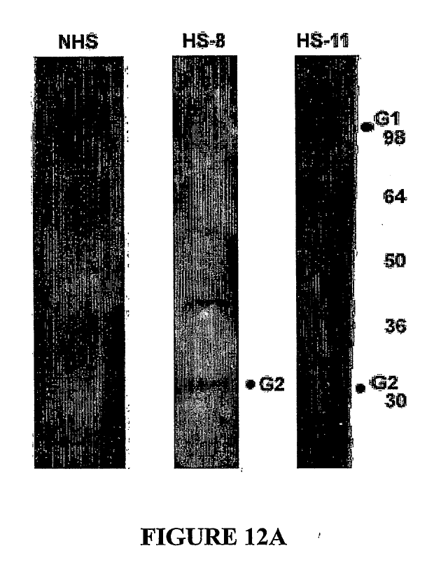

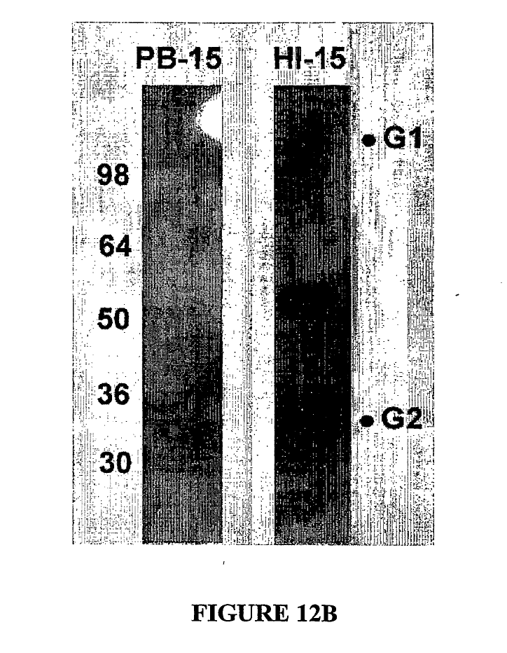

To confirm that LACV G2 was expressed by the LACM constructs, the following experiment was done. Purified, internal LACM protein, produced as described above, was treated with heat and DTT (reducing conditions, 90° C., 5 minutes in 5 mM DTT) and electrophoresed on 4-20%, 0.1% SDS gels. Gels were probed with sera from either LACV-infected individuals (HS-8 and HS-11, FIG. 12A) or normal (NHS, FIG. 12A). A protein of ˜32 Kd, which is the expected size of G2 as reported in the literature, was identified on a blot incubated with both human antisera but not with normal human serum. Similarly, blots probed with sera from a mouse immunized with purified LACM (hyperimmune (HI)-15 in FIG. 12B) reacted with a protein of ˜32 Kd that was not observed in the blot probed serum from the same animal prior to immunization (pre-bleed (PB)-15 in FIG. 12B). The data with human sera (FIG. 12A) showed that the purified, internal LACM material contained both LACV envelope glycoproteins G1 and G2 (HS-11). The data with mouse serum (FIG. 12B) demonstrated that internal LACM was immunogenic and generated antibodies against both LACV envelope proteins G1 and G2 (III-15). Antibodies induced by internal LACM were also shown to have high virus neutralizing titers (see Table 1, mouse 15).

Thus, reagents derived from CAL viruses, such as recombinant CAL immunogens, polynucleotides, inactivated and attenuated viruses, and the like, as well as methods of preparing the reagents and use of the reagents for diagnosis, prevention and treatment of CAL infection is described. Although preferred embodiments of the subject invention have been described in some detail, it is understood that obvious variations can be made without departing from the spirit and the scope of the invention as defined by the appended claims.

We claim:

1. A subunit vaccine composition comprising one or more isolated CAL virus immunogens and a pharmaceutically acceptable vehicle, wherein the one or more immunogens are selected from the group consisting of (a) G1, (b) G2, (c) N, (d) NSm, (e) NSs, (f); immunogenic fragments of (b), (c), (d) or (e); and immunogenic analogs of (a), (b), (c), (d), (e) or (f).

2. The subunit vaccine composition of claim 1, comprising an immunogen with the sequence of amino acids depicted at positions 474-1441 of FIGS. 1A-1E, or a sequence of amino acids with at least 75% sequence identity thereto.

3. The subunit vaccine composition of claim 2, wherein the immunogen comprises a sequence of amino acids with at least 85% sequence identity to the sequence of amino acids depicted at positions 474-1441 of FIGS. 1A-1E.

4. The subunit vaccine composition of claim 2, wherein the immunogen comprises a sequence of amino acids with at least 90% sequence identity to the sequence of amino acids depicted at positions 474-1441 of FIGS. 1A-1E.

5. The subunit vaccine composition of claim 2, wherein the immunogen comprises the sequence of amino acids depicted at positions 474-1441 of FIGS. 1A-1E.

6. The subunit vaccine composition of claim 1, wherein the immunogen comprises the sequence of amino acids depicted at positions 1-1441 of FIGS. 1A-1E, or a sequence of amino acids with at least 75% sequence identity thereto.

7. The subunit vaccine composition of claim 6, wherein the immunogen comprises a sequence of amino acids with at least 85% sequence identity to the sequence of amino acids depicted at positions 1-1441 of FIGS. 1A-1E.

8. The subunit vaccine composition of claim 6, wherein the immunogen comprises a sequence of amino acids with at least 90% sequence identity to the sequence of amino acids depicted at positions 1-1441 of FIGS. 1A-1E.

9. The subunit vaccine composition of claim 6, wherein the immunogen comprises the sequence of amino acids depicted at positions 1-1441 of FIGS. 1A-1E.

10. The subunit vaccine composition of claim 7, comprising an immunogenic fusion polypeptide that comprises a LACV envelope polypeptide fused to at least one other CAL virus polypeptide.

11. An immunogenic composition comprising a CAL virus truncated G1 polypeptide, wherein the truncated G1 polypeptide is truncated at a position between amino acid position 1391 and the C-terminus of the native G1 envelope polypeptide, numbered relative to the G1 polypeptide depicted in FIGS. 1A-1E.

12. The immunogenic composition of claim 11, wherein the truncated G1 polypeptide comprises the sequence of amino acids depicted at amino acid positions 474-1391 of FIGS. 1A-1E.

13. An immunogenic composition comprising at least one isolated CAL virus immunogen, wherein said immunogen is produced intracellularly.

14. The immunogenic composition of claim 13, wherein said immunogen is one or more immunogens selected from the group consisting of (a) G1, (b) G2, (c) N, (d) NSm, (e) NSs, (f); immunogenic fragments of (a), (b), (c), (d) or (e); and immunogenic analogs of (a), (b), (c), (d), (e) or (f).

15. The immunogenic composition of claim 14, comprising a full-length G1.

16. The immunogenic composition of claim 14, comprising a truncated G1 polypeptide.

17. The immunogenic composition of claim 16, wherein the truncated G1 polypeptide comprises a deletion of all or part of a transmembrane binding domain.

18. The immunogenic composition of claim 17, wherein the truncated G1 polypeptide further comprises a deletion of all or part of the cytoplasmic tail.

19. The immunogenic composition of claim 17, wherein the truncated G1 polypeptide comprises all or part of the cytoplasmic tail.

20. The immunogenic composition of claim 17, wherein the truncated G1 polypeptide is truncated at a position between amino acid position 1387 and the C-terminus of the native G1 envelope polypeptide, numbered relative to the G1 polypeptide depicted in FIGS. 1A-1E.

21. The immunogenic composition of claim 17, wherein the truncated G1 polypeptide is truncated at a position between amino acid position 1391 and the C-terminus of the native G1 envelope polypeptide, numbered relative to the G1 polypeptide depicted in FIGS. 1A-1E.

22. The immunogenic composition of claim 20, wherein the truncated G1 polypeptide comprises the sequence of amino acids depicted at amino acid positions 474 to 1387 of FIGS. 1A-1E.

23. The immunogenic composition of claim 21, wherein the truncated G1 polypeptide comprises the sequence of amino acids depicted at amino acid positions 474-1391 of FIGS. 1A-1E.

24. The immunogenic composition of claim 22, wherein the truncated G1 polypeptide comprises a deletion of amino acids 1388-1419, numbered relative to the G1 polypeptide depicted in FIGS. 1A-1E.

25. The immunogenic composition of claim 23, wherein the truncated G1 polypeptide comprises a deletion of amino acids 1392-1419, numbered relative to the G1 polypeptide depicted in FIGS. 1A-1E.

26. The immunogenic composition of claim 13, comprising the protein product of a CAL virus MC region.

27. The immunogenic composition of claim 15, comprising the sequence of amino acids depicted at positions 474-1441 of FIGS. 1A-1E, or a sequence of amino acids with at least 75% sequence identity thereto.

28. The immunogenic composition of claim 27, comprising a sequence of amino acids with at least 85% sequence identity to the sequence of amino acids depicted at positions 474-1441 of FIGS. 1A-1E.

29. The immunogenic composition of claim 27, comprising a sequence of amino acids with at least 90% sequence identity to the sequence of amino acids depicted at positions 474-1441 of FIGS. 1A-1E.

30. The immunogenic composition of claim 27, comprising the sequence of amino acids depicted at positions 474-1441 of FIGS. 1A-1E.

31. The immunogenic composition of claim 26, comprising the sequence of amino acids depicted at positions 1-1441 of FIGS. 1A-1E, or a sequence of amino acids with at least 75% sequence identity thereto.

32. The immunogenic composition of claim 31, comprising a sequence of amino acids with at least 85% sequence identity to the sequence of amino acids depicted at positions 1-1441 of FIGS. 1A-1E.

33. The immunogenic composition of claim 31, comprising a sequence of amino acids with at least 90% sequence identity to the sequence of amino acids depicted at positions 1-1441 of FIGS. 1A-1E.

34. The immunogenic composition of claim 31, comprising the sequence of amino acids depicted at positions 1-1441 of FIGS. 1A-1E.

35. An immunogenic composition comprising an inactivated CAL virus and a pharmaceutically acceptable vehicle.

36. The immunogenic composition of claim 1, wherein the CAL virus is La Crosse virus.

37. An immunogenic composition comprising an attenuated CAL virus and a pharmaceutically acceptable vehicle.

38. The immunogenic composition of claim 3, wherein the CAL virus is La Crosse virus.

39. A method of treating or preventing CAL virus infection in a mammalian subject comprising administering to said subject a therapeutically effective amount of the immunogenic composition of claim 1.

40. A method of treating or preventing CAL virus infection in a mammalian subject comprising administering to said subject a therapeutically effective amount of the immunogenic composition of claim 11.

41. A method of treating or preventing CAL virus infection in a mammalian subject comprising administering to said subject a therapeutically effective amount of the immunogenic composition of claim 13.

42. A method of treating or preventing CAL virus infection in a mammalian subject comprising administering to said subject a therapeutically effective amount of the immunogenic composition of claim 35.

43. A method of treating or preventing CAL virus infection in a mammalian subject comprising administering to said subject a therapeutically effective amount of the subunit vaccine composition of claim 36.

44. A method of treating or preventing CAL virus infection in a mammalian subject comprising administering to said subject a therapeutically effective amount of the immunogenic composition of claim 37.

45. A method of producing an immunogenic composition comprising the steps of
(a) providing an inactivated or attenuated CAL virus; and
(b) combining said inactivated or attenuated CAL virus with a pharmaceutically acceptable vehicle.

46. A method of producing a subunit vaccine composition comprising the steps of
(a) providing one or more CAL virus immunogens, wherein the one or more immunogens are selected from the group consisting of (a) G1, (b) G2, (c) N, (d) NSm, (e) NSs, (f); immunogenic fragments of (b), (c), (d) or (e); and immunogenic analogs of (a), (b), (c), (d), (e) or (f); and
(b) combining said CAL virus immunogen(s) with a pharmaceutically acceptable vehicle.

47. A method of producing an immunogenic composition comprising the steps of
(a) providing a CAL virus immunogen, wherein said immunogen is produced intracellularly
(b) combining said CAL virus immunogen with a pharmaceutically acceptable vehicle.

48. A method of producing an immunogenic composition comprising the steps of
(a) providing a CAL virus truncated G1 polypeptide, wherein the truncated G1 polypeptide is truncated at a position between amino acid position 1391 and the C-terminus of the native G1 envelope polypeptide, numbered relative to the G1 polypeptide depicted in FIGS. 1A-1E; and
(b) combining said CAL virus truncated G1 polypeptide with a pharmaceutically acceptable vehicle.

49. A method for isolating an immunogenic CAL virus envelope polypeptide comprising:
(a) providing a population of mammalian host cells that express said envelope polypeptide intracellularly;
(b) recovering s membrane component of the cells;
(c) treating the membrane component with a non-ionic detergent, thereby to solubilize the membrane component and release the envelope polypeptide; and
(d) isolating the released envelope polypeptide.

50. The method of claim 49, wherein said isolating comprises at least one column purification step wherein said column is selected from the group consisting of a lectin affinity column, a hydroxyapatite column and an ion exchange column.

51. The method of claim 50, wherein said isolating step comprises:
(i) binding the released envelope polypeptide to the ion exchange column; and
(ii) eluting the bound envelope polypeptide from the ion exchange column.

52. The method of claim 50, wherein said isolating step comprises:
(i) binding the released envelope polypeptide to a lectin affinity column;
(ii) eluting the bound polypeptide from the lectin affinity column;
(iii) subjecting the eluted polypeptide to a cation exchange column; and
(iv) eluting the bound envelope polypeptide from the cation exchange column.

53. The method of claim 52, where said lectin affinity column is a concanavalin A lectin column.

54. The method of claim 50, wherein the mammalian cells are CHO or HEK293 cells.

55. The method of claim 49, wherein the CAL virus envelope polypeptide is a G1 and/or a G2 polypeptide, and optionally includes all or a portion of the NSm polypeptide.

56. An immunogenic composition comprising the envelope polypeptide obtained by the method of claim 49.

57. A CAL virus truncated G1 polypeptide, wherein the truncated G1 polypeptide is truncated at a position between amino acid position 1391 and the C-terminus of the native G1 envelope polypeptide, numbered relative to the G1 polypeptide depicted in FIGS. 1A-1E.

58. The truncated G1 polypeptide of claim 57, wherein the polypeptide comprises the sequence of amino acids depicted at amino acid positions 474-1391 of FIGS. 1A-1E.

59. An isolated oligonucleotide not more than 60 nucleotides in length comprising:
(a) a nucleotide sequence of at least 10 contiguous nucleotides from a probe or primer sequence depicted in any of FIG. 5, 6 or 7;
(b) a nucleotide sequence having 90% sequence identity to a nucleotide sequence of (a); or
(c) complements of (a) and (b).

60. The oligonucleotide of claim 59, wherein the nucleotide sequence is a probe sequence depicted in any of FIG. 5, 6 or 7 and further comprises a detectable label at the 5′-end and/or the 3′-end.

61. The oligonucleotide of claim 60, wherein the detectable label is a fluorescent label selected from the group consisting of 6-carboxyfluorescein (6-FAM), tetramethyl rhodamine (TAMRA), and 2′,4′,5′,7′,-tetrachloro-4-7-dichlorofluorescein (TET).

62. An isolated oligonucleotide selected from the group consisting of: (a) the oligonucleotide of SEQ ID NO:7, (b) the oligonucleotide of SEQ ID NO:8, (c) the oligonucleotide of SEQ ID NO:9, (d) the oligonucleotide of SEQ ID NO:10, (e) the oligonucleotide of SEQ ID NO:11, (f) the oligonucleotide of SEQ ID NO:12, (g) the oligonucleotide of SEQ ID NO:13, (h) the oligonucleotide of SEQ ID NO:14, (i) the oligonucleotide of SEQ ID NO:15, (j) SEQ ID NO:16, complements of (a), (b), (c), (d), (e), (f), (g), (h), (i) or (j), and reverse complements of (a), (b), (c), (d), (e), (f), (g), (h), (i) or (j).

63. The oligonucleotide of claim 62, wherein said oligonucleotide is selected from the group consisting of (a) the oligonucleotide of SEQ ID NO:8, (b) the oligonucleotide of SEQ ID NO:9, (c) the oligonucleotide of SEQ ID NO:12, (d) the oligonucleotide of SEQ ID NO:16, complements of (a), (b), (c) or (d), and reverse complements of (a), (b), (c) or (d).

64. The oligonucleotide of claim 62, comprising a detectable label at the 5′-end and/or the 3′-end.

65. The oligonucleotide of claim 63, comprising a detectable label at the 5′-end and/or the 3′-end.

66. The oligonucleotide of claim 64, wherein the detectable label is a fluorescent label selected from the group consisting of 6-carboxyfluorescein (6-FAM), tetramethyl rhodamine (TAMRA), and 2′,4′,5′,7′,-tetrachloro-4-7-dichlorofluorescein (TET).

67. The oligonucleotide of claim 65, wherein the detectable label is a fluorescent label selected from the group consisting of 6-carboxyfluorescein (6-FAM), tetramethyl rhodamine (TAMRA), and 2′,4′,5′,7′,-tetrachloro-4-7-dichlorofluorescein (TET).

68. A method for detecting CAL virus infection in a biological sample, the method comprising:
(a) isolating nucleic acid from a biological sample suspected of containing CAL virus RNA, wherein if CAL virus is present, said nucleic acid comprises a target sequence;
(b) reacting the CAL virus nucleic acid with a detectably labeled probe sufficiently complementary to and capable of hybridizing with the target sequence, wherein said reacting is done under conditions that provide for the formation of a probe/target sequence complex; and
(c) detecting the presence or absence of label as an indication of the presence or absence of the target sequence.

69. A method for detecting La Crosse virus (LACV) infection in a biological sample, the method comprising:
(a) isolating nucleic acid from a biological sample suspected of containing LACV RNA, wherein if LACV is present, said nucleic acid comprises a target sequence;
(b) reacting the LACV nucleic acid with a detectably labeled probe sufficiently complementary to and capable of selectively hybridizing with the target sequence, wherein said reacting is done under conditions that provide for the formation of a probe/target sequence complex; and
(c) detecting the presence or absence of label as an indication of the presence or absence of the target sequence.

70. The method of claim 69, wherein the probe is selected from the group consisting of (a) the oligonucleotide of SEQ ID NO:8, (b) the oligonucleotide of SEQ ID NO:9, (c) the oligonucleotide of SEQ ID NO:12, (d) the oligonucleotide of SEQ ID NO:16, complements of (a), (b), (c) or (d), and reverse complements of (a), (b), (c) or (d).

71. A method for detecting CAL virus infection in a biological sample, the method comprising:
isolating nucleic acids from a biological sample suspected of containing CAL virus;
amplifying the nucleic acids using at least two primers wherein (a) each of the primers is not more than about 50 nucleotides in length and each of the primers is sufficiently complementary to a portion of the sense and antisense strands, respectively, of CAL virus isolated nucleic acid, if present, to hybridize therewith; and
detecting the presence of the amplified nucleic acids as an indication of the presence or absence of CAL virus in the sample.

72. The method of claim 71, wherein amplifying comprises RT-PCR, transcription-mediated amplification (TMA) or a fluorogenic 5′ nuclease assay, or a combination thereof.

73. The method of claim 72, wherein amplifying uses a fluorogenic 5′ nuclease assay using the sense primer and the antisense primer and detecting is done using at least one detectably labeled probe sufficiently complementary to and capable of hybridizing with the CAL virus nucleic acid if present.

74. A method for detecting La Crosse virus (LACV) infection in a biological sample, the method comprising:
isolating nucleic acids from a biological sample suspected of containing LACV wherein if LACV is present, said nucleic acid comprises a target sequence;
amplifying the nucleic acids using at least two primers wherein (a) each of the primers is not more than about 50 nucleotides in length and each of the primers is sufficiently complementary to a portion of the sense and antisense strands, respectively, of LACV isolated nucleic acid, if present, to hybridize therewith, and further wherein at least one of the primers is capable of selectively hybridizing to the target sequence; and
detecting the presence of the amplified nucleic acids as an indication of the presence or absence of LACV in the sample.

75. The method of claim 74, wherein amplifying comprises RT-PCR, transcription-mediated amplification (TMA) or a fluorogenic 5′ nuclease assay, or a combination thereof.

76. The method of claim 75, wherein amplifying uses a fluorogenic 5′ nuclease assay using the sense primer and the antisense primer and detecting is done using at least one detectably labeled probe sufficiently complementary to and capable of hybridizing with the LACV nucleic acid if present.

77. The method of claim 74, wherein one of the primers is selected from the group consisting of (a) the oligonucleotide of SEQ ID NO:8, (b) the oligonucleotide of SEQ ID NO:9, (c) the oligonucleotide of SEQ ID NO:12, (d) the oligonucleotide of SEQ ID NO:16, complements of (a), (b), (c) or (d), and reverse complements of (a), (b), (c) or (d).

78. A method for detecting La Crosse virus (LACV) infection in a biological sample, the method comprising:
isolating nucleic acids from a biological sample suspected of containing LACV wherein if LACV is present, said nucleic acid comprises a target sequence;
amplifying the nucleic acids using at least two primers wherein (a) each of the primers is not more than about 50 nucleotides in length and each of the primers is sufficiently complementary to a portion of the sense and antisense strands, respectively, of LACV isolated nucleic acid, if present, to hybridize therewith; and
detecting the presence of the amplified nucleic acids using at least one detectably labeled probe sufficiently complementary to and capable of hybridizing with the LACV nucleic acid if present, as an indication of the presence or absence of LACV in the sample, wherein at least one of the primers and/or the probe is capable of selectively hybridizing to the target sequence.

79. The method of claim 78, wherein one of the primers is selected from the group consisting of (a) the oligonucleotide of SEQ ID NO: 8, (b) the oligonucleotide of SEQ ID NO:9, (c) the oligonucleotide of SEQ ID NO:12, (d) the oligonucleotide of SEQ ID NO:16, complements of (a), (b), (c) or (d), and reverse complements of (a), (b), (c) or (d).

80. A kit for detecting a CAL virus infection in a biological sample, the kit comprising:
primer oligonucleotides wherein the primer oligonucleotides are not more than about 60 nucleotides in length, wherein each of the primers is sufficiently complementary to a portion of the sense and antisense strands, respectively, to CAL virus nucleic acid to hybridize therewith; and
written instructions for identifying the presence of a CAL virus.

81. The kit of claim 66, further comprising a polymerase and buffers.

82. The kit of claim 66, further comprising at least one detectably labeled probe oligonucleotide of not more than about 60 nucleotides in length and sufficiently complementary to and capable of hybridizing with CAL virus nucleic acid.

83. A kit for detecting a La Crosse virus (LACV) infection in a biological sample, the kit comprising:
primer oligonucleotides wherein the primer oligonucleotides are not more than about 60 nucleotides in length, wherein each of the primers is sufficiently complementary to a portion of the sense and antisense strands, respectively, to LACV nucleic acid to hybridize therewith and further wherein at least one of the primers is capable of selectively hybridizing to LACV nucleic acid; and
written instructions for identifying the presence of a LACV.

84. The kit of claim 83, further comprising a polymerase and buffers.

85. The kit of claim 83, wherein one of the primers is selected from the group consisting of (a) the oligonucleotide of SEQ ID NO:8, (b) the oligonucleotide of SEQ ID NO:9, (c) the oligonucleotide of SEQ ID NO:12, (d) the oligonucleotide of SEQ ID NO:16, complements of (a), (b), (c) or (d), and reverse complements of (a), (b), (c) or (d).

86. The kit of claim 83, further comprising at least one detectably labeled probe oligonucleotide of not more than about 60 nucleotides in length and sufficiently complementary to and capable of hybridizing with LACV nucleic acid.

87. A kit for detecting a La Crosse virus (LACV) infection in a biological sample, the kit comprising:
primer oligonucleotides wherein the primer oligonucleotides are not more than about 60 nucleotides in length, wherein each of the primers is sufficiently complementary to a portion of the sense and antisense strands, respectively, to LACV nucleic acid to hybridize therewith;
at least one detectably labeled probe oligonucleotide of not more than about 60 nucleotides in length and sufficiently complementary to and capable of hybridizing with LACV nucleic acid, wherein at least one of the primers and/or the probe is capable of selectively hybridizing to the target sequence; and
written instructions for identifying the presence of LACV.

88. The kit of claim 87, further comprising a polymerase and buffers.

89. The kit of claim 87, wherein one of the primers and/or probes is selected from the group consisting of (a) the oligonucleotide of SEQ ID NO:8, (b) the oligonucleotide of SEQ ID NO:9, (c) the oligonucleotide of SEQ ID NO:12, (d) the oligonucleotide of SEQ ID NO:16, complements of (a), (b), (c) or (d), and reverse complements of (a), (b), (c) or (d).

90. The subunit vaccine composition of claim 1, wherein the immunogen is produced by recombinant expression of a polynucleotide encoding a polypeptide with the sequence of amino acids depicted at positions 474-1441 of Figures.

91. The subunit vaccine composition of claim 1, wherein the immunogen is produced by recombinant expression of a polynucleotide encoding a polypeptide with the sequence of amino acids depicted at positions 474-1441 of FIGS. 1A-1E.

92. The subunit vaccine composition of claim 1, wherein the immunogen is produced by recombinant expression of a polynucleotide encoding a polypeptide with the sequence of amino acids depicted at positions 1-1441 of FIGS. 1A-1E.

93. The immunogenic composition of claim 11, wherein the truncated G1 polypeptide is produced by recombinant expression of a polynucleotide encoding a polypeptide with the sequence of amino acids depicted at positions 474-1391 of FIGS. 1A-1E.

94. The immunogenic composition of claim 13, wherein the immunogen is produced by recombinant expression of a polynucleotide encoding a polypeptide with the sequence of amino acids depicted at positions 474-1441 of FIGS. 1A-1E.

95. The immunogenic composition of claim 13, wherein the immunogen is produced by recombinant expression of a polynucleotide encoding a polypeptide with the sequence of amino acids depicted at positions 1-1441 of FIGS. 1A-1E.

96. The immunogenic composition of claim 13, wherein the immunogen is produced by recombinant expression of a polynucleotide encoding a polypeptide with the sequence of amino acids depicted at positions 474-1391 of FIGS. 1A-1E.
